SEQUENCE LISTING (61 sequences)

SEQ ID NO: 1 - DNA, 3182 bp, Hepatitis B virus
  source 1..3182: mol_type = genomic DNA; note = Hepatitis B Virus genome - NCBI Reference Sequence NC_003977.2 (GenBank Accession No. GI:21326584)
aattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtgg

SEQ ID NO: 2 - DNA, 18 bp, Hepatitis B virus
  source 1..18: mol_type = other DNA; note = Target sequence, nucleotides 1579-1597 of NC_003977.2 (GenBank Accession No. GI:21326584)
gtgtgcacttcgcttcac

SEQ ID NO: 3 - RNA, 19 nt, synthetic construct
  source 1..19: mol_type = other RNA; note = SIRNA01, sense strand, unmodified
gtgtgcacttcgcttcaca

SEQ ID NO: 4 - RNA, 21 nt, synthetic construct
  source 1..21: mol_type = other RNA; note = SIRNA01, antisense strand, unmodified
tgtgaagcgaagtgcacactt

SEQ ID NO: 5 - RNA, 19 nt, synthetic construct
  source 1..19: mol_type = other RNA; note = SIRNA01, sense strand
gtgtgcacttcgcttcaca

SEQ ID NO: 6 - RNA, 21 nt, synthetic construct
  source 1..21: mol_type = other RNA; note = SIRNA01, antisense strand
tgtgaagcgaagtgcacactt

SEQ ID NO: 7 - RNA, 19 nt, synthetic construct
  source 1..19: mol_type = other RNA; note = SIRNA02, sense strand
gtgtgcacttcgcttcaca

SEQ ID NO: 8 - RNA, 21 nt, synthetic construct
  source 1..21: mol_type = other RNA; note = SIRNA02, antisense strand
tgtgaagcgaagtgcacactt

SEQ ID NO: 9 - protein, 16 aa, synthetic construct
  source 1..16: note = Membrane translocation sequence-containing peptide RFGF
AAVALLPAVLLALLAP

SEQ ID NO: 10 - protein, 11 aa, synthetic construct
  source 1..11: note = Membrane translocation sequence-containing peptide RFGF analogue
AALLPVLLAAP

SEQ ID NO: 11 - protein, 13 aa, synthetic construct
  source 1..13: note = HIV Tat protein
GRKKRRQRRRPPQ

SEQ ID NO: 12 - protein, 15 aa, synthetic construct
  source 1..15: note = Drosophila Antennapedia protein
RQIKIWFQNRRMKWK

SEQ ID NO: 13 - protein, 226 aa, Hepatitis B virus
  source 1..226: note = HBsAg S domain
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 14 - protein, 72 aa, Hepatitis B virus
  source 1..72: note = J02203 (D, ayw3) HBsAg Antigenic Loop Sequence
QGMLPVCPLIPGSSTTSTGPCRTCMTTAQGTSMYPSCCCTKPSDGNCTCIPIPSSWAFGKFLWEWASARFSW

SEQ ID NO: 15 - protein, 72 aa, Hepatitis B virus
  source 1..72: note = FJ899792 (D, adw2) HBsAg Antigenic Loop Sequence
QGMLPVCPLIPGSSTTGTGPCRTCTTPAQGTSMYPSCCCTKPSDGNCTCIPIPSSWAFGKFLWEWASARFSW

SEQ ID NO: 16 - protein, 72 aa, Hepatitis B virus
  source 1..72: note = AM282986 (A) HBsAg Antigenic Loop Sequence
QGMLPVCPLIPGTTTTSTGPCKTCTTPAQGNSMFPSCCCTKPSDGNCTCIPIPSSWAFAKYLWEWASVRFSW

SEQ ID NO: 17 - protein, 72 aa, Hepatitis B virus
  source 1..72: note = D23678 (B1) HBsAg Antigenic Loop Sequence
QGMLPVCPLIPGSSTTSTGPCKTCTTPAQGTSMFPSCCCTKPTDGNCTCIPIPSSWAFAKYLWEWASVRFSW

SEQ ID NO: 18 - protein, 72 aa, Hepatitis B virus
  source 1..72: note = AB117758 (C1) HBsAg Antigenic Loop Sequence
QGMLPVCPLLPGTSTTSTGPCKTCTIPAQGTSMFPSCCCTKPSDGNCTCIPIPSSWAFARFLWEWASVRFSW

SEQ ID NO: 19 - protein, 74 aa, Hepatitis B virus
  source 1..74: note = AB205192(E) HBsAg Antigenic Loop Sequence
QGMLPVCPLIPGSSTTSTGPCRTCTTLAQGTSMFPSCCCSKPSDGNCTCIPIPSSWAFGKFLWEWASARFSWLS

SEQ ID NO: 20 - protein, 71 aa, Hepatitis B virus
  source 1..71: note = X69798 (F4) HBsAg Antigenic Loop Sequence
QGMLPVCPLLPGSTTTSTGPCTCTTLAQGTSMFPSCCCSKPSDGNCTCIPIPSSWALGKYLWEWASARFSW

SEQ ID NO: 21 - protein, 70 aa, Hepatitis B virus
  source 1..70: note = AF1 60501 (G) HBsAg Antigenic Loop Sequence
QGMLPVCPLIPGSSTTSTGPCTCTTPAQGNSMYPSCCCTPSDGNCTCIPIPSSWAFAKYLWEWASVRFSW

SEQ ID NO: 22 - protein, 72 aa, Hepatitis B virus
  source 1..72: note = AY090454 (H) HBsAg Antigenic Loop Sequence
QGMLPVCPLLPGSTTTSTGPCKTCTTLAQGTSMFPSCCCTKPSDGNCTCIPIPSSWAFGKYLWEWASARFSW

SEQ ID NO: 23 - protein, 72 aa, Hepatitis B virus
  source 1..72: note = AF241409 (I) HBsAg Antigenic Loop Sequence
QGMLPVCPLIPGSSTTSTGPCKTCTTPAQGNSMYPSCCCTKPSDGNCTCIPIPSSWAFAKYLWEWASARFSW

SEQ ID NO: 24 - protein, 72 aa, Hepatitis B virus
  source 1..72: note = AB486012 (J) HBsAg Antigenic Loop Sequence
QGMLPVCPLLPGSTTTSTGPCRTCTITAQGTSMFPSCCCTKPSDGNCTCIPIPSSWAFAKFLWEWASVRFSW

SEQ ID NO: 25 - protein, 72 aa, Hepatitis B virus
  source 1..72: note = HBsAg Y100C/P120T HBsAg Antigenic Loop Sequence
CQGMLPVCPLIPGSSTTGTGTCRTCTTPAQGTSMYPSCCCTKPSDGNCTCIPIPSSWAFGFLWEWASARFSW

SEQ ID NO: 26 - protein, 72 aa, Hepatitis B virus
  source 1..72: note = HBsAg P120T HBsAg Antigenic Loop Sequence
QGMLPVCPLIPGSSTTGTGTCRTCTTPAQGTSMYPSCCCTKPSDGNCTCIPIPSSWAFGKFLWEWASARFSW

SEQ ID NO: 27 - protein, 72 aa, Hepatitis B virus
  source 1..72: note = HBsAg P120T/S143L HBsAg Antigenic Loop Sequence
QGMLPVCPLIPGSSTTGTGTCRTCTTPAQGTSMYPSCCCTKPLDGNCTCIPIPSSWAFGKFLWEWASARFSW

SEQ ID NO: 28 - protein, 72 aa, Hepatitis B virus
  source 1..72: note = HBsAg C121 S HBsAg Antigenic Loop Sequence
QGMLPVCPLIPGSSTTGTGPSRTCTTPAQGTSMYPSCCCTKPSDGNCTCIPIPSSWAFGKFLWEWASARFSW

SEQ ID NO: 29 - protein, 72 aa, Hepatitis B virus
  source 1..72: note = HBsAg R122D HBsAg Antigenic Loop Sequence
QGMLPVCPLIPGSSTTGTGPCDTCTTPAQGTSMYPSCCCTKPSDGNCTCIPIPSSWAFGKFLWEWASARFSW

SEQ ID NO: 30 - protein, 71 aa, Hepatitis B virus
  source 1..71: note = HBsAg R122I HBsAg Antigenic Loop Sequence
QGMLPVCPLIPGSSTTGTGPCITCTTPAQGTSMYPSCCCTPSDGNCTCIPIPSSWAFGKFLWEWASARFSW

SEQ ID NO: 31 - protein, 72 aa, Hepatitis B virus
  source 1..72: note = HBsAg T123N HBsAg Antigenic Loop Sequence
QGMLPVCPLIPGSSTTGTGPCRNCTTPAQGTSMYPSCCCTKPSDGNCTCIPIPSSWAFGKFLWEWASARFSW

SEQ ID NO: 32 - protein, 72 aa, Hepatitis B virus
  source 1..72: note = HBsAg Q129H HBsAg Antigenic Loop Sequence
QGMLPVCPLIPGSSTTGTGPCRTCTTPAHGTSMYPSCCCTKPSDGNCTCIPIPSSWAFGKFLWEWASARFSW

SEQ ID NO: 33 - protein, 72 aa, Hepatitis B virus
  source 1..72: note = HBsAg Q129L HBsAg Antigenic Loop Sequence
QGMLPVCPLIPGSSTTGTGPCRTCTTPALGTSMYPSCCCTKPSDGNCTCIPIPSSWAFGKFLWEWASARFSW

SEQ ID NO: 34 - protein, 72 aa, Hepatitis B virus
  source 1..72: note = HBsAg M1 33H HBsAg Antigenic Loop Sequence
QGMLPVCPLIPGSSTTGTGPCRTCTTPAQGTSHYPSCCCTKPSDGNCTCIPIPSSWAFGKFLWEWASARFSW

SEQ ID NO: 35 - protein, 72 aa, Hepatitis B virus
  source 1..72: note = HBsAg M1 33L HBsAg Antigenic Loop Sequence
QGMLPVCPLIPGSSTTGTGPCRTCTTPAQGTSLYPSCCCTKPSDGNCTCIPIPSSWAFGKFLWEWASARFSW

SEQ ID NO: 36 - protein, 72 aa, Hepatitis B virus
  source 1..72: note = HBsAg M133T HBsAg Antigenic Loop Sequence
QGMLPVCPLIPGSSTTGTGPCRTCTTPAQGTSTYPSCCCTKPSDGNCTCIPIPSSWAFGKFLWEWASARFSW

SEQ ID NO: 37 - protein, 72 aa, Hepatitis B virus
  source 1..72: note = HBsAg K141 E HBsAg Antigenic Loop Sequence
QGMLPVCPLIPGSSTTGTGPCRTCTTPAQGTSMYPSCCCTEPSDGNCTCIPIPSSWAFGKFLWEWASARFSW

SEQ ID NO: 38 - protein, 72 aa, Hepatitis B virus
  source 1..72: note = HBsAg P142S HBsAg Antigenic Loop Sequence
QGMLPVCPLIPGSSTTGTGPCRTCTTPAQGTSMYPSCCCTKSSDGNCTCIPIPSSWAFGKFLWEWASARFSW

SEQ ID NO: 39 - protein, 72 aa, Hepatitis B virus
  source 1..72: note = HBsAg S143K HBsAg Antigenic Loop Sequence
QGMLPVCPLIPGSSTTGTGPCRTCTTPAQGTSMYPSCCCTKPKDGNCTCIPIPSSWAFGKFLWEWASARFSW

SEQ ID NO: 40 - protein, 71 aa, Hepatitis B virus
  source 1..71: note = HBsAg D144A HBsAg Antigenic Loop Sequence
QGMLPVCPLIPGSSTTGTGPCRTCTTPAQGTSMYPSCCCTPSAGNCTCIPIPSSWAFGKFLWEWASARFSW

SEQ ID NO: 41 - protein, 72 aa, Hepatitis B virus
  source 1..72: note = HBsAg G145R HBsAg Antigenic Loop Sequence
QGMLPVCPLIPGSSTTGTGPCRTCTTPAQGTSMYPSCCCTKPSDRNCTCIPIPSSWAFGKFLWEWASARFSW

SEQ ID NO: 42 - protein, 72 aa, Hepatitis B virus
  source 1..72: note = HBsAg N146A HBsAg Antigenic Loop Sequence
QGMLPVCPLIPGSSTTGTGPCRTCTTPAQGTSMYPSCCCTKPSDGACTCIPIPSSWAFGKFLWEWASARFSW

SEQ ID NO: 43 - protein, 226 aa, synthetic construct
  source 1..226: note = HBsAg Genotype D S domain (Genbank accession no. FJ899792)
MENVTSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTGTGPCRTCTTPAQGTSMYPSCCCTKPSDGNCTCIPIPSSWAFGKFLWEWASARFSWLSLLVPFVQWFVGLSPTVWLSVIWMMWYWGPSLYSTLSPFLPLLPIFFCLWVYI

SEQ ID NO: 44 - protein, 8 aa, synthetic construct
  source 1..8: note = HBC34 CDRH1
GRIFRSFY

SEQ ID NO: 45 - protein, 7 aa, synthetic construct
  source 1..7: note = HBC34 short CDRH2
NQDGSEK

SEQ ID NO: 46 - protein, 8 aa, synthetic construct
  source 1..8: note = HBC34 long CDRH2
INQDGSEK

SEQ ID NO: 47 - protein, 12 aa, synthetic construct
  source 1..12: note = HBC34 CDRH3
AAWSGNSGGMDV

SEQ ID NO: 48 - protein, 6 aa, synthetic construct
  source 1..6: note = HBC34 CDRL1
KLGNKN

SEQ ID NO: 49 - skipped

SEQ ID NO: 50 - protein, 9 aa, synthetic construct
  source 1..9: note = HBC34 long CDRL2
VIYEVKYRP

SEQ ID NO: 51 - protein, 9 aa, synthetic construct
  source 1..9: note = HBC34 CDRL3
QTWDSTTVV

SEQ ID NO: 52 - protein, 9 aa, synthetic construct
  source 1..9: note = HBC34v35 CDRL3
QTFDSTTVV

SEQ ID NO: 53 - protein, 119 aa, synthetic construct
  source 1..119: note = HBC34 VH
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSS

SEQ ID NO: 54 - protein, 106 aa, synthetic construct
  source 1..106: note = HBC34 VL
SYELTQPPSVSVSPGQTVSIPCSGDKLGNKNVCWFQHKPGQSPVLVIYEVKYRPSGIPERFSGSNSGNTATLTISGTQAMDEAAYFCQTWDSTTVVFGGGTRLTVL

SEQ ID NO: 55 - protein, 106 aa, synthetic construct
  source 1..106: note = HBC34v35 VL
SYELTQPPSVSVSPGQTVSIPCSGDKLGNKNVAWFQHKPGQSPVLVIYEVKYRPSGIPERFSGSNSGNTATLTISGTQAMDEAAYFCQTFDSTTVVFGGGTRLTVL

SEQ ID NO: 56 - protein, 449 aa, synthetic construct
  source 1..449: note = HBC34v35 HC
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 449 aa, synthetic construct
  source 1..449: note = HBC34v35-MLNS-GAALIE (g1M17, 1) HC
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 58 - protein, 212 aa, synthetic construct
  source 1..212: note = HBC34 LC
SYELTQPPSVSVSPGQTVSIPCSGDKLGNKNVCWFQHKPGQSPVLVIYEVKYRPSGIPERFSGSNSGNTATLTISGTQAMDEAAYFCQTWDSTTVVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - protein, 212 aa, synthetic construct
  source 1..212: note = HBC34v35 LC
SYELTQPPSVSVSPGQTVSIPCSGDKLGNKNVAWFQHKPGQSPVLVIYEVKYRPSGIPERFSGSNSGNTATLTISGTQAMDEAAYFCQTFDSTTVVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 60 - protein, 217 aa, Homo sapiens
  source 1..217: note = WT hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 19 aa, synthetic construct
  source 1..19: note = Chimeric hinge
ESKYGPPCPPCPAPPVAGP

(19) United States
(12) Patent Application Publication
(10) Pub. No.: US 2025/0302951 A1
(43) Pub. Date: Oct. 2, 2025

(54) COMPOSITIONS AND METHODS FOR TREATING HEPATITIS D VIRUS (HDV) INFECTION AND ASSOCIATED DISEASES

(71) Applicant: Vir Biotechnology, Inc., San Francisco, CA (US)

(72) Inventors: Michael A. Chattergoon, Towson, MD (US); Daniel J. Cloutier, San Mateo, CA (US); Sophia E. Elie, Rocklin, CA (US); Sneha V. Gupta, Boulder, CO (US); Carey K. Hwang, Skillman, NJ (US); Audrey H. Lau, San Mateo, CA (US)

(21) Appl. No.: 18/866,888

(22) PCT Filed: May 18, 2023
(86) PCT No.: PCT/US2023/067179

§ 371 Date: Nov. 18, 2024

Related U.S. Application Data
(60) Provisional application No. 63/343,908, filed on May 19, 2022.

(51) Int. Cl. A61K 39/42; A61K 39/00; A61P 31/14; A61P 37/04; C07K 16/10; C12N 15/113

(52) U.S. Cl. CPC A61K 39/42; A61P 31/14; A61P 37/04; C07K 16/10; C12N 15/1131; A61K 2039/505; C07K 2317/24; C07K 2317/52; C07K 2317/76; C12N 2310/14; C12N 2320/31

(57) ABSTRACT

The present disclosure provides methods for treating hepatitis D virus (HDV) infection and/or an HDV-associated disease using combination therapies, and related kits and compositions for use in such methods. The components of the combination therapies may include one or more of an anti-HBV antibody; an siRNA that targets an HBV mRNA; and a nucleos(t)ide reverse transcriptase inhibitor (NRTI).

### REFERENCE TO AN ELECTRONIC SEQUENCE LISTING

The contents of the electronic sequence listing (444WO_SeqListing.xml; Size: 70.5 KB; and Date of Creation: May 5, 2023) is herein incorporated by reference in its entirety.

### BACKGROUND

Hepatitis D, also known as “delta hepatitis,” is a viral infection caused by the hepatitis D virus (HDV). HDV is a defective RNA satellite virus that does not encode its own envelope proteins and is dependent on the expression of the hepatitis B virus (HBV) surface antigen (HBsAg) to complete its life cycle and produce infectious HDV virions. Thus, HDV must either coinfect or superinfect with HBV.

Approximately 300 million people are living with chronic HBV infection worldwide (Polaris Observatory Collaborators, Global prevalence, treatment, and prevention of hepatitis B virus infection in 2016: a modelling study, Lancet Gastroenterol Hepatol. 2018 June, 3(6):383-403) and are at risk of serious sequelae, including cirrhosis, liver failure, hepatocellular carcinoma (HCC). Worldwide prevalence of HDV has been estimated in 3 recent meta-analyses, with results ranging from 12 million people (Stockdale A J et al., The global prevalence of hepatitis D virus infection: Systematic review and meta-analysis, J Hepatol. 2020 September, 73(3):523-532) to 60 to 72 million (Chen H Y et al., Prevalence and burden of hepatitis D virus infection in the global population: a systematic review and meta-analysis, Gut 2019 March, 68(3):512-521; Miao Z et al., Estimating the Global Prevalence, Disease Progression, and Clinical Outcome of Hepatitis Delta Virus Infection, J Infect Dis. 2020 Apr. 27, 221(10):1677-1687). HDV infection is the most aggressive form of viral hepatitis due to rapid progression to liver failure, cirrhosis, HCC, and death observed in persons with chronic HBV/HDV coinfection (. and Lee C, Hepatitis D Review: Challenges for the Resource-Poor Setting, Viruses. 2021 Sep. 23, 13(10):1912; Stockdale et al., 2020, supra). Among persons with chronic HDV infection, 85 to 95% may develop cirrhosis and liver failure within 10 years of infection with some developing these complications as early as 1 to 2 years after infection (˜15%) (Kamili S et al., Delta hepatitis: Toward improved diagnostics, Hepatology 2017 December, 66(6):1716-1718; NIH, National Institute of Diabetes and Digestive and Kidney Diseases, United States 2017, Department of Health and Human Services, Accessed Apr. 10, 2020; Rizzetto M, Hepatitis D Virus: Introduction and Epidemiology, Cold Spring Harb Perspect Med. 2015 July 1, 5(7), a021576; WHO, Hepatitis B, https://www.who.int/news-room/factsheets/detail/hepatitis-b, Published July 2021, Accessed Apr. 10, 2022). Notably, approximately 50% of HDV-infected patients are cirrhotic at diagnosis (Fattovich G et al., Influence of hepatitis delta virus infection on progression to cirrhosis in chronic hepatitis type B, J Infect Dis. 1987 May, 155(5):931-5).

Hepatitis B virus (HBV) is a DNA virus that infects, replicates, and persists in human hepatocytes (Protzer U et al., Living in the liver: hepatic infections, Nature Reviews Immunology 2012, 12:201-213). The small viral genome (3.2 kb), consists of partially double-stranded, relaxed-circular DNA (rcDNA) and has 4 open reading frames encoding 7 proteins: HBcAg (HBV core antigen, viral capsid protein), HBeAg (hepatitis B e-antigen), HBV Pol/RT (polymerase, reverse transcriptase), PreS1/PreS2/HBsAg (large, medium, and small surface envelope glycoproteins), and HBx (HBV×antigen, regulator of transcription required for the initiation of infection) (Seeger C et al., Molecular biology of hepatitis B virus infection, Virology 2015, 479-480:672-686; Tong S et al., Overview of viral replication and genetic variability, Journal of Hepatology, 2016, 64(1):S4-S16).

In hepatocytes, rcDNA, the form of HBV nucleic acid that is introduced by the infection virion, is converted into a covalently closed circular DNA (cccDNA), which persists in the host cell's nucleus as an episomal chromatinized structure (Allweiss L et al., The Role of cccDNA in HBV Maintenance, Viruses 2017, 9:156). The cccDNA serves as a transcription template for all viral transcripts (Lucifora J et al., Attacking hepatitis B virus cccDNA—The holy grail to hepatitis B cure, Journal of Hepatology 2016, 64(1):S41-S48). Pregenomic RNA (pgRNA) transcripts are reverse transcribed into new rcDNA for new virions, which are secreted without causing cytotoxicity. In addition to infectious virions, infected hepatocytes secrete large amounts of genome-free subviral particles that may exceed the number of secreted virions by 10,000-fold (Seeger et al., 2015, supra). Random integration of the virus into the host genome can occur as well, a mechanism that contributes to hepatocyte transformation (Levrero M et al., Mechanisms of HBV-induced hepatocellular carcinoma, Journal of Hepatology 2016, 64(1):S84-S101). HBV persists in hepatocytes in the form of cccDNA and integrated DNA (intDNA).

Hepatitis B infection is characterized by serologic viral markers and antibodies. In acute resolving infections, the virus is cleared by effective innate and adaptive immune responses that include cytotoxic T cells leading to death of infected hepatocytes, and induction of B cells producing neutralizing antibodies that prevent the spread of the virus (Bertoletti A, Adaptive immunity in HBV infection, Journal of Hepatology 2016, 64(1):S71-S83; Maini M K et al., The role of innate immunity in the immunopathology and treatment of HBV infection, Journal of Hepatology 2016, 64(1): S60-S70; Li Y et al., Genome-wide association study identifies 8p21.3 associated with persistent hepatitis B virus infection among Chinese, Nature Communications 2016, 7:11664). In contrast, chronic infection is associated with T and B cell dysfunction, mediated by multiple regulatory mechanisms including presentation of viral epitopes on hepatocytes and secretion of subviral particles (Bertoletti et al., 2016, supra; Maini et al., 2016, supra; Burton A R et al., Dysfunctional surface antigen specific memory B cells accumulate in chronic hepatitis B infection, EASL International Liver Congress, Paris, France 2018). Thus, the continued expression and secretion of viral proteins due to cccDNA persistence in hepatocytes is considered a key step in the inability of the host to clear the infection.

Treatment options for HDV infection are limited to pegylated interferon alpha (PEG-IFNα) and buleviritide. PEG-IFNα leads to sustained virologic response (SVR (clearance of serum HDV maintained 6 months after stopping treatment)) in only around 25 to 30% of individuals treated for 48 weeks. Late relapses were observed in approximately 50% of those patients reducing the long-term efficacy to approx. 15% (Abbas Z et al., Interferon alpha for chronic hepatitis D. Cochrane Database Syst Rev. 2011 Dec. 7, 2011(12):CD006002; Heidrich B et al., Late HDV RNA relapse after peginterferon alpha-based therapy of chronic hepatitis delta, Hepatology 2014; 60:87-97). Current guidelines from the American Association for the Study of Liver Diseases (AASLD), the Asia Pacific Association for the Study of the Liver (APASL) and the European Association for the Study of the Liver (EASL) recommend administering PEG-IFNα for at least 48 weeks to patients with chronic HDV infection (European Association for the Study of the Liver (EASL), EASL 2017 Clinical Practice Guidelines on the management of hepatitis B virus infection. J Hepatol. 2017 August, 67(2):370-398; Sarin S K et al., Asian-Pacific clinical practice guidelines on the management of hepatitis B: a 2015 update, Hepatol Int. 2016 January, 10(1):1-98; Terrault N A et al., Update on prevention, diagnosis, and treatment of chronic hepatitis B: AASLD 2018 hepatitis B guidance, Hepatology 2018 April, 67(4):1560-1599). In addition to low SVR rates, the major adverse reactions of PEG-IFNα therapy are well described and PEG-IFNα is contraindicated in patients with autoimmune diseases, major psychiatric syndromes and Child-Pugh-Turcotte (CPT)-B or CPT-C stage cirrhotic patients (Rizzetto M, Hepatitis D Virus: Introduction and Epidemiology, Cold Spring Harb Perspect Med. 2015 Jul. 1, 5(7):a021576; Sleijfer S et al., Side effects of interferon-alpha therapy, Pharm World Sci. 2005 December, 27(6):423-31). Further limiting its usefulness, reduced PEG-IFNα efficacy has been observed when treating cirrhotic patients with chronic HDV (Gunsar F et al., Two-year interferon therapy with or without ribavirin in chronic delta hepatitis, Antivir Ther. 2005, 10(6):721-6). Limitations about the use of PEG-IFNα in cirrhotic HDV patients is particularly notable as 50% of HDV-infected patients are cirrhotic at diagnosis (Fattovich et al., 1987, supra). These limitations and poor efficacy of current agents highlights an unmet need for patients with chronic HDV infection in all stages of the disease. Bulevirtide (BLV), an entry inhibitor that targets the sodium taurocholate co-transporting polypeptide (NTCP) receptor, has conditional approval in the EU for chronic HDV therapy. Interim results from the Phase 3 MYR301 study indicate that after 24 weeks of 2 mg daily subcutaneous (SC) dose of BLV, 36.7% of participants achieve the combined virological and biochemical endpoint. Nucleoside reverse transcriptase inhibitors (NRTI) therapy can suppress HBV replication but do not directly impact HBsAg production, intrahepatic HDV replication, or HDV viremia.

### BRIEF SUMMARY

In some aspects, the present disclosure provides methods of treating hepatitis D virus (HDV) infection or an HDV-associated disease in a subject in need thereof, comprising administering to the subject: (a) an anti-HBV antibody; and (b) an siRNA that targets an HBV mRNA. In some embodiments, the subject is cirrhotic, e.g., has a liver biopsy with METAVIR F4 or Liver elastography (Fibroscan®)≥12 kilopascal (kPa) within the 12 months prior to treatment; a creatine clearance (CLcr)≥60 mL/min as calculated by the Cockcroft-Gault formula prior to treatment; a Child-Pugh-Turcotte (CPT) score of 5 or higher prior to treatment.

In some aspects, compositions for use in treatment, compositions for use in the manufacture of medicaments, and kits are provided.

### BRIEF DESCRIPTION OF THE DRAWINGS

FIG. 1 depicts the study schema for Cohort 1 of the clinical study in Example 1.

FIG. 2 depicts the study schema for Cohorts 2-4 for the clinical study in Example 1.

FIGS. 3A-3D depict the schedule of activities during the Induction Period for Cohort 1a and Cohort 1b in the clinical study in Example 1.

FIGS. 4A-4C depict the schedule of activities during the Maintenance Period for Cohort 1a and Cohort 1b in the clinical study in Example 1.

FIGS. 5A-5D depict the schedule of activities during the Induction Period for Cohorts 2 to 4 in the clinical study in Example 1.

FIGS. 6A-6C depict the schedule of activities during the Maintenance Period for Cohorts 2 to 4, for Participants Transitioning to the Maintenance Period at Week 24, in the clinical study in Example 1.

FIGS. 7A-7C depict the schedule of activities during the Maintenance Period for Cohorts 2 to 4, for Participants Transitioning to the Maintenance Period at Week 48, in the clinical study in Example 1.

FIGS. 8A-8C depict the schedule of activities for the Follow-Up Period for all cohorts in the clinical study in Example 1.

FIG. 9 shows in vitro HBV neutralization activity of AB01.

FIG. 10 shows in vitro HDV neutralization activity of AB01.

FIG. 11 shows in vitro HBV antiviral activity of SIRNA01.

FIGS. 12A-12B depict the study design for Cohorts 1-4 for the clinical study in Example 3.

FIGS. 13A-13D depict the schedule of activities during the Induction Period for Cohort 1a and Cohort 1b in the clinical study in Example 3.

FIGS. 14A-14D depict the schedule of activities during the Maintenance Period for Cohort 1a and Cohort 1b in the clinical study in Example 3.

FIGS. 15A-15D depict the schedule of activities during the first 48 weeks of the Treatment Period for Cohorts 2a, 2b1, 2b2, and 2c in the clinical study in Example 3.

FIGS. 16A-16D depict the schedule of activities during weeks 49-96 of the Treatment Period for Cohorts 2a, 2b1, 2b2, and 2c in the clinical study in Example 3.

FIGS. 17A-17E depict the schedule of activities during the first 48 weeks of the Treatment Period for Cohort 3 in the clinical study in Example 3.

FIGS. 18A-18D depict the schedule of activities during weeks 49-96 of the Treatment Period for Cohort 3 in the clinical study in Example 3.

FIGS. 19A-19D depict the schedule of activities during the Delayed Treatment Period for Cohort 4 in the clinical study in Example 3.

FIGS. 20A-20B depict the schedule of activities for the Follow-Up Period for all cohorts in the clinical study in Example 3.

FIGS. 21A-21G depict the schedule of activities for pharmacokinetics analyses in the clinical study in Example 3.

FIGS. 22A-22B depict the schedule of activities for optional studies in the clinical study in Example 3.

FIG. 23 shows HDV RNA change from baseline over the first 8 weeks for six subjects in Cohort 1b of the clinical study in Example 3.

### DETAILED DESCRIPTION

The instant disclosure provides methods and compositions for use in treating hepatitis D virus (HDV) infection or a HDV-associated disease, wherein the subject is administered one or more of an anti-HBV antibody and an anti-HBV siRNA, and related kits.

### I. GLOSSARY

The following sections provide a detailed description of combination therapies for treating HDV infection or a HDV-associated disease, and kits related to the combination therapies. Prior to setting forth this disclosure in more detail, it may be helpful to an understanding thereof to provide definitions of certain terms to be used herein. Additional definitions are set forth throughout this disclosure.

In the present description, the term “about” means±20% of the indicated range, value, or structure, unless otherwise indicated.

The term “comprise” (and similar terms such as “comprising of” and “comprised of”) means the presence of the stated features, integers, steps, or components as referred to in the claims, but that it does not preclude the presence or addition of one or more other features, integers, steps, components, or groups thereof. The term “consisting essentially of” limits the scope of a claim to the specified materials or steps and those that do not materially affect the basic and novel characteristics of the claimed invention.

It should be understood that the terms “a” and “an” as used herein refer to “one or more” of the enumerated components. The use of the alternative (e.g., “or”) should be understood to mean either one, both, or any combination thereof of the alternatives, and may be used synonymously with “and/or”. As used herein, the terms “include” and “have” are used synonymously, which terms and variants thereof are intended to be construed as non-limiting or open-ended.

The word “substantially” does not exclude “completely”; e.g., a composition which is “substantially free” from Y may be completely free from Y. Where necessary, the word “substantially” may be omitted from definitions provided herein.

The term “disease” as used herein is intended to be generally synonymous, and is used interchangeably with, the terms “disorder” and “condition” (as in “medical condition”), in that all reflect an abnormal condition of the human or animal body or of one of its parts that impairs normal functioning, is typically manifested by distinguishing signs and symptoms, and causes the human or animal to have a reduced duration or quality of life.

As used herein, the terms “peptide”, “polypeptide”, and “protein” and variations of these terms refer to a molecule, in particular a peptide, oligopeptide, polypeptide, or protein including fusion protein, respectively, comprising at least two amino acids joined to each other by a normal peptide bond, or by a modified peptide bond, such as for example in the cases of isosteric peptides. For example, a peptide, polypeptide, or protein may be composed of amino acids selected from the 20 amino acids defined by the genetic code, linked to each other by a normal peptide bond (“classical” polypeptide). A peptide, polypeptide, or protein can be composed of L-amino acids and/or D-amino acids. In particular, the terms “peptide”, “polypeptide”, and “protein” also include “peptidomimetics,” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide, or protein may comprise amino acids other than the 20 amino acids defined by the genetic code in addition to these amino acids, or it can be composed of amino acids other than the 20 amino acids defined by the genetic code. In particular, a peptide, polypeptide, or protein in the context of the present disclosure can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain, or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, or “protein” in the context of the present disclosure in particular also include modified peptides, polypeptides, and proteins. For example, peptide, polypeptide, or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation, or ubiquitination. Such modifications are fully detailed in the literature (Proteins Structure and Molecular Properties, 2nd Ed., T. E. Creighton, New York (1993); Post-translational Covalent Modifications of Proteins, B. C. Johnson, Ed., Academic Press, New York (1983); Seifter et al., Analysis for protein modifications and nonprotein cofactors, Meth. Enzymol. 1990, 182:626-46; and Rattan et al., Protein Synthesis: Post-translational Modifications and Aging, Ann NY Acad Sci 1992, 663:48-62). Accordingly, the terms “peptide”, “polypeptide”, and “protein” include for example lipopeptides, lipoproteins, glycopeptides, glycoproteins, and the like.

As used herein a “(poly)peptide” comprises a single chain of amino acid monomers linked by peptide bonds as explained above. A “protein”, as used herein, comprises one or more, e.g., 1, 2, 3, 4, 5, 6, 7, 8, 9 or 10 (poly)peptides, i.e., one or more chains of amino acid monomers linked by peptide bonds as explained above. In particular embodiments, a protein according to the present disclosure comprises 1, 2, 3, or 4 polypeptides.

The term “recombinant”, as used herein (e.g., a recombinant antibody, a recombinant protein, a recombinant nucleic acid, etc.), refers to any molecule (antibody, protein, nucleic acid, siRNA, etc.) that is prepared, expressed, created, or isolated by recombinant means, and which is not naturally occurring. As used herein, the terms “nucleic acid”, “nucleic acid molecule,” and “polynucleotide” are used interchangeably and are intended to include DNA molecules and RNA molecules. A nucleic acid molecule may be single-stranded or double-stranded. In particular embodiments, the nucleic acid molecule is double-stranded RNA.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

As used herein, the term “sequence variant” refers to any sequence having one or more alterations in comparison to a reference sequence, whereby a reference sequence is any of the sequences listed in the sequence listing, i.e., SEQ ID NO:1 to SEQ ID NO:61. Thus, the term “sequence variant” includes nucleotide sequence variants and amino acid sequence variants. For a sequence variant in the context of a nucleotide sequence, the reference sequence is also a nucleotide sequence, whereas for a sequence variant in the context of an amino acid sequence, the reference sequence is also an amino acid sequence. A “sequence variant” as used herein is at least 80%, at least 85%, at least 90%, at least 95%, at least 98%, or at least 99% identical to the reference sequence. Sequence identity is usually calculated with regard to the full length of the reference sequence (i.e., the sequence recited in the application), unless otherwise specified. Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=1 1 and gap extension penalty=1]. A “sequence variant” in the context of a nucleic acid (nucleotide) sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleotide sequence can either result in a change in the respective reference amino acid sequence, i.e., in an amino acid “sequence variant” or not. In certain embodiments, the nucleotide sequence variants are variants that do not result in amino acid sequence variants (i.e., silent mutations). However, nucleotide sequence variants leading to “non-silent” mutations are also within the scope, in particular such nucleotide sequence variants, which result in an amino acid sequence, which is at least 80%, at least 85%, at least 90%, at least 95%, at least 98%, or at least 99% identical to the reference amino acid sequence. A “sequence variant” in the context of an amino acid sequence has an altered sequence in which one or more of the amino acids is deleted, substituted or inserted in comparison to the reference amino acid sequence. As a result of the alterations, such a sequence variant has an amino acid sequence which is at least 80%, at least 85%, at least 90%, at least 95%, at least 98%, or at least 99% identical to the reference amino acid sequence. For example, per 100 amino acids of the reference sequence a variant sequence having no more than 10 alterations, i.e., any combination of deletions, insertions, or substitutions, is “at least 90% identical” to the reference sequence.

While it is possible to have non-conservative amino acid substitutions, in certain embodiments, the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine, and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Unless otherwise stated, alterations in the sequence variants do not abolish the functionality of the respective reference sequence, for example, in the present case, the functionality of a sequence of an anti-HBV antibody or an siRNA to sufficiently neutralize infection of HBV or reduce HBV protein expression, respectively. Guidance in determining which nucleotides and amino acid residues, respectively, may be substituted, inserted, or deleted without abolishing such functionality can be found by using computer programs well known in the art.

As used herein, a nucleic acid sequence or an amino acid sequence “derived from” a designated nucleic acid, peptide, polypeptide, or protein refers to the origin of the nucleic acid, peptide, polypeptide, or protein. In some embodiments, the nucleic acid sequence or amino acid sequence which is derived from a particular sequence has an amino acid sequence that is essentially identical to that sequence or a portion thereof, from which it is derived, whereby “essentially identical” includes sequence variants as defined above. In certain embodiments, the nucleic acid sequence or amino acid sequence which is derived from a particular peptide or protein is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins, and nucleic acids are thus identifiable to one of ordinary skill in the art. Likewise, sequences “derived from” other sequence are usually identifiable to one of ordinary skill in the art as having its origin in the sequence.

In some embodiments, a nucleic acid sequence or an amino acid sequence derived from another nucleic acid, peptide, polypeptide, or protein may be identical to the starting nucleic acid, peptide, polypeptide, or protein (from which it is derived).

However, a nucleic acid sequence or an amino acid sequence derived from another nucleic acid, peptide, polypeptide, or protein may also have one or more mutations relative to the starting nucleic acid, peptide, polypeptide, or protein (from which it is derived), in particular a nucleic acid sequence or an amino acid sequence derived from another nucleic acid, peptide, polypeptide, or protein may be a functional sequence variant as described above of the starting nucleic acid, peptide, polypeptide, or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g., a corresponding genomic sequence. A mutation, e.g., in comparison to a genomic sequence, may be, for example, a (naturally occurring) somatic mutation, a spontaneous mutation, an induced mutation, e.g., induced by enzymes, chemicals, or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g., in a nucleic acid sequence or in an amino acid sequence. A mutation includes substitution, deletion, and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved, e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein, the term “coding sequence” is intended to refer to a polynucleotide molecule, which encodes the amino acid sequence of a protein product.

The boundaries of the coding sequence are generally determined by an open reading frame, which usually begins with an ATG start codon.

The term “expression” as used herein refers to any step involved in the production of the polypeptide, including transcription, post-transcriptional modification, translation, post-translational modification, secretion, or the like.

The term “vaccine” as used herein is typically understood to be a prophylactic or therapeutic material providing at least one antigen or immunogen, including viral vector vaccines that include nucleic acids encoding the antigen(s) or immunogen(s). The antigen or immunogen may be derived from any material that is suitable for vaccination. For example, the antigen or immunogen may be derived from a pathogen, such as from bacteria or virus particles, etc., or from a tumor or cancerous tissue. The antigen or immunogen stimulates the body's adaptive immune system to provide an adaptive immune response. In particular, an “antigen” or an “immunogen” refers typically to a substance which may be recognized by the immune system (e.g., the adaptive immune system), and which is capable of triggering an antigen-specific immune response, e.g., by formation of antibodies and/or antigen-specific T cells as part of an adaptive immune response. Typically, an antigen may be or may comprise a peptide or protein that may be presented by the MHC to T-cells.

As used herein, “Hepatitis B virus,” used interchangeably with the term “HBV” refers to the well-known non-cytopathic, liver-tropic DNA virus belonging to the Hepadnaviridae family. The HBV genome is partially double-stranded, circular DNA with four overlapping reading frames (that may be referred to herein as “genes,” “open reading frames,” or “transcripts”): C, X, P, and S. The core protein is coded for by gene C (HBcAg). Hepatitis B e antigen (HBeAg) is produced by proteolytic processing of the pre-core (pre-C) protein. The DNA polymerase is encoded by gene P. Gene S is the gene that codes for the surface antigens (HBsAg). The HBsAg gene is one long open reading frame which contains three in frame “start” (ATG) codons resulting in polypeptides of three different sizes called large, middle, and small S antigens, pre-S1+pre-S2+S, pre-S2+S, or S. Surface antigens, in addition to decorating the envelope of HBV, are also part of subviral particles, which are produced at large excess as compared to virion particles, and play a role in immune tolerance and in sequestering anti-HBsAg antibodies, thereby allowing for infectious particles to escape immune detection. The protein coded for by gene X plays a role in transcriptional transactivation and replication and is associated with the development of liver cancer.

Nine genotypes of HBV, designated A to I, have been determined, and an additional genotype J has been proposed, each having a distinct geographical distribution (Velkov S et al., The Global Hepatitis B Virus Genotype Distribution Approximated from Available Genotyping Data, Genes 2018, 9(10):495). The term “HBV” includes any of the genotypes of HBV (A to J). The complete coding sequence of the reference sequence of the HBV genome may be found in for example, GenBank Accession Nos. GI:21326584 and GI:3582357. Amino acid sequences for the C, X, P, and S proteins can be found at, for example, NCBI Accession numbers YP_009173857.1 (C protein); YP_009173867.1 and BAA32912.1 (X protein); YP_009173866.1 and BAA32913.1 (P protein); and YP_009173869.1, YP_009173870.1, YP_009173871.1, and BAA32914.1 (S protein). Additional examples of HBV messenger RNA (mRNA) sequences are available using publicly available databases, e.g., GenBank, UniProt, and OMIM. The International Repository for Hepatitis B Virus Strain Data can be accessed at http://www.hpa-bioinformatics.org.uk/HepSEQ/main.php. The term “HBV,” as used herein, also refers to naturally occurring DNA sequence variations of the HBV genome, i.e., genotypes A-J and variants thereof.

In some embodiments, the present disclosure provides combination therapy to treat HBV that includes an anti-HBV siRNA. siRNA mediates the targeted cleavage of an RNA transcript via an RNA-induced silencing complex (RISC) pathway, thereby effecting inhibition of gene expression. This process is frequently termed “RNA interference” (RNAi). Without wishing to be bound to a particular theory, long double-stranded RNA (dsRNA) introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair siRNAs with characteristic two base 3′ overhangs (Bernstein et al., Nature 2001, 409:363). The siRNAs are then incorporated into RISC where one or more helicases unwind the siRNA duplex, enabling the complementary antisense strand to guide target recognition (Nykanen et al., Cell 2001 107:309). Upon binding to the appropriate target mRNA, one or more endonucleases within RISC cleaves the target to induce silencing (Elbashir et al., Genes Dev. 2001, 15:188).

The terms “silence,” “inhibit the expression of,” “down-regulate the expression of,” “suppress the expression of,” and the like, in so far as they refer to an HBV gene, herein refer to the at least partial reduction of the expression of an HBV gene, as manifested by a reduction of the amount of HBV mRNA which can be isolated from or detected in a first cell or group of cells in which an HBV gene is transcribed and which has or have been treated with an inhibitor of HBV gene expression, such that the expression of the HBV gene is inhibited, as compared to a second cell or group of cells substantially identical to the first cell or group of cells but which has or have not been so treated (control cells). The degree of inhibition can be measured, by example, as the difference between the degree of mRNA expression in a control cell minus the degree of mRNA expression in a treated cell. Alternatively, the degree of inhibition can be given in terms of a reduction of a parameter that is functionally linked to HBV gene expression, e.g., the amount of protein encoded by an HBV gene, or the number of cells displaying a certain phenotype, e.g., an HBV infection phenotype. In principle, HBV gene silencing can be determined in any cell expressing the HBV gene, e.g., an HBV-infected cell or a cell engineered to express the HBV gene, and by any appropriate assay.

The level of HBV RNA that is expressed by a cell or group of cells, or the level of circulating HBV RNA, may be determined using any method known in the art for assessing mRNA expression, such as the rtPCR method provided in Example 2 of International Application Publication No. WO 2016/077321A1 and U.S. Patent Application Publication No. US2017/0349900A1, which methods are incorporated herein by reference. In some embodiments, the level of expression of an HBV gene (e.g., total HBV RNA, an HBV transcript, e.g., HBV 3.5 kb transcript) in a sample is determined by detecting a transcribed polynucleotide, or portion thereof, e.g., RNA of the HBV gene. RNA may be extracted from cells using RNA extraction techniques including, for example, using acid phenol/guanidine isothiocyanate extraction (RNAzol B; Biogenesis), RNeasy RNA preparation kits (Qiagen®), or PAXgene (PreAnalytix, Switzerland). Typical assay formats utilizing ribonucleic acid hybridization include nuclear run-on assays, RT-PCR, RNase protection assays (Melton D A et al., Efficient in vitro synthesis of biologically active RNA and RNA hybridization probes from plasmids containing a bacteriophage SP6 promoter, Nuc. Acids Res. 1984, 12:7035-56), northern blotting, in situ hybridization, and microarray analysis. Circulating HBV mRNA may be detected using methods the described in International Application Publication No. WO 2012/177906A1 and U.S. Patent Application Publication No. US2014/0275211A1, which methods are incorporated herein by reference.

As used herein, “target sequence” refers to a contiguous portion of the nucleotide sequence of an mRNA molecule formed during the transcription of an HBV gene, including mRNA that is a product of RNA processing of a primary transcription product. The target portion of the sequence will be at least long enough to serve as a substrate for RNAi-directed cleavage at or near that portion. For example, the target sequence will generally be from 9-36 nucleotides in length, e.g., 15-30 nucleotides in length, including all sub-ranges there between. As non-limiting examples, a target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an siRNA as described herein include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, an siRNA comprising one oligonucleotide 21 nucleotides in length, and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary,” and “substantially complementary” herein can be used with respect to the base matching between the sense strand and the antisense strand of an siRNA, or between the antisense strand of an siRNA agent and a target sequence, as will be understood from the context of their use.

As used herein, a polynucleotide that is “substantially complementary” to at least part of a mRNA refers to a polynucleotide that is substantially complementary to a contiguous portion of the mRNA of interest (e.g., an mRNA encoding an HBV protein). For example, a polynucleotide is complementary to at least a part of an HBV mRNA if the sequence is substantially complementary to a non-interrupted portion of the HBV mRNA.

The term “siRNA,” as used herein, refers to an RNA interference molecule that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range there between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, and 21-22 base pairs. siRNAs generated in the cell by processing with Dicer and similar enzymes are generally in the range of 19-22 base pairs in length.

One strand of the duplex region of an siRNA comprises a sequence that is substantially complementary to a region of a target RNA. The two strands forming the duplex structure can be from a single RNA molecule having at least one self-complementary region, or can be formed from two or more separate RNA molecules.

Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of an siRNA are comprised by separate RNA molecules, those molecules need not, but can be covalently connected. Where the two strands are connected covalently by means other than a hairpin loop, the connecting structure is referred to as a “linker.”

An siRNA as described herein can be synthesized by standard methods known in the art, e.g., by use of an automated DNA synthesizer, such as are commercially available from, for example, Biosearch, Applied Biosystems, Inc.

The term “antisense strand” or “guide strand” refers to the strand of an siRNA that includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand” or “passenger strand” as used herein, refers to the strand of an siRNA that includes a region that is substantially complementary to a region of the antisense strand as that term is defined herein.

The term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure or in the ribose-phosphate backbone structure, e.g., as described in greater detail below. However, siRNA molecules comprising ribonucleoside analogs or derivatives retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate, or a non-natural base comprising nucleoside, or any combination thereof. In another example, an RNA molecule can comprise at least two modified ribonucleosides, at least 3, at least 4, at least 5, at least 6, at least 7, at least 8, at least 9, at least 10, at least 15, at least 20, or more, up to the entire length of the siRNA molecule. The modifications need not be the same for each of such a plurality of modified ribonucleosides in an RNA molecule. In some embodiments, a modified ribonucleoside includes a deoxyribonucleoside. For example, an siRNA can comprise one or more deoxynucleosides, including, for example, a deoxynucleoside overhang(s), or one or more deoxynucleosides within the double-stranded portion of an siRNA. However, the term “siRNA” as used herein does not include a fully DNA molecule.

As used herein, the term “nucleotide overhang” refers to at least one unpaired nucleotide that protrudes from the duplex structure of an siRNA. For example, when a 3′-end of one strand of an siRNA extends beyond the 5′-end of the other strand, or vice versa, there is a nucleotide overhang. An siRNA can comprise an overhang of at least one nucleotide; alternatively the overhang can comprise at least two nucleotides, at least three nucleotides, at least four nucleotides, at least five nucleotides, or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′ end, 3′ end, or both ends of either an antisense or sense strand of an siRNA.

The terms “blunt” or “blunt ended” as used herein in reference to an siRNA mean that there are no unpaired nucleotides or nucleotide analogs at a given terminal end of an siRNA, i.e., no nucleotide overhang. One or both ends of an siRNA can be blunt. Where both ends of an siRNA are blunt, the siRNA is said to be “blunt ended.” A “blunt ended” siRNA is an siRNA that is blunt at both ends, i.e., has no nucleotide overhang at either end of the molecule. Often such a molecule will be double-stranded over its entire length.

In some embodiments, the present disclosure provides combination therapy to treat HBV that includes an anti-HBV antibody. In certain embodiments, the anti-HBV antibody or an antigen binding fragment thereof binds to the antigenic loop region of HBsAg and neutralizes infection with hepatitis B virus. In certain embodiments, the anti-HBV antibody or an antigen binding fragment thereof binds to the antigenic loop region of HBsAg and neutralizes infection with hepatitis D virus.

As used herein, the term “antibody” encompasses various forms of antibodies including, without being limited to, whole antibodies, antibody fragments, antigen binding fragments, human antibodies, chimeric antibodies, humanized antibodies, recombinant antibodies, and genetically engineered antibodies (variant or mutant antibodies) as long as the characteristic properties of the antibody are retained. In some embodiments, the antibodies are human antibodies and/or monoclonal antibodies. In particular embodiments, the antibodies are human monoclonal antibodies. In certain particular embodiments, the antibodies are recombinant human monoclonal antibodies. As used herein, the terms “antigen binding fragment,” “fragment,” and “antibody fragment” are used interchangeably to refer to any fragment of an antibody of the combination therapy that retains the antigen-binding activity of the antibody. Examples of antibody fragments include, but are not limited to, a single chain antibody, Fab, Fab′, F(ab′)2, Fv, or scFv. Further, the term “antibody” as used herein includes both antibodies and antigen binding fragments thereof.

As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

Human antibodies are well-known in the state of the art (van Dijk M A and van de Winkel J C, Curr. Opin. Chem. Biol. 2001, 5:368-74). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits A et al., Proc. Natl. Acad. Sci. USA 1993, 90:2551-55; Jakobovits A. et al., Nature 1993, 362:255-258; Bruggemann M. et al., Year Immunol. 1993, 7:3340). Human antibodies can also be produced in phage display libraries (Hoogenboom H R and Winter G, Mol. Biol. 1992, 227:381-88; Marks J D et al., Mol Biol. 1991, 222:581-97). The techniques of Cole et al. and Boerner et al. are also available for the preparation of human monoclonal antibodies (Cole et al., Monoclonal Antibodies and Cancer Therapy, Alan R. Liss, p. 77 (1985); Boerner P et al., Immunol. 1991, 147:86-95). In some embodiments, human monoclonal antibodies are prepared by using improved EBV-B cell immortalization as described in Traggiai E et al. (Nat Med. 2004, 10(8):871-5). The term “human antibody” as used herein also comprises such antibodies that are modified, e.g., in the variable region, to generate properties as described herein.

Antibodies of the combination therapy can be of any isotype (e.g., IgA, IgG, IgM, i.e., a κ, γ, or μ heavy chain), but in certain particular embodiments, the antibodies are IgG. Within the IgG isotype, antibodies may be IgG1, IgG2, IgG3, or IgG4 subclass. In particular embodiments, the antibodies are IgG1. Antibodies of the combination therapy may have a κ or a λ light chain. HBsAg-specific antibodies of the IgG-type may advantageously also block the release of HBV and HBsAg from infected cells, based on antigen-independent uptake of IgG through FcRN-IgG receptors into hepatocytes. Therefore, HBsAg-specific antibodies of the IgG-type can bind intracellularly and thereby block the release of HBV virions and HBsAg.

As used herein, the term “variable region” (variable region of a light chain (VL), variable region of a heavy chain (V_{H})) denotes the portion of an antibody light chain (LC) or heavy chain (HC) (typically around the 105-120 amino-terminal amino acids of a mature antibody heavy chain or light chain) that comprises complementarity determining regions (“CDRs”) and framework regions (“FRs”), and that is involved directly in binding the antibody to the antigen. The terms “complementarity determining region,” and “CDR,” are synonymous with “hypervariable region” or “HVR,” and are known in the art to refer to non-contiguous sequences of amino acids within antibody variable regions, which confer antigen specificity and/or binding affinity. In general, there are three CDRs in each variable region of an immunoglobulin binding protein; e.g., for antibodies, the V_{H} and V_{L} regions generally comprise six CDRs (CDRH1, CDRH2, CDRH3; CDRL1, CDRL2, CDRL3). Immunoglobulin sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (Bioinformatics 2016, 15:298-300). It will be understood that in certain embodiments, an antibody or antigen binding fragment of the present disclosure can comprise all or part of a heavy chain (HC), a light chain (LC), or both. For example, a full-length intact IgG antibody monomer typically includes a V_{H}, a CH1, a CH2, a CH3, a V_{L}, and a CL.

In certain embodiments, the anti-HBV antibodies of the combination therapy, according to the present disclosure, or the antigen binding fragment thereof, is a purified antibody, a single chain antibody, a Fab, a Fab′, a F(ab′)2, a Fv, or an scFv. The antibodies of the combination therapy may thus be human antibodies, monoclonal antibodies, human monoclonal antibodies, recombinant antibodies, and/or purified antibodies. The present disclosure also provides fragments of the antibodies, particularly fragments that retain the antigen-binding activity of the antibodies. Such fragments include, but are not limited to, single chain antibodies, Fab, Fab′, F(ab′)2, Fv, or scFv. Although in some places, the present disclosure may refer explicitly to antigen binding fragment(s), antibody fragment(s), variant(s) and/or derivative(s) of antibodies, as used herein the term “antibody” or “antibody of the combination therapy” includes all categories of antibodies, namely, antigen binding fragment(s), antibody fragment(s), variant(s), and derivative(s) of antibodies.

Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. The present disclosure also encompasses single-chain Fv fragments (scFv) derived from the heavy and light chains of an antibody of the disclosure. For example, the disclosure includes a scFv comprising the CDRs from an antibody of the disclosure. Also included are heavy or light chain monomers and dimers, single domain heavy chain antibodies, single domain light chain antibodies, as well as single chain antibodies, e.g., single chain Fv in which the heavy and light chain variable domains are joined by a peptide linker.

Antibody fragments of the present disclosure may impart monovalent or multivalent interactions and be contained in a variety of structures as described above. For instance, scFv molecules may be synthesized to create a trivalent “triabody” or a tetravalent “tetrabody.” The scFv molecules may include a domain of the Fc region resulting in bivalent minibodies. In addition, the sequences of the antibody/antibody fragment may be a component of a multispecific molecule in which the sequences target the epitopes as described herein, and other regions of the multispecific molecule bind to other targets. Exemplary multispecific molecules include, but are not limited to, bispecific Fab2, trispecific Fab3, bispecific scFv, and diabodies (Holliger and Hudson, Nature Biotechnology 2005, 9:1126-36).

Antibodies according to the present disclosure may be provided in purified form. Typically, the antibody will be present in a composition that is substantially free of other polypeptides e.g., where less than 90% (by weight), usually less than 60% and more usually less than 50% of the composition is made up of other polypeptides.

Antibodies and antigen binding fragments of the present disclosure may, in embodiments, be multispecific (e.g., bispecific, trispecific, tetraspecific, or the like), and may be provided in any multispecific format, as disclosed herein. In certain embodiments, an antibody or antigen-binding fragment of the present disclosure is a multispecific antibody, such as a bispecific or trispecific antibody. Formats for bispecific antibodies are disclosed in, for example, Spiess et al. (Mol. Immunol. 2015, 67(2):95), and Brinkmann and Kontermann (mAbs 2017, 9(2):182-212), which bispecific formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-IgGs, IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, and DVI-IgG (four-in-one). A bispecific or multispecific antibody may comprise a HBV- and/or HDV-specific binding domain of the instant disclosure in combination with another such binding domain of the instant disclosure, or in combination with a different binding domain that specifically binds to HBV and/or HDV (e.g., at a same or a different epitope), or with a binding domain that specifically binds to a different antigen.

### II. SIRNA TARGETING HBV

In some embodiments, the present disclosure provides methods of treatment involving administering an siRNA that targets HBV mRNA, and related compositions and kits. As HDV is a defective satellite virus that depends on HBsAg for its life cycle completion, siRNA that reduces HBsAg production interferes with the production of infectious HDV virions. In in vitro experiments, treatment of HBV/HDV coinfected primary human hepatocytes with an siRNA that targets HBV mRNA (SIRNA01) led to a dose-dependent reduction of secreted infectious HDV virions. In HBV/HDV coinfected patients, HBsAg levels correlate with HDV RNA levels, indicating that lowering serum HBsAg may lead to a reduction of circulating HDV (Zachou K et al., HIDT-1 Study Group. Quantitative HBsAg and HDV-RNA levels in chronic delta hepatitis, Liver Int. 2010 March, 30(3):430-7). Further, in preclinical models, lowering intrahepatic HBsAg with siRNA agents decreased HDV viremia (Ye X et al., Hepatitis B Virus Therapeutic Agent ARB-1740 Has Inhibitory Effect on Hepatitis Delta Virus in a New Dually-Infected Humanized Mouse Model, ACS Infect Dis. 2019 May 10, 5(5):738-749) and limited viral spread to uninfected hepatocytes.

In some embodiments described herein, the siRNA that targets HBV mRNA is SIRNA01. SIRNA01 is a synthetic, chemically modified siRNA targeting HBV RNA with a covalently attached triantennary N-acetyl-galactosamine (GalNAc) ligand that allows for specific uptake by hepatocytes. SIRNA01 targets mRNA encoded by a region of the HBV genome that is common to all HBV viral transcripts and is pharmacologically active against HBV genotypes A through J. In preclinical models, SIRNA01 has been shown to inhibit viral replication, translation, and secretion of HBsAg, and may provide or contribute to a functional cure of chronic HBV infections. An SIRNA can have multiple antiviral effects, including degradation of the pgRNA, thus inhibiting viral replication, and degradation of all viral mRNA transcripts, thereby preventing expression of viral proteins. This may result in the return of a functional immune response directed against HBV, either alone or in combination with other therapies. The ability of SIRNA01 to reduce HBsAg-containing noninfectious subviral particles also distinguishes it from currently available treatments.

SIRNA01 targets and inhibits expression of an mRNA encoded by an HBV genome according to NCBI Reference Sequence NC_003977.2 (GenBank Accession No. GI:21326584) (SEQ ID NO:1). More specifically, SIRNA01 targets an mRNA encoded by a portion of the HBV genome comprising the sequence GTGTGCACTTCGCTTCAC (SEQ ID NO:2), which corresponds to nucleotides 1579-1597 of SEQ ID NO:1. Because transcription of the HBV genome results in polycistronic, overlapping RNAs, SIRNA01 results in significant inhibition of expression of most or all HBV transcripts. Exemplary methods for synthesizing SIRNA01, and experimental data demonstrating silencing of HBV gene expression, are described in International Application Publication No. WO 2020/036862A1, which methods and data are incorporated herein by reference.

SIRNA01 has a sense strand comprising 5′-GUGUGCACUUCGCUUCACA-3′ (SEQ ID NO:3) and an antisense strand comprising 5′-UGUGAAGCGAAGUGCACACUU-3′ (SEQ ID NO:4), wherein the nucleotides include 2′-fluoro (2′F) and 2′-O-methoxy (2′OMe) ribose sugar modifications, phosphorothioate backbone modifications, a glycol nucleic acid (GNA) modification, and conjugation to a triantennary N-acetyl-galactosamine (GalNAc) ligand at the 3′ end of the sense strand, to facilitate delivery to hepatocytes through the asialoglycoprotein receptor (ASGPR). Including modifications, the sense strand of SIRNA01 comprises 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO: 6), wherein the modifications are abbreviated as shown in Table 1.

TABLE 1
Abbreviations of nucleotide monomers used in modified nucleic
acid sequence representation. It will be understood that, unless
otherwise indicated, these monomers, when present in an oligonucleotide,
are mutually linked by 5′-3′-phosphodiester bonds.

| Abbreviation | Nucleotide(s) |
|---|---|
| A | adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | uridine-3′-phosphate |
| Uf | 2′-fluorouridine-3′-phosphate |
| Ufs | 2′-fluorouridine-3′-phosphorothioate |
| Us | uridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | (“Hyp-(GalNAc-alkyl)3”) |
| (Agn) | adenosine-glycol nucleic acid (GNA) |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |

In some embodiments, the siRNA used in the methods, compositions, or kits described herein is SIRNA01. In some embodiments, the siRNA used in the methods, compositions, or kits described herein comprises a sequence variant of SIRNA01. In particular embodiments, the portion of the HBV transcript(s) targeted by the sequence variant of SIRNA01overlaps with the portion of the HBV transcript(s) targeted by SIRNA01.

In some embodiments, the siRNA comprises a sense strand and an antisense strand, wherein (1) the sense strand comprises SEQ ID NO:3 or SEQ ID NO:5, or a sequence that differs by not more than 4, not more than 3, not more than 2, or not more than 1 nucleotide from SEQ ID NO:3 or SEQ ID NO:5, respectively; or (2) the antisense strand comprises SEQ ID NO:4 or SEQ ID NO:6, or a sequence that differs by not more than 4, not more than 3, not more than 2, or not more than 1 nucleotide from SEQ ID NO:4 or SEQ ID NO:6, respectively.

In some embodiments, shorter duplexes having one of the sequences of SEQ ID NO:4 or SEQ ID NO:6 minus only a few nucleotides on one or both ends are used. Hence, siRNAs having a partial sequence of at least 15, 16, 17, 18, 19, 20, or more contiguous nucleotides from one or both of SEQ ID NO:4 and SEQ ID NO:6, and differing in their ability to inhibit the expression of an HBV gene by not more than 5, 10, 15, 20, 25, or 30% inhibition from an siRNA comprising the full sequence, are contemplated herein. In some embodiments, an siRNA having a blunt end at one or both ends, formed by removing nucleotides from one or both ends of SIRNA01, is provided.

In some embodiments, the siRNA comprises a sense strand and an antisense strand, wherein (1) the sense strand comprises SEQ ID NO:7, or a sequence that differs by not more than 4, not more than 3, not more than 2, or not more than 1 nucleotide from SEQ ID NO:7, respectively; or (2) the antisense strand comprises SEQ ID NO:8, or a sequence that differs by not more than 4, not more than 3, not more than 2, or not more than 1 nucleotide from SEQ ID NO:8, respectively.

In some embodiments, shorter duplexes having the sequence of SEQ ID NO:8 minus only a few nucleotides on one or both ends are used. Hence, siRNAs having a partial sequence of at least 15, 16, 17, 18, 19, 20, or more contiguous nucleotides from SEQ ID NO:8, and differing in their ability to inhibit the expression of an HBV gene by not more than 5, 10, 15, 20, 25, or 30% inhibition from an siRNA comprising the full sequence, are contemplated herein. In some embodiments, an siRNA having a blunt end at one or both ends, formed by removing nucleotides from one or both ends of SEQ ID NO:8, is provided.

In some embodiments, an siRNA as described herein can contain one or more mismatches to the target sequence. In some embodiments, an siRNA as described herein contains no more than 3 mismatches. In some embodiments, if the antisense strand of the siRNA contains mismatches to a target sequence, the area of mismatch is not located in the center of the region of complementarity. In particular embodiments, if the antisense strand contains mismatches to the target sequence, the mismatch is restricted to within the last 5 nucleotides from either the 5′ or 3′ end of the region of complementarity. For example, for a 23 nucleotide siRNA strand that is complementary to a region of an HBV gene, the RNA strand may not contain any mismatch within the central 13 nucleotides. The methods described herein or methods known in the art can be used to determine whether an siRNA containing a mismatch to a target sequence is effective in inhibiting the expression of an HBV gene.

In some embodiments, the siRNA used in the methods, compositions, and kits described herein include two oligonucleotides, where one oligonucleotide is described as the sense strand, and the second oligonucleotide is described as the corresponding antisense strand of the sense strand. As described herein and as known in the art, the complementary sequences of an siRNA can also be contained as self-complementary regions of a single nucleic acid molecule, as opposed to being on separate oligonucleotides.

In some embodiments, a single-stranded antisense RNA molecule comprising the antisense strand of siRNAs described herein is used in the methods, compositions, and kits described herein. The antisense RNA molecule can have 15-30 nucleotides complementary to the target.

In some embodiments, a single-stranded antisense RNA molecule comprising the antisense strand of SIRNA01 or sequence variant thereof is used in the methods, compositions, and kits described herein. The antisense RNA molecule can have 15-30 nucleotides complementary to the target. For example, the antisense RNA molecule may have a sequence of at least 15, 16, 17, 18, 19, 20, 21, or more contiguous nucleotides from SEQ ID NO:4 or SEQ ID NO:6.

In some embodiments, the siRNA comprises a sense strand and an antisense strand, wherein the sense strand comprises SEQ ID NO:5 and the antisense strand comprises SEQ ID NO:6, and further comprises additional nucleotides, modifications, or conjugates as described herein. For example, in some embodiments, the siRNA can include further modifications in addition to those indicated in SEQ ID NOs: 5 and 6. Such modifications can be generated using methods established in the art, such as those described in “Current protocols in nucleic acid chemistry,” Beaucage S L et al. (Edrs.), John Wiley & Sons, Inc., New York, NY, USA, which methods are incorporated herein by reference. Examples of such modifications are described in more detail below.

In some embodiments, substantially all or all of the nucleotides of the sense strand of the siRNA and substantially all or all of the nucleotides of the antisense strand are modified nucleotides. The nucleotides may be modified as described below.

a. Modified siRNAs

Modifications disclosed herein include, for example, (a) sugar modifications (e.g., at the 2′ position or 4′ position) or replacement of the sugar; (b) backbone modifications, including modification or replacement of the phosphodiester linkages; (c) base modifications, e.g., replacement with stabilizing bases, destabilizing bases, or bases that base pair with an expanded repertoire of partners, removal of bases (abasic nucleotides), or conjugated bases; and (d) end modifications, e.g., 5′ end modifications (phosphorylation, conjugation, inverted linkages, etc.), 3′ end modifications (conjugation, DNA nucleotides, inverted linkages, etc.). Some specific examples of modifications that can be incorporated into siRNAs of the present application are shown in Table 1.

Modifications include substituted sugar moieties. The siRNAs featured herein can include one of the following at the 2′ position: OH; F; O-, S-, or N-alkyl; O-, S-, or N-alkenyl; O-, S- or N-alkynyl; or O-alkyl-O-alkyl; wherein the alkyl, alkenyl, and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)·ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In some other embodiments, siRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an siRNA, or a group for improving the pharmacodynamic properties of an siRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta 1995, 78:486-504), i.e., an alkoxy-alkoxy group. Another exemplary modification is 2′-dimethylaminooxyethoxy, i.e., a O(CH₂)₂ON(CH₃)₂ group, also known as 2′-DMAOE, and 2′-dimethylaminoethoxyethoxy (also known in the art as 2*-O-dimethylaminoethoxyethyl or 2*-DMAEOE), i.e., 2*-O—CH₂—O—CH₂—N(CH₂)₂. Other exemplary modifications include 2′-methoxy (2′-OCH₃), 2′-aminopropoxy (2-OCH₂CH₂CH₂NH₂), and 2′-fluoro (2′-F). Similar modifications can also be made at other positions on the RNA of an siRNA, particularly the 3′ position of the sugar on the 3′ terminal nucleotide or in 2′-5′ linked siRNAs and the 5′ position of the 5′ terminal nucleotide. Modifications can also include sugar mimetics, such as cyclobutyl moieties, in place of the pentofuranosyl sugar.

Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920; each of which is incorporated herein by reference for teachings relevant to methods of preparing such modifications.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6, 239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464; each of which is herein incorporated herein by reference for teachings relevant to methods of preparing such modifications.

RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439; each of which is herein incorporated by reference for teachings relevant to methods of preparing such modifications.

In some embodiments, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262; each of which is incorporated herein by reference for teachings related to such methods of preparation. Further teaching of PNA compounds can be found, for example, in Nielsen et al. (Science 1991, 254:1497-1500).

Some embodiments featured in the technology described herein include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂—, and —N(CH₃)—CH₂—CH₂— [wherein the native phosphodiester backbone is represented as —O—P—O—CH₂-] of U.S. Pat. No. 5,489,677, and the amide backbones of U.S. Pat. No. 5,602,240. In some embodiments, the RNAs featured herein have morpholino backbone structures of U.S. Pat. No. 5,034,506.

Modifications of siRNAs disclosed herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl, and other 5-substituted uracils and cytosines, 7-methylguanine and 7-methyladenine, 8-azaguanine and 8-azaadenine, 7-deazaguanine and 7-daazaadenine, and 3-deazaguanine and 3-deazaadenine. Further nucleobases include those disclosed in U.S. Pat. No. 3,687,808, those disclosed in Modified Nucleosides in Biochemistry, Biotechnology and Medicine (Herdewijn P, ed., Wiley-VCH, 2008); those disclosed in The Concise Encyclopedia Of Polymer Science And Engineering (pages 858-859, Kroschwitz J L, ed., John Wiley & Sons, 1990), those disclosed by Englisch et al. (Angewandte Chemie, International Edition, 30, 613, 1991), and those disclosed by Sanghvi Y S (Chapter 15, dsRNA Research and Applications, pages 289-302, Crooke S T and Lebleu B, ed., CRC Press, 1993). Certain of these nucleobases are particularly useful for increasing the binding affinity of the oligomeric compounds featured in the technology described herein. These include 5-substituted pyrimidines, 6-azapyrimidines and N-2, N-6, and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi Y S et al., Eds., dsRNA Research and Applications, CRC Press, Boca Raton, pp. 276-278, 1993) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, U.S. Pat. Nos. 3,687,808; 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088; each of which is incorporated herein by reference for teachings relevant to methods of preparing such modifications.

siRNAs can also be modified to include one or more glycol nucleic acid, such as adenosine-glycol nucleic acid (GNA). A description of adenosine-GNA can be found, for example, in Zhang et al. (JACS 2005, 127(12):4174-75) which is incorporated herein by reference for teachings relevant to methods of preparing GNA modifications.

The RNA of an siRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen J et al., Nucleic Acids Research 2005, 33(1):439-47; Mook O R et al., Mol Cane Ther 2007, 6(3):833-43; Grunweller A et al., Nucleic Acids Research 2003, 31(12):3185-93).

Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845; each of which is incorporated herein by reference for teachings relevant to methods of preparing such modifications.

In some embodiments, the siRNA includes modifications involving chemically linking to the RNA one or more ligands, moieties, or conjugates that enhance the activity, cellular distribution, or cellular uptake of the siRNA. Such moieties include but are not limited to lipid moieties such as a cholesterol moiety (Letsinger et al., Proc. Natl. Acid. Sci. USA 1989, 86:6553-56), cholic acid (Manoharan et al., Biorg. Med. Chem. Let. 1990, 4:1053-60), a thioether, e.g., beryl-S-tritylthiol (Manoharan et al., Ann. N.Y. Acad. Sci. 1992, 660:306-9); Manoharan et al., Biorg. Med. Chem. Let. 1993, 3:2765-70), a thiocholesterol (Oberhauser et al., Nucl. Acids Res. 1992, 20:533-38), an aliphatic chain, e.g., dodecandiol or undecyl residues (Saison-Behmoaras et al., EMBO J 1991, 10:1111-18; Kabanov et al., FEBS Lett. 1990, 259:327-30; Svinarchuk et al., Biochimie 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett. 1995, 36:3651-54; Shea et al., Nucl. Acids Res. 1990, 18:3777-83), a polyamine or a polyethylene glycol chain (Manoharan et al., Nucleosides & Nucleotides 1995, 14:969-73), or adamantane acetic acid (Manoharan et al., Tetrahedron Lett. 1995, 36:3651-54), a palmityl moiety (Mishra et al., Biochim. Biophys. Acta 1995, 1264:229-37), or an octadecylamine or hexylamino-carbonyloxycholesterol moiety (Crooke et al., J. Pharmacol. Exp. Ther. 1996, 277:923-37).

In some embodiments, a ligand alters the distribution, targeting, or lifetime of an siRNA into which it is incorporated. In some embodiments, a ligand provides an enhanced affinity for a selected target, e.g., molecule, cell, or cell type, compartment, e.g., a cellular or organ compartment, tissue, organ, or region of the body, as, e.g., compared to a species absent such a ligand. In such embodiments, the ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Examples of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, and alpha helical peptide.

Ligands can also include targeting groups, e.g., a cell or tissue targeting agent, e.g., a lectin, glycoprotein, lipid or protein, e.g., an antibody, that binds to a specified cell type such as a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. Other examples of ligands include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules (e.g., cholesterol, cholic acid, adamantane acetic acid, 1-pyrene butyric acid, dihydrotestosterone, 1,3-Bis-0(hexadecyl)glycerol, geranyloxyhexyl group, hexadecylglycerol, borneol, menthol, 1,3-propanediol, heptadecyl group, palmitic acid, myristic acid,03-(oleoyl)lithocholic acid, 03-(oleoyl)cholenic acid, dimethoxytrityl, or phenoxazine), peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, and AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, and multivalent fucose. The ligand can be, for example, a lipopolysaccharide, an activator of p38 MAP kinase, or an activator of NF-KB.

The ligand can be a substance, e.g., a drug, which can increase the uptake of the siRNA into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, the ligand is a moiety, e.g., a vitamin, which is taken up by a target cell, e.g., a liver cell. Exemplary vitamins include vitamin A, E, and K. Other exemplary vitamins include are B vitamin, e.g., folic acid, B12, riboflavin, biotin, pyridoxal, or other vitamins or nutrients taken up by target cells such as liver cells. Also included are HSA and low density lipoprotein (LDL).

In some embodiments, a ligand attached to an siRNA as described herein acts as a pharmacokinetic (PK) modulator. As used herein, a “PK modulator” refers to a pharmacokinetic modulator. PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin, etc. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the technology described herein as ligands (e.g., as PK modulating ligands). In addition, aptamers that bind serum components (e.g., serum proteins) are also suitable for use as PK modulating ligands in the embodiments described herein.

(i) Lipid conjugates. In some embodiments, the ligand or conjugate is a lipid or lipid-based molecule. A lipid or lipid-based ligand can (a) increase resistance to degradation of the conjugate, (b) increase targeting or transport into a target cell or cell membrane, and/or (c) can be used to adjust binding to a serum protein, e.g., HSA. Such a lipid or lipid-based molecule may bind a serum protein, e.g., human serum albumin (HSA). An HSA-binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In some embodiments, the lipid based ligand binds HSA. The lipid based ligand may bind to HSA with a sufficient affinity such that the conjugate will be distributed to a non-kidney tissue. In certain particular embodiments, the HSA-ligand binding is reversible.

In some embodiments, the lipid based ligand binds HSA weakly or not at all, such that the conjugate will be distributed to the kidney. Other moieties that target to kidney cells can also be used in place of or in addition to the lipid based ligand.

(ii) Cell Permeation Peptide and Agents. In another aspect, the ligand is a cell-permeation agent, such as a helical cell-permeation agent. In some embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In some embodiments, the helical agent is an alpha-helical agent. In certain particular embodiments, the helical agent has a lipophilic and a lipophobic phase.

A “cell permeation peptide” is capable of permeating a cell, e.g., a microbial cell, such as a bacterial or fungal cell, or a mammalian cell, such as a human cell. A microbial cell-permeating peptide can be, for example, an alpha-helical linear peptide (e.g., LL-37 or Ceropin PI), a disulfide bond-containing peptide (e.g., a-defensin, β--defensin, or bactenecin), or a peptide containing only one or two dominating amino acids (e.g., PR-39 or indolicidin).

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to siRNA can affect pharmacokinetic distribution of the RNAi, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF, which has the amino acid sequence AAVALLPAVLLALLAP (SEQ ID NO:9). An RFGF analogue (e.g., amino acid sequence AALLPVLLAAP (SEQ ID NO:10) containing a hydrophobic MTS can also be a targeting moiety. The peptide moiety can be a “delivery” peptide, which can carry large polar molecules including peptides, oligonucleotides, and proteins across cell membranes. For example, sequences from the HIV Tat protein (GRKKRRQRRRPPQ (SEQ ID NO:11) and the Drosophila Antennapedia protein (RQIKIWFQNRRMKWK (SEQ ID NO:12) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature 1991, 354:82-84).

A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 1993, 31:2717-24).

(iii) Carbohydrate Conjugates. In some embodiments, the siRNA oligonucleotides described herein further comprise carbohydrate conjugates. The carbohydrate conjugates may be advantageous for the in vivo delivery of nucleic acids, as well as compositions suitable for in vivo therapeutic use. As used herein, “carbohydrate” refers to a compound which is either a carbohydrate per se made up of one or more monosaccharide units having at least 6 carbon atoms (which can be linear, branched, or cyclic) with an oxygen, nitrogen, or sulfur atom bonded to each carbon atom; or a compound having as a part thereof a carbohydrate moiety made up of one or more monosaccharide units each having at least six carbon atoms (which can be linear, branched, or cyclic), with an oxygen, nitrogen, or sulfur atom bonded to each carbon atom. Representative carbohydrates include the sugars (mono-, di-, tri-, and oligosaccharides containing from about 4-9 monosaccharide units), and polysaccharides such as starches, glycogen, cellulose, and polysaccharide gums. Specific monosaccharides include C5 and above (in some embodiments, C5-C8) sugars; and di- and trisaccharides include sugars having two or three monosaccharide units (in some embodiments, C5-C8).

In some embodiments, the carbohydrate conjugate is selected from the group consisting of:

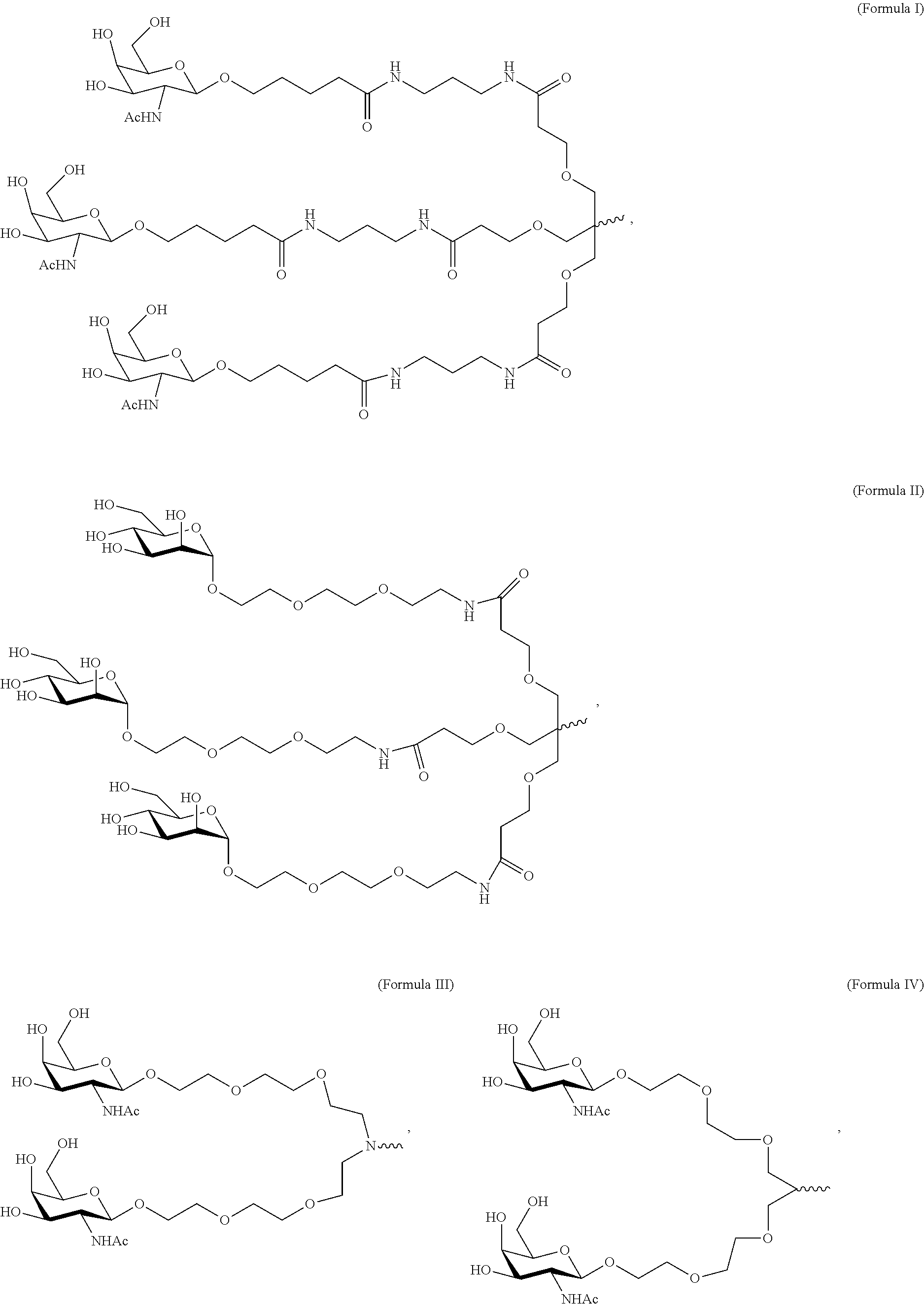

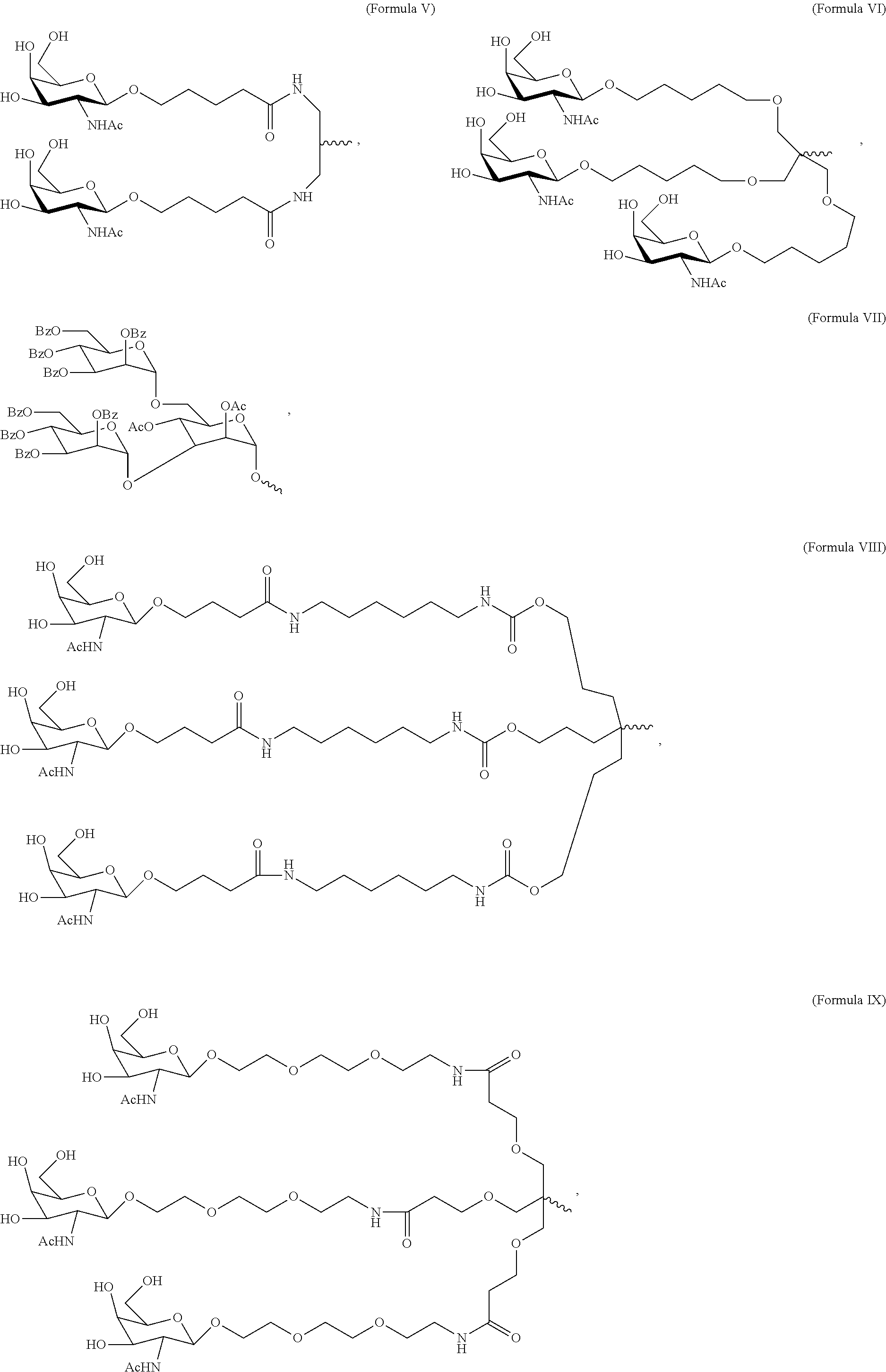

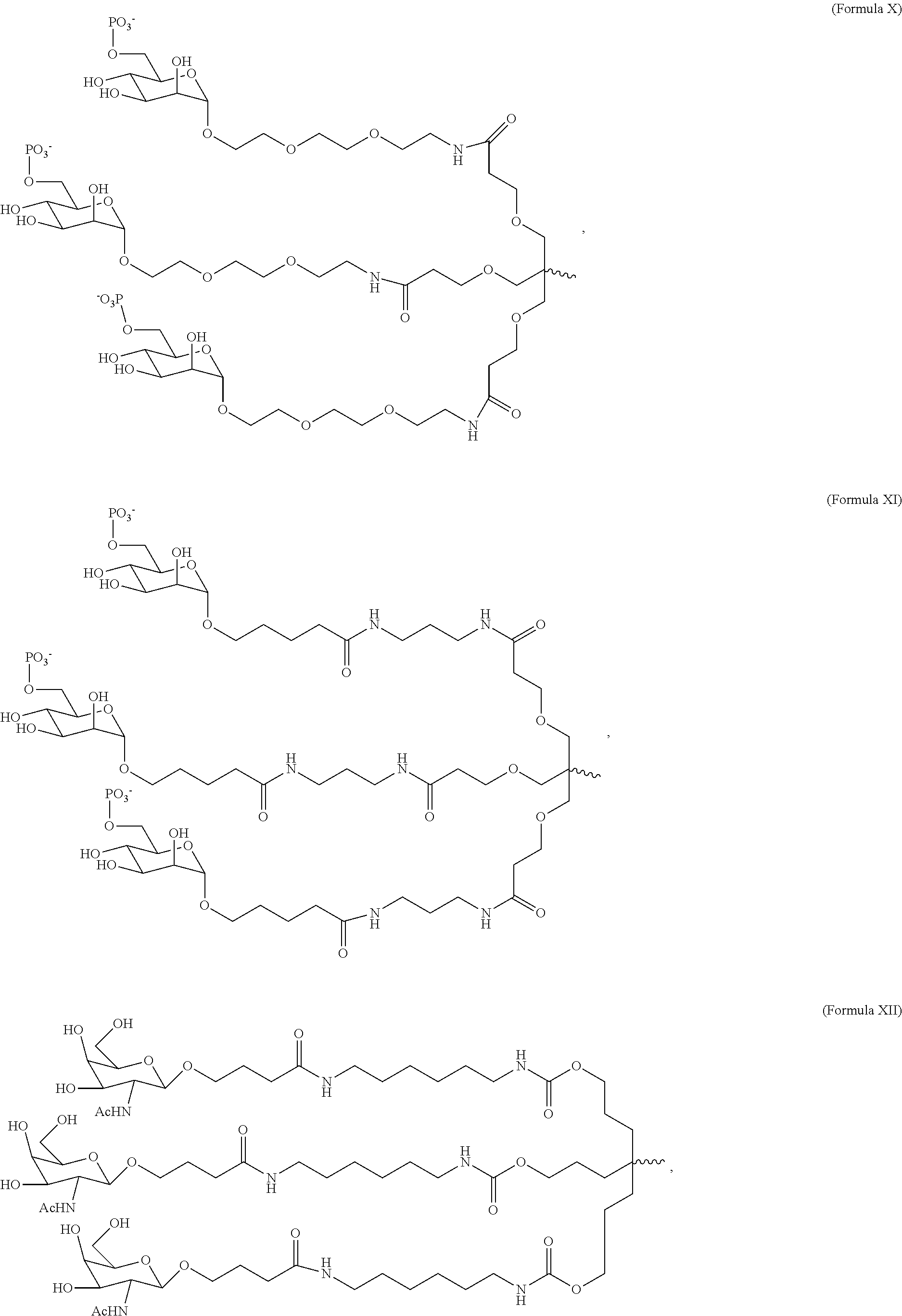

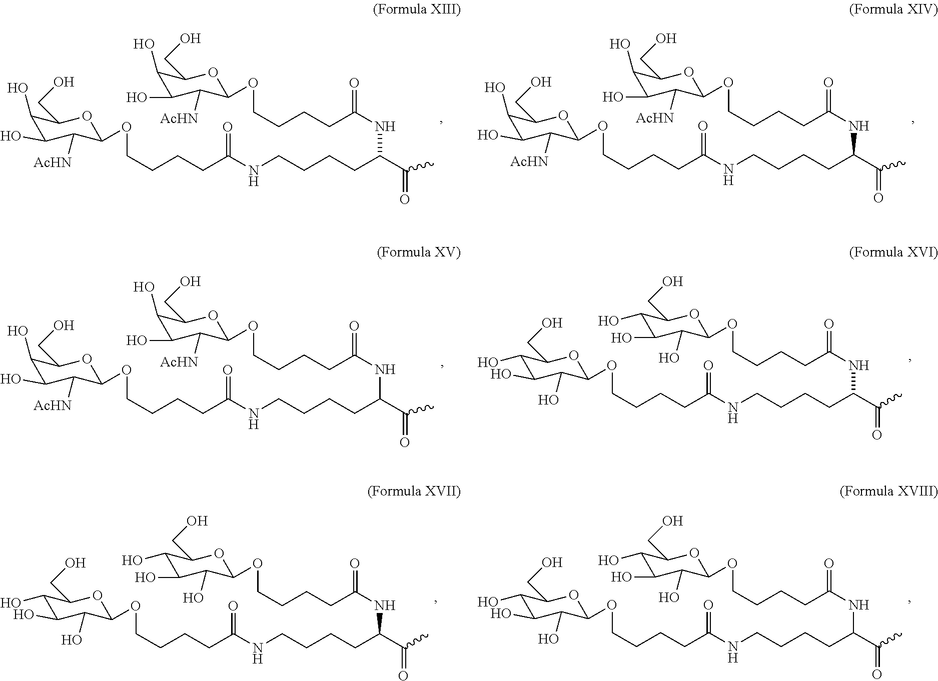

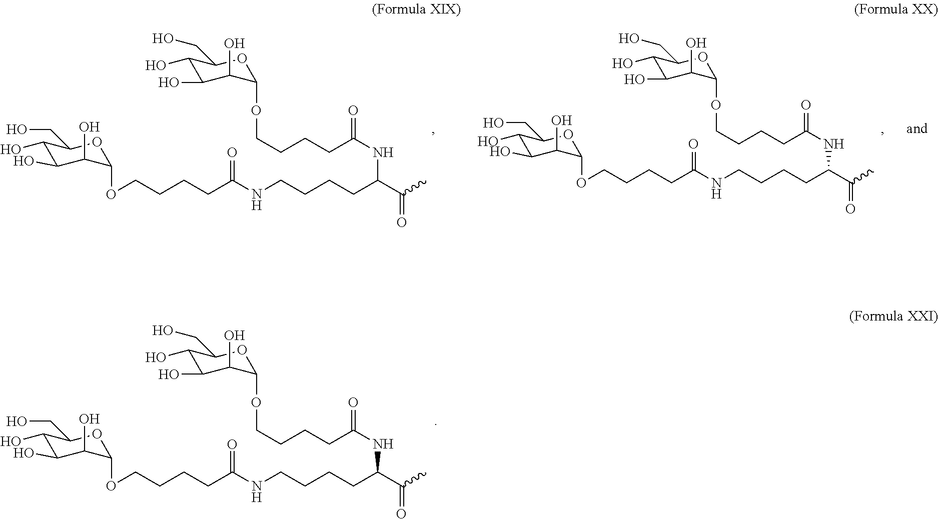

Another representative carbohydrate conjugate for use in the embodiments described herein includes

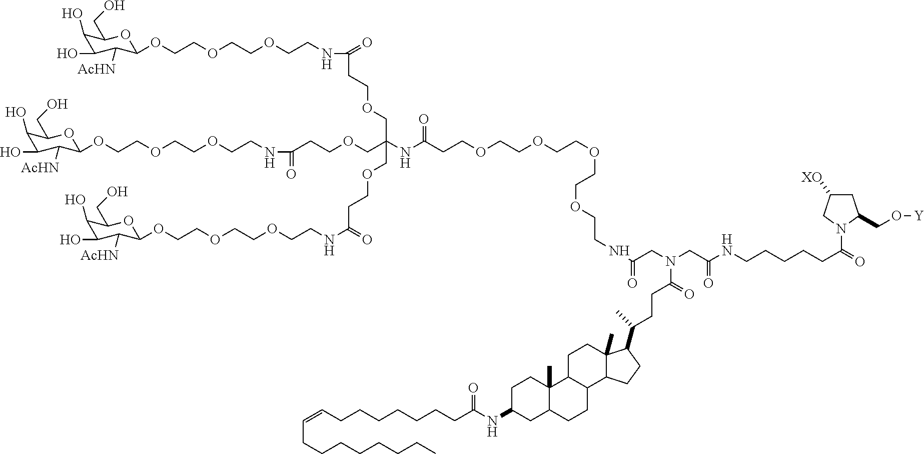

(Formula XXII), wherein when one of X or Y is an oligonucleotide, the other is a hydrogen.

In some embodiments, the carbohydrate conjugate further comprises another ligand such as, but not limited to, a PK modulator, an endosomolytic ligand, or a cell permeation peptide.

(iv) Linkers. In some embodiments, the conjugates described herein can be attached to the siRNA oligonucleotide with various linkers that can be cleavable or non-cleavable.

The term “linker” or “linking group” means an organic moiety that connects two parts of a compound. Linkers typically comprise a direct bond or an atom such as oxygen or sulfur, a unit such as NR8, C(O), C(O)NH, SO, SO2, SO2NH, or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, and alkynylhereroaryl, which one or more methylenes can be interrupted or terminated by O, S, S(O), SO2, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In certain embodiments, the linker is between 1-24 atoms, between 4-24 atoms, between 6-18 atoms, between 8-18 atoms, or between 8-16 atoms.

A cleavable linking group is one which is sufficiently stable outside the cell, but which upon entry into a target cell is cleaved to release the two parts the linker is holding together. In certain embodiments, the cleavable linking group is cleaved at least 10 times, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases. A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a particular pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver-targeting ligands can be linked to the cationic lipids through a linker that includes an ester group. Liver cells are rich in esterases, and therefore the linker will be cleaved more efficiently in liver cells than in cell types that are not esterase-rich. Other cell types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It can be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell-free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least 2, at least 4, at least 10 or at least 100 times faster in the cell (or under in vitro conditions selected to mimic intracellular conditions) as compared to blood or serum (or under in vitro conditions selected to mimic extracellular conditions).

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular RNAi moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In some embodiments, candidate compounds are cleaved by at most 10% in the blood. In certain embodiments, useful candidate compounds are degraded at least 2, at least 4, at least 10, or at least 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. In certain embodiments, the phosphate-based linking groups are selected from: —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—. In particular embodiments, the phosphate-linking group is —O—P(O)(OH)—O—. These candidates can be evaluated using methods analogous to those described above.

Acid cleavable linking groups are linking groups that are cleaved under acidic conditions. In some embodiments, acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes, can provide a cleaving environment for acid cleavable linking groups. Examples of acid cleavable linking groups include but are not limited to hydrazones, esters, and esters of amino acids. Acid cleavable groups can have the general formula —C═N—, C(O)O, or —OC(O). In some embodiments, the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups are cleaved by enzymes such as esterases and amidases in cells. Examples of ester-based cleavable linking groups include but are not limited to esters of alkylene, alkenylene, and alkynylene groups. Ester cleavable linking groups have the general formula —C(O)O—, or —OC(O)—. These candidates can be evaluated using methods analogous to those described above.

Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides, etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

Representative carbohydrate conjugates with linkers include, but are not limited to,

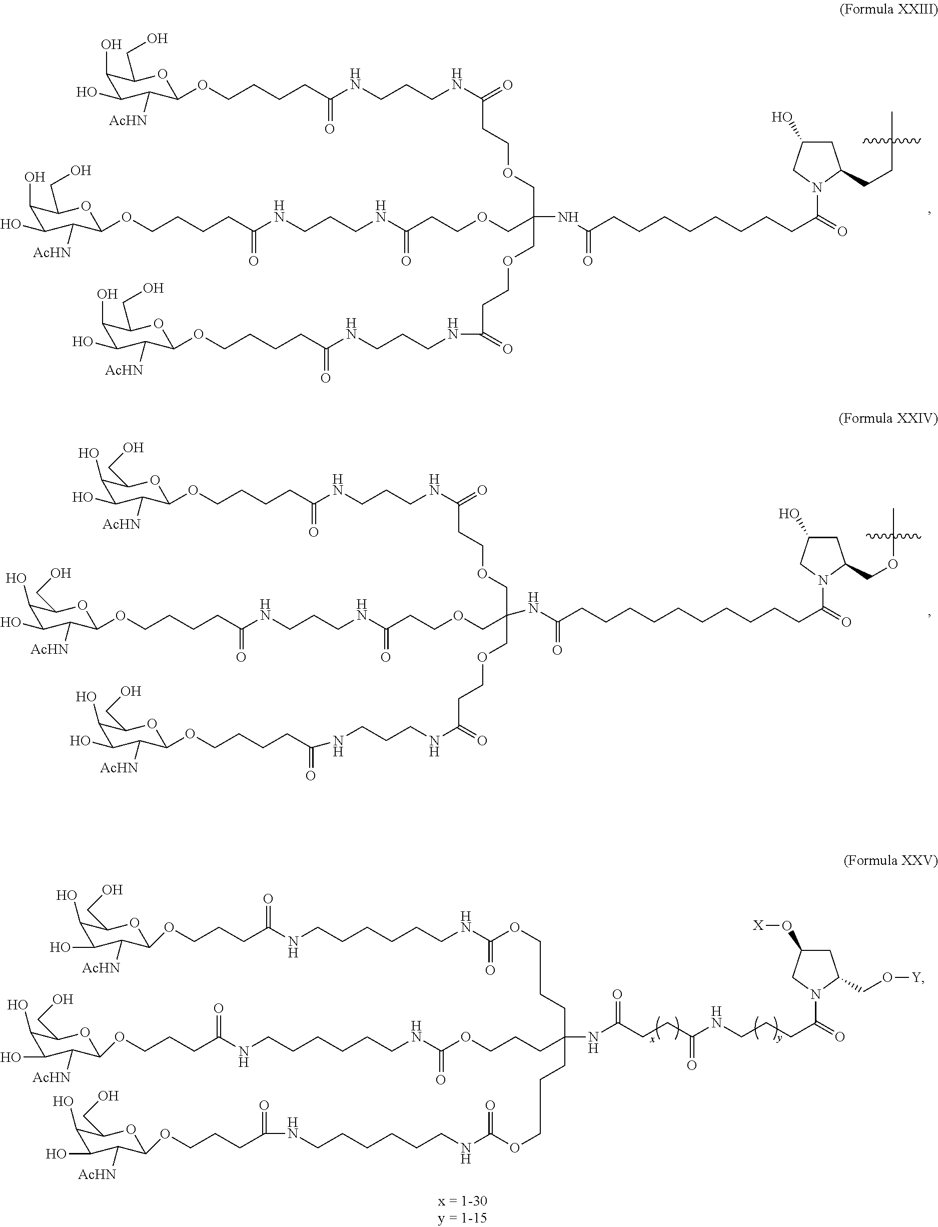

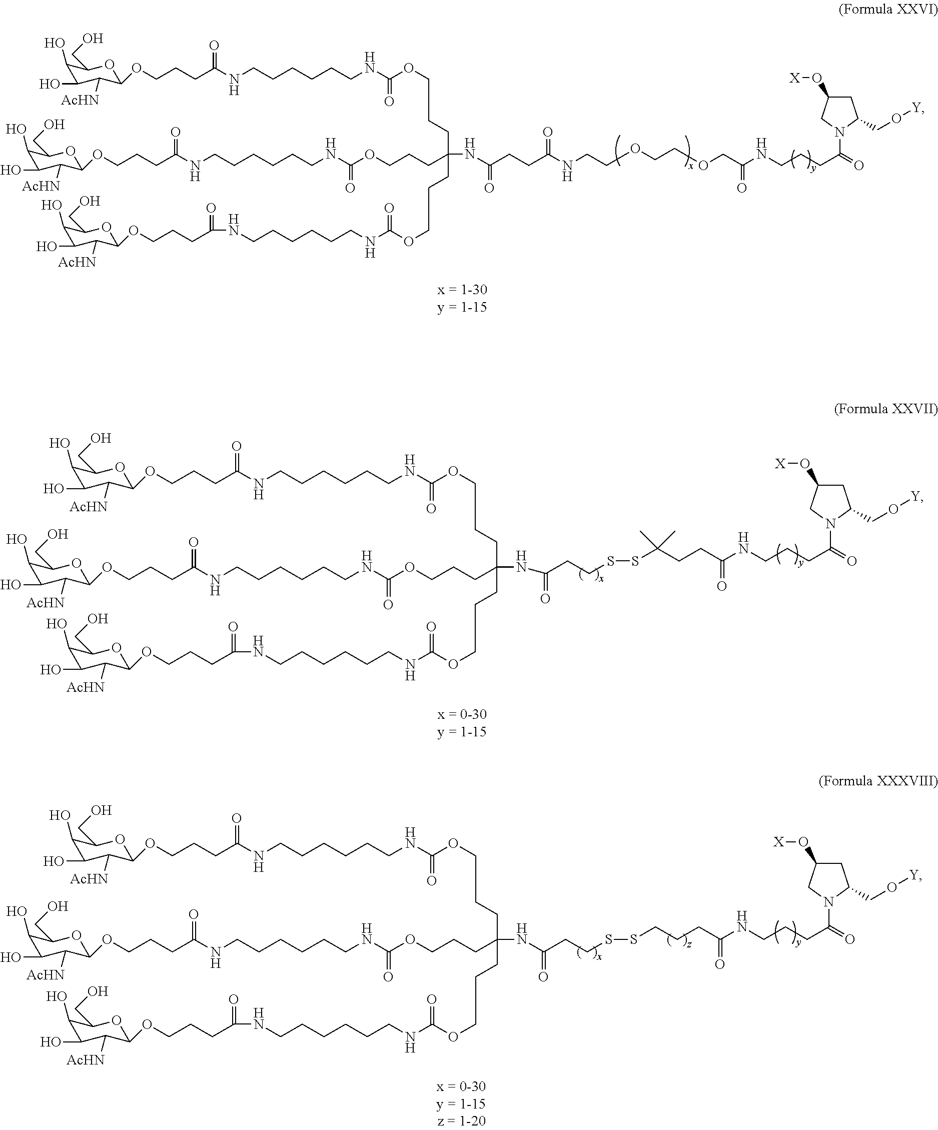

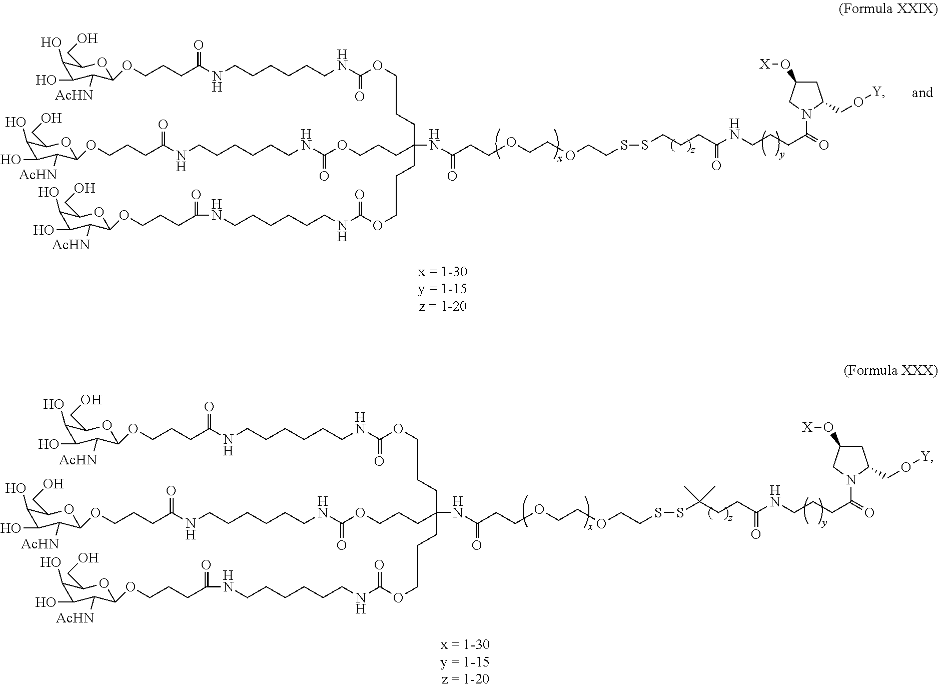

wherein when one of X or Y is an oligonucleotide, the other is a hydrogen.

In certain embodiments of the compositions and methods described herein, a ligand is one or more “GalNAc” (N-acetylgalactosamine) derivatives attached through a bivalent or trivalent branched linker. For example, in some embodiments the siRNA is conjugated to a GalNAc ligand as shown in the following schematic.

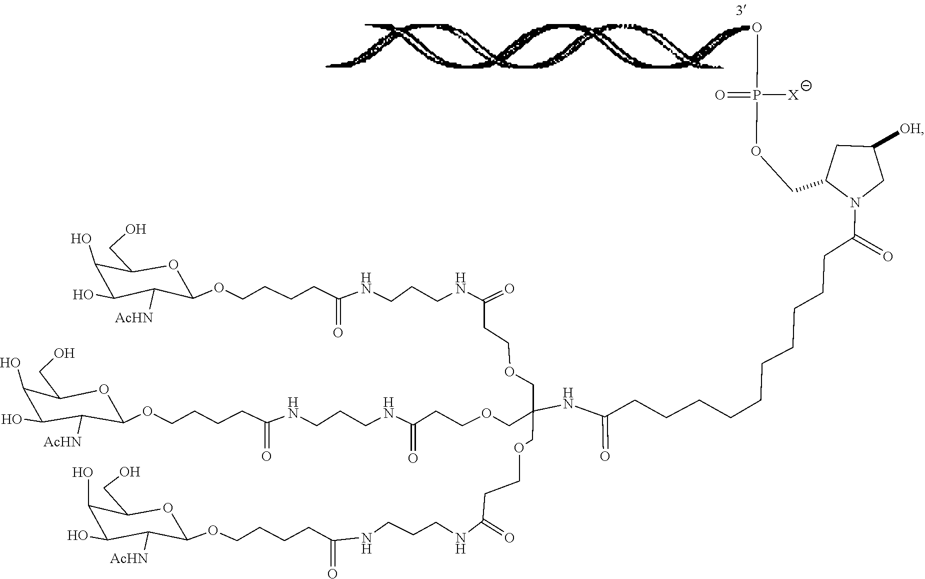

wherein X is O or S. In some of these embodiments, X is O.

In some embodiments, the combination therapy includes an siRNA that is conjugated to a bivalent or trivalent branched linker selected from the group of structures shown in any of formula (XXXI)-(XXXIV):

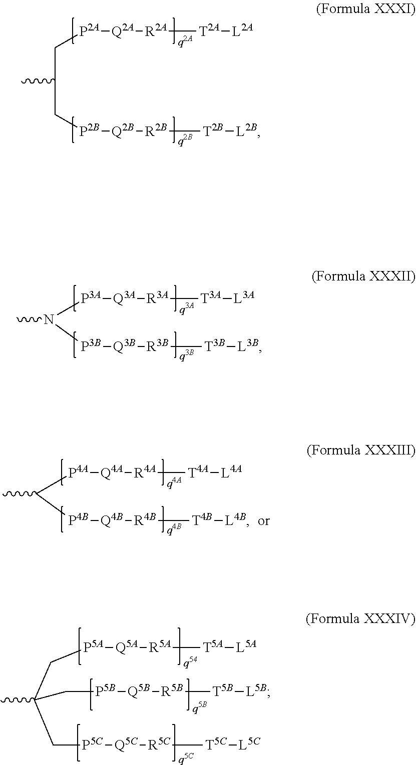

wherein:
q2A, q2B, q3A, q3B, q4A, q4B, q5A, q5B, and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, and T^{5C} are each independently for each occurrence absent, CO, NH, O, S, OC(O), NHC(O), CH₂, CH₂NH, or CH₂O;
Q^{2A}, Q^{2B}, Q^{3A}, Q^{3B}, Q^{4A}, Q^{4B}, Q^{5A}, Q^{5B}, and Q^{5C} are independently for each occurrence absent, alkylene, or substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, and R^{5C} are each independently for each occurrence absent, NH, O, S, CH₂, C(O)O, C(O)NH, NHCH(R^{a})C(O), —C(O)—CH(R^{a})—NH—, CO, CH═N—O,

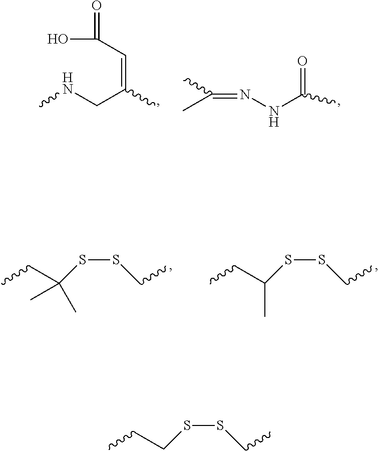

or heterocyclyl;
L^{2A}, L^{2B}, L^{3A}, L^{3B}, L^{4A}, L^{4B}, L^{5A}, L^{5B}, and L^{5C} represent the ligand; i.e., each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with siRNAs for inhibiting the expression of a target gene, such as those of formula (XXXV):

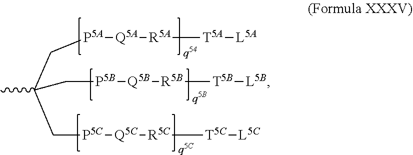

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas I, VI, X, IX, and XII.

Representative U.S. patents that teach the preparation of RNA conjugates include U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; and 7,037,646; each of which is incorporated herein by reference for the teachings relevant to such methods of preparation.

In certain instances, the RNA of an siRNA can be modified by a non-ligand group. A number of non-ligand molecules have been conjugated to siRNAs in order to enhance the activity, cellular distribution or cellular uptake of the siRNAs, and procedures for performing such conjugations are available in the scientific literature. Such non-ligand moieties have included lipid moieties, such as cholesterol (Kubo T et al., Biochem. Biophys. Res. Comm. 2007, 365(1):54-61; Letsinger et al., Proc. Natl. Acad. Sci. USA 1989, 86:6553), cholic acid (Manoharan et al., Bioorg. Med. Chem. Lett. 1994, 4:1053), a thioether, e.g., hexyl-S-tritylthiol (Manoharan et al., Ann. N.Y. Acad. Sci. 1992, 660:306; Manoharan et al., Bioorg. Med. Chem. Let. 1993, 3:2765), a thiocholesterol (Oberhauser et al., Nucl. Acids Res. 1992, 20:533), an aliphatic chain, e.g., dodecandiol or undecyl residues (Saison-Behmoaras et al., EMBO J. 1991, 10:111; Kabanov et al., FEBS Lett. 1990, 259:327; Svinarchuk et al., Biochimie 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36:3651; Shea et al., Nucl. Acids Res. 1990, 18:3777), a polyamine or a polyethylene glycol chain (Manoharan et al., Nucleosides & Nucleotides 1995, 14:969), or adamantane acetic acid (Manoharan et al., Tetrahedron Lett. 1995, 36:3651), a palmityl moiety (Mishra et al., Biochim. Biophys. Acta 1995, 1264:229), or an octadecylamine or hexylamino-carbonyl-oxycholesterol moiety (Crooke et al., J. Pharmacol. Exp. Ther. 1996, 277:923).

Typical conjugation protocols involve the synthesis of an RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

B. Pharmaceutical Compositions and Delivery of siRNA

In some embodiments, pharmaceutical compositions containing an siRNA, as described herein, and a pharmaceutically acceptable carrier or excipient are provided. The pharmaceutical composition containing the siRNA can be used to treat HBV infection. Such pharmaceutical compositions are typically formulated based on the mode of delivery. For example, compositions may be formulated for systemic administration via parenteral delivery, e.g., by subcutaneous (SC) delivery.

A “pharmaceutically acceptable carrier” or “excipient” is a pharmaceutically acceptable solvent, suspending agent, or any other pharmacologically inert vehicle for delivering one or more agents, such as nucleic acids, to an animal. The excipient can be liquid or solid and is selected, with the planned manner of administration in mind, so as to provide for the desired bulk, consistency, etc., when combined with the agent (e.g., a nucleic acid) and the other components of a given pharmaceutical composition. Typical pharmaceutically acceptable carriers or excipients include, but are not limited to, binding agents (e.g., pregelatinized maize starch, polyvinylpyrrolidone, hydroxypropyl methylcellulose); fillers (e.g., lactose and other sugars, microcrystalline cellulose, pectin, gelatin, calcium sulfate, ethyl cellulose, polyacrylates, calcium hydrogen phosphate); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate); disintegrants (e.g., starch, sodium starch glycolate); and wetting agents (e.g., sodium lauryl sulphate).

Pharmaceutically acceptable organic or inorganic excipients suitable for non-parenteral administration that do not deleteriously react with nucleic acids can also be used to formulate siRNA compositions. Suitable pharmaceutically acceptable carriers for formulations used in non-parenteral delivery include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents, and other suitable additives. Pharmaceutically acceptable organic or inorganic excipients suitable for non-parenteral administration that do not deleteriously react with nucleic acids can be used.

In some embodiments, administration of pharmaceutical compositions and formulations described herein can be topical (e.g., by a transdermal patch), pulmonary (e.g., by inhalation or insufflation of powders or aerosols, including by nebulizer); intratracheal; intranasal; epidermal and transdermal; oral; or parenteral. Parenteral administration includes intravenous, intraarterial, subcutaneous, intraperitoneal, and intramuscular injection or infusion; subdermal administration (e.g., via an implanted device); or intracranial administration (e.g., by intraparenchymal, intrathecal, or intraventricular, administration).

In some embodiments, the pharmaceutical composition comprises a sterile solution of an siRNA (e.g., SIRNA01) formulated in water for subcutaneous injection. In some embodiments, the pharmaceutical composition comprises a sterile solution of SIRNA01 formulated in water for subcutaneous injection at a free acid concentration of 200 mg/mL.

In some embodiments, the pharmaceutical compositions containing an siRNA described herein are administered in dosages sufficient to inhibit expression of an HBV gene. In some embodiments, a dose of an siRNA is in the range of 0.001 to 200.0 milligrams per kilogram body weight of the recipient per day, or in the range of 1 to 50 milligrams per kilogram body weight per day. For example, an siRNA can be administered at 0.01 mg/kg, 0.05 mg/kg, 0.5 mg/kg, 1 mg/kg, 1.5 mg/kg, 2 mg/kg, 3 mg/kg, 10 mg/kg, 20 mg/kg, 30 mg/kg, 40 mg/kg, or 50 mg/kg per single dose. The pharmaceutical composition can be administered once daily, or it can be administered as two, three, or more sub-doses at appropriate intervals throughout the day or even using continuous infusion or delivery through a controlled release formulation. In that case, the siRNA contained in each sub-dose must be correspondingly smaller in order to achieve the total daily dosage. The dosage unit can also be compounded for delivery over several days, e.g., using a conventional sustained release formulation which provides sustained release of the siRNA over a several day period. Sustained release formulations are well known in the art and are particularly useful for delivery of agents at a particular site, such as could be used with the agents of the technology described herein. In such embodiments, the dosage unit contains a corresponding multiple of the daily dose.

In some embodiments, a pharmaceutical composition comprising an siRNA that targets HBV mRNA described herein (e.g., SIRNA01) contains the siRNA at a dose of 0.8 mg/kg, 1.7 mg/kg, 3.3 mg/kg, 6.7 mg/kg, or 15 mg/kg.

In some embodiments, a pharmaceutical composition comprising an siRNA described herein (e.g., SIRNA01) contains the siRNA at a dose of 20 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg. In some embodiments, a pharmaceutical composition comprising an siRNA described herein (e.g., SIRNA01) contains the siRNA at a dose of from 20 mg to 900 mg. In some embodiments, a pharmaceutical composition comprising an siRNA described herein (e.g., SIRNA01) contains the siRNA at a dose of from 100 mg to 300 mg.

In some embodiments, a pharmaceutical composition comprising an siRNA described herein (e.g., SIRNA01) contains the siRNA at a dose of 20 mg, 50 mg, 100 mg, 150 mg, 200 mg, 250 mg, 300 mg, 400 mg, or 450 mg.

In some embodiments, a pharmaceutical composition comprising an siRNA described herein (e.g., SIRNA01) contains the siRNA at a dose of 200 mg.

### II. ANTI-HBV ANTIBODIES

The present disclosure also provides anti-HBV antibodies for use in a combination therapy for treating HDV or an HDV-associated disease. Antibodies that bind to the antigenic loop of HBsAg on the surface of the HDV virion may act as entry inhibitors by blocking interactions between HBsAg and its receptor NTCP. Inhibiting viral entry prevents new rounds of HDV infection in the liver and ultimately reduces HDV viremia. Antibodies may also promote HDV clearance by opsonizing virions. Antibodies may also have indirect antiviral activity on HDV by stimulating an immune responses against HBsAg, which will be present in coinfected hepatocytes producing new HDV virions.

A. Antibodies that Bind to HBV Proteins

In some embodiments, the anti-HBV antibody of the combination therapy, or the antigen binding fragment thereof, binds to the antigenic loop region of HBsAg. The envelope of the hepatitis B virus contains three “HBV envelope proteins” (also known as “HBsAg”, “hepatitis B surface antigen”): S protein (for “small”, also referred to as S-HBsAg), M protein (for “middle”, also referred to as M-HBsAg), and L protein (for “large”, also referred to as L-HBsAg). S-HBsAg, M-HBsAg, and L-HBsAg share the same C-terminal extremity (also referred to as “S domain”, 226 amino acids), which corresponds to the S protein (S-HBsAg) and which is involved in virus assembly and infectivity. S-HBsAg, M-HBsAg, and L-HBsAg are synthesized in the endoplasmic reticulum (ER), assembled, and secreted as particles through the Golgi apparatus. The S domain comprises four predicted transmembrane (TM) domains, whereby both the N-terminus and the C-terminus of the S domain are exposed to the lumen. The transmembrane domains TM1 and TM2 are both necessary for cotranslational protein integration into the ER membrane and the transmembrane domains TM3 and TM4 are located in the C-terminal third of the S domain. The “antigenic loop region” of HBsAg is located between the predicted TM3 and TM4 transmembrane domains of the S domain of HBsAg, whereby the antigenic loop region comprises amino acids 101-172 of the S domain (Salisse J and Sureau C, Journal of Virology 2009, 83:9321-8). An important determinant of infectivity resides in the antigenic loop region of HBV envelope proteins. In particular, residues between 119 and 125 of the HBsAg contain a CXXC motif, which has been demonstrated to be the most important sequence required for the infectivity of HBV (Jaoude G A and Sureau C, Journal of Virology 2005, 79:10460-6).

As used herein, the S domain of HBsAg refers to an amino acid sequence as set forth in SEQ ID NO:13 (shown below) or to natural or artificial sequence variants thereof.

MENITSGFLGPLLVLQAGFFLLTRILTIPQSLDSWWTSLNFLGGTT
VCLGQNSQSPTSNHSPTSCPPTCPGYRWMCLRRFIIFLFILLLCLIF
LLVLLDYQGMLPVCPLIPGSSTTSTGPCRTCMTTAQGTSMYPSCC
CTKPSDGNCTCIPIPSSWAFGKFLWEWASARFSWLSLLVPFVQWF
VGLSPTVWLSVIWMMWYWGPSLYSILSPFLPLLPIFFCLWVYI
(SEQ ID NO: 13; amino acids 101-172 are shown
underlined)

For example, the expression “amino acids 101-172 of the S domain” refers to the amino acid residues from positions 101-172 of the polypeptide according to SEQ ID NO:13. However, a person skilled in the art will understand that mutations or variations (including, but not limited to, substitution, deletion and/or addition, for example, HBsAg of a different genotype or a different HBsAg mutant as described herein) may occur naturally in the amino acid sequence of the S domain of HBsAg or be introduced artificially into the amino acid sequence of the S domain of HBsAg without affecting its biological properties. Therefore, the term “S domain of HBsAg” comprises all such polypeptides, for example, including the polypeptide according to SEQ ID NO:13 and its natural or artificial mutants. In addition, when sequence fragments of the S domain of HBsAg are described herein (e.g., amino acids 101-172 or amino acids 120-130 of the S domain of HBsAg), they include not only the corresponding sequence fragments of SEQ ID NO:13, but also the corresponding sequence fragments of its natural or artificial mutants. For example, the expression “amino acid residues from positions 101-172 of the S domain of HBsAg” includes amino acid residues from positions 101-172 of SEQ ID NO:13 and the corresponding fragments of its mutants (natural or artificial mutants).

As used herein, the expression “corresponding sequence fragments” or “corresponding fragments” refers to fragments that are located in equal positions of sequences when the sequences are subjected to optimized alignment, namely, the sequences are aligned to obtain a highest percentage of identity. The M protein (M-HBsAg) corresponds to the S protein extended by an N-terminal domain of 55 amino acids called “pre-S2”. The L protein (L-HBsAg) corresponds to the M protein extended by an N-terminal domain of 108 amino acids called “pre-S1” (genotype D). The pre-S1 and pre-S2 domains of the L protein can be present either at the inner face of viral particles (on the cytoplasmic side of the ER), playing a crucial role in virus assembly, or on the outer face (on the luminal side of the ER), available for the interaction with target cells and necessary for viral infectivity. Moreover, HBV surface proteins (HBsAgs) are not only incorporated into virion envelopes but also spontaneously bud from ER-Golgi intermediate compartment membranes to form empty “subviral particles” (SVPs) that are released from the cell by secretion.

Since all three HBV envelope proteins S-HBsAg, M-HBsAg, and L-HBsAg comprise the S domain, all three HBV envelope proteins S-HBsAg, M-HBsAg, and L-HBsAg also comprise the “antigenic loop region”. Accordingly, an antibody or an antigen binding fragment thereof that binds to the antigenic loop region of HBsAg binds to all three HBV envelope proteins: S-HBsAg, M-HBsAg, and L-HBsAg.

Moreover, in some embodiments, the anti-HBV antibody of the combination therapy, or an antigen binding fragment thereof, neutralizes infection with hepatitis B virus. In other words, the antibody, or the antigen binding fragment thereof, may reduce viral infectivity of hepatitis B virus. In some embodiments, the anti-HBV antibody of the combination therapy, or an antigen binding fragment thereof, neutralizes infection with hepatitis D virus. In other words, the antibody, or the antigen binding fragment thereof, may reduce viral infectivity of hepatitis D virus (see below for further explanation of hepatitis D virus as an obligate satellite of hepatitis B virus).

To study and quantitate virus infectivity (or “neutralization”) in the laboratory the person skilled in the art knows various standard “neutralization assays.” For a neutralization assay, animal viruses are typically propagated in cells and/or cell lines. In the context of the present disclosure, for a neutralization assay, cultured cells may be incubated with a fixed amount of HBV in the presence (or absence) of the antibody to be tested. As a readout, the levels of hepatitis B surface antigen (HBsAg) or hepatitis B e antigen (HBeAg) secreted into the cell culture supernatant may be used and/or HBcAg staining may be assessed. In one embodiment of a HBV neutralization assay, cultured cells, for example HepaRG cells, in particular differentiated HepaRG cells, are incubated with a fixed amount of HBV in the presence or absence of the antibody to be tested, for example for 16 hours at 37° C. The incubation may be performed in a medium (e.g., supplemented with 4% PEG 8000). After incubation, cells may be washed and further cultivated. To measure virus infectivity, the levels of hepatitis B surface antigen (HBsAg) and hepatitis B e antigen (HBeAg) secreted into the culture supernatant, e.g., from day 7 to day 11 post-infection, may be determined by enzyme-linked immunosorbent assay (ELISA). Additionally, HBcAg staining may be assessed in an immunofluorescence assay.

In some embodiments, the antibody and antigen binding fragment have high neutralizing potency. The concentration of an antibody of the present disclosure required for 50% neutralization of hepatitis B virus (HBV) is, for example, about 10 μg/ml or less. In certain embodiments, the concentration of an antibody of the present disclosure required for 50% neutralization of HBV is about 5 μg/ml, about 1 μg/ml, or about 750 ng/ml. In certain embodiments, the concentration of an antibody of the present disclosure required for 50% neutralization of HBV is 500 ng/ml or less, e.g., 450, 400, 350, 300, 250, 200, 175, 150, 125, 100, 90, 80, 70, 60, or about 50 ng/ml or less. This means that only low concentrations of the antibody are required for 50% neutralization of HBV. Specificity and potency can be measured using standard assays as known to one of skill in the art.

In some embodiments, the anti-HBV antibody, as a component of the combination therapy, is useful in the prevention and/or treatment of hepatitis B or hepatitis B-associated diseases.

In some embodiments, an antibody according to the present disclosure, or an antigen binding fragment thereof, promotes clearance of HBsAg and HBV. In particular, an antibody according to the present disclosure, or an antigen binding fragment thereof, may promote clearance of both HBV and subviral particles of hepatitis B virus (SVPs). Clearance of HBsAg or of subviral particles may be assessed by measuring the level of HBsAg for example in a blood sample, e.g., from a hepatitis B patient. Similarly, clearance of HBV may be assessed by measuring the level of HBV for example in a blood sample, e.g., from a hepatitis B patient.

In the sera of patients infected with HBV, in addition to infectious particles (HBV), there is typically an excess (typically 1,000- to 100,000-fold) of empty subviral particles (SVP) composed solely of HBV envelope proteins (HBsAg) in the form of relatively smaller spheres and filaments of variable length. Subviral particles were shown to strongly enhance intracellular viral replication and gene expression of HBV (Bruns M et al., J Virol 1998, 72(2):1462-8). This is also important in the context of infectivity of sera containing HBV, since the infectivity depends not only on the number of viruses but also on the number of SVPs (Bruns et al., 1998, supra). Moreover, an excess of subviral particles can serve as a decoy by absorbing neutralizing antibodies and therefore delay the clearance of infection. Typically, achievement of hepatitis B surface antigen (HBsAg) loss is thus considered to be an ideal endpoint of treatment and the closest outcome to cure chronic hepatitis B (CHB). Accordingly, in some embodiments, an antibody according to the present disclosure, or an antigen binding fragment thereof, which promotes clearance of HBsAg, and in particular, clearance of subviral particles of hepatitis B virus and HBV, enables improved treatment of hepatitis B, in particular in the context of chronic hepatitis B. Thereby, an antibody according to the present disclosure, or an antigen binding fragment thereof, may potently neutralize HBV since less of the antibody is absorbed by SVPs acting as a decoy. In addition, in certain embodiments, an antibody according to the present disclosure, or an antigen binding fragment thereof, promotes clearance of subviral particles of hepatitis B virus, and decreases infectivity of HBV in sera.

HBV is differentiated into many genotypes, according to genome sequence. To date, eight well-known genotypes (A-H) of the HBV genome have been defined. Moreover, two new genotypes, I and J, have also been identified (Sunbul M, World J Gastroenterol 2014, 20(18):5427-34). The genotype is known to affect the progression of the disease, and differences between genotypes in response to antiviral treatment have been determined. For example, genotype A has a tendency for chronicity, whereas viral mutations are frequently encountered in genotype C. Both chronicity and mutation frequency are common in genotype D. Moreover, the genotypes of HBV are differentially distributed over the world (Sunbul, 2014, supra). In certain embodiments, an antibody according to the present disclosure, or an antigen binding fragment thereof, binds to at least 6, to at least 8, or to all 10 of the HBsAg genotypes A, B, C, D, E, F, G, H, I, and J. In certain embodiments, an antibody according to the present disclosure, or an antigen binding fragment thereof, binds to 1, 2, 3, 4, 5, 6, 7, 8, 9, or 10 of the HBsAg genotypes A, B, C, D, E, F, G, H, I, and J. Examples for the different genotypes of HBsAg include the following: GenBank accession number J02203 (HBV-D, ayw3), GenBank accession number FJ899792.1 (HBV-D, adw2), GenBank accession number AM282986 (HBV-A), GenBank accession number D23678 (HBV-B1 Japan), GenBank accession number AB1 1 7758 (HBV-C1 Cambodia), GenBank accession number AB205192 (HBV-E Ghana), GenBank accession number X69798 (HBV-F4 Brazil), GenBank accession number AF160501 (HBV-G USA), GenBank accession number AY090454 (HBV-H Nicaragua), GenBank accession number AF241409 (HBV-I Vietnam), and GenBank accession number AB486012 (HBV-J Borneo). The amino acid sequences of the antigenic loop region of the S domain of HBsAg of the different genotypes are shown in Table 2 (SEQ ID NOs:14-42).

TABLE 2
Antigenic Loop Sequences from various HBV genotypes.

| HBsAg Antigenic Loop Sequence | Strain | SEQ ID NO: |
|---|---|---|
| QGMLPVCPLIPGSSTTSTGPCRTCMTTAQGTS | J02203 (D, | 14 |
| MYPSCCCTKPSDGNCTCIPIPSSWAFGKFLWE | ayw3) | |
| WASARFSW | | |
| QGMLPVCPLIPGSSTTGTGPCRTCTTPAQGTS | FJ899792 (D, | 15 |
| MYPSCCCTKPSDGNCTCIPIPSSWAFGKFLWE | adw2) | |
| WASARFSW | | |
| QGMLPVCPLIPGTTTTSTGPCKTCTTPAQGNS | AM282986 | 16 |
| MFPSCCCTKPSDGNCTCIPIPSSWAFAKYLWE | (A) | |
| WASVRFSW | | |
| QGMLPVCPLIPGSSTTSTGPCKTCTTPAQGTS | D23678 (B1) | 17 |
| MFPSCCCTKPTDGNCTCIPIPSSWAFAKYLWE | | |
| WASVRFSW | | |
| QGMLPVCPLLPGTSTTSTGPCKTCTIPAQGTS | AB117758 | 18 |
| MFPSCCCTKPSDGNCTCIPIPSSWAFARFLWE | (C1) | |
| WASVRFSW | | |
| QGMLPVCPLIPGSSTTSTGPCRTCTTLAQGTS | AB205192(E) | 19 |
| MFPSCCCSKPSDGNCTCIPIPSSWAFGKFLWE | | |
| WASARFSWLS | | |
| QGMLPVCPLLPGSTTTSTGPCTCTTLAQGTSM | X69798 (F4) | 20 |
| FPSCCCSKPSDGNCTCIPIPSSWALGKYLWEW | | |
| ASARFSW | | |
| QGMLPVCPLIPGSSTTSTGPCTCTTPAQGNSM | AF1 60501 (G) | 21 |
| YPSCCCTPSDGNCTCIPIPSSWAFAKYLWEWA | | |
| SVRFSW | | |
| QGMLPVCPLLPGSTTTSTGPCKTCTTLAQGTS | AY090454 (H) | 22 |
| MFPSCCCTKPSDGNCTCIPIPSSWAFGKYLWE | | |
| WASARFSW | | |
| QGMLPVCPLIPGSSTTSTGPCKTCTTPAQGNS | AF241409 (I) | 23 |
| MYPSCCCTKPSDGNCTCIPIPSSWAFAKYLWE | | |
| WASARFSW | | |
| QGMLPVCPLLPGSTTTSTGPCRTCTITAQGTS | AB486012 (J) | 24 |
| MFPSCCCTKPSDGNCTCIPIPSSWAFAKFLWE | | |
| WASVRFSW | | |
| CQGMLPVCPLIPGSSTTGTGTCRTCTTPAQGT | HBsAg | 25 |
| SMYPSCCCTKPSDGNCTCIPIPSSWAFG | Y100C/P120T | |
| FLWEWASARFSW | | |
| QGMLPVCPLIPGSSTTGTGTCRTCTTPAQGTS | HBsAg P120T | 26 |
| MYPSCCCTKPSDGNCTCIPIPSSWAFGKFLWE | | |
| WASARFSW | | |
| QGMLPVCPLIPGSSTTGTGTCRTCTTPAQGTS | HBsAg | 27 |
| MYPSCCCTKPLDGNCTCIPIPSSWAFGKFLWE | P120T/S143L | |
| WASARFSW | | |
| QGMLPVCPLIPGSSTTGTGPSRTCTTPAQGTS | HBsAg C121S | 28 |
| MYPSCCCTKPSDGNCTCIPIPSSWAFGKFLWE | | |
| WASARFSW | | |
| QGMLPVCPLIPGSSTTGTGPCDTCTTPAQGTS | HBsAg R122D | 29 |
| MYPSCCCTKPSDGNCTCIPIPSSWAFG | | |
| KFLWEWASARFSW | | |
| QGMLPVCPLIPGSSTTGTGPCITCTTPAQGTSM | HBsAg R122I | 30 |
| YPSCCCTPSDGNCTCIPIPSSWAFGKFLWEWA | | |
| SARFSW | | |
| QGMLPVCPLIPGSSTTGTGPCRNCTTPAQGTS | HBsAg T123N | 31 |
| MYPSCCCTKPSDGNCTCIPIPSSWAFGKFLWE | | |
| WASARFSW | | |
| QGMLPVCPLIPGSSTTGTGPCRTCTTPAHGTS | HBsAg Q129H | 32 |
| MYPSCCCTKPSDGNCTCIPIPSSWAFGKF | | |
| LWEWASARFSW | | |
| QGMLPVCPLIPGSSTTGTGPCRTCTTPALGTS | HBsAg Q129L | 33 |
| MYPSCCCTKPSDGNCTCIPIPSSWAFGKFLWE | | |
| WASARFSW | | |
| QGMLPVCPLIPGSSTTGTGPCRTCTTPAQGTS | HBsAg MI33H | 34 |
| HYPSCCCTKPSDGNCTCIPIPSSWAFGKFLWE | | |
| WASARFSW | | |
| QGMLPVCPLIPGSSTTGTGPCRTCTTPAQGTS | HBsAg M133L | 35 |
| LYPSCCCTKPSDGNCTCIPIPSSWAFGKFLWE | | |
| WASARFSW | | |
| QGMLPVCPLIPGSSTTGTGPCRTCTTPAQGTS | HBsAg M133T | 36 |
| TYPSCCCTKPSDGNCTCIPIPSSWAFGKFLWE | | |
| WASARFSW | | |
| QGMLPVCPLIPGSSTTGTGPCRTCTTPAQGTS | HBsAg K141E | 37 |
| MYPSCCCTEPSDGNCTCIPIPSSWAFGKFLWE | | |
| WASARFSW | | |
| QGMLPVCPLIPGSSTTGTGPCRTCTTPAQGTS | HBsAg P142S | 38 |
| MYPSCCCTKSSDGNCTCIPIPSSWAFGKFLWE | | |
| WASARFSW | | |
| QGMLPVCPLIPGSSTTGTGPCRTCTTPAQGTS | HBsAg S143K | 39 |
| MYPSCCCTKPKDGNCTCIPIPSSWAFGKFLWE | | |
| WASARFSW | | |
| QGMLPVCPLIPGSSTTGTGPCRTCTTPAQGTS | HBsAg D144A | 40 |
| MYPSCCCTPSAGNCTCIPIPSSWAFGKFLWEW | | |
| ASARFSW | | |
| QGMLPVCPLIPGSSTTGTGPCRTCTTPAQGTS | HBsAg G14SR | 41 |
| MYPSCCCTKPSDRNCTCIPIPSSWAFGKFLWE | | |
| WASARFSW | | |
| QGMLPVCPLIPGSSTTGTGPCRTCTTPAQGTS | HBsAg N146A | 42 |
| MYPSCCCTKPSDGACTCIPIPSSWAFGKFLWE | | |
| WASARFSW | | |

In certain embodiments, an antibody according to the present disclosure, or an antigen binding fragment thereof, binds to 1, 2, 3, 4, 5, 6, 7, 8, 9, 10, 11, 12, 13, 14, 15, 16, 17 or 18 of the HBsAg mutants having mutations in the antigenic loop region: HBsAg Y100C/P120T, HBsAg P120T, HBsAg P120T/S143L, HBsAg C121 S, HBsAg R122D, HBsAg R122I, HBsAg T123N, HBsAg Q129H, HBsAg Q129L, HBsAg M133H, HBsAg M133L, HBsAg M133T, HBsAg K141 E, HBsAg P142S, HBsAg S143K, HBsAg D144A, HBsAg G145R, and HBsAg N146A. These mutants are naturally occurring mutants based on the S domain of HBsAg Genotype D (SEQ ID NO:43), Genbank accession no. FJ899792 (whereby the mutated amino acid residue(s) are indicated in the name).

MENVTSGFLGPLLVLQAGFFLLTRILTIPQSLDSWWTSLNFLGGT
TVCLGQNSQSPTSNHSPTSCPPTCPGYRWMCLRRFIIFLFILLLCLI
FLLVLLDYQGMLPVCPLIPGSSTTGTGPCRTCTTPAQGTSMYPSC
CCTKPSDGNCTCIPIPSSWAFGKFLWEWASARFSWLSLLVPFVQ
WFVGLSPTVWLSVIWMMWYWGPSLYSTLSPFLPLLPIFFCLWVYI
(SEQ ID NO: 43) (the antigenic loop region,
i.e., amino acids 101-172, is shown underlined).

In particular embodiments, an antibody according to the present disclosure, or an antigen binding fragment thereof, binds to at least 12, to at least 15, or to all 18 of the infectious HBsAg mutants having mutations in the antigenic loop region: HBsAg Y100C/P120T, HBsAg P120T, HBsAg P120T/S143L, HBsAg C121 S, HBsAg R122D, HBsAg R122I, HBsAg T123N, HBsAg Q129H, HBsAg Q129L, HBsAg M1 33H, HBsAg M133L, HBsAg M133T, HBsAg K141 E, HBsAg P142S, HBsAg S143K, HBsAg D144A, HBsAg G145R, and HBsAg N146A.

In certain embodiments, an antibody according to the present disclosure, or an antigen binding fragment thereof, binds to an epitope comprising at least one, at least two, at least three amino acids, or e at least four amino acids of the antigenic loop region of HBsAg, wherein the at least two, at least three, or at least four amino acids are selected from amino acids 115-133 of the S domain of HBsAg, amino acids 120-133 of the S domain of HBsAg, or amino acids 120-130 of the S domain of HBsAg. Of note, the position of the amino acids (e.g., 115-133, 120-133, 120-130) refers to the S domain of HBsAg as described above, which is present in all three HBV envelope proteins S-HBsAg, M-HBsAg, and L-HBsAg.

In particular embodiments, an antibody according to the present disclosure, or an antigen binding fragment thereof, binds to an epitope in the antigenic loop region of HBsAg, whereby the epitope is formed by one or more amino acids located at positions selected from amino acid positions 115-133, amino acid positions 120-133, or amino acid positions 120-130 of the S domain of HBsAg.

The term “formed by” as used herein in the context of an epitope means, that the epitope to which an antibody of the present disclosure, or an antigen binding fragment thereof, binds to may be linear (continuous) or conformational (discontinuous). A linear or a sequential epitope is an epitope that is recognized by antibodies by its linear sequence of amino acids, or primary structure. In contrast, a conformational epitope has a specific three-dimensional shape and protein structure. Accordingly, if the epitope is a linear epitope and comprises more than one amino acid located at positions selected from amino acid positions 115-133, or amino acid positions 120-133 of the S domain of HBsAg, the amino acids comprised by the epitope may be located in adjacent positions of the primary structure (i.e., consecutive amino acids in the amino acid sequence). In the case of a conformational epitope (3D structure), in contrast, the amino acid sequence typically forms a 3D structure as epitope and, thus, the amino acids forming the epitope (or the amino acids “comprised by” the epitope) may be or may be not located in adjacent positions of the primary structure (i.e., may or may not be consecutive amino acids in the amino acid sequence). In certain embodiments, the epitope to which an antibody of the present disclosure, or an antigen binding fragment thereof, binds is only formed by amino acid(s) selected from amino acid positions 115-133, amino acid positions 120-133, or amino acid positions 120-130 of the S domain of HBsAg. In particular embodiments, no (further) amino acids-which are located outside the positions 115-133, positions 120-133, or positions 120-130—are required to form the epitope to which an antibody of the present disclosure, or an antigen binding fragment thereof, binds.

In certain embodiments, the epitope in the antigenic loop region of HBsAg to which an antibody of the present disclosure, or an antigen binding fragment thereof, binds is formed by two or more amino acids located at positions selected from amino acid positions 115-133, amino acid positions 120-133, or amino acid positions 120-130 of the S domain of HBsAg. In certain embodiments, the epitope in the antigenic loop region of HBsAg to which an antibody of the present disclosure, or an antigen binding fragment thereof, binds is formed by three or more amino acids located at positions selected from amino acid positions 115-133, amino acid positions 120-133, and amino acid positions 120-130 of the S domain of HBsAg. In some embodiments, the epitope in the antigenic loop region of HBsAg to which an antibody of the present disclosure, or an antigen binding fragment thereof, binds is formed by four or more amino acids located at positions selected from amino acid positions 115-133, amino acid positions 120-133, or amino acid positions 120-130 of the S domain of HBsAg. As such, an antibody according to the present disclosure, or an antigen binding fragment thereof, may bind to at least one, at least two, at least three, or at least four amino acids of the antigenic loop region of HBsAg selected from amino acids 115-133 of the S domain of HBsAg, amino acids 120-133 of the S domain of HBsAg, or amino acids 120-130 of the S domain of HBsAg. In particular embodiments, an antibody according to the present disclosure, or the antigen binding fragment thereof, binds to an epitope comprising at least two, at least three, or at least four amino acids of the antigenic loop region of HBsAg, wherein the at least two, at least three, or at least four amino acids are selected from amino acids 120-133, or amino acids 120-130 of the S domain of HBsAg and wherein the at least two, at least three, or at least four amino acids are located in adjacent positions (i.e., are consecutive amino acids in the amino acid sequence/primary structure).

In certain embodiments, the epitope to which an antibody according to the present disclosure, or an antigen binding fragment thereof, binds to, is a conformational epitope. Accordingly, an antibody according to the present disclosure, or an antigen binding fragment thereof, may bind to an epitope comprising at least two, at least three, or at least four amino acids of the antigenic loop region of HBsAg, wherein the at least two, at least three, or at least four amino acids are selected from amino acids 120-133, or amino acids 120-130, of the S domain of HBsAg and wherein at least two, or at least three, or at least four amino acids are not located in adjacent positions (of the primary structure).

In certain specific embodiments, an antibody of the present disclosure is a bispecific antibody, with a first specificity for HBsAg, and a second specificity that stimulates an immune effector cell (e.g., by targeting a T cell surface protein such as, for example, a CD3 protein extracellular portion). The second specificity may cause, for example, a cytotoxic effect or a vaccinal effect.

### B. Fc Moieties

In some embodiments, a binding protein (e.g., antibody or an antigen binding fragment thereof) comprises an Fc moiety. In certain embodiments, the Fc moiety may be derived from human origin, e.g., from human IgG1, IgG2, IgG3, and/or IgG4. In specific embodiments, an antibody or antigen binding fragments can comprise an Fc moiety derived from human IgG1.

As used herein, the term “Fc moiety” refers to a sequence comprising or derived from a portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. In certain embodiments, a complete Fc moiety comprises a hinge domain, a CH₂ domain, and a CH₃ domain (e.g., EU amino acid positions 216-446). An additional lysine residue (K) is sometimes present at the extreme C-terminus of the Fc moiety, but is often cleaved from a mature antibody. Amino acid positions within an Fc moiety have been numbered according to the EU numbering system of Kabat (see, e.g., Kabat et al., Sequences of Proteins of Immunological Interest, U.S. Dept. Health and Human Services, 1983 and 1987). Amino acid positions of an Fc moiety can also be numbered according to the IMGT numbering system (including unique numbering for the C-domain and exon numbering) and the Kabat numbering system.

In some embodiments, an Fc moiety comprises at least one of: a hinge (e.g., upper, middle, and/or lower hinge region) domain, a CH₂ domain, a CH₃ domain, or a variant, portion, or fragment thereof. In some embodiments, an Fc moiety comprises at least a hinge domain, a CH₂ domain, or a CH₃ domain. In further embodiments, the Fc moiety is a complete Fc moiety. The amino acid sequence of an exemplary Fc moiety of human IgG1 isotype is provided in SEQ ID NO:60. The Fc moiety may also comprise one or more amino acid insertions, deletions, or substitutions relative to a naturally occurring Fc moiety. For example, at least one of a hinge domain, CH₂ domain, or CH₃ domain, or a portion thereof, may be deleted. For example, an Fc moiety may comprise or consist of: (i) hinge domain (or a portion thereof) fused to a CH₂ domain (or a portion thereof), (ii) a hinge domain (or a portion thereof) fused to a CH₃ domain (or a portion thereof), (iii) a CH₂ domain (or a portion thereof) fused to a CH₃ domain (or a portion thereof), (iv) a hinge domain (or a portion thereof), (v) a CH₂ domain (or a portion thereof), or (vi) a CH₃ domain or a portion thereof.

An Fc moiety of the present disclosure may be modified such that it varies in amino acid sequence from the complete Fc moiety of a naturally occurring immunoglobulin molecule, while retaining (or enhancing) at least one desirable function conferred by the naturally occurring Fc moiety. Such functions include, for example, Fc receptor (FcR) binding, antibody half-life modulation (e.g., by binding to FcRn), ADCC function, protein A binding, protein G binding, and complement binding. Portions of naturally occurring Fc moieties which are involved with such functions have been described in the art.

For example, to activate the complement cascade, the C1q protein complex can bind to at least two molecules of IgG1 or one molecule of IgM when the immunoglobulin molecule(s) is attached to the antigenic target (Ward E S and Ghetie, V, Ther. Immunol. 1995, 277-94). The heavy chain region comprising amino acid residues 318 to 337 is involved in complement fixation (Burton D R, Mol. Immunol. 1985, 22:161-206). Duncan A R and Winter G. (Nature 1988, 332:738-40), using site directed mutagenesis, reported that Glu318, Lys320, and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g., tumor cells) coated with the corresponding antibody, via antibody dependent cell mediated cytotoxicity (ADCC; Van de Winkel J G and Anderson C L, J. Leukoc. Biol. 1991, 49:511-24). FcRs are defined by their specificity for immunoglobulin classes; Fc receptors for IgG antibodies are referred to as FcγR, for IgE as FcεR, for IgA as FcαR, and so on, and neonatal Fc receptors are referred to as FcRn. Fc receptor binding is described in, for example, Ravetch J V and Kinet J P, Annu. Rev. Immunol. 1991, 9:457-92; Capel P J et al., Immunomethods 1994, 4:25-34; de Haas M et al., J Lab. Clin. Med. 1995, 126:330-41; and Gessner J E et al., Ann. Hematol. 1998, 76:231-48.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized: (i) FcγRI (CD64), which binds monomeric IgG with high affinity and is expressed on macrophages, monocytes, neutrophils, and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB, and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g., macrophages, monocytes, neutrophils) and seems able to activate the killing process. FcγRIIB seems to play a role in inhibitory processes and is found on B-cells, macrophages and on mast cells and eosinophils. It has been shown that 75% of all FcγRIIB is found in the liver (Ganesan L P et al., Journal of Immunology 2012, 189:4981-8). FcγRIIB is abundantly expressed on Liver Sinusoidal Endothelium, called LSEC, and in Kupffer cells in the liver, and LSEC are the major site of small immune complexes clearance (Ganesan et al., 2012, supra).

In some embodiments the antibodies disclosed herein and the antigen binding fragments thereof comprise an Fc moiety for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu S Y et al. (Molecular Immunology 2008, 45:3926-33). Thereby, the clearance of immune complexes can be enhanced (Chu S et al., Am J Respir Crit, American Thoracic Society International Conference Abstracts 2014). In some embodiments, the antibodies of the present disclosure, or the antigen binding fragments thereof, comprise an engineered Fc moiety with the mutations S267E and L328F, in particular as described by Chu S Y et al. (2008, supra).

On B cells, FcγRIIB seems to function to suppress further immunoglobulin production and isotype switching to, for example, the IgE class. On macrophages, FcγRIIB is thought to inhibit phagocytosis as mediated through FcγRIIA. On eosinophils and mast cells, the b form may help to suppress activation of these cells through IgE binding to its separate receptor.

Regarding FcγRI binding, modification in native IgG of at least one of E233-G236, P238, D265, N297, A327, and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour K L et al., Eur. J. Immunol. 1999, 29:2613-2624).

Regarding FcγRII binding, reduced binding for FcγRIIA is found, e.g., for IgG mutation of at least one of E233-G236, P238, D265, N297, A327, P329, D270, Q295, A327, R292, and K414.

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338, and D376.

Mapping of the binding sites on human IgG1 for Fc receptors, the above mentioned mutation sites, and methods for measuring binding to FcγRI and FcγRIIA, are described in Shields R L et al. (J. Biol. Chem. 2001, 276:6591-6604).

Regarding binding to FcγRII, two regions of native IgG Fc appear to be involved in interactions between FcγRIIs and IgGs, namely (i) the lower hinge site of IgG Fc, in particular amino acid residues L, L, G, and G (234-237, EU numbering), and (ii) the adjacent region of the CH₂ domain of IgG Fc, in particular a loop and strands in the upper CH₂ domain adjacent to the lower hinge region, e.g., in a region of P331 (Wines B D et al., J. Immunol. 2000, 164:5313-8). Moreover, FcγRI appears to bind to the same site on IgG Fc, whereas FcRn and Protein A bind to a different site on IgG Fc, which appears to be at the CH₂—CH₃ interface (Wines B D et al., 2000, supra).

Also contemplated are mutations that increase binding affinity of an Fc moiety of the present disclosure to a (i.e., one or more) Fcγ receptor (e.g., as compared to a reference Fc moiety or antibody that does not comprise the mutation(s)). See, e.g., Delillo and Ravetch, Cell 2015, 161(5):1035-45 and Ahmed et al., J. Struc. Biol. 2016, 194(1):78, the Fc mutations and techniques of which are incorporated herein by reference. In any of the herein disclosed embodiments, a binding protein can comprise a Fc moiety comprising a mutation selected from G236A; S239D; A330L; and 1332E; or a combination comprising the same; e.g., S239D/I332E; S239D/A330L/I332E; G236A/S239D/I332E; G236A/A330L/I332E; and G236A/S239D/A330L/I332E.

In certain embodiments, the Fc moiety may comprise or consist of at least a portion of an Fc moiety that is involved in binding to FcRn binding. In certain embodiments, the Fc moiety comprises one or more amino acid modifications that improve binding affinity for FcRn and, in some embodiments, thereby extend in vivo half-life of a molecule comprising the Fc moiety (e.g., as compared to a reference Fc moiety or antibody that does not comprise the modification(s)). In certain embodiments, Fc moiety comprises or is derived from a IgG Fc and a half-life-extending mutation comprises any one or more of: M428L; N434S; N434H; N434A; N434S; M252Y; S254T; T256E; T250Q; P257I; Q311I; D376V; T307A; and E380A (EU numbering). In certain embodiments, a half-life-extending mutation comprises M428L/N434S. In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In particular embodiments, a binding protein includes an Fc moiety that comprises the substitution mutations: M428L/N434S and G236A/A330L/I332E. In certain embodiments, an antibody or antigen binding fragment includes a Fc moiety that comprises the substitution mutations: M428L/N434S and G236A/S239D/A330L/I332E.

In particular embodiments, a binding protein includes an Fc moiety that comprises the substitution mutations: G236A/A330L/I332E. In certain embodiments, an antibody or antigen binding fragment includes a Fc moiety that comprises the substitution mutations: G236A/S239D/A330L/I332E.

Alternatively or additionally, the Fc moiety of a binding protein of the disclosure can comprise at least a portion known in the art to be required for Protein A binding; and/or the Fc moiety of an antibody of the disclosure comprises at least the portion of an Fc molecule known in the art to be required for protein G binding. In some embodiments, a retained function comprises the clearance of HBsAg and HBVg.

Accordingly, in certain embodiments, an Fc moiety comprises at least a portion known in the art to be required for FcγR binding. As outlined above, an Fc moiety may thus at least comprise (i) the lower hinge site of native IgG Fc, in particular amino acid residues L, L, G, and G (234-237, EU numbering), and (ii) the adjacent region of the CH₂ domain of native IgG Fc, in particular a loop and strands in the upper CH₂ domain adjacent to the lower hinge region, e.g., in a region of P331, for example a region of at least 3, 4, 5, 6, 7, 8, 9, or 10 consecutive amino acids in the upper CH₂ domain of native IgG Fc around P331, e.g., between amino acids 320 and 340 (EU numbering) of native IgG Fc.

In some embodiments, a binding protein according to the present disclosure comprises an Fc region. As used herein, the term “Fc region” refers to the portion of an immunoglobulin formed by two or more Fc moieties of antibody heavy chains. For example, an Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2, and are incorporated herein by reference. The Fc region can be or comprise a dimeric Fc region. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties (e.g., one Fc monomer of the dimeric Fc region comprises at least one amino acid modification (e.g., substitution, deletion, insertion, or chemical modification) that is not present in the other Fc monomer, or one Fc monomer may be truncated as compared to the other).

Presently disclosed Fc moieties may comprise Fc sequences or regions of the same or different class and/or subclass. For example, Fc moieties may be derived from an immunoglobulin (e.g., a human immunoglobulin) of an IgG1, IgG2, IgG3, or IgG4 subclass, or from any combination thereof. In certain embodiments, the Fc moieties of Fc region are of the same class and subclass. However, the Fc region (or one or more Fc moieties of an Fc region) may also be chimeric, whereby a chimeric Fc region may comprise Fc moieties derived from different immunoglobulin classes and/or subclasses. For example, at least two of the Fc moieties of a dimeric or single-chain Fc region may be from different immunoglobulin classes and/or subclasses. In certain embodiments, a dimeric Fc region can comprise sequences from two or more different isotypes or subclasses; e.g., a SEEDbody (“strand-exchange engineered domains”) (see Davis et al., Protein Eng. Des. Sel. 2010, 23(4):195).

Additionally or alternatively, chimeric Fc regions may comprise one or more chimeric Fc moieties. For example, the chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH₂ and/or CH₃ domain derived from an immunoglobulin of a first subclass (e.g., an IgG1, IgG2, or IgG4 subclass) and a hinge region from an immunoglobulin of a second subclass (e.g., an IgG3 subclass). For example, the Fc region or moiety may comprise a hinge and/or CH₂ domain derived from an immunoglobulin of a first subclass (e.g., an IgG4 subclass) and a CH₃ domain from an immunoglobulin of a second subclass (e.g., an IgG1, IgG2, or IgG3 subclass). For example, the chimeric Fc region may comprise an Fc moiety (e.g., a complete Fc moiety) from an immunoglobulin for a first subclass (e.g., an IgG4 subclass) and an Fc moiety from an immunoglobulin of a second subclass (e.g., an IgG1, IgG2, or IgG3 subclass). For example, the Fc region or moiety may comprise a CH₂ domain from an IgG4 immunoglobulin and a CH₃ domain from an IgG1 immunoglobulin. For example, the Fc region or moiety may comprise a CH₁ domain and a CH₂ domain from an IgG4 molecule and a CH₃ domain from an IgG1 molecule. For example, the Fc region or moiety may comprise a portion of a CH₂ domain from a particular subclass of antibody, e.g., EU positions 292-340 of a CH₂ domain. For example, an Fc region or moiety may comprise amino acids a positions 292-340 of CH₂ derived from an IgG4 moiety and the remainder of CH₂ derived from an IgG1 moiety (alternatively, 292-340 of CH₂ may be derived from an IgG1 moiety and the remainder of CH₂ derived from an IgG4 moiety).

Moreover, an Fc region or moiety may (additionally or alternatively) for example comprise a chimeric hinge region. For example, the chimeric hinge may be derived, e.g., in part, from an IgG1, IgG2, or IgG4 molecule (e.g., an upper and lower middle hinge sequence) and, in part, from an IgG3 molecule (e.g., an middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In some other embodiments, the chimeric hinge can comprise amino acids at EU positions 233-236 are from an IgG2 antibody and/or the Ser228Pro mutation, wherein the remaining amino acids of the hinge are from an IgG4 antibody (e.g., a chimeric hinge of the sequence ESKYGPPCPPCPAPPVAGP (SEQ ID NO:61)). Further chimeric hinges, which may be used in the Fc moiety of an antibody according to the present disclosure, are described in US 2005/0163783 A1.

In some embodiments, an Fc moiety or Fc region, comprises or consists of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region.

c. HBC34 Antibodies

In certain embodiments, the anti-HBV antibody is HBC34 or an engineered variant thereof. HBC34 are human antibodies against HBsAg with high neutralizing activity. HBC34 binds to the antigenic loop of HBsAg with high affinity (in the pM range), recognizes all 10 HBV genotypes and 18 mutants, and binds to spherical SVPs with low stoichiometry. The activity of HBC34, as measured diagnostically with an immunoassay, is 5000 IU/mg. As a comparison, the activity of HBIG is ˜1 IU/mg.

As referred to herein, the terms “an HBC34 antibody” and “HBC antibodies” can include the wild-type HBC34 antibody or an engineered variant thereof (e.g., HBC34 and HBC34 variants described in Table 3), unless stated otherwise.

Table 3 shows the amino acid sequences of the CDRs, heavy chain variable regions (V_{H}), and light chain variable regions (V_{L}) of HBC34 and engineered variants thereof. Also shown are full-length heavy chain (HC) and light chain (LC) amino acid sequences of exemplary antibodies of the present disclosure.

TABLE 3
Sequences for HBC34 antibodies.

| | | | SEQ |
|---|---|---|---|
| Antibodies | Antibody Region | Amino Acid Sequence | ID NO |
| HBC34, | CDRH1 | GRIFRSFY | 44 |
| HBC34v35-MLNS- | | | |
| GAALIE (“AB01”) | | | |
| HBC34, | (short) CDRH2 | NQDGSEK | 45 |
| HBC34v35-MLNS- | | | |
| GAALIE (AB01) | | | |
| HBC34, | (long) CDRH2 | INQDGSEK | 46 |
| HBC34v35-MLNS- | | | |
| GAALIE (AB01) | | | |
| HBC34, | CDRH3 | AAWSGNSGGMDV | 47 |
| HBC34v35-MLNS- | | | |
| GAALIE (AB01) | | | |
| HBC34, | CDRL1 | KLGNKN | 48 |
| HBC34v35-MLNS- | | | |
| GAALIE (AB01) | | | |
| HBC34, | (short) CDRL2 | EVK | 49 |
| HBC34v35-MLNS- | | | |
| GAALIE (AB01) | | | |
| HBC34, | (long) CDRL2 | VIYEVKYRP | 50 |
| HBC34v35-MLNS- | | | |
| GAALIE (AB01) | | | |
| HBC34 | CDRL3 | QTWDSTTVV | 51 |
| HBC34v35-MLNS- | CDRL3 | QTFDSTTVV | 52 |
| GAALIE (AB01) | | | |
| HBC34, | V_{H} | ELQLVESGGGWVQP | 53 |
| HBC34v35-MLNS- | | GGSQRLSCAASGRIF | |
| GAALIE (AB01) | | RSFYMSWVRQAPGK | |
| | | GLEWVATINQDGSE | |
| | | KLYVDSVKGRFTISR | |
| | | DNAKNSLFLQMNNL | |
| | | RVEDTAVYYCAAWS | |
| | | GNSGGMDVWGQGT | |
| | | TVSVSS | |
| HBC34 | V_{L} | SYELTQPPSVSVSPG | 54 |
| | | QTVSIPCSGDKLGNK | |
| | | NVCWFQHKPGQSPV | |
| | | LVIYEVKYRPSGIPER | |
| | | FSGSNSGNTATLTISG | |
| | | TQAMDEAAYFCQT | |
| | | WDSTTVVFGGGTRL | |
| | | TVL | |
| HBC34v35-MLNS- | V_{L} | SYELTQPPSVSVSPG | 55 |
| GAALIE (AB01) | | QTVSIPCSGDKLGNK | |
| | | NVAWFQHKPGQSPV | |
| | | LVIYEVKYRPSGIPER | |
| | | FSGSNSGNTATLTISG | |
| | | TQAMDEAAYFCQTF | |
| | | DSTTVVFGGGTRLTV | |
| | | L | |
| HBC34 | HC | ELQLVESGGGWVQP | 56 |
| | | GGSQRLSCAASGRIF | |
| | | RSFYMSWVRQAPGK | |
| | | GLEWVATINQDGSE | |
| | | KLYVDSVKGRFTISR | |
| | | DNAKNSLFLQMNNL | |
| | | RVEDTAVYYCAAWS | |
| | | GNSGGMDVWGQGT | |
| | | TVSVSSASTKGPSVF | |
| | | PLAPSSKSTSGGTAA | |
| | | LGCLVKDYFPEPVTV | |
| | | SWNSGALTSGVHTFP | |
| | | AVLQSSGLYSLSSVV | |
| | | TVPSSSLGTQTYICN | |
| | | VNHKPSNTKVDKKV | |
| | | EPKSCDKTHTCPPCP | |
| | | APELLGGPSVFLFPP | |
| | | KPKDTLMISRTPEVT | |
| | | CVVVDVSHEDPEVK | |
| | | FNWYVDGVEVHNA | |
| | | KTKPREEQYNSTYR | |
| | | VVSVLTVLHQDWLN | |
| | | GKEYKCKVSNKALP | |
| | | APIEKTISKAKGQPRE | |
| | | PQVYTLPPSRDELTK | |
| | | NQVSLTCLVKGFYPS | |
| | | DIAVEWESNGQPEN | |
| | | NYKTTPPVLDSDGSF | |
| | | FLYSKLTVDKSRWQ | |
| | | QGNVFSCSVMHEAL | |
| | | HNHYTQKSLSLSPGK | |
| HBC34v35-MLNS- | HC | ELQLVESGGGWVQP | 57 |
| GAALIE (AB01) | | GGSQRLSCAASGRIF | |
| (g1M17, 1) | | RSFYMSWVRQAPGK | |
| | | GLEWVATINQDGSE | |
| | | KLYVDSVKGRFTISR | |
| | | DNAKNSLFLQMNNL | |
| | | RVEDTAVYYCAAWS | |
| | | GNSGGMDVWGQGT | |
| | | TVSVSSASTKGPSVF | |
| | | PLAPSSKSTSGGTAA | |
| | | LGCLVKDYFPEPVTV | |
| | | SWNSGALTSGVHTFP | |
| | | AVLQSSGLYSLSSVV | |
| | | TVPSSSLGTQTYICN | |
| | | VNHKPSNTKVDKKV | |
| | | EPKSCDKTHTCPPCP | |
| | | APELLAGPSVFLFPP | |
| | | KPKDTLMISRTPEVT | |
| | | CVVVDVSHEDPEVK | |
| | | FNWYVDGVEVHNA | |
| | | KTKPREEQYNSTYR | |
| | | VVSVLTVLHQDWLN | |
| | | GKEYKCKVSNKALP | |
| | | LPEEKTISKAKGQPR | |
| | | EPQVYTLPPSRDELT | |
| | | KNQVSLTCLVKGFY | |
| | | PSDIAVEWESNGQPE | |
| | | NNYKTTPPVLDSDGS | |
| | | FFLYSKLTVDKSRW | |
| | | QQGNVFSCSVLHEA | |
| | | LHSHYTQKSLSLSPG | |
| | | K | |
| HBC34 | LC | SYELTQPPSVSVSPG | 58 |
| | | QTVSIPCSGDKLGNK | |
| | | NVCWFQHKPGQSPV | |
| | | LVIYEVKYRPSGIPER | |
| | | FSGSNSGNTATLTISG | |
| | | TQAMDEAAYFCQT | |
| | | WDSTTVVFGGGTRL | |
| | | TVL | |
| | | GQPKAAPSVTLFPPS | |
| | | SEELQANKATLVCLI | |
| | | SDFYPGAVTVAWKA | |
| | | DSSPVKAGVETTTPS | |
| | | KQSNNKYAASSYLS | |
| | | LTPEQWKSHRSYSC | |
| | | QVTHEGSTVEKTVA | |
| | | PTECS | |
| HBC34v35-MLNS- | LC | SYELTQPPSVSVSPG | 59 |
| GAALIE (AB01) | | QTVSIPCSGDKLGNK | |
| | | NVAWFQHKPGQSPV | |
| | | LVIYEVKYRPSGIPER | |
| | | FSGSNSGNTATLTISG | |
| | | TQAMDEAAYFCQTF | |
| | | DSTTVVFGGGTRLTV | |
| | | LGQPKAAPSVTLFPP | |
| | | SSEELQANKATLVCL | |
| | | ISDFYPGAVTVAWK | |
| | | ADSSPVKAGVETTTP | |
| | | SKQSNNKYAASSYLS | |
| | | LTPEQWKSHRSYSC | |
| | | QVTHEGSTVEKTVA | |
| | | PTECS | |
| WT hIgG1 Fc | Fc | APELLGGPSVFLFPP | 60 |
| | | KPKDTLMISRTPEVT | |
| | | CVVVDVSHEDPEVK | |
| | | FNWYVDGVEVHNA | |
| | | KTKPREEQYNSTYR | |
| | | VVSVLTVLHQDWLN | |
| | | GKEYKCKVSNKALP | |
| | | APIEKTISKAKGQPRE | |
| | | PQVYTLPPSRDELTK | |
| | | NQVSLTCLVKGFYPS | |
| | | DIAVEWESNGQPEN | |
| | | NYKTTPPVLDSDGSF | |
| | | FLYSKLTVDKSRWQ | |
| | | QGNVFSCSVMHEAL | |
| | | HNHYTQKSLSLSPGK | |

In certain embodiments, the anti-HBV antibody comprises one or more amino acid sequences as set forth in Table 3. In certain embodiments, the antibody, or the antigen-binding fragment thereof, according to the present disclosure comprises an amino acid sequence having at least 70%, at least 75%, at least 80%, at least 85%, at least 88%, at least 90%, at least 92%, at least 95%, at least 96%, at least 97%, at least 98%, or at least 99% identity to a CDR sequence, a V_{H} sequence, a V_{L} sequence, an HC sequence, and/or an LC sequence as shown in Table 3. In any of the presently disclosed embodiments, an antibody or antigen-binding fragment can comprise a CDR, V_{H}, V_{L}, HC, and/or LC sequence as set forth in Table 3. Exemplary methods for synthesizing antibodies having sequences shown in Table 3, and experimental data demonstrating binding and neutralization by AB01, are described in International Application Publication No. WO 2020/132091A2, which methods and data are incorporated herein by reference.

In some embodiments, an antibody or antigen-binding fragment of the present disclosure comprises: (i) CDRH1, CDRH2, and CDRH3 amino acid sequences according to SEQ ID NOs:44, 45 or 46, and 47, respectively; and (ii) CDRL1, CDRL2, and CDRL3 amino acid sequences according to SEQ ID NOs:48, 49 or 50, and 51 or 52, respectively.

Accordingly, in some embodiments, CDRH1, CDRH2, and CDRH3 are according to SEQ ID NOs:44, 45, and 47, respectively. In some embodiments, CDRH1, CDRH2, and CDRH3 are according to SEQ ID NOs:44, 46, and 47, respectively. In some embodiments, CDRL1, CDRL2, and CDRL3 are according to SEQ ID NOs:48, 49, and 52, respectively. In some embodiments, CDRL1, CDRL2, and CDRL3 are according to SEQ ID NOs:48, 50, and 52, respectively.

It will be understood that an antibody or antigen-binding fragment of the present disclosure can comprise any combination of the CDRH1, CDRH2, CDRH3, CDRL1, CDRL2, and CDRL3 amino acid sequences according to SEQ ID NOs:44-50 and 52.

In particular embodiments, an antibody or antigen-binding fragment of the present disclosure comprises: CDRH1, CDRH2, and CDRH3 amino acid sequences according to SEQ ID NOs:44, 45, and 47, respectively; and CDRL1, CDRL2, and CDRL3 amino acid sequences according to SEQ ID NOs:48, 49, and 52, respectively.

In certain embodiments, an antibody or antigen-binding fragment of the present disclosure comprises: (a) a light chain variable domain (V_{L}) comprising or consisting of an amino acid sequence that is at least 90%, at least 95%, or 100% identical to the amino acid sequence according to SEQ ID NO:55; and (b) a heavy chain variable domain (V_{H}) comprising or consisting of an amino acid sequence that is at least 90%, at least 95%, or 100% identical to the amino acid sequence according to SEQ ID NO:53.

In particular embodiments, an antibody or antigen-binding fragment of the present disclosure comprises (a) a light chain comprising or consisting of the amino acid sequence set forth in SEQ ID NO:59, and (b) a heavy chain comprising or consisting of the amino acid sequence set forth in SEQ ID NO:57.

### D. Pharmaceutical Compositions

In some embodiments, an antibody or antigen binding fragment thereof of the combination therapy is provided as a pharmaceutical composition, which includes the anti-HBV antibody and optionally, a pharmaceutically acceptable carrier. In some embodiments, a composition may include an anti-HBV antibody, wherein the antibody may make up at least 50% by weight (e.g., 60%, 70%, 75%, 80%, 85%, 90%, 95%, 96%, 97%, 98%, 99%, or more) of the total protein in the composition. In such a composition, the antibody may be in purified form.

Pharmaceutical compositions of the anti-HBV antibody may include an antimicrobial, particularly if packaged in a multiple dose format. They may comprise detergent, e.g., a Tween (polysorbate), such as Tween 80. When present, detergents are typically present at low levels, e.g., less than 0.01%. Compositions may also include sodium salts (e.g., sodium chloride) for tonicity. For example, in some embodiments, a pharmaceutical composition comprises NaCl at a concentration of 10±2 mg/ml.

Further, pharmaceutical compositions may comprise a sugar alcohol (e.g., mannitol) or a disaccharide (e.g., sucrose or trehalose), e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

An antibody composition of the present disclosure may also comprise one or more immunoregulatory agents. In some embodiments, one or more of the immunoregulatory agents include(s) an adjuvant.

Methods of preparing a pharmaceutical composition of the anti-HBV antibody may include the steps: (i) preparing the antibody; and (ii) admixing the purified antibody with one or more pharmaceutically acceptable carriers.

In some embodiments, a pharmaceutical composition comprising an anti-HBV antibody described herein (e.g., AB01) contains the antibody at a dose of 100 mg, 150 mg, 200 mg, 250 mg, or 300 mg. In some embodiments, a pharmaceutical composition comprising an anti-HBV antibody described herein (e.g., AB01) contains the antibody at a dose of from 100 mg to 300 mg. In some embodiments, a pharmaceutical composition comprising an anti-HBV antibody described herein (e.g., AB01) contains the antibody at a dose of from 100 mg to 200 mg.

In some embodiments, a pharmaceutical composition comprising an anti-HBV antibody described herein (e.g., AB01) contains the antibody at a dose of 200 mg.

### IV. METHODS OF TREATMENT USING COMBINATION THERAPIES

In some embodiments, the present disclosure provides methods for treating HDV infection or a HDV-associated disease in a subject.

In some embodiments, methods for treating HDV infection or a HDV-associated disease in a subject are provided, wherein the method comprises administering an siRNA and an antibody as described herein to the subject. In some embodiments, SIRNA01 and AB01 are administered to the subject.

In some embodiments, methods for treating HDV infection or a HDV-associated disease in a subject are provided, wherein the method comprises administering an siRNA and an antibody as described herein to the subject, and also administering a nucleoside/nucleotide reverse transcriptase inhibitor to the subject. As used herein, “nucleoside/nucleotide reverse transcriptase inhibitor” or “nucleos(t)ide reverse transcriptase inhibitor” (NRTI) refers to an inhibitor of DNA replication that is structurally similar to a nucleotide or nucleoside and specifically inhibits replication of the HBV cccDNA by inhibiting the action of HBV polymerase, and does not significantly inhibit the replication of the host (e.g., human) DNA. Such inhibitors include tenofovir, tenofovir disoproxil fumarate (TDF), tenofovir disoproxil (TD), tenofovir alafenamide (TAF), lamivudine, adefovir, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, ganciclovir, besifovir (ANA-380/LB-80380), and tenofvir-exaliades (TLX/CMX157). In some embodiments, the NRTI is tenofovir. In some embodiments, the NRTI is tenofovir disoproxil fumarate (TDF). In some embodiments, the NRTI is disoproxil (TD). In some embodiments, the NRTI is entecavir (ETV). In some embodiments, the NRTI is lamivudine. In some embodiments, the NRTI is adefovir or adefovir dipivoxil.

As used herein, a “subject” is an animal, such as a mammal, including any mammal that can be infected with HBV, e.g., a primate (such as a human, a non-human primate, e.g., a monkey, or a chimpanzee), or an animal that is considered an acceptable clinical model of HBV infection, HBV-AAV mouse model (see, e.g., Yang et al., Cell and Mol Immunol 2014, 11:71) or the HBV 1.3×fs transgenic mouse model (Guidotti et al., J. Virol. 1995, 69:6158). In some embodiments, the subject has a hepatitis B virus (HBV) infection. In some other embodiments, the subject has both a hepatitis B virus (HBV) infection and a hepatitis D virus (HDV) infection. In some other embodiments, the subject is a human, such as a human being having an HBV infection, especially a chronic hepatitis B virus (CHBV) infection.

As used herein, the terms “treating” or “treatment” refer to a beneficial or desired result including, but not limited to, alleviation or amelioration of one or more signs or symptoms associated with unwanted HBV gene expression or HBV replication, e.g., the presence of serum or liver HBV cccDNA, the presence of serum HBV DNA, the presence of serum or liver HBV antigen, e.g., HBsAg or HBeAg, elevated ALT, elevated AST (normal range is typically considered about 10 to 34 U/L), the absence of or low level of anti-HBV antibodies; a liver injury; cirrhosis; delta hepatitis; acute hepatitis B; acute fulminant hepatitis B; chronic hepatitis B; liver fibrosis; end-stage liver disease; hepatocellular carcinoma; serum sickness-like syndrome; anorexia; nausea; vomiting, low-grade fever; myalgia; fatigability; disordered gustatory acuity and smell sensations (aversion to food and cigarettes); or right upper quadrant and epigastric pain (intermittent, mild to moderate); hepatic encephalopathy; somnolence; disturbances in sleep pattern; mental confusion; coma; ascites; gastrointestinal bleeding; coagulopathy; jaundice; hepatomegaly (mildly enlarged, soft liver); splenomegaly; palmar erythema; spider nevi; muscle wasting; spider angiomas; vasculitis; variceal bleeding; peripheral edema; gynecomastia; testicular atrophy; abdominal collateral veins (caput medusa); ALT levels higher than AST levels; elevated gamma-glutamyl transpeptidase (GGT) (normal range is typically considered about 8 to 65 U/L) and alkaline phosphatase (ALP) levels (normal range is typically considered about 44 to 147 IU/L (international units per liter), not more than 3 times the ULN); slightly low albumin levels; elevated serum iron levels; leukopenia (i.e., granulocytopenia); lymphocytosis; increased erythrocyte sedimentation rate (ESR); shortened red blood cell survival; hemolysis; thrombocytopenia; a prolongation of the international normalized ratio (INR); presence of serum or liver HBsAg, HBeAg, Hepatitis B core antibody (anti-HBc) immunoglobulin M (IgM); hepatitis B surface antibody (anti-HBs), hepatitis B e antibody (anti-HBe), or HBV DNA; increased bilirubin levels; hyperglobulinemia; the presence of tissue-nonspecific antibodies, such as anti-smooth muscle antibodies (ASMAs) or antinuclear antibodies (ANAs) (10-20%); the presence of tissue-specific antibodies, such as antibodies against the thyroid gland (10-20%); elevated levels of rheumatoid factor (RF); low platelet and white blood cell counts; lobular, with degenerative and regenerative hepatocellular changes, and accompanying inflammation; and predominantly centrilobular necrosis, whether detectable or undetectable. The likelihood of developing, e.g., liver fibrosis, is reduced, for example, when an individual having one or more risk factors for liver fibrosis, e.g., chronic hepatitis B infection, either fails to develop liver fibrosis or develops liver fibrosis with less severity relative to a population having the same risk factors and not receiving treatment as described herein. “Treatment” can also mean prolonging survival as compared to expected survival in the absence of treatment.

As used herein, the terms “preventing” or “prevention” refer to the failure to develop a disease, disorder, or condition, or the reduction in the development of a sign or symptom associated with such a disease, disorder, or condition (e.g., by a clinically relevant amount), or the exhibition of delayed signs or symptoms delayed (e.g., by days, weeks, months, or years). Prevention may require the administration of more than one dose.

Doses are often expressed in relation to bodyweight. Thus, a dose which is expressed as [g, mg, or other unit]/kg (or g, mg, etc.) usually refers to [g, mg, or other unit]“per kg (or g, mg, etc.) bodyweight,” even if the term “bodyweight” is not explicitly mentioned.

In some embodiments, treatment of HBV infection results in a “functional cure” of hepatitis B. As used herein, functional cure is understood as clearance of circulating HBsAg and is may be accompanied by conversion to a status in which HBsAg antibodies become detectable using a clinically relevant assay. For example, detectable antibodies can include a signal higher than 10 mIU/ml as measured by Chemiluminescent Microparticle Immunoassay (CMIA) or any other immunoassay. Functional cure does not require clearance of all replicative forms of HBV (e.g., cccDNA from the liver). Anti-HBs seroconversion occurs spontaneously in about 0.2-1% of chronically infected patients per year. However, even after anti-HBs seroconversion, low level persistence of HBV is often observed for decades indicating that a functional rather than a complete cure occurs. Without being bound to a particular mechanism, the immune system may be able to keep HBV in check under conditions in which a functional cure has been achieved. A functional cure permits discontinuation of any treatment for the HBV infection. However, it is understood that a “functional cure” for HBV infection may not be sufficient to prevent or treat diseases or conditions that result from HBV infection, e.g., liver fibrosis, HCC, or cirrhosis. In some specific embodiments, a “functional cure” can refer to a sustained reduction in serum HBsAg, such as <1 IU/mL, for at least 3 months, at least 6 months, or at least one year following the initiation of a treatment regimen or the completion of a treatment regimen.

As used herein, the term “Hepatitis B virus-associated disease” or “HBV-associated disease,” is a disease or disorder that is caused by, or associated with HBV infection or replication. The term “HBV-associated disease” includes a disease, disorder or condition that would benefit from reduction in HBV gene expression or replication. Non-limiting examples of HBV-associated diseases include, for example, hepatitis D virus infection, delta hepatitis, acute hepatitis B; acute fulminant hepatitis B; chronic hepatitis B; liver fibrosis; end-stage liver disease; and hepatocellular carcinoma.

In some embodiments, an HBV-associated disease is chronic hepatitis B. Chronic hepatitis B is defined by one of the following criteria: (1) positive serum HBsAg, HBV DNA, or HBeAg on two occasions at least 6 months apart (any combination of these tests performed 6 months apart is acceptable); or (2) negative immunoglobulin M (IgM) antibodies to HBV core antigen (IgM anti-HBc) and a positive result on one of the following tests: HBsAg, HBeAg, or HBV DNA. Chronic HBV typically includes inflammation of the liver that lasts more than six months. Subjects having chronic HBV are HBsAg positive and have either high viremia (≥10⁴ HBV-DNA copies/ml blood) or low viremia (<10³ HBV-DNA copies/ml blood). In certain embodiments, subjects have been infected with HBV for at least five years. In certain embodiments, subjects have been infected with HBV for at least ten years. In certain embodiments, subjects became infected with HBV at birth. Subjects having chronic hepatitis B disease can be immune tolerant or have an inactive chronic infection without any evidence of active disease, and they are also asymptomatic. Patients with chronic active hepatitis, especially during the replicative state, may have symptoms similar to those of acute hepatitis. Subjects having chronic hepatitis B disease may have an active chronic infection accompanied by necroinflammatory liver disease, have increased hepatocyte turn-over in the absence of detectable necroinflammation, or have an inactive chronic infection without any evidence of active disease, and they are also asymptomatic. The persistence of HBV infection in chronic HBV subjects is the result of cccHBV DNA. In some embodiments, a subject having chronic HBV is HBeAg positive. In some other embodiments, a subject having chronic HBV is HBeAg negative. Subjects having chronic HBV have a level of serum HBV DNA of less than 105 and a persistent elevation in transaminases, for examples ALT, AST, and gamma-glutamyl transferase. A subject having chronic HBV may have a liver biopsy score of less than 4 (e.g., a necroinflammatory score). In some embodiments, ALT ULN values are 34 IU/mL for females and 43 IU/mL for males.

In some embodiments, an HBV-associated disease is hepatitis D virus infection. Hepatitis D virus or hepatitis delta virus (HDV) is a human pathogen. However, the virus is defective and depends on obligatory helper functions provided by HBV for transmission; indeed, HDV requires an associated or pre-existing HBV infection to become infectious and thrive, in particular, the viral envelope containing the surface antigen of hepatitis B. HDV can lead to severe acute and chronic forms of liver disease in association with HBV. Hepatitis D infection or delta hepatitis is highly endemic to several African countries, the Amazonian region, and the Middle East, while its prevalence is low in industrialized countries, except in the Mediterranean.

Transmission of HDV can occur either via simultaneous infection with HBV (coinfection) or superimposed on chronic hepatitis B or hepatitis B carrier state (superinfection). Both superinfection and coinfection with HDV typically result in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest fatality rate of all the hepatitis infections, at 20%.

In some embodiments, an HBV-associated disease is acute hepatitis B. Acute hepatitis B includes inflammation of the liver that lasts less than six months. Typical symptoms of acute hepatitis B are fatigue, anorexia, nausea, and vomiting. Very high aminotransferase values (>1000 U/L) and hyperbilirubinemia are often observed. Severe cases of acute hepatitis B may progress rapidly to acute liver failure, marked by poor hepatic synthetic function. This is often defined as a prothrombin time (PT) of 16 seconds or an international normalized ratio (INR) of 1.5 in the absence of previous liver disease. Acute hepatitis B may evolve into chronic hepatitis B.

In some embodiments, an HBV-associated disease is acute fulminant hepatitis B. A subject having acute fulminant hepatitis B has symptoms of acute hepatitis and the additional symptoms of confusion or coma (due to the liver's failure to detoxify chemicals) and bruising or bleeding (due to a lack of blood clotting factors).

Subjects having an HBV infection, e.g., chronic HBV, may develop liver fibrosis. Accordingly, in some embodiments, an HBV-associated disease is liver fibrosis. Liver fibrosis, or cirrhosis, is defined histologically as a diffuse hepatic process characterized by fibrosis (excess fibrous connective tissue) and the conversion of normal liver architecture into structurally abnormal nodules.

Subjects having an HBV infection, e.g., chronic HBV, may develop end-stage liver disease. Accordingly, in some embodiments, an HBV-associated disease is end-stage liver disease. For example, liver fibrosis may progress to a point where the body may no longer be able to compensate for, e.g., reduced liver function, as a result of liver fibrosis (i.e., decompensated liver), and result in, e.g., mental and neurological symptoms and liver failure.

Subjects having an HBV infection, e.g., chronic HBV, may develop hepatocellular carcinoma (HCC), also referred to as malignant hepatoma. Accordingly, in some embodiments, an HBV-associated disease is HCC. HCC commonly develops in subjects having CHB and may be fibrolamellar, pseudoglandular (adenoid), pleomorphic (giant cell), or clear cell.

An “HDV-associated disease” or a Hepatitis D-virus-associated disease” is a disease or disorder associated with expression of an HDV. Exemplary HDV-associated diseases include hepatitis B virus infection, acute hepatitis B, acute hepatitis D; acute fulminant hepatitis D; chronic hepatitis D; liver fibrosis; end-stage liver disease; and hepatocellular carcinoma.

“Therapeutically effective amount,” as used herein, is intended to include the amount of an siRNA, an anti-HBV antibody, or other active agent (e.g., tenofovir), that, when administered to a patient for treating a subject having an HBV infection or HBV-associated disease, is sufficient to effect treatment of the disease (e.g., by diminishing or maintaining the existing disease or one or more symptoms of disease). The “therapeutically effective amount” may vary depending on the active agent(s), how they are administered, the disease and its severity, and the history, age, weight, family history, genetic makeup, stage of pathological processes mediated by HBV gene expression, the types of preceding or concomitant treatments, if any, and other individual characteristics of the patient to be treated. A therapeutically effective amount may require the administration of more than one dose.

A “therapeutically-effective amount” also includes an amount of an siRNA, an anti-HBV antibody, or other active agent that produces some desired effect at a reasonable benefit/risk ratio applicable to any treatment. Therapeutic agents (e.g., siRNAs, anti-HBV antibodies) used in the methods of the present disclosure may be administered in a sufficient amount to produce a reasonable benefit/risk ratio applicable to such treatment.

The term “sample,” as used herein, includes a collection of similar fluids, cells, or tissues isolated from a subject, as well as fluids, cells, or tissues present within a subject. Examples of biological fluids include blood, serum, and serosal fluids, plasma, lymph, urine, saliva, and the like. Tissue samples may include samples from tissues, organs or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In certain embodiments, a “sample derived from a subject” refers to blood, or plasma or serum obtained from blood drawn from the subject. In further embodiments, a “sample derived from a subject” refers to liver tissue (or subcomponents thereof) or blood tissue (or subcomponents thereof, e.g., serum) derived from the subject.

In some embodiments of the methods described herein, the anti-HBV antibody is administered subcutaneously. In some embodiments, the anti-HBV antibody is administered every 4 weeks. In some embodiments, the anti-HBV antibody is administered every 8 weeks. In some embodiments, the anti-HBV antibody is administered at a dose of from 100 mg to 300 mg. In some embodiments, the anti-HBV antibody is administered at a dose of 100 mg. In some embodiments, the anti-HBV antibody is administered at a dose of 150 mg. In some embodiments, the anti-HBV antibody is administered at a dose of 200 mg. In some embodiments, the anti-HBV antibody is administered at a dose of 250 mg. In some embodiments, the anti-HBV antibody is administered at a dose of 300 mg. In some embodiments, the subject is administered the anti-HBV antibody for a period of 8 weeks, 24 weeks, 48 weeks, or 88 weeks.

In some embodiments of the methods described herein, the siRNA is administered subcutaneously. In some embodiments, the siRNA is administered every 4 weeks. In some embodiments, the siRNA is administered every 8 weeks. In some embodiments, the siRNA is administered at a dose of from 20 mg to 900 mg. In some embodiments, the siRNA is administered at a dose of from 100 mg to 300 mg. In some embodiments, the siRNA is administered at a dose of 20 mg, 50 mg, 100 mg, 150 mg, 200 mg, 250 mg, 300 mg, 400 mg, or 450 mg. In some embodiments, the siRNA is administered at a dose of 200 mg. In some embodiments, the subject is administered the siRNA for a period of 8 weeks, 24 weeks, 48 weeks, or 88 weeks.

In some embodiments of the methods described herein, the NRTI is administered orally. In some embodiments, the NRTI is administered daily. In some embodiments, the NRTI is administered at a dose of 300 mg.

In some embodiments of the methods described herein, the subject is administered the siRNA and the anti-HBV antibody beginning on the same day.

In some embodiments of the methods described herein, the subject is administered the siRNA and the anti-HBV antibody beginning on the same day and for a period of 8 weeks, 24 weeks, 48 weeks, or 88 weeks.

In some embodiments of the methods described herein, the subject achieves one or more of: a ≥2 log 10 decrease in HDV RNA compared to baseline (i.e., prior to treatment); a HDV RNA<LOQ at week 24; and a ALT<upper limit of normal (ULN) at Week 24. In some embodiments, ALT ULN values are 34 IU/mL for females and 43 IU/mL for males.

The present disclosure also provides antibodies, siRNAs, and/or NRTIs described herein, and pharmaceutical compositions comprising the same, for use in the aforementioned methods. Uses of the antibodies, siRNAs, and/or NRTIs.

### V. KITS FOR COMBINATION THERAPIES

Also provided herein are kits including components of the therapy for treating HDV infection or a HDV-associated disease. The kits may include an siRNA (e.g., SIRNA01) an anti-HBV antibody (e.g., AB01). The kits may include an siRNA (e.g., SIRNA01), an anti-HBV antibody (e.g., AB01), and a NRTI (e.g., tenofovir disoproxil fumarate, tenofovir disoproxil). Kits may additionally include instructions for preparing and/or administering the components of the HBV combination therapy.

### VI. EXAMPLE EMBODIMENTS

In some embodiments, the present disclosure provides:
1. A method of treating hepatitis D virus (HDV) infection in a subject in need thereof, comprising administering to the subject:
(a) an anti-HBV antibody; and
(b) an siRNA that targets an HBV mRNA.
2. A method of treating an HDV-associated disease in a subject in need thereof, comprising administering to the subject:
(a) an anti-HBV antibody; and
(b) an siRNA that targets an HBV mRNA.
3. The method of embodiment 2, wherein the HDV-associated disease is chronic hepatitis; acute hepatitis D; acute fulminant hepatitis D; chronic hepatitis D; liver fibrosis; end-stage liver disease; or hepatocellular carcinoma.
4. The method any one of the preceding embodiments, wherein the subject has chronic hepatitis B virus (HBV)/HDV coinfection.
5. The method of any one of the preceding embodiments, wherein the subject has a positive serum HBsAg, HBV DNA, or HBeAg on 2 occasions at least 6 months apart prior to treatment.
6. The method of any one of the preceding embodiments, wherein the subject has tested positive for HDV antibody or HDV RNA prior to treatment.
7. The method of any one of the preceding embodiments, wherein the subject has tested positive for HDV antibody and HDV RNA prior to treatment.
8. The method of any one of the preceding embodiments, wherein the subject has tested positive for HDV antibody for at least 6 months prior to treatment.
9. The method of any one of the preceding embodiments, wherein the subject has a HDV RNA≥500 IU/mL prior to treatment.
10. The method of any one of the preceding embodiments, wherein the subject has a HBsAg level>0.05 IU/mL prior to treatment.
11. The method of any one of the preceding embodiments, wherein the subject has a HBsAg level>10,000 IU/mL prior to treatment.
12. The method of any one of the preceding embodiments, wherein the subject has chronic hepatitis.
13. The method of any one of the preceding embodiments, wherein the subject has an alanine aminotransferase (ALT) level>the upper limit of normal (ULN) and <5 times ULN prior to treatment.
14. The method of any one of the preceding embodiments, wherein the subject has an ALT level>ULN and an aspartate aminotransferase (AST) level>ULN prior to treatment.
15. The method of any one of the preceding embodiments, wherein the subject has an ALT level<5 times ULN and an AST level<5 times ULN prior to treatment.
16. The method of any one of the preceding embodiments, wherein the subject is noncirrhotic.
17. The method of embodiment 16, wherein the subject has a liver biopsy with Meta-Analysis of Histological Data in Viral Hepatitis (METAVIR) F0-F3 or Liver elastography (Fibroscan®)<12 kilopascal (kPa) within the 12 months prior to treatment.
18. The method of embodiment 16 or embodiment 17, wherein the subject has a creatine clearance (CLcr)≥30 mL/min as calculated by the Cockcroft-Gault formula prior to treatment.
19. The method of any one of embodiments 1-15, wherein the subject is cirrhotic.
20. The method of embodiment 19, wherein the subject has a liver biopsy with METAVIR F4 or Liver elastography (Fibroscan®)≥12 kilopascal (kPa) within the 12 months prior to treatment.
21. The method of embodiment 19 or embodiment 20, wherein the subject has a creatine clearance (CLcr)≥60 mL/min as calculated by the Cockcroft-Gault formula prior to treatment.
22. The method of any one of embodiments 19-21, wherein the subject has a Child-Pugh-Turcotte (CPT) score of 5 or higher prior to treatment.
23. The method of any one of embodiments 19-21, wherein the subject has a Child-Pugh-Turcotte (CPT) score of 5 or 6 prior to treatment.
24. The method of any one of embodiments 19-21, wherein the subject has a Child-Pugh-Turcotte (CPT) score of 7 or higher prior to treatment.
25. The method of any one of embodiments 19-21, wherein the subject has a Child-Pugh-Turcotte (CPT) score of 7 to 9 prior to treatment.
26. The method of any one of embodiments 19-21, wherein the subject has a Child-Pugh-Turcotte (CPT) score of ≥10 prior to treatment.
27. The method of any one of the preceding embodiments, wherein the subject has not previously been administered an anti-HIV antibody.
28. The method of any one of preceding embodiments, wherein the subject has not previously been administered an siRNA that targets an HBV mRNA.
29. The method according to any one of the preceding embodiments, wherein the anti-HBV antibody recognizes HBV genotypes A, B, C, D, E, F, G, H, I, and J.
30. The method according to any one of the preceding embodiments, wherein the anti-HBV antibody is a human antibody.
31. The method according to any one of the preceding embodiments, wherein the antibody is HBC34 or a non-natural variant of HBC34.
32. The method according to any one of the preceding embodiments, wherein the anti-HBV antibody comprises:
(i) CDRH1, CDRH2, and CDRH3 amino acid sequences according to SEQ ID NOs:44, 45 or 46, and 47, respectively; and
(ii) CDRL1, CDRL2, and CDRL3 amino acid sequences according to SEQ ID NOs:48, 49 or 50, and 52, respectively.
33. The method according to embodiment 32, wherein the anti-HBV antibody comprises:
(i) CDRH1, CDRH2, and CDRH3 amino acid sequences according to SEQ ID NOs:44, 45, and 47, respectively; and
(ii) CDRL1, CDRL2, and CDRL3 amino acid sequences according to SEQ ID NOs:48, 49, and 52, respectively.
34. The method according to embodiment 32, wherein the anti-HBV antibody comprises:
(i) CDRH1, CDRH2, and CDRH3 amino acid sequences according to SEQ ID NOs:44, 46, and 47, respectively; and
(ii) CDRL1, CDRL2, and CDRL3 amino acid sequences according to SEQ ID NOs:48, 50, and 52, respectively.
35. The method according to any one of the preceding embodiments, wherein the anti-HBV antibody comprises:
(a) a light chain variable domain (V_{L}) that is at least 90%, at least 95%, or 100% identical to the amino acid sequence according to SEQ ID NO:55; and (b) a heavy chain variable domain (V_{H}) that is at least 90%, at least 95%, or 100% identical to the amino acid sequence according to SEQ ID NO:53.
36. The method according to any one of the preceding embodiments, wherein the anti-HBV antibody comprises:
(a) a light chain variable domain (V_{L}) amino acid sequence according to SEQ ID NO:55; and (b) a heavy chain variable domain (V_{H}) amino acid sequence according to SEQ ID NO:53.
37. The method according to any one of the preceding embodiments, wherein the anti-HBV antibody comprises:
(a) a light chain that is at least 90%, at least 95%, or 100% identical to the amino acid sequence set forth in SEQ ID NO:59, and (b) a heavy chain that is at least 90%, at least 95%, or 100% identical to the amino acid sequence according to SEQ ID NO:57.
38. The method according to any one of the preceding embodiments, wherein the anti-HBV antibody comprises:
(a) a light chain amino acid sequence according to SEQ ID NO:59, and (b) a heavy chain amino acid sequence according to SEQ ID NO:57.
39. The method according to any one of the preceding embodiments, wherein the anti-HBV antibody is a monoclonal antibody.
40. The method according to any one of the preceding embodiments, wherein the anti-HBV antibody is a bispecific antibody, with a first specificity for HBsAg, and a second specificity that stimulates an immune effector.
41. The method according to embodiment 40, wherein the second specificity stimulates cytotoxicity or a vaccinal effect.
42. The method according to any one of the preceding embodiments, wherein the subject is a human and a therapeutically effective amount of the anti-HBV antibody is administered; wherein the therapeutically effective amount is from about 3 mg/kg to about 30 mg/kg.
43. The method according to any one of the preceding embodiments, wherein the siRNA inhibits expression of an HBV transcript that encodes an HBsAg protein, an HBcAg protein, and HBx protein, or an HBV DNA polymerase protein.
44. The method according to any one of the preceding embodiments, wherein the siRNA comprises a sense strand and an antisense strand forming a double-stranded region, wherein the sense strand comprises at least 15 contiguous nucleotides differing by no more than 3 nucleotides from nucleotides 1579-1597 of SEQ ID NO:1 wherein T is replaced with U.
45. The method according to any one of the preceding embodiments, wherein the siRNA comprises a sense strand and an antisense strand, wherein the sense strand comprises nucleotides 1579-1597 of SEQ ID NO:1 wherein T is replaced with U.
46. The method according to any one of the preceding embodiments, wherein the siRNA binds to at least 15 contiguous nucleotides of a target encoded by: P gene, nucleotides 2309-3182 and 1-1625 of NC_003977.2; S gene (encoding L, M, and S proteins), nucleotides 2850-3182 and 1-837 of NC_003977.2; HBx, nucleotides 1376-1840 of NC_003977.2; or C gene, nucleotides 1816-2454 of NC_003977.2.
47. The method according to any one of the preceding embodiments, wherein the antisense strand of the siRNA comprises at least 15 contiguous nucleotides of the nucleotide sequence of 5′-UGUGAAGCGAAGUGCACACUU-3′ (SEQ ID NO:4).
48. The method according to any one of the preceding embodiments, wherein the antisense strand of the siRNA comprises at least 19 contiguous nucleotides of the nucleotide sequence of 5′-UGUGAAGCGAAGUGCACACUU-3′ (SEQ ID NO:4).
49. The method according to any one of the preceding embodiments, wherein the antisense strand of the siRNA comprises the nucleotide sequence of 5′-UGUGAAGCGAAGUGCACACUU-3′ (SEQ ID NO:4).
50. The method according to any one of the preceding embodiments, wherein the antisense strand of the siRNA consists of the nucleotide sequence of 5′-UGUGAAGCGAAGUGCACACUU-3′ (SEQ ID NO:4).
51. The method according to any one of the preceding embodiments, wherein the sense strand of the siRNA comprises the nucleotide sequence of 5′-GUGUGCACUUCGCUUCACA-3′ (SEQ ID NO:3).
52. The method according to any one of the preceding embodiments, wherein the sense strand of the siRNA consists of the nucleotide sequence of 5′-GUGUGCACUUCGCUUCACA-3′ (SEQ ID NO:3).
53. The method according to any one of the preceding embodiments, wherein at least one strand of the siRNA comprises a 3′ overhang of at least 1 nucleotide.
54. The method according to any one of the preceding embodiments, wherein at least one strand of the siRNA comprises a 3′ overhang of at least 2 nucleotides.
55. The method according to any one of the preceding embodiments, wherein the double-stranded region of the siRNA is 15-30 nucleotide pairs in length.
56. The method according to any one of the preceding embodiments, wherein the double-stranded region of the siRNA is 17-23 nucleotide pairs in length.
57. The method according to any one of the preceding embodiments, wherein the double-stranded region of the siRNA is 17-25 nucleotide pairs in length.
58. The method according to any one of the preceding embodiments, wherein the double-stranded region of the siRNA is 23-27 nucleotide pairs in length.
59. The method according to any one of the preceding embodiments, wherein the double-stranded region of the siRNA is 19-21 nucleotide pairs in length.
60. The method according to any one of the preceding embodiments, wherein the double-stranded region of the siRNA is 21-23 nucleotide pairs in length.
61. The method according to any one of the preceding embodiments, wherein each strand of the RNAi agent has 15-30 nucleotides.
62. The method according to any one of the preceding embodiments, wherein each strand of the RNAi agent has 19-30 nucleotides.
63. The method according to any one of the preceding embodiments, wherein substantially all of the nucleotides of the sense strand of the siRNA and substantially all of the nucleotides of the antisense strand of the siRNA are modified nucleotides, and wherein the sense strand is conjugated to a ligand attached at the 3′-terminus.
64. The method according to embodiment 63, wherein the ligand is one or more GalNAc derivatives attached through a monovalent linker, bivalent branched linker, or trivalent branched linker.
65. The method according to embodiment 63 or embodiment 64, wherein the ligand is

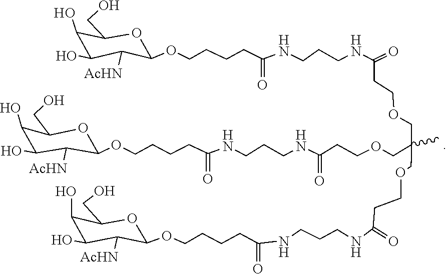 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

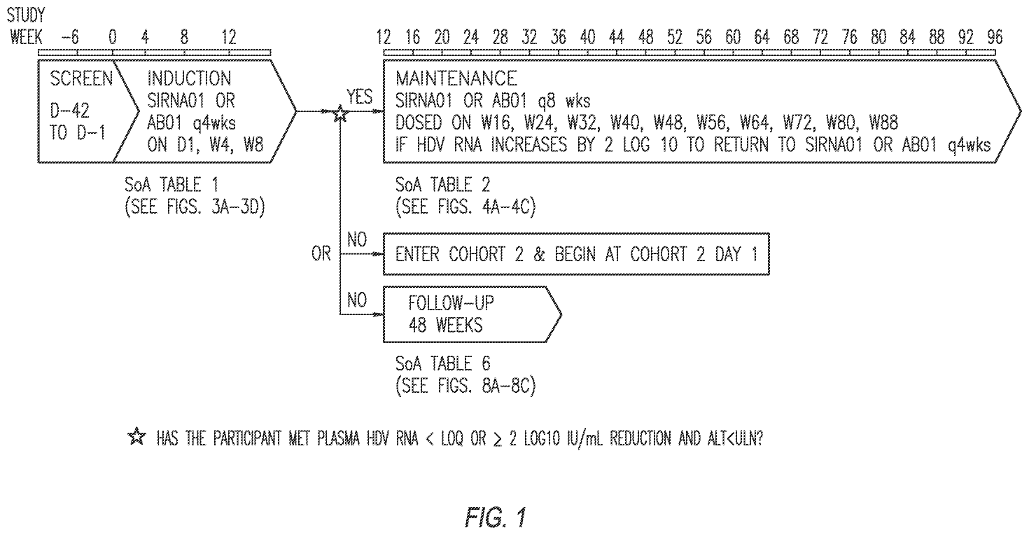

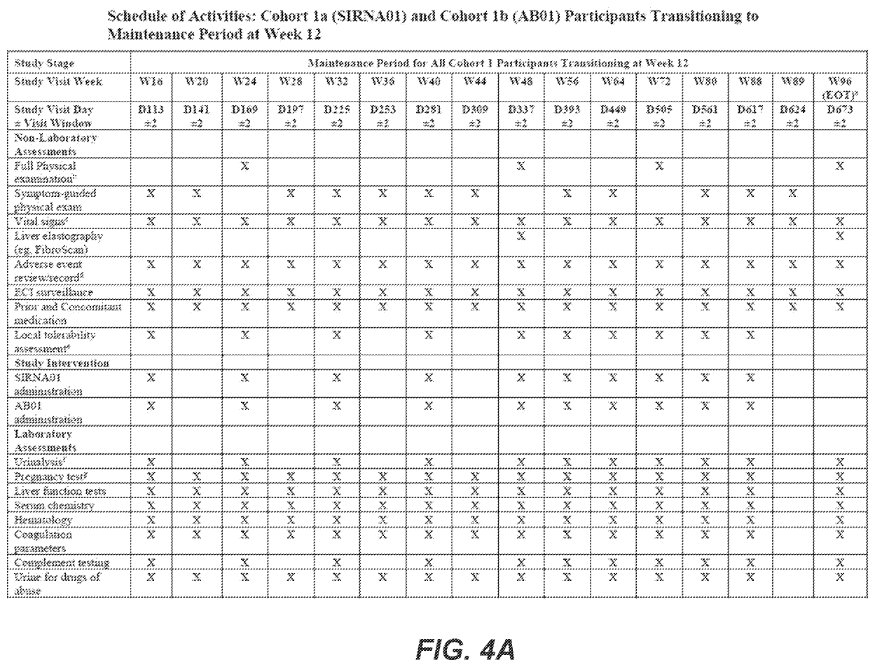

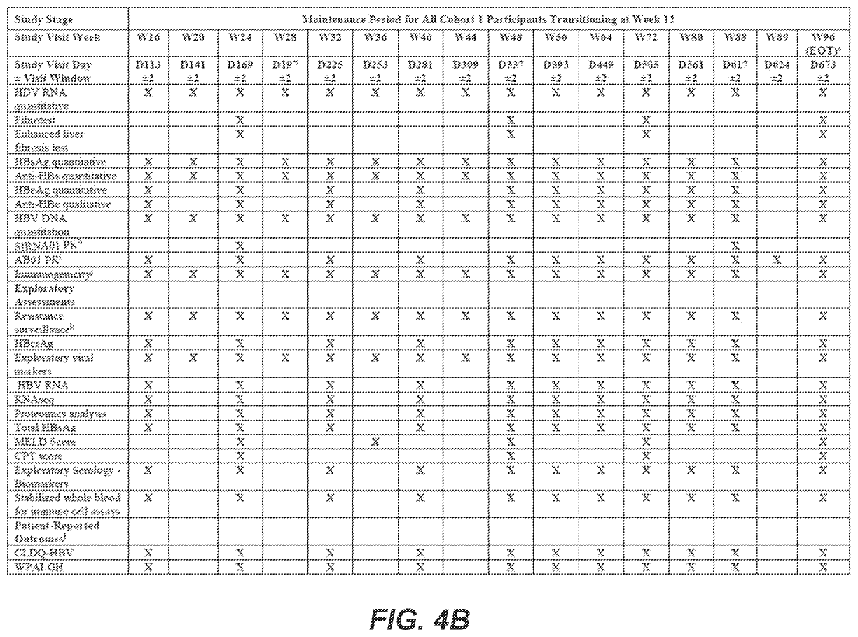

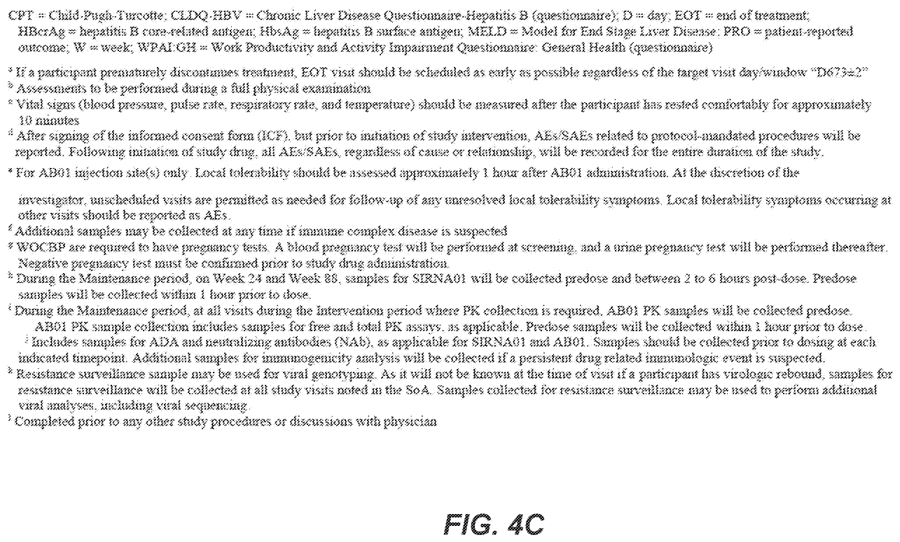

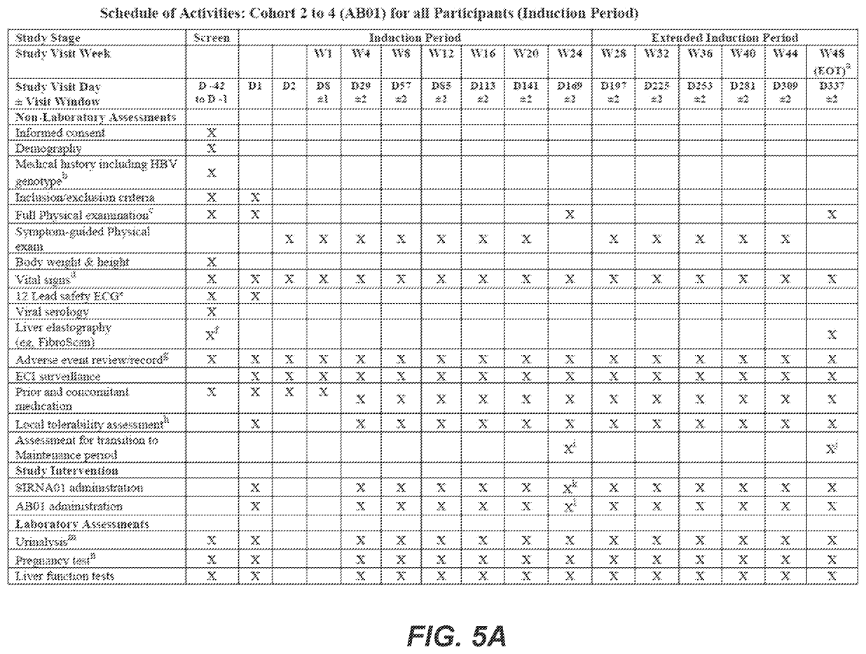

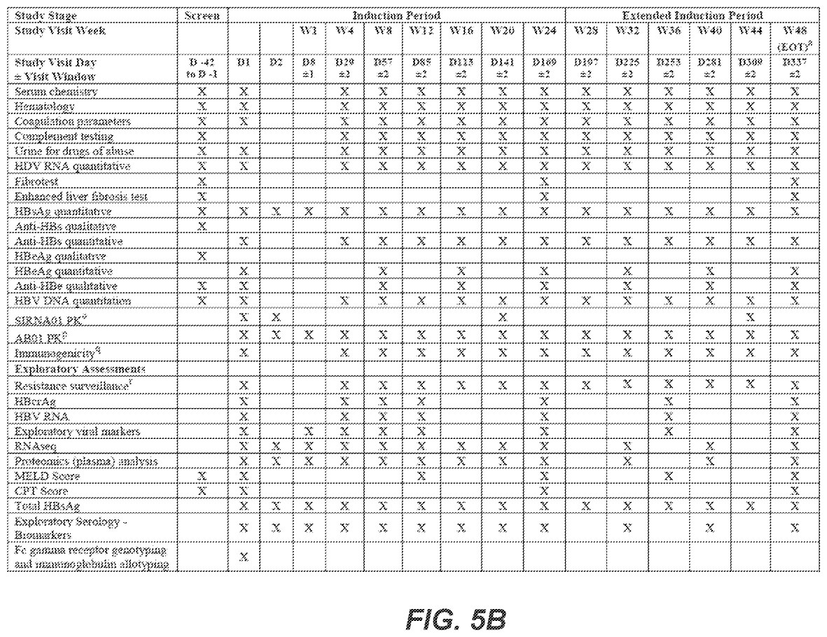

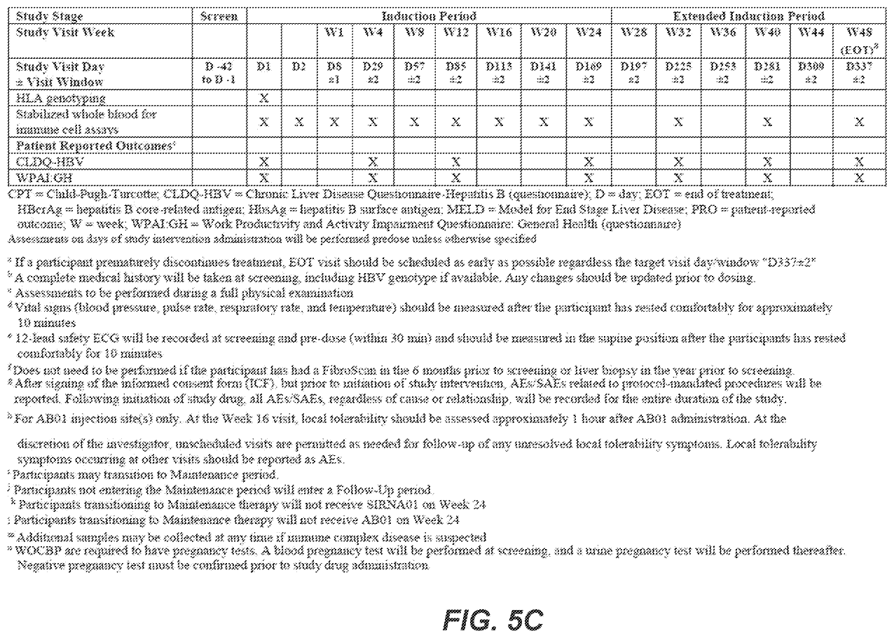

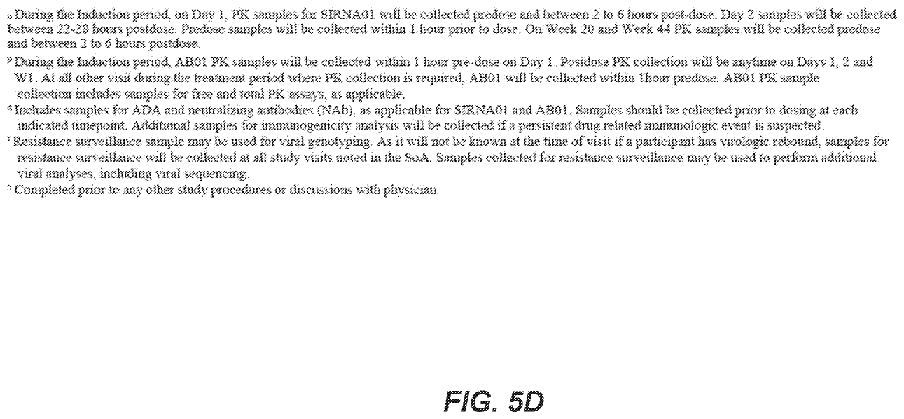

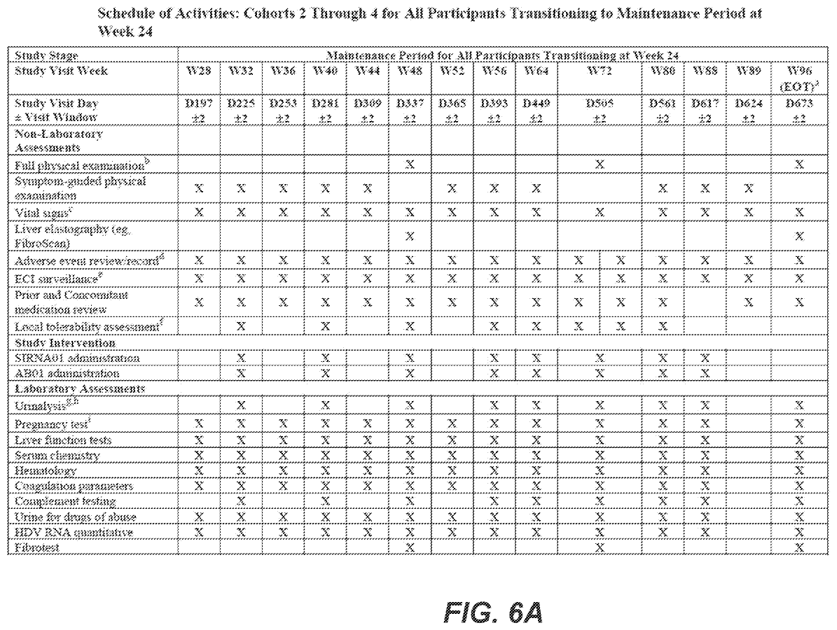

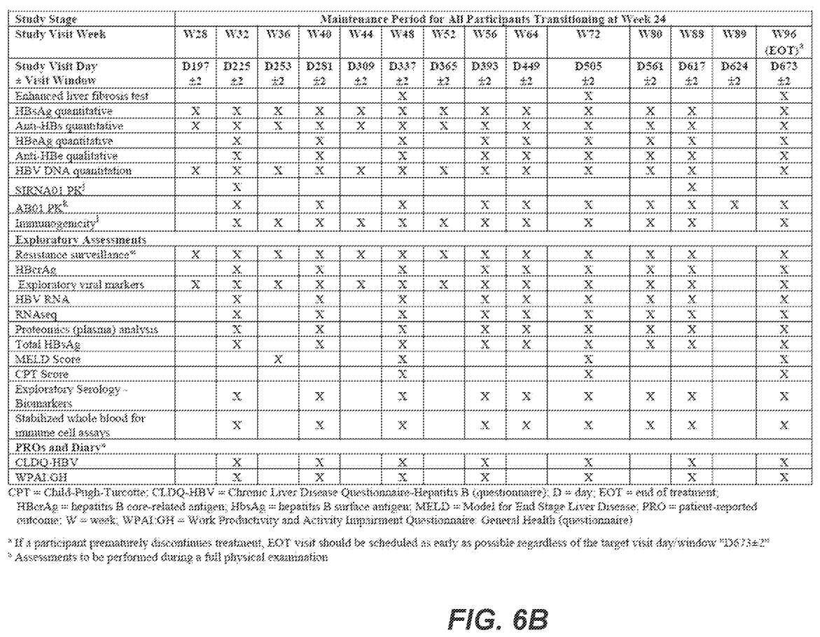

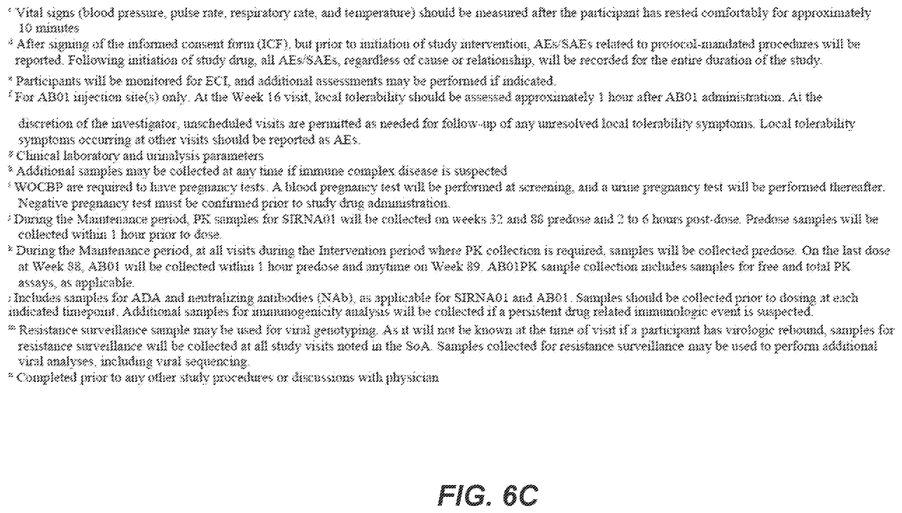

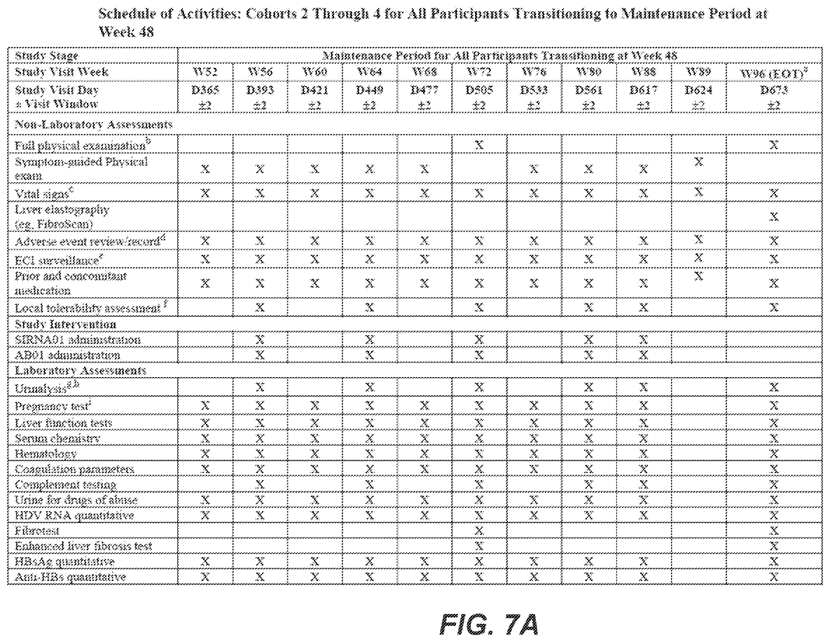

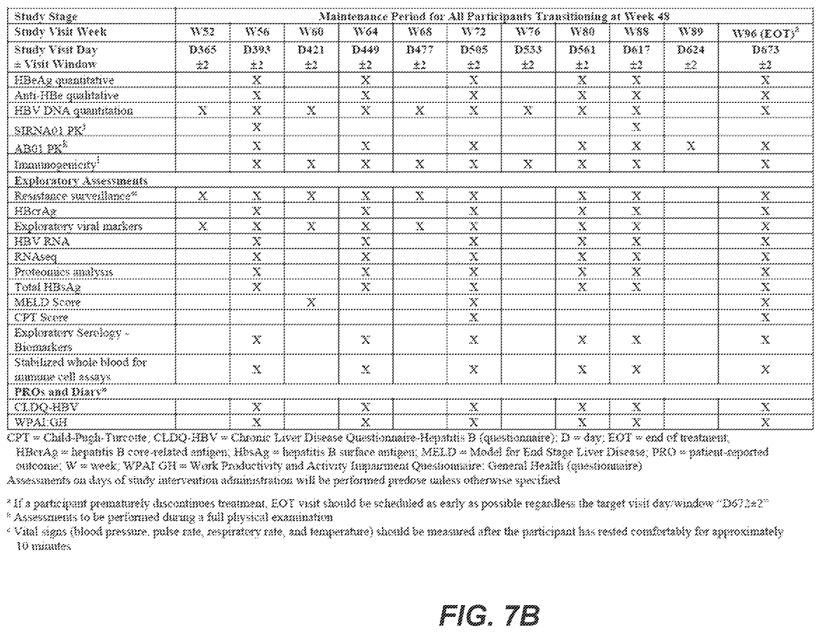

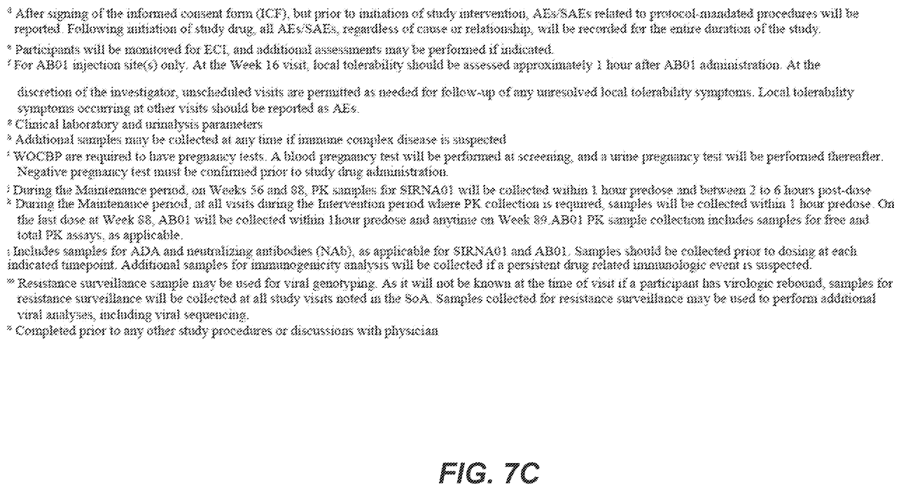

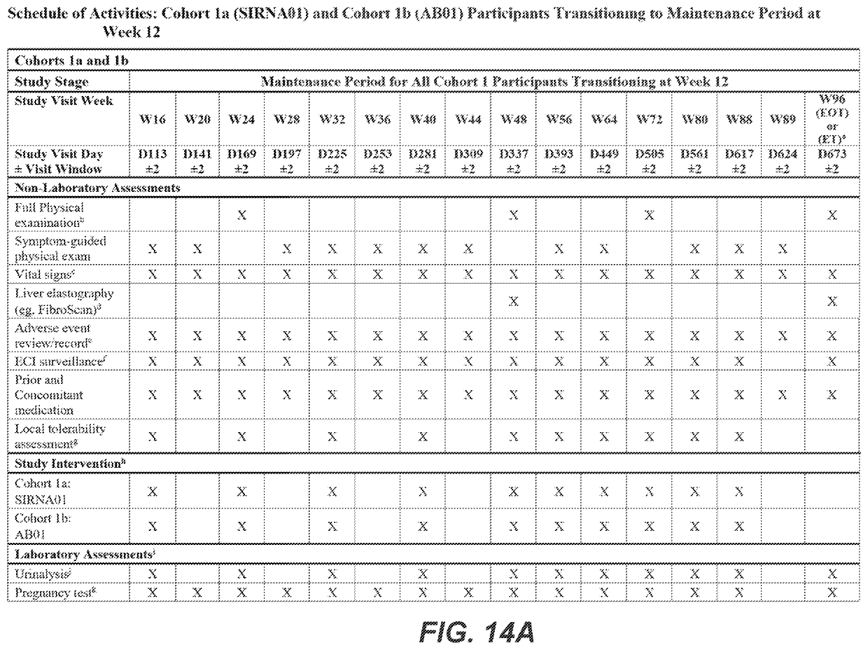

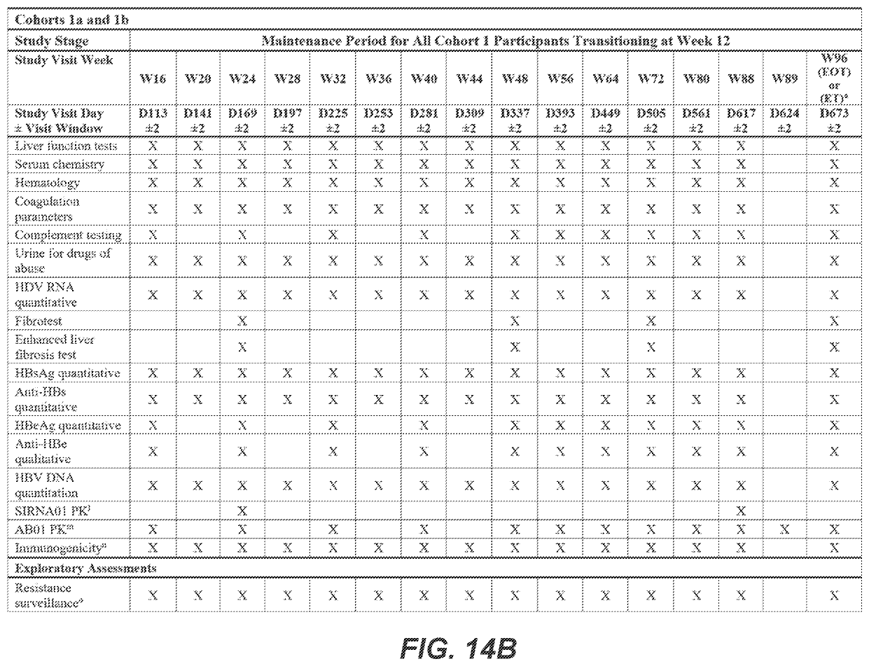

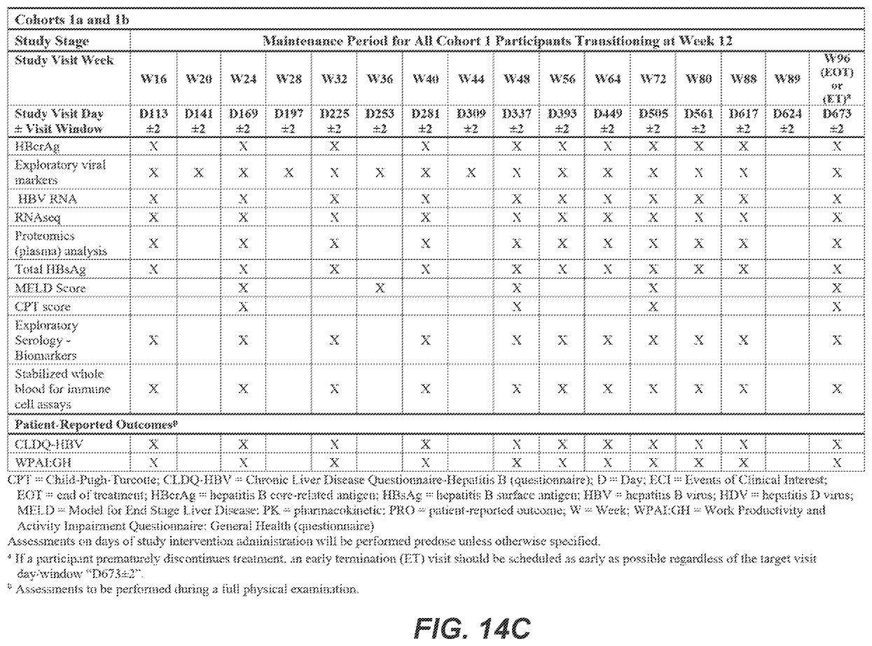

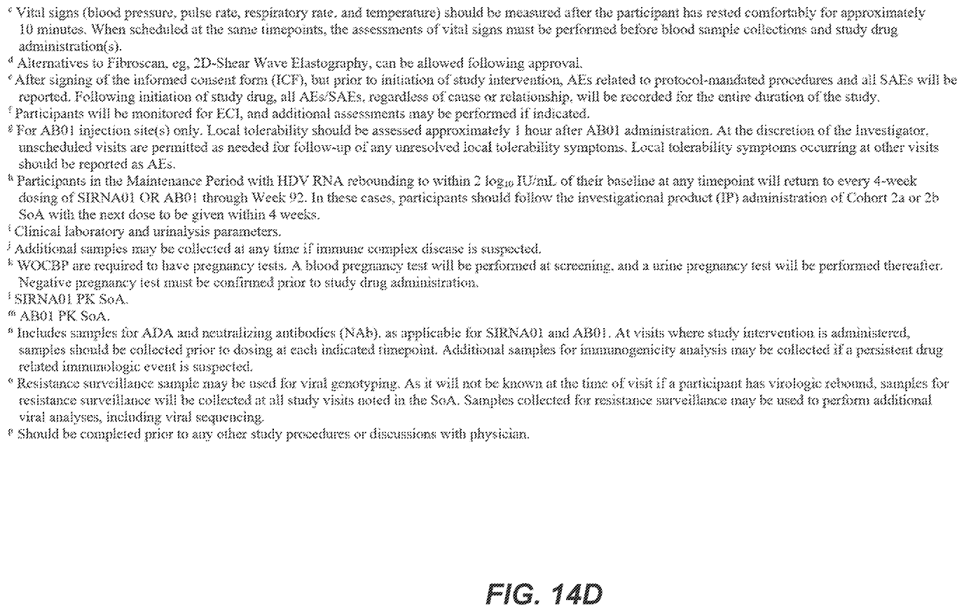

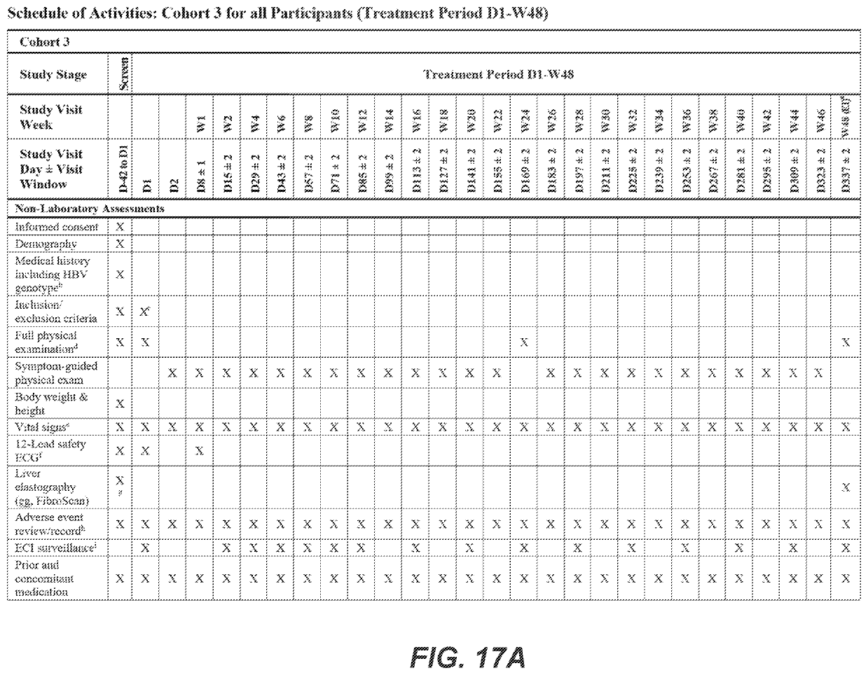

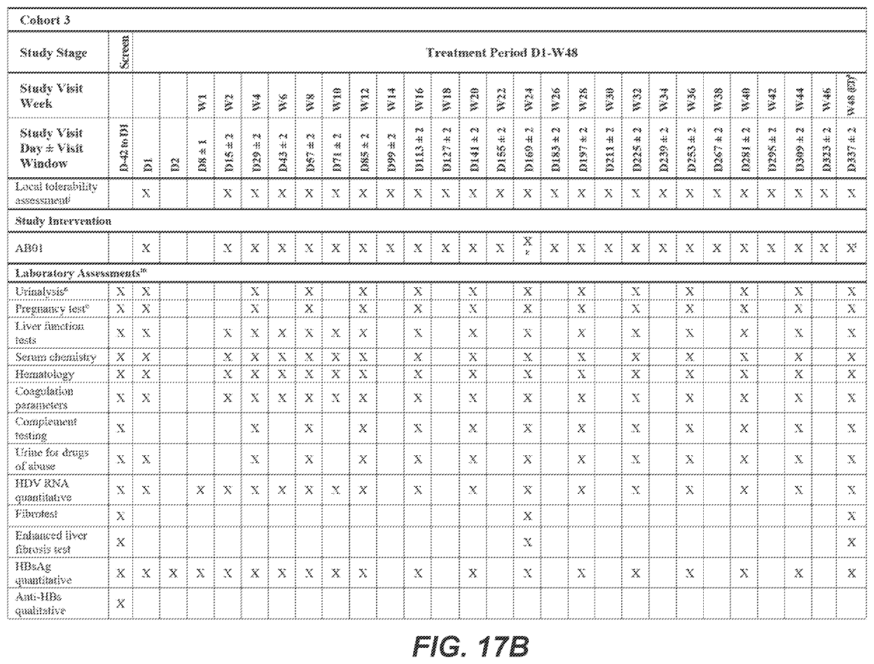

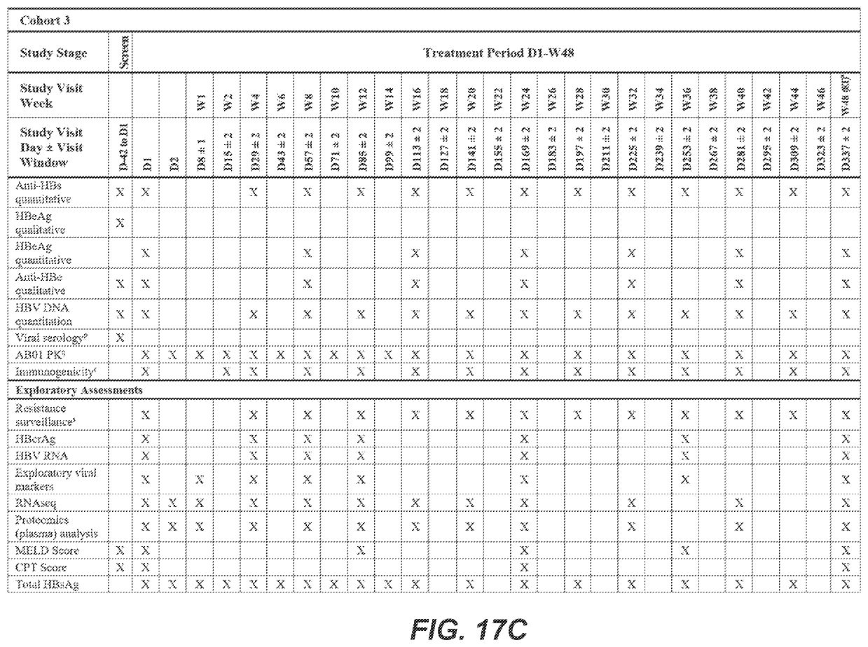

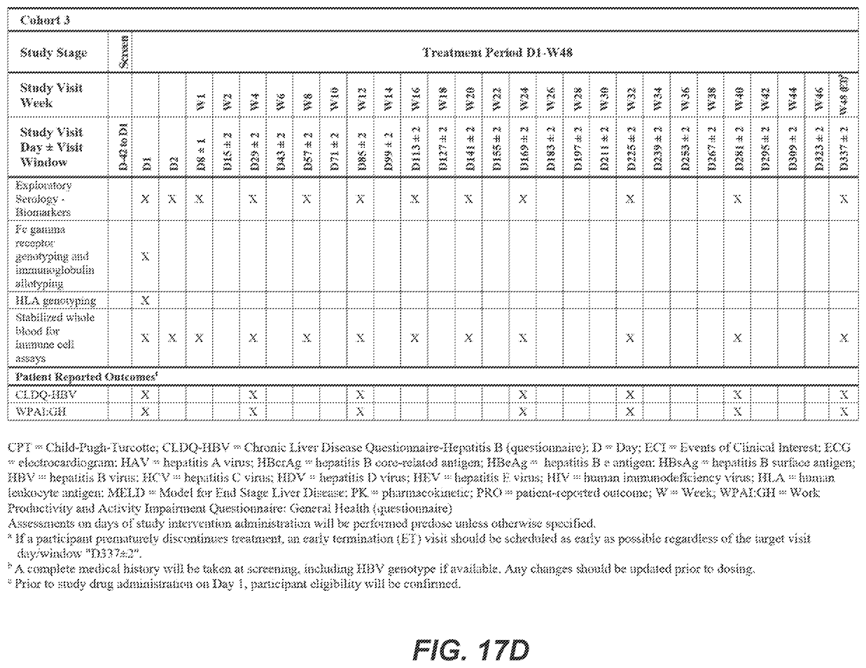

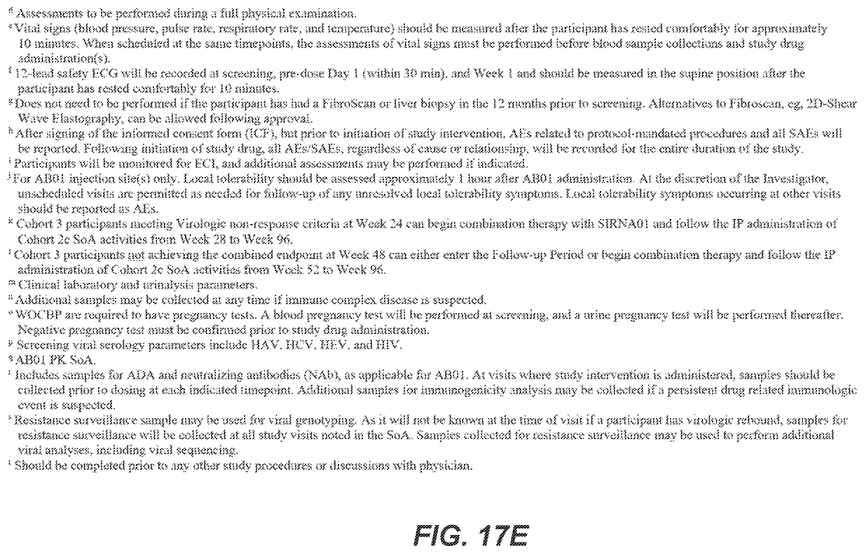

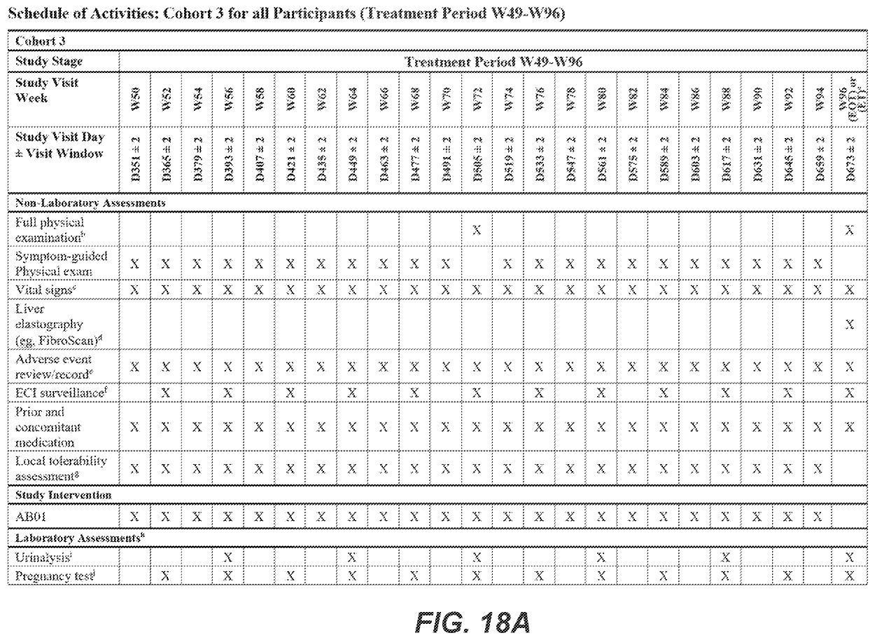

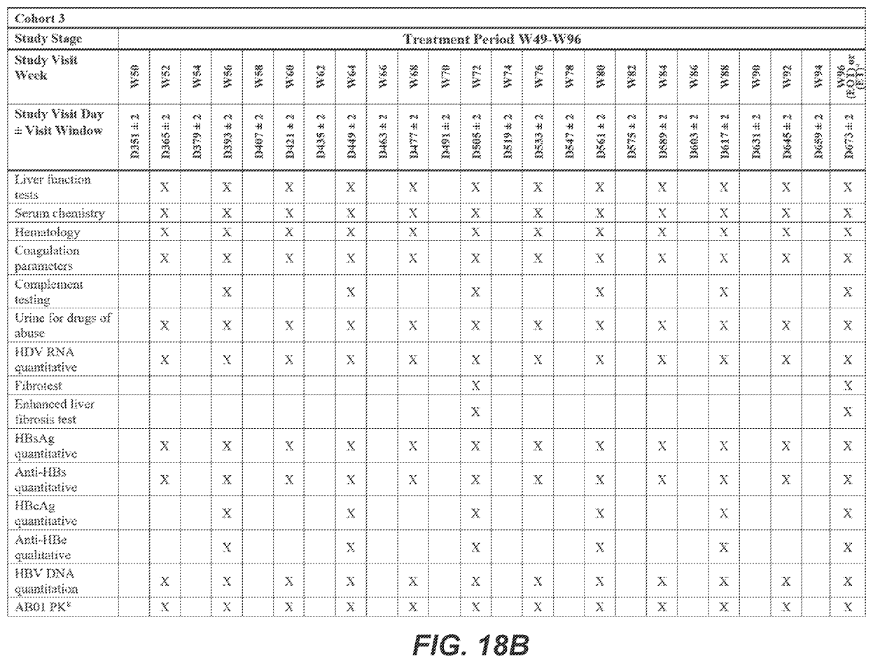

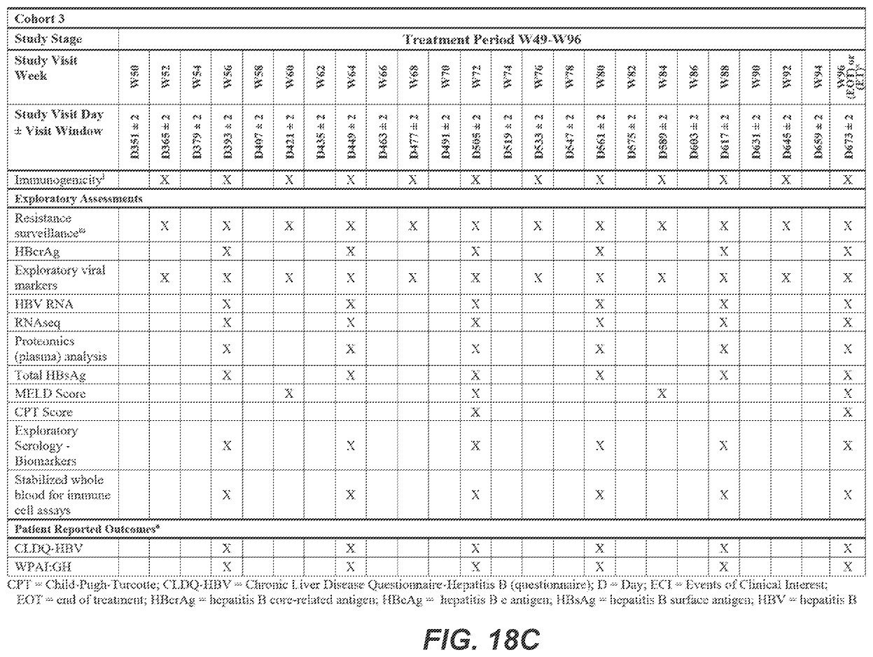

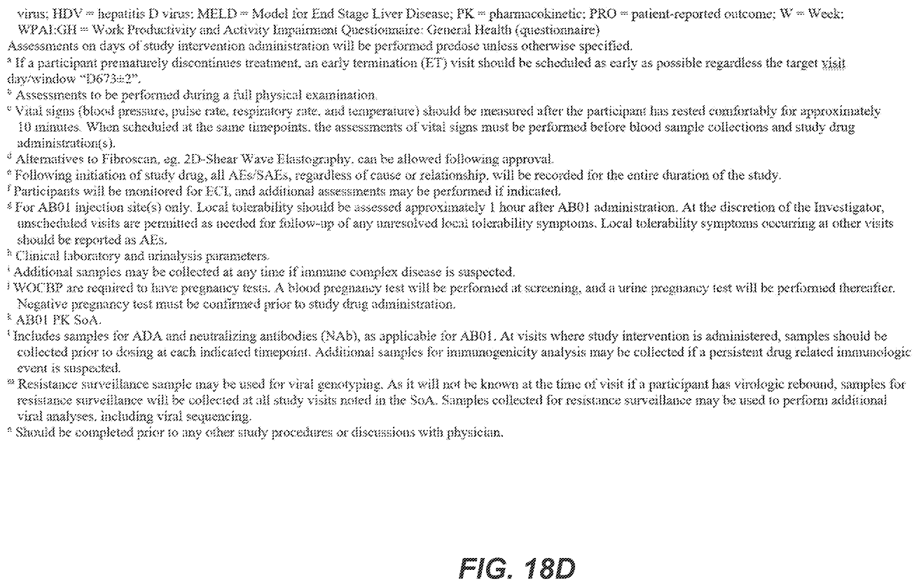

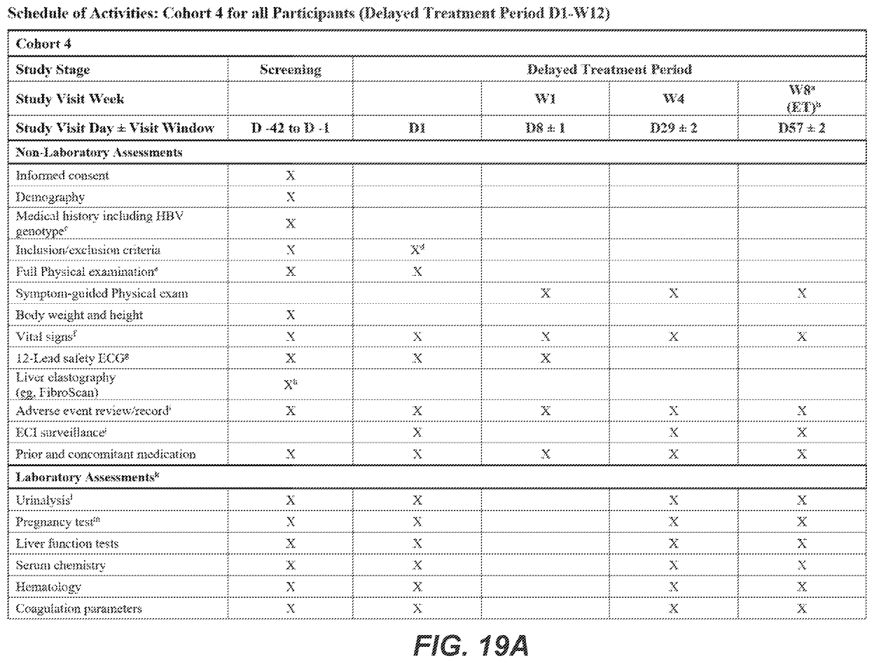

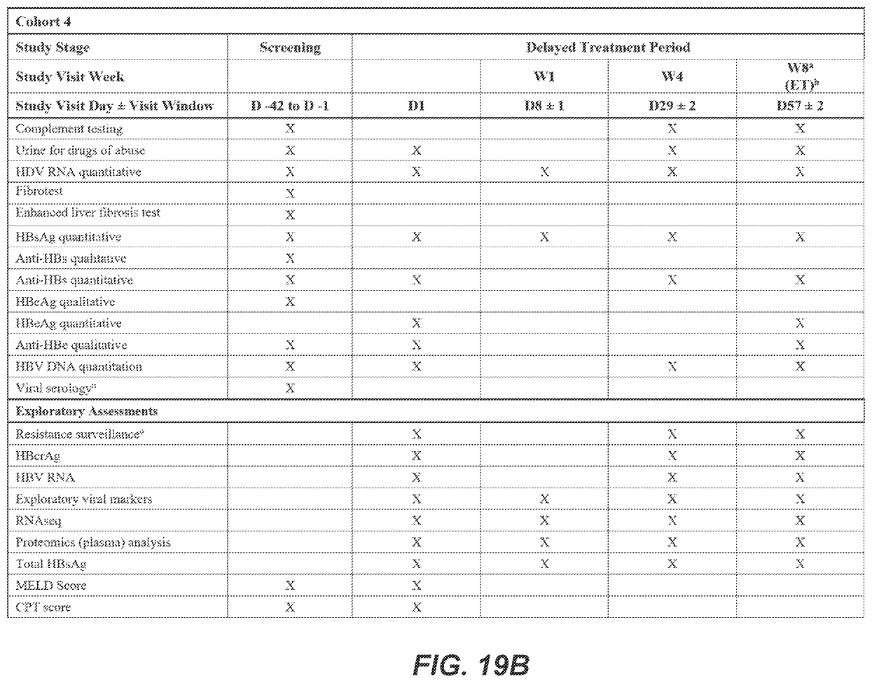

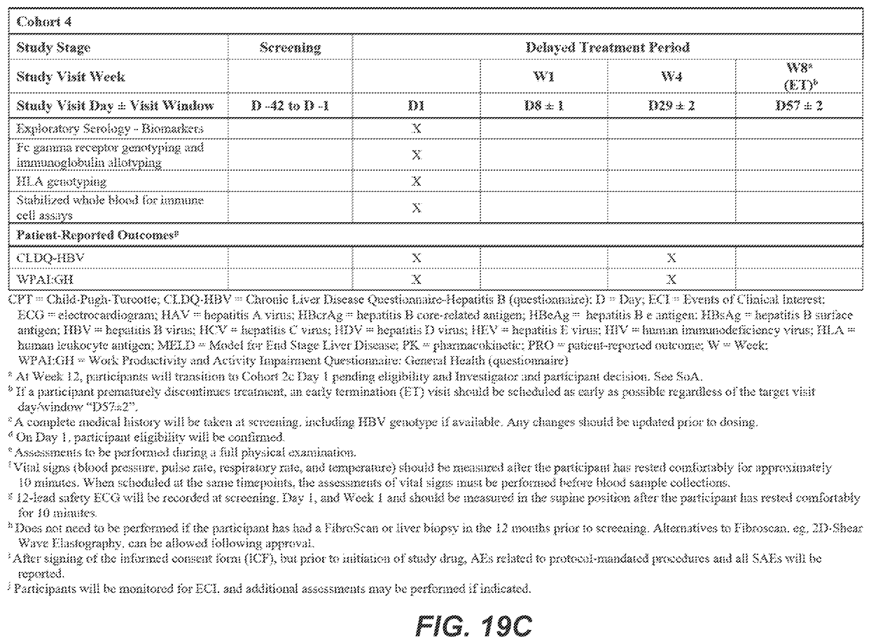

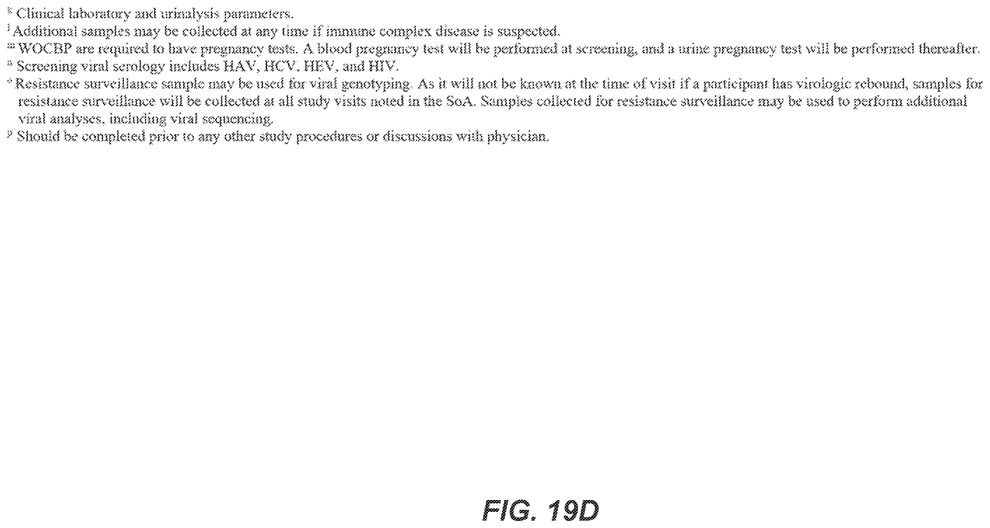

66. The method according to embodiment 65, wherein the siRNA is conjugated to the ligand as shown in the following structure:

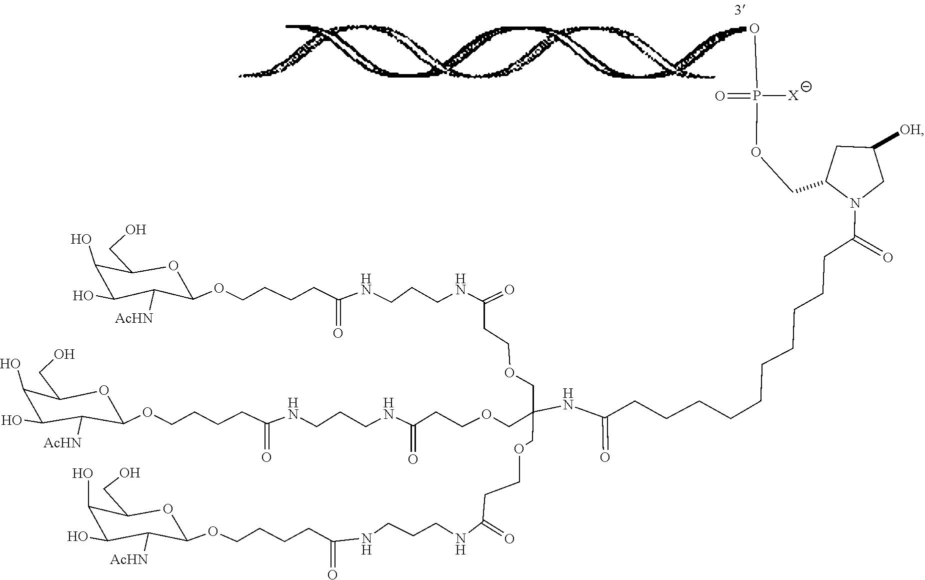

wherein X is O or S.
67. The method according to embodiment 66, wherein X is O.
68. The method according to any one of the preceding embodiments, wherein at least one nucleotide of the siRNA is a modified nucleotide comprising a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, an adenosine-glycol nucleic acid, or a nucleotide comprising a 5′-phosphate mimic.
69. The method according to any one of the preceding embodiments, wherein the siRNA comprises a phosphate backbone modification, a 2′ ribose modification, 5′ triphosphate modification, or a GalNAc conjugation modification.
70. The method according to any one of the preceding embodiments, wherein the phosphate backbone modification comprises a phosphorothioate bond.
71. The method according to any one of the preceding embodiments, wherein the siRNA comprises a 2′-fluoro or 2′-O-methyl substitution.
72. The method according to any one of the preceding embodiments, wherein all of the nucleotides of the sense strand of the siRNA and all of the nucleotides of the antisense strand are modified nucleotides.
73. The method according to one of the preceding embodiments, wherein the siRNA comprises a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6)
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
(Agn) is adenosine-glycol nucleic acid (GNA);
s is a phosphorothioate linkage; and
L96 is N-[tris(GalNAc-alkyl)-amidodecanoyl)]-4-hydroxyprolinol.
74. The method according to any one of the preceding embodiments, wherein the siRNA comprises a sense strand comprising 5′-gsusguGfcAfCfUfucgcuucacaL96-3′ (SEQ ID NO:7) and an antisense strand comprising 5′-usGfsugaAfgCfGfaaguGfcAfcacsusu-3′ (SEQ ID NO:8),
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
s is a phosphorothioate linkage; and
L96 is N-[tris(GalNAc-alkyl)-amidodecanoyl)]-4-hydroxyprolinol.
75. The method according to embodiment 67 or embodiment 68, wherein the L96 is conjugated to the sense strand as shown in the following structure:

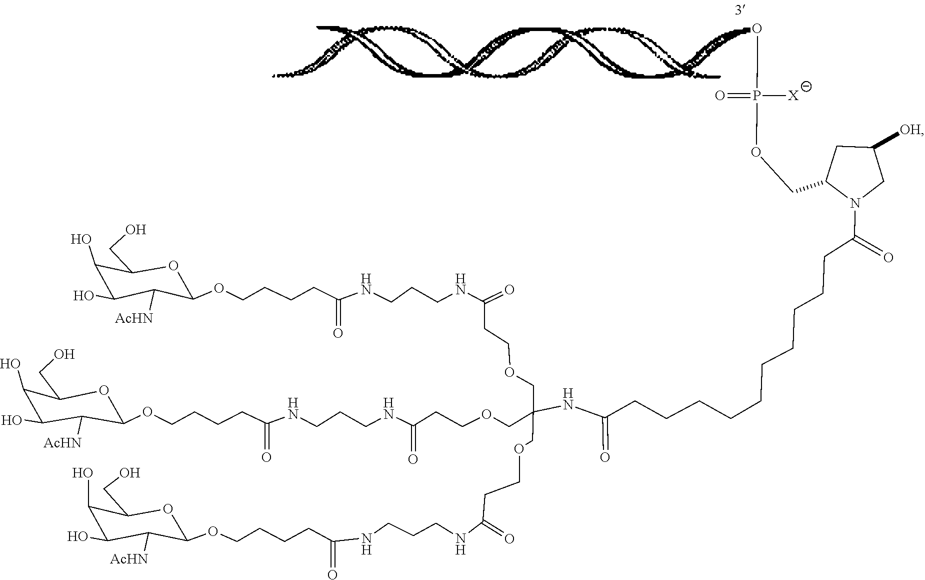

wherein X is O.
76. The method according to any preceding embodiment, wherein the subject is a human and a therapeutically effective amount of siRNA is administered to the subject; and wherein the effective amount of the siRNA is from about 1 mg/kg to about 8 mg/kg.
77. The method according to any preceding embodiment, wherein the siRNA is administered to the subject twice daily, once daily, every two days, every three days, twice per week, once per week, every other week, every four weeks, or once per month.
78. The method of any one of the preceding embodiments, further comprising administering to the subject a nucleos(t)ide reverse transcriptase inhibitor (NRTI).
79. The method of any one of the preceding embodiments, wherein the subject has previously been administered an NRTI.
80. The method according to any one of embodiment 78 or embodiment 79, wherein the NRTI is tenofovir, tenofovir disoproxil fumarate (TDF), tenofovir disoproxil (TD), tenofovir alafenamide (TAF), lamivudine, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, and ganciclovir, besifovir (ANA-380/LB-80380), or tenofvir-exaliades (TLX/CMX157).
81. The method according to any one of embodiments 78-80, wherein the NRTI is tenofovir, tenofovir disoproxil fumarate (TDF), or tenofovir disoproxil (TD).
82. The method according to any one of embodiments 78-81, wherein the NRTI is tenofovir disoproxil fumarate (TDF).
83. The method according to any one of the preceding embodiments, wherein:
(a) the anti-HBV antibody comprises or consists of: a light chain amino acid sequence according to SEQ ID NO: 59, and a heavy chain amino acid sequence according to SEQ ID NO: 57; and
(b) the siRNA comprises or consists of: a sense strand comprising or consisting of 5′-gsusguGfcAfCfUfucgcuucacaL96-3′ (SEQ ID NO:5) and an antisense strand comprising or consisting of 5′-usGfsuga(Agn)gCfGfaaguGfcAfcacsusu-3′ (SEQ ID NO:6),
   wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
   Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
   (Agn) is adenosine-glycol nucleic acid (GNA);
   s is a phosphorothioate linkage; and
   L96 is N-[tris(GalNAc-alkyl)-amidodecanoyl)]-4-hydroxyprolinol.
84. The method according to any one of embodiments 72-76, wherein:
(a) the anti-HBV antibody comprises or consists of: a light chain amino acid sequence according to SEQ ID NO:59, and a heavy chain amino acid sequence according to SEQ ID NO:57;
(b) the siRNA comprises or consists of: a sense strand comprising or consisting of 5′-gsusguGfcAfCfUfucgcuucacaL96-3′ (SEQ ID NO:5) and an antisense strand comprising or consisting of 5′-usGfsuga(Agn)gCfGfaaguGfcAfcacsusu-3′ (SEQ ID NO:6),
   wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
   Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
   (Agn) is adenosine-glycol nucleic acid (GNA);
   s is a phosphorothioate linkage; and
   L96 is N-[tris(GalNAc-alkyl)-amidodecanoyl)]-4-hydroxyprolinol; and
(c) the NRTI is tenofovir disoproxil fumarate (TDF) or entecavir.
85. The method according to any one of the preceding embodiments, wherein:
(a) the anti-HBV antibody comprises: a light chain amino acid sequence according to SEQ ID NO:59, and a heavy chain amino acid sequence according to SEQ ID NO:57; and
(b) the siRNA comprises: a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6),
   wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
   Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
   (Agn) is adenosine-glycol nucleic acid (GNA);
   s is a phosphorothioate linkage; and
   L96 is N-[tris(GalNAc-alkyl)-amidodecanoyl)]-4-hydroxyprolinol.
86. The method according to any one of the preceding embodiments, wherein:
(a) the anti-HBV antibody consists of: a light chain amino acid sequence according to SEQ ID NO:59, and a heavy chain amino acid sequence according to SEQ ID NO:57; and
(b) the siRNA consists of: a sense strand consisting of 5′-gsusguGfcAfCfUfucgcuucacaL96-3′ (SEQ ID NO:5) and an antisense strand consisting of 5′-usGfsuga(Agn)gCfGfaaguGfcAfcacsusu-3′ (SEQ ID NO:6),
   wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
   Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
   (Agn) is adenosine-glycol nucleic acid (GNA);
   s is a phosphorothioate linkage; and
   L96 is N-[tris(GalNAc-alkyl)-amidodecanoyl)]-4-hydroxyprolinol.
87. The method according to any one of the preceding embodiments, wherein the anti-HBV antibody and the siRNA are administered to the subject according to procedures set forth in any of FIGS. 1-7C and 12A-19D.
88. The method according to any one of the preceding embodiments, wherein the anti-HBV antibody is administered subcutaneously.
89. The method according to any one of the preceding embodiments, wherein the siRNA is administered subcutaneously.
90. The method according to any one of embodiments 78-89, wherein the NRTI is administered orally.
91. The method according to any one of the preceding embodiments, wherein the anti-HBV antibody is administered every 2 weeks.
92. The method according to any one of the preceding embodiments, wherein the anti-HBV antibody is administered every 4 weeks.
93. The method according to any one of the preceding embodiments, wherein the anti-HBV antibody is administered every 8 weeks.
94. The method according to any one of the preceding embodiments, wherein the siRNA is administered every 4 weeks.
95. The method according to any one of the preceding embodiments, wherein the siRNA is administered every 8 weeks.
96. The method according to any one of embodiments 78-95, wherein the NRTI is administered daily.
97. The method according to any one of the preceding embodiments, wherein the anti-HBV antibody is administered at a dose of 300 mg.
98. The method according to any one of the preceding embodiments, wherein the siRNA is administered at a dose of 200 mg.
99. The method according to any one of embodiments 78-98, wherein the NRTI is administered at a dose of 300 mg.
100. The method according to any one of embodiments 78-98, wherein the NRTI is administered at a dose of 245 mg.
101. The method according to any one of the preceding embodiments, wherein the subject is administered the siRNA and the anti-HBV antibody beginning on the same day.
102. The method according to any one of the preceding embodiments, wherein the subject is a human.
103. The method according to any one of the preceding embodiments, wherein the subject is administered the siRNA and the anti-HBV antibody for up to 96 weeks, 96 weeks, at least 96 weeks, or 96 weeks or longer.
104. An anti-HBV antibody; and an siRNA that targets an HBV mRNA; for use in the method according to any one of embodiments 1-103.
105. Use of an anti-HBV antibody; and an siRNA that targets an HBV mRNA; in the manufacture of a medicament for use in the method according to any one of embodiments 1-103.
106. Use of an anti-HBV antibody in the manufacture of a first medicament; and use of an siRNA that targets an HBV mRNA in the manufacture of a second medicament; wherein the first and second medicaments are to be used in a combination therapy according to the method of any one of embodiments 1-103.
107. An anti-HBV antibody; an siRNA that targets an HBV mRNA; and a NRTI; for use in the method according to any one of embodiments 78-103.
108. Use of an anti-HBV antibody; an siRNA that targets an HBV mRNA; and a NRTI; in the manufacture of a medicament for use in the method according to any one of embodiments 78-103.
109. Use of an anti-HBV antibody in the manufacture of a first medicament; use of an siRNA that targets an HBV mRNA in the manufacture of a second medicament; and use of a NRTI in the manufacture of third medicament; wherein the first, second, and third medicaments are to be used in a combination therapy according to the method of any one of embodiments 78-103.
110. A kit comprising:
a pharmaceutical composition comprising an anti-HBV antibody, and a pharmaceutically acceptable excipient; and
a pharmaceutical composition comprising an siRNA that targets an HBV mRNA, and a pharmaceutically acceptable excipient.
111. The kit according to embodiment 110, further comprising instructions for completing the method according to any one of embodiments 1-103.
112. A kit comprising:
a pharmaceutical composition comprising an anti-HBV antibody, and a pharmaceutically acceptable excipient;
a pharmaceutical composition comprising an siRNA that targets an HBV mRNA, and a pharmaceutically acceptable excipient; and
a pharmaceutical composition comprising a NRTI, and a pharmaceutically acceptable excipient.
113. The kit according to embodiment 104, further comprising instructions for completing the method according to any one of embodiments 78-103.

### EXAMPLES

### Example 1

### Clinical Evaluation of Monotherapy or Combination Therapy to Treat Chronic HDV Infection

The safety, efficacy, and tolerability of an anti-HBV antibody in combination with an anti-HBV siRNA is evaluated in a Phase 2, multi-center, open-label study clinical study in noncirrhotic and compensated cirrhotic (CPT-A through CPT-C) human patients with chronic HBV/HDV coinfection not currently on treatment for HDV.

### Background

The only virus in the genus, Deltaviridae, the hepatitis D virus (HDV) requires hepatitis B virus (HBV) coinfection for entry into hepatocytes, intrahepatic spread, and dissemination. HBV/HDV coinfection is associated with a more rapid and severe course of liver diseases compared to other forms of viral hepatitis (Lempp 2016; Lucifora 2020). Based on sequence variations in HDV isolates, 8 genotypes have been classified (Le Gal 2017). Genotype 1 is globally distributed, with infections varying from fulminant hepatitis to asymptomatic chronic liver disease. HDV genotypes 2-8 have limited geographic distribution (Niro 2012). Median HDV prevalence in hepatitis B surface antigen (HBsAg) carriers is estimated to be about 5%; however, in certain locations, up to 80% of HBsAg carriers have laboratory evidence of current or past HDV infection (Rizzetto 2021). Due to the significant knowledge gaps, epidemiological data on HDV prevalence may be profoundly underestimated, ranging from 12 to 72 million persons (Chen 2019; Miao 2020; Stockdale 2020).

HDV is a defective RNA satellite virus that does not encode an envelope protein and depends on the HBsAg to complete its life cycle. Thus, HDV must either co- or superinfect HBV-infected hepatocytes. HDV virions are roughly spherical 35 to 43 nm structures with no distinct nucleocapsid. The ribonucleoprotein comprises 60 large and small delta antigens, the only proteins encoded by HDV. Extracellular HDV virions contain genomic HDV RNA, a singlestranded negative-sense, covalently closed circular RNA molecule of 1,668-1,697 nucleotides, depending on the genotype (Le Gal 2017). HDV does not encode an RNA-dependent-RNA polymerase but instead relies on host DNA-dependent RNA polymerases to facilitate RNA-directed RNA synthesis for transcribing and replicating its genome in the nucleus of hepatocytes using a double-rolling circle mechanism (Chang 2008; Modahl 2000; Sureau 2016; Urban 2021).

HDV virions can assemble in hepatocytes using all forms of HBsAg derived from cccDNA (in the case of HBV coinfected hepatocytes) and HBsAg from integrated HBV. Virion assembly depends on an interaction between HBsAg and the farnesylated N-terminus of the large form of the HDV Delta antigen (L-HDAg) (Freitas 2014; Shirvani-Dastgerdi 2015). The large form of HBsAg (L-HBsAg) is needed to form infectious virions, as the preS1 domain of the L-HBsAg mediates the interaction between HBsAg and NTCP. NTCP is a basolateral Na-dependent bile salt transporter that is localized exclusively at the basolateral membrane of differentiated mammalian hepatocytes and is hepatocyte-specific. Therefore, although HDV can efficiently replicate its genome and express the hepatitis Delta antigen, L-HBsAg is needed to form infectious progeny virions.

HDV infection can occur in 2 ways: HBV-HDV coinfection and HDV superinfection. Coinfection occurs when HBV and HDV are transmitted simultaneously to an HBV susceptible individual, whereas superinfection occurs when an HBsAg positive person (typically chronic HBV infection) acquires HDV. HBV/HDV coinfection occurs in 5 to 15% of cases of HDV infection and often leads to acute hepatitis, which is frequently more severe than acute HBV; however, progression to chronic HDV infection only occurs in at 2 to 5% (Bahcecioglu 2017; Raimondo 1982; Romeo 2009; Vlachogiannakos 2020). In contrast, HDV superinfection of HBV carriers occurs in approximately 75% of cases and frequently worsens the pre-existing liver disease with fulminant hepatitis developing in 7 to 15% of cases (Farci 1983).

Chronic HDV infection is defined by the persistence of HDV RNA or hepatitis delta antigen (HDAg) in serum for at least 6 months after HDV infection. Chronic HBV/HDV infection causes more severe disease than chronic HBV, with faster fibrosis progression rates (Mathurin 2000; Sagnelli 1989). Additionally, patients with chronic HDV infection are 2-fold more likely to develop and die of hepatic decompensation or HCC than those with HBV mono-infection (Fattovich 2000; Niro 2010; Romeo 2009). Persistent HDV replication is the only factor associated with an increased risk of mortality (Romeo 2009).

Given HDV's dependency on HBV, strategies to protect from HBV infection (e.g., prophylactic vaccines) also protect against HDV infection. However, some regions in the world have poor access to HBV vaccines (WHO 2021, Hepatitis D). Additionally, vaccine nonresponders, persons with waning immunity, and immuno-compromised patients comprise a group that remains susceptible to chronic HDV infection. Currently, the only available curative treatment for HDV infection is pegylated interferon alpha (PEG-IFNα). Current guidelines from the American Association for the Study of Liver Diseases (AASLD), the Asia Pacific Association for the Study of the Liver (APASL), and the European Association for the Study of the Liver (EASL) recommend administering PEG-IFNα for at least 48 weeks to patients with chronic HDV infection (EASL 2017; Sarin 2016; Terrault 2018). PEGIFNα treatment establishes a sustained virologic response (SVR, clearance of serum HDV maintained 6 months after stopping treatment) in around 25 to 30% of patients however relapses occur in ˜50% of patients after long-term follow-up (Heidrich 2014). In addition to low SVR rates, adverse reactions associated with PEG-INFα therapy are well described, and PEG-INFα is contraindicated in patients with autoimmune diseases, some psychiatric syndromes, and Child-Pugh-Turcotte (CPT)-B or CPT-C stage cirrhotic patients (Rizzetto 2015; Sleijfer 2005). Further limiting its usefulness, reduced efficacy of PEG-INFα has been observed when treating cirrhotic patients with chronic HDV (Gunsar 2005). Limitations of the inability to use peg-INFα in cirrhotic HDV patients are particularly notable as 50% of HDV-infected patients are cirrhotic at diagnosis (Fattovich 1987). NRTIs with activity against HBV, such as adefovir, entecavir, famciclovir, and tenofovir disoproxil fumarate (TDF) when used alone or in combination with PEG-INFα do not affect HDV (Wedemeyer 2011). Despite this limitation, nucleos(t)ide reverse transcriptase inhibitors (NRTIs) are used in HBV/HDV coinfected persons for their ability to modulate HBV replication.

BLV, an entry inhibitor that targets the NTCP receptor, was recently granted Conditional Marketing Authorization by the European Commission and PRIority Medicines (PRIME) scheme eligibility by the European Medicines Agency as the first approved treatment in Europe for adults with chronic HDV and compensated liver disease. Interim results from the Phase 3 MYR301 study indicate that after 24 weeks of 2 mg daily subcutaneous (SC) dose of BLV given every day, 36.7% of participants with chronic HDV achieve the combined virological (percentage of participants with undetectable (<limit of detection (LOD) HDV RNA or decrease by ≥2 log 10 IU/ml from baseline) and biochemical response (ALT normalization). Additional planned follow-up studies will examine the durability of these responses. No participants demonstrated HBsAg loss, as such, lifelong treatment with BLV may be needed to successfully manage HDV. Additionally, BLV has not been studied in and cannot be administered to persons with CPT-B or CPT-C hepatic impairment (Hepcludex Summary of Product Characteristics 2020) who comprise a significant portion of patients with HDV infection.

Despite the recent introduction of BLV, methods to treat chronic HDV remain unsatisfactory for several reasons. Efficacy with either PEG-IFNα or BLV is not complete, with only ˜17% of PEG-IFNα treated individuals achieving long-term virologic response and BLV, while better, still offers only ˜37% of participants meeting the endpoints of HDV RNA suppression and ALTnormalization (Wedemeyer 2021). PEG-IFN-α and BLV require weekly and daily subcutaneous administration, respectively, which impacts adherence to treatment. PEG-IFN-α is commonly associated with several adverse reactions that further limit adherence as well as drug-related toxicities that often require intensive laboratory monitoring and dose adjustments. BLV has been associated with treatment discontinuations related to adverse events in 10% of patients in real world study (de Lédinghen 2021). Further, several potential drug interactions have been identified for BLV based on its interaction with the NTCP receptor and hepatic transport proteins OATP1B1/3 (Hepcludex Summary of Product Characteristics 2020).

Finally, another area that current treatments do not address is the treatment of patients with moderate to severe hepatic impairment. This group comprises a significant portion of patients with chronic HDV. PEG-IFN-α or BLV cannot be safely prescribed to these patients, thus leaving them with no treatment options.

Targets of current and emerging treatment strategies against HDV include inhibiting HDV RNA transcription, suppressing HBsAg production, or blocking infection of susceptible hepatocytes (Lok 2021; Yurdaydin 2019). In ongoing studies, HBV targeting small interfering ribonucleic acids (siRNA) (SIRNA01) and HBsAg targeting monoclonal antibody (mAb) (AB01) have demonstrated the ability to suppress HBsAg in HBV monoinfected persons. Additionally, in preclinical models, lowering HBsAg with an siRNA (SIRNA01) or an HBsAg targeting monoclonal antibody (AB01) have both resulted in decreased HDV viremia (Lempp 2021).

Single doses of SIRNA01 up to 900 mg in healthy volunteers and 6 doses of SIRNA01 200 mg administered every 4 weeks in participants with chronic HBV infection were well tolerated and exhibited safety profiles supportive of continued clinical development. Regardless of hepatitis B e-antigen (HBeAg) status, SIRNA01 is associated with substantial reductions (up to 2 log 10 IU/mL) in HBsAg but does not lead to serologic clearance to HBsAg (Gane 2021). As HDV replication depends on HBsAg, SIRNA01 is expected to reduce (or possibly clear) HDV viremia in parallel with the reduction in HBsAg. Independent of the antiviral activity of SIRNA01, AB01 has the potential to reduce HBsAg further and consequently further deepen suppression of HDV viremia. AB01 is being evaluated in participants with chronic HBV in an ongoing Phase 1 study. Participants received single doses of AB01. The largest and most durable HBsAg reductions (−2.42 log 10 IU/mL mean change from baseline HBsAg) were observed in the 300 mg dose cohort. By neutralizing HDV virions, AB01 will inhibit infection of new hepatocytes, and engineered modifications to this mAb designed to recruit immune effector cells should accelerate the elimination of HBV/HDV coinfected hepatocytes.

The objectives of this study are to evaluate the safety of SIRNA01 and AB01 in participants with HBV/HDV coinfection and evaluate whether monotherapy or combination therapy with the investigation agents can durably suppress HDV replication and normalize ALT when given on a monthly or bimonthly schedule to participants with all degrees of liver disease severity.

### Study Design

Table 4 shows the treatment groups for the study.

TABLE 4
Treatment groups for clinical study.

| | | | | Induction Period | | Maintenance Period | |
|---|---|---|---|---|---|---|---|
| | Study | | | Number | Frequency | Number | Frequency |
| Cohort | intervention | Dose | Route | of Doses | of Dosing | of Doses | of Dosing |
| 1a | SIRNA01 | 200 mg | SC | 3 | Every 4 | 10^{a} | Every 8 |
| | | | | | weeks | | weeks |
| 1b | AB01 | 300 mg | SC | 3 | Every 4 | 10^{a} | Every 8 |
| | | | | | weeks | | weeks |
| 2 | SIRNA01 | 200 mg | SC | 7 or | Every 4 | 8 or 5^{d} | Every 8 |
| | | | | 13^{b, c} | weeks | | weeks |
| | AB01 | 300 mg | SC | 7 or | Every 4 | 8 or 5^{d} | Every 8 |
| | | | | 13^{b, c} | weeks | | weeks |
| 3 | SIRNA01 | 200 mg | SC | 7 or | Every 4 | 8 or 5^{d} | Every 8 |
| | | | | 13^{b, c} | weeks | | weeks |
| | AB01 | 300 mg | SC | 7 or | Every 4 | 8 or 5^{d} | Every 8 |
| | | | | 13^{b ,c} | weeks | | weeks |
| 4 | SIRNA01 | 200 mg | SC | 7 or | Every 4 | 8 or 5^{d} | Every 8 |
| | | | | 13^{b, c} | weeks | | weeks |
| | AB01 | 300 mg | SC | 7 or | Every 4 | 8 or 5^{d} | Every 8 |
| | | | | 13^{b, c} | weeks | | weeks |

SC = subcutaneous
^{a}Participants meeting the Primary endpoint after 3 doses over 4 weeks will receive 10 additional doses × 8 weeks.
^{b}Participants meeting the Primary endpoint after 7 doses over 4 weeks will receive 8 additional doses × 8 weeks.
^{c}Participants not meeting the Primary endpoint after 7 doses over 4 weeks will receive 5 additional doses × 4 weeks.
^{d}Participants meeting the Primary endpoint after 13 doses over 4 weeks will receive 5 additional doses × 8 weeks.

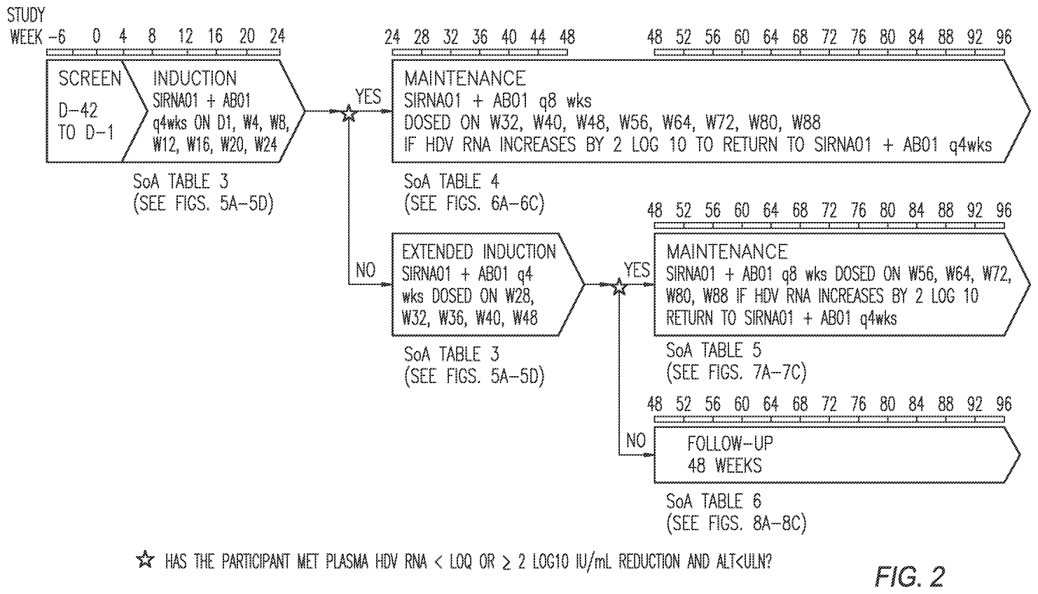

The study scheme for Cohort 1 is shown in FIG. 1, and the study scheme for Cohorts 2-4 is shown in FIG. 2.

Up to 58 participants will be enrolled in the study. In Cohorts 1a and 1b, a total of approximately 10 participants with HBV/HDV coinfection with liver fibrosis staging of Meta-Analysis of Histological Data in Viral Hepatitis (METAVIR) F0-F3 will be enrolled, with 5 participants per cohort. In Cohort 2, a minimum of 12 and a maximum of 22 participants will be enrolled, composed of 3 groups: (i) HBV/HDV coinfection with liver fibrosis staging of METAVIR F0-F3 who enter from Cohort 1 (maximum 10 participants); (ii) newly enrolled participants with METAVIR F0-F3; and (iii) newly enrolled participants with METAVIR F4 and mild hepatic impairment (CPT-A). Additionally, a combined total of up to 8 floater participants may be added to Cohorts 1 or 2. In Cohort 3, a total 12 participants with HBV/HDV coinfection with liver fibrosis staging of METAVIR F4 and moderate hepatic impairment (CPT-B) will be enrolled. In Cohort 4, a total of 6 participants with HBV/HDV coinfection with liver fibrosis staging of METAVIR F4 and severe hepatic impairment (CPT-C) will be enrolled.

Cohorts 1, 2, 3, and 4 will enroll sequentially based on review of the safety data. Cohorts may be closed or discontinued at the sponsor's discretion. Cohort 2 will start with the enrollment of participants entering from Cohort 1 after a review of their 12-week monotherapy safety data, discussion with the investigator and re-consent by the participant. Cohort 1 participants are eligible to enter Cohort 2 after (or at the time of) their original study Week 16. Cohort 2 will also enroll additional participants after a safety review of 12-week monotherapy safety data from the first 10 participants in Cohort 1. Cohort 3 will start enrollment after (1) review of safety outcomes of other studies and (2) review of the safety data in the first 5 participants in Cohort 2 completing study Week 12. Cohort 4 will start enrollment after (1) review of safety outcomes of Cohort 3 of other studies and (2) review of the safety data in the first 5 participants in Cohort 3 completing study Week 12.

The total study duration is planned to be up to 102 weeks. For all cohorts, the intervention period is composed of 2 periods: Induction and Maintenance periods. For Cohort 1, this includes a Screening period (up to 6 weeks), Induction period (12 weeks), and Maintenance period (84 weeks). At the end of the Induction period, participants who do not transition to the Maintenance period or enter Cohort 2 will enter a Follow-Up period (48 weeks). Participants who prematurely discontinue study treatments during the Induction period will enter the Follow-Up period (48 weeks). Participants who prematurely discontinue study treatment during the Maintenance period will enter the Follow-Up period for 48 weeks after the last dose of the study intervention or until Week 96 whichever is earlier. For Cohorts 2 to 4, this includes a Screening period (up to 6 weeks), Induction period (24 or 48 weeks), and Maintenance period (72 or 48 weeks). At the end of the Induction period, participants who do not transition to the Maintenance period will enter a Follow-Up period (48 weeks). Participants who prematurely discontinue study treatments during the Induction period will enter the Follow-Up period (48 weeks). Participants who prematurely discontinue study treatment during the Maintenance period will enter the Follow-Up period for 48 weeks after the last dose of the study intervention or until Week 96 whichever is earlier.

The Screening Period for all participants will be up to 42 days.

The Intervention period will consist of 2 Periods: Induction and Maintenance. Participants in Cohorts 1a and 1b will receive 3 doses of study treatment in the Induction period (on Day 1, Week 4 and Week 8) and can continue into the Maintenance period for an additional 84 weeks if achieving a ≥2 log 10 decrease in HDV RNA compared to baseline or HDV RNA<LOQ and ALT<upper limit of normal (ULN) (combined endpoint) at the Week 12 visit. Participants not meeting the combined endpoint at Week 12 can either enter Cohort 2 or the Follow-up period. Participants in Cohorts 2 to 4 will receive 24 or 48 weeks of treatment in the Induction period. Participants meeting the combined endpoint at the Week 24 visit will transition to the Maintenance period for 72 weeks and receive their next dose at Week 32. Participants not meeting the combined endpoint at Week 24 visit will continue in the Induction period until Week 48. Participants meeting the combined endpoint at Week 48 visit will transition to the Maintenance period for 48 weeks and receive their next dose at Week 56. Participants not meeting these criteria at Week 48 will conclude the Intervention period and enter the Follow-Up period. Participants in the Maintenance period with HDV RNA rebounding to within 1 log 10 IU/mL of their baseline will return to every 4-week dosing of SIRNA01+AB01 through Week 88. In these cases, the next dose should be administered at the next scheduled visit per the Schedule of Activities (SoA) table. If the next scheduled visit will not occur for 8 weeks, the next dose should be given in 4 weeks through an unscheduled visit and doses should then be given every 4 weeks through Week 88.

The maximum duration of the Follow-Up period is 48 weeks after the last dose of the study intervention or Week 96 whichever is earlier. Participants will enter the Follow-Up period if: (1) Enrolled in Cohort 1 and not transitioning to the Maintenance period after meeting the combined Week 12 endpoint; (2) Enrolled in Cohort 1 and not entering Cohort 2 after failing to meet the combined Week 12 endpoint; (3) Enrolled in Cohort 2 to 4 and not transitioning to the Maintenance period at Week 48; or (4) Enrolled in any cohort and prematurely discontinuing study drugs.

AB01 is provided as a reconstituted lyophilized powder administered subcutaneously (SC) at 300 mg every 4 weeks or 8 weeks (see Table 4). AB01 (HBC34v35-MLNS-GAALIE) comprises a light chain amino acid sequence of SEQ ID NO:59, and a heavy chain amino acid sequence of SEQ ID NO:57. SIRNA01 is provided as a liquid administered SC at 200 mg every 4 weeks or 8 weeks (see Table 4). SIRNA01 has a sense strand comprising the nucleotide sequence of SEQ ID NO:5 and an antisense strand comprising the nucleotide sequence of SEQ ID NO:6.

Study objectives and associated endopoints are shown in Table 5.

TABLE 5
Study objectives and associated endpoints.

| Objectives | Endpoints |
|---|---|
| To evaluate the efficacy of | Cohorts 2 to 4: Proportion of participants |
| SIRNA01 and AB01 in | achieving either ≥2 log10 decrease in HDV |
| participants with chronic | RNA compared to baseline or HDV RNA < |
| HBV/HDV coinfection in a 24- | LOQ and ALT < upper limit of normal (ULN) |
| week Induction period | at Week 24 |
| To evaluate the safety of | Proportion of participants with treatment- |
| SIRNA01 and AB01 in | emergent adverse events (TEAEs) and serious |
| participants with chronic | adverse events (SAEs) |
| HBV/HDV coinfection | |
| To evaluate the efficacy of | (i) Cohorts 2 to 4: Proportion of participants |
| SIRNA01 and AB01 in | achieving either ≥2 log10 decrease in HDV |
| participants with chronic | RNA compared to baseline or HDV RNA < |
| HBV/HDV coinfection in different | LOQ and ALT < ULN at the end of the |
| Induction periods | Induction period; |
| | (ii) Cohort 1: Proportion of participants |
| | achieving either ≥2 log10 decrease in HDV |
| | RNA compared to baseline or HDV RNA < |
| | LOQ and ALT < ULN at Week 12 |
| To evaluate the effects of | Number of participants in the Maintenance |
| SIRNA01 and AB01 on HDV | period with sustained ≥2 log10 decrease in |
| viremia and ALT in the | plasma HDV RNA or plasma HDV RNA < |
| Maintenance period | LOQ and ALT < upper limit of normal (ULN) |
| | at Week 48, Week 72, and Week 96 |
| To evaluate the effects of | (i) Proportion of participants with ≥2 log10 |
| SIRNA01 and AB01 on HDV | decrease in HDV RNA compared to baseline or |
| viremia in Induction and | HDV RNA < LOQ at the end of Induction |
| Maintenance periods | period, Week 48, Week 72, and Week 96; |
| | (ii) Change of HDV RNA from baseline at the |
| | end of Induction period, Week 12, Week 24, |
| | Week 48, Week 72, and Week 96 |
| To evaluate the effects of | Proportion of participants with ALT < ULN at |
| SIRNA01 and AB01 on ALT in | the end of Induction period, Week 12, Week 24, |
| Induction and Maintenance | Week 48, Week 72, and Week 96 |
| periods | |
| To assess the immunogenicity of | Incidence and titers of anti-drug antibodies |
| AB01 (for cohorts with AB01) | (ADA; if applicable) to AB01 across study |
| | timepoints (for cohorts with AB01) |
| To assess the effects of SIRNA01 | (i) Change from baseline in liver fibrosis at |
| and AB01 on liver fibrosis and | Week 48 and Week 96; |
| hepatic function | (ii) Change from baseline Model for End Stage |
| | Liver Disease (MELD) score at Week 12, Week |
| | 24, Week 36, Week 48, Week 60, Week 72, |
| | Week 84, and Week 96; |
| | (iii) Change from baseline Child-Pugh-Turcotte |
| | (CPT) score at Week 24, Week 48, Week 72, |
| | and Week 96 |
| To evaluate the host immune | Evaluation of host immune responses and |
| response and exploratory | exploratory biomarkers related to HBV and/or |
| biomarkers related to infection | investigational therapy(ies), including but not |
| and/or investigational therapy(ies) | limited to genetic, metabolic, cellular, |
| | and proteomic parameters |
| To evaluate the effects of | (i) Proportion of participants in the |
| SIRNA01 and AB01 on HDV | Maintenance period with an increase of HDV |
| RNA during the Maintenance | RNA to within 1 log10 IU/mL of their baseline; |
| period | (ii) Change from baseline serum HDV RNA |
| | after return to SIRNA01 + AB01 every 4 weeks |
| | among participants with rebound HDV viremia |
| | during the Maintenance period |
| To assess the effects of SIRNA01 | (i) Change of HBsAg from baseline at the end |
| and AB01 on serum HBsAg | of Induction period, Week 12, Week 24, Week |
| | 48, and Week 96; |
| | (ii) Number of participants with HBsAg loss at |
| | the end of Induction period, Week 12, Week 24, |
| | Week 48, and Week 96 |
| To evaluate the emergence of viral | Emergence of HBV or HDV viral resistance to |
| resistance to investigational | investigational therapy(ies) |
| therapy(ies) | |
| To evaluate additional viral | Assessment of additional viral parameters |
| parameters associated with HBV | associated with HBV, HDV, and/or |
| and/or investigational therapy(ies) | investigational therapy(ies) |
| To characterize the | AB01 PK parameters (for cohorts with |
| pharmacokinetics (PK) of AB01 | AB01) |
| (for cohorts with AB01) | |
| To characterize the PK of | SIRNA01 PK parameters (for cohorts with |
| SIRNA01 (for cohorts with | SIRNA01) |
| SIRNA01) | |
| To assess the immunogenicity of | Incidence and titers of ADA (if applicable) to |
| SIRNA01 (for cohorts with | SIRNA01 (for cohorts with SIRNA01) |
| SIRNA01) | |
| To assess the effects of SIRNA01 | Incidence of HCC and Progression to liver |
| and AB01 on end-stage liver | failure requiring transplantation or resulting in |
| disease outcomes | death |
| To assess health-related quality of | Change from baseline in health-related quality |
| life measurements | of life as measured by the Chronic Liver |
| | Disease Questionnaire-Hepatitis B (CLDQ- |
| | HBV) questionnaire |
| To assess the change from baseline | Change from baseline in work productivity and |
| in work productivity and daily | daily activities as measured by the Work |
| activities measurements | Productivity and Activity Impairment (WPAI) |
| | questionnaire |

The Child-Pugh Turcotte (CPT) Score Assessment of Liver Disease is shown in Table 6.

TABLE 6
Child-Pugh Turcotte (CPT) Score Assessment of Liver Disease.

| | Points Scored for Observed Findings | | |
|---|---|---|---|
| | 1 | 2 | 3 |
| Encephalopathy grade* | none | 1 or 2 | 3 or 4 |
| Ascites | absent | slight | moderate |
| Serum bilirubin, mg/dL | <2 | 2 to 3 | >3 |
| Serum albumin, g/dL | >3.5 | 2.8 to 3.5 | <2.8 |
| Prothrombin time, sec | <4 | 4 to 6 | >6 |
| prolonged | | | |

*Grade 0: normal consciousness, personality, neurological examination electroencephalogram
Grade 1: restless, sleep disturbed, irritable/agitated, tremor, impaired handwriting, 5 cycle per second waves
Grade 2: lethargic, time-disoriented, inappropriate, asterixis, ataxia, slow triphasic waves
Grade 3: somnolent, stuporous, place-disoriented, hyperactive reflexes, rigidity, slower waves
Grade 4: unrousable coma, no personality/behavior, decerebrate, slow 2-3 cycle per second delta activity
Source: FDA 2003
Assessment: Class A (mild hepatic impairment) if 5 or 6 points; B (moderate hepatic impairment) if 7 to 9 points; and C (severe hepatic impairment) if 10 to 15 points.

ULN values for ALT may be, for example, 34 IU/mL for females and 43 IU/mL for males.

PK parameters (free and total PK, as applicable) of SIRNA01 and AB01 will be computed using standard noncompartmental methods as applicable. Parameters may include, but not be limited to, Cₘₐₓ, Cₗₐₛₜ, Tₘₐₓ, Tₗₐₛₜ, AUC_{inf}, AUCₗₐₛₜ, % AUCₑₓₚ, t_{1/2}, λ₂, V_{z}/F, and CL/F.

Immunogenicity data may include, but not be limited to, incidence, titers, and neutralization data.

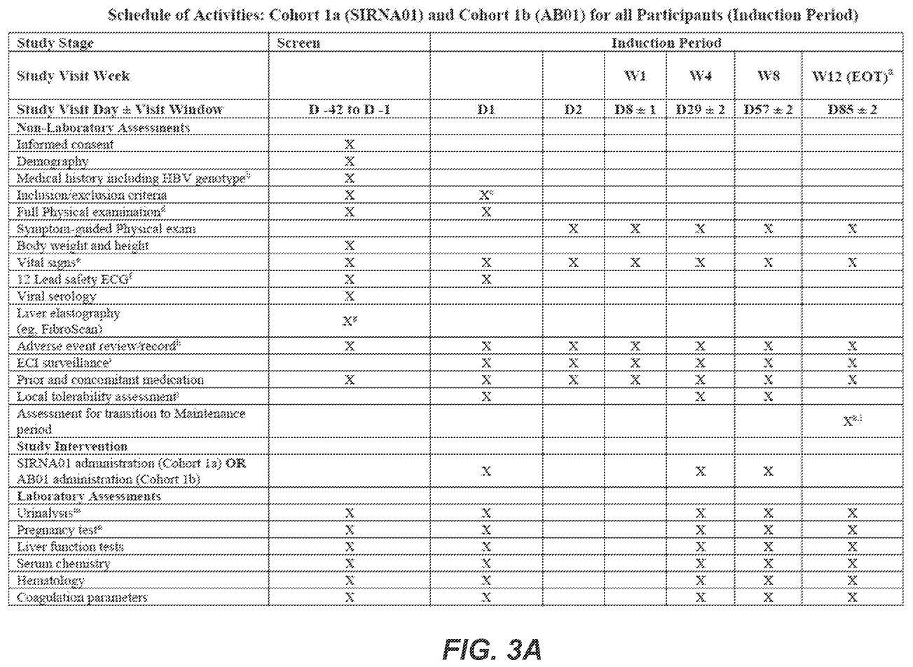

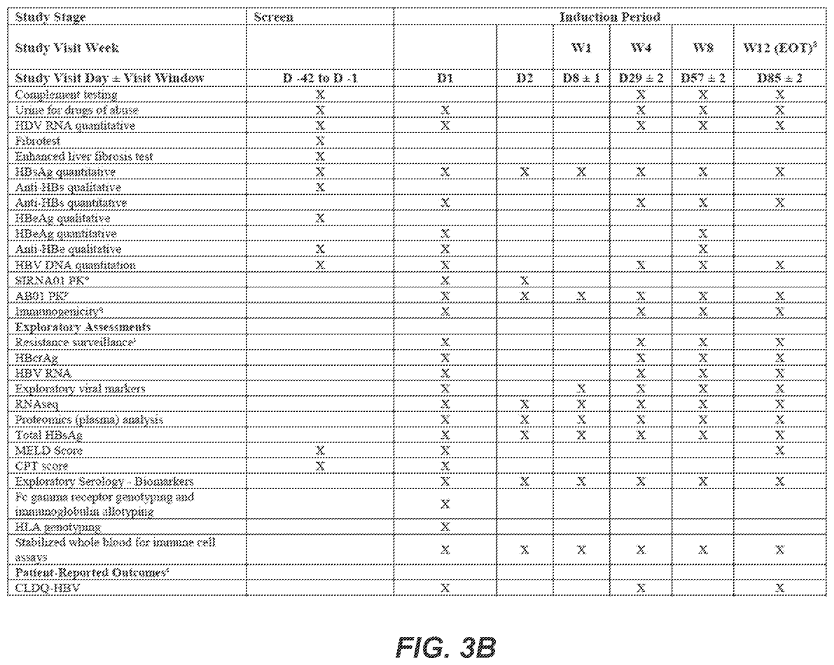

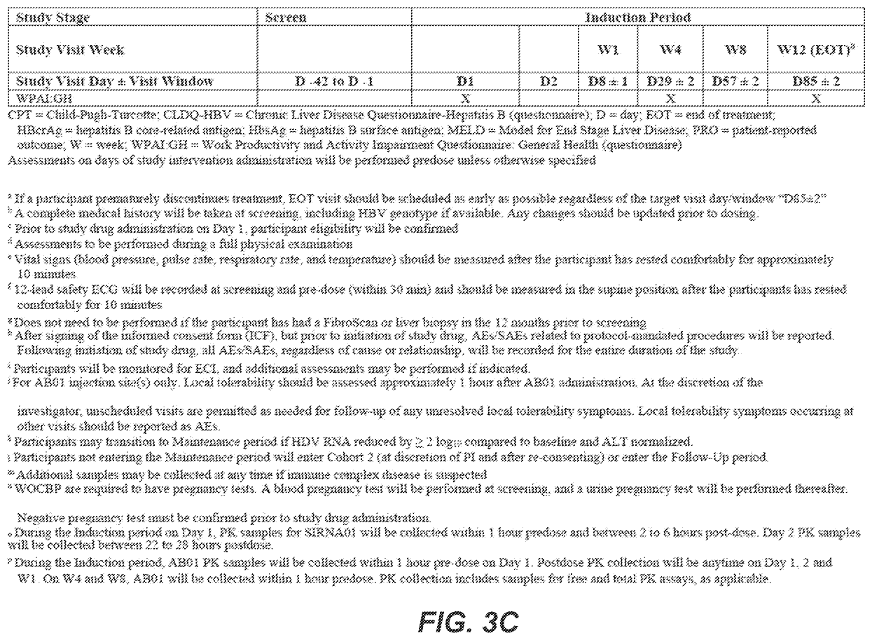

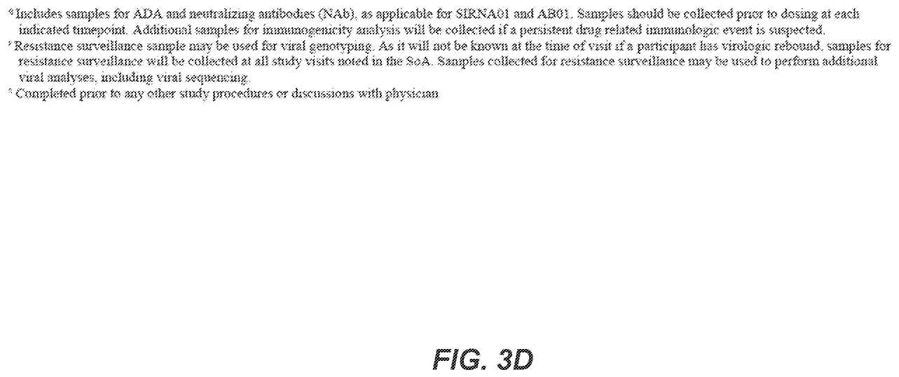

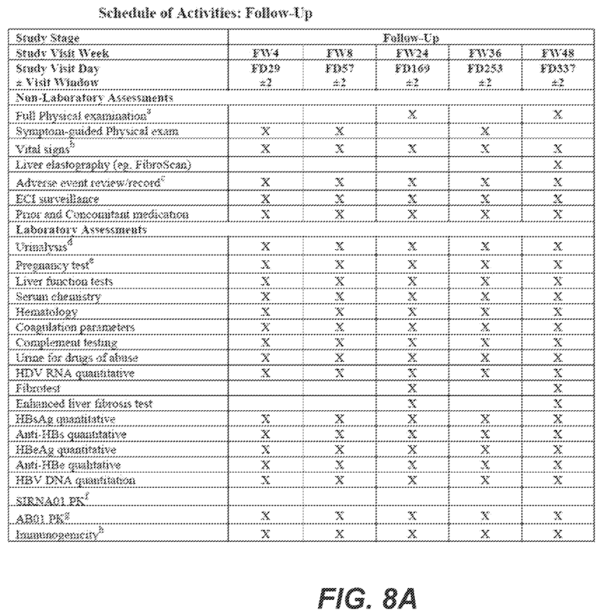

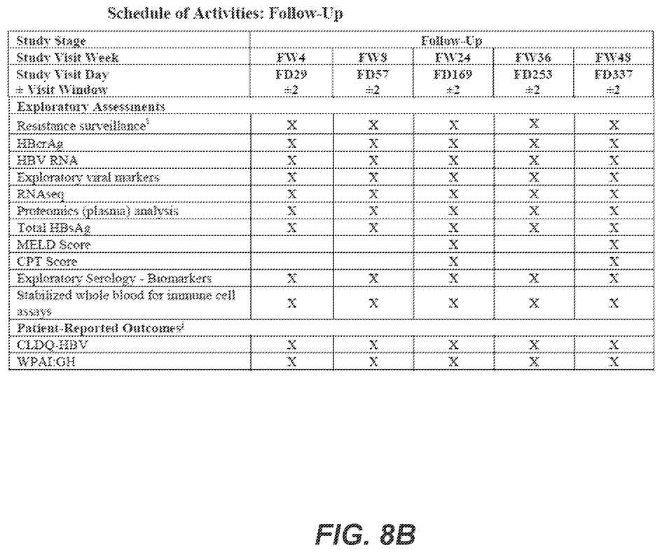

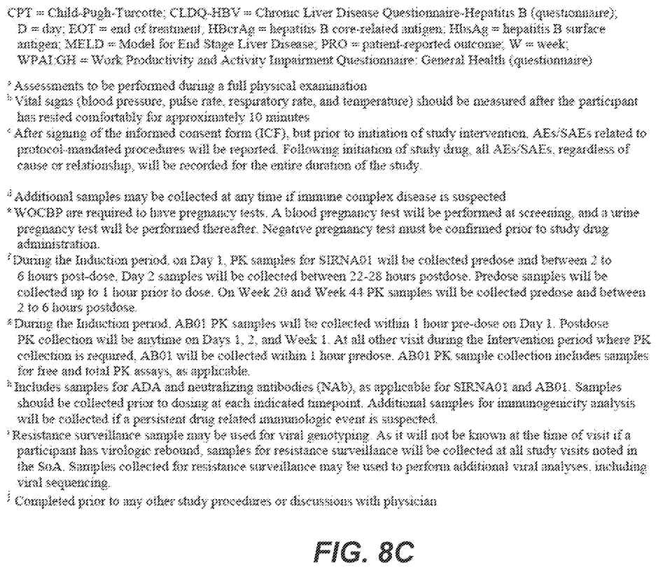

The schedules of activities (“SoA”) for the cohorts and Follow-Up Period are provided in FIGS. 3A-8C.

### Participant Population

Participants are male and female participants 18 to 70 years of age with chronic HBV/HDV coinfection with all degrees of hepatic impairment and currently on NRTI therapy. “Chronic HBV infection” for purposes of the study is defined as a positive serum HBsAg, HBV DNA, or HBeAg on 2 occasions at least 6 months apart based on previous (within the past 12 months) or current laboratory documentation (any combination of these tests performed 6 months apart is acceptable). Enrollment in each cohort will target baseline HBsAg>10,000 IU/mL at screening in approximately 40% of participants. Participants will be on locally approved NRTI therapy for at least 12 weeks prior to Day 1. Participants will also have HBsAg>0.05 IU/mL at screening, clinical evidence for chronic hepatitis and positive HDV antibody for at least 6 months, and positive HDV RNA at least 3 months before screening. Serum alanine aminotransferase (ALT) and aspartate aminotransferase (AST)>ULN and <5×ULN.

Participants are age≥18 (or age of legal consent, whichever is older) to <70 years at screening. Participants also have a Body Mass Index (BMI)≥18 kg/m² to ≤40 kg/m².

Additional inclusion criteria include the following:
Female participants must have a negative pregnancy test or confirmation of postmenopausal status. Postmenopausal status is defined as 12 months with no menses without an alternative medical cause. Confirmation of negative follicle stimulating hormone [FSH] is required to confirm postmenopausal status. Women of childbearing potential (WOCBP) must have a negative blood pregnancy test at screening and a negative urine pregnancy test on Day 1, cannot be breast feeding, and must be willing to use highly effective methods of contraception 14 days before study drug administration through 48 weeks after the last dose of SIRNA01 or AB01. Female participants must also agree to refrain from egg donation and in vitro fertilization from the time of study drug administration through 48 weeks after the last dose of SIRNA01 or AB01.
Male participants with female partners of childbearing potential must agree to meet 1 of the following contraception requirements from the time of study drug administration through 48 weeks after the last dose of SIRNA01 or AB01: documentation of vasectomy or azoospermia, or male condom use plus partner use of 1 of the contraceptive options listed for contraception for WOCBP. Male participants must also agree to not donate sperm from the time of first study drug administration through 48 weeks after the last dose of SIRNA01 or AB01.
Capable of giving signed informed consent
12-lead electrocardiogram (ECG) within normal limits; or, with no clinically significant abnormalities at screening, as determined by the clinical investigator.
Agrees not to donate blood during the duration of the study and for an additional 3 months after the last dose study drug.
Cohorts 1 and 2 specific inclusion criteria
   Noncirrhotic
      Liver biopsy with METAVIR F0-F3 or Liver elastography (Fibroscan®)≤12 kilopascal (kPa) (Cohort 1 and 2) within the 12 months prior to screening
      Creatinine clearance (CLcr)≥30 mL/min as calculated by the Cockcroft-Gault formula at screening
   Cirrhotic
      Liver biopsy with METAVIR F4 or Liver elastography (Fibroscan®)≥12 kPa (Cohort 2) within the 12 months prior to screening
      CLcr≥60 mL/min as calculated by the Cockcroft-Gault formula at screening
      CPT score of 5 or 6, inclusive at screening and at start of study
Cohort 3 (moderate impairment of liver function) specific inclusion criteria
   Apart from hepatic insufficiency, the participant must, in the opinion of the investigator, be sufficiently healthy for study participation based upon medical history, physical examination, vital signs, and screening laboratory evaluations
   Liver biopsy with METAVIR F4 or Liver elastography (Fibroscan®)≥12 kPa within the 12 months prior to screening
   CLcr≥60 mL/min as calculated by the Cockcroft-Gault formula at screening
   CPT score of 7 to 9, inclusive at screening and at start of study
Cohort 4 (severe impairment of liver function) specific inclusion criteria
   Apart from hepatic insufficiency, the participant must, in the opinion of the investigator, be sufficiently healthy for study participation based upon medical history, physical examination, vital signs, and screening laboratory evaluations
   Liver biopsy with METAVIR F4 or Liver elastography (Fibroscan®) readout≥12 kilopascal (kPa)
   CLcr≥60 mL/min as calculated by the Cockcroft-Gault formula at screening CPT score≥10 at screening and at start of study

Exclusion criteria include the following:
History of clinically significant liver disease from non-HBV and non-HDV etiology as determined by the investigator.
History of immune complex disease
History of an autoimmune disorder
History of HBV-related extrahepatic disease, including but not limited to HBV-related rash, arthritis, or glomerulonephritis
History of allergic reactions, hypersensitivity, or intolerance to study drug, its metabolites or excipients
Anti-HBs>10 IU/L at screening
Corrected QT interval (QTc)>450 milliseconds
AST or ALT>5×ULN
Total bilirubin>3×ULN (Cohorts 1 to 3), >5×ULN (Cohort 4)
Serum albumin<28 g/L (Cohorts 1 to 3), <25 g/L (Cohorts 4)
Absolute neutrophil count<1,000/mm3
Platelets<20,000/mm3
Hemoglobin<8 g/dL
History of anaphylaxis
History of malignancy diagnosed or treated within 5 years (localized treatment of squamous or noninvasive basal cell skin cancers is permitted; cervical carcinoma in situ is allowed if appropriately treated prior to screening); participants under evaluation for malignancy are not eligible
History of or listed for bone marrow or solid organ transplant
Known active infection other than chronic HBV and HDV infection or any clinically significant acute condition such as fever (>38° C.) or acute respiratory illness within 7 days prior to Day 1
Coinfection with human immunodeficiency virus (HIV), hepatitis A virus (HAV), hepatitis C virus (HCV) or hepatitis E virus (HEV). Participants who are HCV antibody positive and HCV RNA negative are eligible. Participants who are HAV or HEV immunoglobulin M antibody (IgM) positive are not eligible. Participants who are asymptomatic and HAV or HEV immunoglobulin G antibody (IgG) positive are eligible.
Any clinically significant medical or psychiatric condition that may interfere with study intervention, assessment, or compliance with the protocol or otherwise makes the participant unsuitable for participation in the study, as determined by the investigator. Participants with controlled Diabetes Mellitus are eligible.
Acute or worsening chronic hepatitis, fluctuating or rapidly deteriorating hepatic function or use of any therapy known to exacerbate hepatic dysfunction in the opinion of the investigator.
Therapy with an immunomodulatory agent, IFN-α (eg, IFN-alfa-2a or IFN-alfa-2b, or pegylated IFN-alfa-2a or alfa 2b), cytotoxic or chemotherapeutic agent, or chronic systemic corticosteroids within 6 months of screening.
Received an HDV active agent (including lonafarnib and bulevirtide) within 90 days or 5 half-lives (if known), whichever is longer, before study drug administration or are active in the Follow-Up period of another clinical study involving interventional treatment. Participants must also agree not to take part in any other interventional study at any time during their participation in this study, inclusive of the Follow-Up Period.
Receipt of an oligonucleotide (e.g., siRNA, antisense oligonucleotide) with activity against HBV within 48 weeks before study drug administration
For newly enrolled participants: receipt of AB01 within 24 weeks prior to Study Day 1.
History or clinical evidence of alcohol or drug abuse within the 12 months before screening or a positive drug screen at screening unless it can be explained by a prescribed medication (the diagnosis and prescription must be approved by the investigator). Cannabis use is permitted.
Additional Exclusions Criteria for Hepatically Impaired Participants
   Participants requiring paracentesis>1 time per month.
   Participants with refractory encephalopathy or significant Central Nervous System disease as judged by the investigator.
   History of gastric or esophageal variceal bleeding within the past 6 months.
   Participants with Transjugular Intrahepatic Portosystemic Shunt (TIPS) placement.
   Presence of hepatopulmonary or hepatorenal syndrome.
   Presence of primary cholestatic liver diseases.
   Inability or unwillingness to comply with dietary recommendations for liver cirrhosis and hepatic impairment as advised by the investigator and lifestyle considerations outlined in this protocol.

### Concomitant Therapy

Concomitant Therapy Not Permitted During the Study
   Participants must abstain from taking prescription drugs outside the care of a prescribing physician and nonprescription drugs (including vitamins, recreational drugs, and dietary or herbal supplements) within 7 days (or 14 days if the drug is a potential enzyme inducer) or 5 half-lives (whichever is longer) before the start of study intervention until completion of the follow-up visit, unless, in the opinion of the investigator and sponsor, the medication will not interfere with the study.
   Participants with hepatic impairment with co-morbid diseases requiring medication(s) must be taking the medication(s) without a change in dose for >3 months prior to screening. All concomitant medications must be approved by the Medical Monitor prior to study enrollment.
   Use of any of the following systemic medications is prohibited within 14 days before study drug administration and throughout the study:
      Chronic systemic steroids (prednisone equivalent of >10 mg/day) or other immunosuppressive agents (Note: corticosteroid administration for the treatment of immune-mediated AEs and short courses of corticosteroids for chronic obstructive pulmonary disease or asthma exacerbations is allowed)
      Paracetamol (acetaminophen)≥3 g/day
      Isoniazid
   Additionally, the administration of any potentially hepatotoxic medications during the study should be considered only if no therapeutic alternative can be identified and after a careful consideration of the potential risks and benefits for the participant. Medications that are potentially hepatotoxic or associated with drug-induced liver injury include, but are not limited to, the following (Bjornsson 2016): Aspirin>3 g/day or ibuprofen≥1.2 g/day; Tricyclic antidepressants; Valproate; Phenytoin; Amiodarone; Anabolic steroids; Allopurinol; Amoxicillin-clavulanate; Minocycline Nitrofurantoin; Sulfamethoxazole/trimethoprim; Erythromycin; Rifampin; Azole antifungals; and Herbal or natural remedies.

### REFERENCES

Abbas Z, Khan M A, Salih M, Jafri W. Interferon alpha for chronic hepatitis D. Cochrane Database Syst Rev. 2011 Dec. 7; 2011(12):CD006002. doi: 10.1002/14651858.CD006002.pub2. PMID: 22161394; PMCID: PMC6823236.
Agarwal K, Yuen M F, Wedemeyer H, Arizpe A, Shen L, Cloutier D, Tay C, Gupta S V, Fanget M, Dholakiya S L, Seu L, Cathcart A L, Hwang C, Pang P S, Gane E. Rapid hbsag reduction in chronic hepatitis B virus infection: preliminary results from a Phase 1 Study evaluating a single dose of VIR-3434, a novel neutralizing, vaccinal monoclonal antibody. AASLD: The Liver Meeting 2021, November 12-15.
Bahcecioglu I H, Sahin A. Treatment of Delta Hepatitis: Today and in the Future—A review. Infect Dis (Lond). 2017 April; 49(4):241-250. doi: 10.1080/23744235.2016.1271998. Epub 2017 Jan. 16. PMID: 28092214.
Björnsson E S. Hepatotoxicity by Drugs: The Most Common Implicated Agents. Int J Mol Sci. 2016 Feb. 6; 17(2):224. doi: 10.3390/ijms17020224. PMID: 26861310; PMCID: PMC4783956.
Chang J, Nie X, Chang H E, Han Z, Taylor J. Transcription of hepatitis delta virus RNA by RNA polymerase II. J Virol. 2008 February; 82(3):1118-27. doi: 10.1128/JVI.01758-07. Epub 2007 Nov. 21. PMID: 18032511; PMCID: PMC2224410.
Chen H Y, Shen D T, Ji D Z, Han P C, Zhang W M, Ma J F, Chen W S, Goyal H, Pan S, Xu H G. Prevalence and burden of hepatitis D virus infection in the global population: a systematic review and meta-analysis. Gut. 2019 March; 68(3):512-521. doi: 10.1136/gutjnl-2018-316601. Epub 2018 Sep. 18. PMID: 30228220.
de Lédinghen V et. al. Safety and Efficacy of 2 mg Bulevirtide in Patients with Chronic HBV/HDV Co-Infection: First Real-World Results AASLD 2021 Nov. 12-15.
European Association for the Study of the Liver. European Association for the Study of the Liver. EASL 2017 Clinical Practice Guidelines on the management of hepatitis B virus infection. J Hepatol. 2017 August; 67(2):370-398. doi: 10.1016/j.jhep.2017.03.021. Epub 2017 Apr. 18. PMID: 28427875.
Farci P, Smedile A, Lavarini C, Piantino P, Crivelli O, Caporaso N, Toti M, Bonino F, Rizzetto M. Delta hepatitis in inapparent carriers of hepatitis B surface antigen. A disease simulating acute hepatitis B progressive to chronicity. Gastroenterology. 1983 September; 85(3):669-73. PMID: 6873613.
Fattovich G, Boscaro S, Noventa F, Pornaro E, Stenico D, Alberti A, Ruol A, Realdi G. Influence of hepatitis delta virus infection on progression to cirrhosis in chronic hepatitis type B. J Infect Dis. 1987 May; 155(5):931-5. doi: 10.1093/infdis/155.5.931. PMID: 3559292.
Fattovich G, Giustina G, Christensen E, Pantalena M, Zagni I, Realdi G, Schalm S W. Influence of hepatitis delta virus infection on morbidity and mortality in compensated cirrhosis type B. The European Concerted Action on Viral Hepatitis (Eurohep). Gut. 2000 March; 46(3):420-6. doi: 10.1136/gut.46.3.420. PMID: 10673308; PMCID: PMC1727859.
FDA: Pharmacokinetics in Patients with Impaired Hepatic Function: Study Design, Data Analysis, and Impact on Dosing and Labeling. Center for Drug Evaluation and Research, Center for Biologics Evaluation and Research. May 2003. https://www.fda.gov/media/71311/download
Freitas N, Abe K, Cunha C, Menne S, Gudima S O. Support of the infectivity of hepatitis delta virus particles by the envelope proteins of different genotypes of hepatitis B virus. J Virol. 2014 June; 88(11):6255-67. doi: 10.1128/JVI.00346-14. Epub 2014 Mar. 19. PMID: 24648462; PMCID: PMC4093875.
Galun E, Eren R, Safadi R, Ashour Y, Terrault N, Keeffe E B, Matot E, Mizrachi S, Terkieltaub D, Zohar M, Lubin I, Gopher J, Shouval D, Dagan S. Clinical evaluation (phase I) of a combination of two human monoclonal antibodies to HBV: safety and antiviral properties. Hepatology. 2002 March; 35(3):673-9. doi: 10.1053/jhep.2002.31867. Erratum in: Hepatology 2002 April; 35(4):986. PMID: 11870383.
Gane E, Lim Y S, Yuen M F, Cloutier D, Thanawala V et al. Longer treatment duration of monthly VIR-2218 results in deeper and more sustained reductions in hepatitis B surface antigen in participants with chronic hepatitis B infection. Abstract submitted at the EASL International Liver Conference, June 22-26, 202.
Gane E, Lim Y S, Cloutier D, Shen L, et al. Safety and Antiviral Activity of VIR-2218, An XTargeting RNAi Therapeutic, In Participants With Chronic Hepatitis B Infection: Week 48 Follow-Up Results. Oral Presentation. Presented at the EASL Digital International Liver Conference, Jun. 23-26, 2021.
Gunsar F, Akarca U S, Ersoz G, Kobak A C, Karasu Z, Yuce G, Ilter T, Batur Y. Two-year interferon therapy with or without ribavirin in chronic delta hepatitis. Antivir Ther. 2005; 10(6):721-6. PMID: 16218171.
Gupta S V, Fanget M C, MacLauchlin C, Clausen V A, Li J, Cloutier D, Shen L, Robbie G J, Mogalian E. Clinical and Preclinical Single-Dose Pharmacokinetics of VIR-2218, an RNAi Therapeutic Targeting HBV Infection. Drugs R D. 2021 December; 21(4):455-465. doi: 10.1007/s40268-021-00369-w. Epub 2021 Nov. 6. PMID: 34741731; PMCID: PMC8602582.
Gupta S V, Arizpe A, Fanget M, Dholakiya S L, et al. Preliminary Pharmacokinetics and Safety in Healthy Volunteers of VIR-3434, a Monoclonal Antibody for the Treatment of Chronic Hepatitis
B Infection. Poster. Presented at the EASL Digital International Liver Conference, Jun. 23-26, 2021.
Heidrich B, Yurdaydin C, Kabaçam G, Ratsch B A, Zachou K, Bremer B, et al. Late HDV RNA relapse after peginterferon alpha-based therapy of chronic hepatitis delta. Hepatology 2014; 60:87-97. Hepcludex Summary of Product Characteristics. Hepcludex, 2 mg powder for solution for injection. Ireland: Gilead Sciences Ireland UC; 2020. Available from: http://www.ema.europa.eu.
Kamili S, Drobeniuc J, Mixson-Hayden T, Kodani M. Delta hepatitis: Toward improved diagnostics. Hepatology. 2017 December; 66(6):1716-1718. doi: 10.1002/hep.29564. Epub 2017 Oct. 30. PMID: 28961326.
Le Gal F, Brichler S, Drugan T, Alloui C, Roulot D, Pawlotsky J M, Dény P, Gordien E. Genetic diversity and worldwide distribution of the deltavirus genus: A study of 2,152 clinical strains. Hepatology. 2017 December; 66(6):1826-1841. doi: 10.1002/hep.29574. Epub 2017 Oct. 30. PMID: 28992360.
Lee A U, Lee C. Hepatitis D Review: Challenges for the Resource-Poor Setting. Viruses. 2021 Sep. 23; 13(10):1912. doi: 10.3390/v13101912. PMID: 34696341; PMCID: PMC8538672.
Lempp F A, Ni Y, Urban S. Hepatitis delta virus: insights into a peculiar pathogen and novel treatment options. Nat Rev Gastroenterol Hepatol. 2016 October; 13(10):580-9. doi: 10.1038/nrgastro.2016.126. Epub 2016 Aug. 18. PMID: 27534692.
Lempp F A, Vincenzetti L, Volz T, et al., editors. Preclinical Characterization of VIR-3434, a Monoclonal Antibody Neutralizing Hepatitis B Virus That Facilitates FcγR-Mediated Elimination of HBsAg. American Association for the Study of Liver Diseases (AASLD); 2021 Nov. 12-15, 2021; Anaheim, CA, PO-838.
Lok A S, Negro F, Asselah T, Farci P, Rizzetto M. Endpoints and New Options for Treatment of Chronic Hepatitis D. Hepatology. 2021 December; 74(6):3479-3485. doi: 10.1002/hep.32082. Epub 2021 Sep. 16. PMID: 34331781.
Lucifora J, Delphin M. Current knowledge on Hepatitis Delta Virus replication. Antiviral Res. 2020 July; 179:104812. doi: 10.1016/j.antiviral.2020.104812. Epub 2020 Apr. 29. PMID: 32360949.
Mathurin P, Thibault V, Kadidja K, Ganne-Carrie N, Moussalli J, El Younsi M, Di Martino V, Lunel F, Charlotte F, Vidaud M, Opolon P, Poynard T. Replication status and histological features of patients with triple (B, C, D) and dual (B, C) hepatic infections. J Viral Hepat. 2000 January; 7(1):15-22. doi: 10.1046/j.1365-2893.2000.00195.x. PMID: 10718938.
Miao Z, Zhang S, Ou X, Li S, Ma Z, Wang W, Peppelenbosch M P, Liu J, Pan Q. Estimating the Global Prevalence, Disease Progression, and Clinical Outcome of Hepatitis Delta Virus Infection. J Infect Dis. 2020 Apr. 27; 221(10):1677-1687. doi: 10.1093/infdis/jiz633. PMID: 31778167; PMCID: PMC7184909.
Modahl L E, Macnaughton T B, Zhu N, Johnson D L, Lai M M. RNA-Dependent replication and transcription of hepatitis delta virus RNA involve distinct cellular RNA polymerases. Mol Cell Biol. 2000 August; 20(16):6030-9. doi: 10.1128/MCB.20.16.6030-6039.2000. PMID: 10913185; PMCID: PMC86079.
National Institute for Health. National Institute of Diabetes and Digestive and Kidney Diseases. United States 2017. Department of Health and Human Services. Accessed Apr. 10, 2020.
NCI Common Terminology Criteria for Adverse Events (CTCAE) Version 5.0 Published: Nov. 27, 2017, U.S. Department of Health and Human Services, National Institutes of Health. National Cancer Institute. https://ctep.cancer.gov/protocoldevelopment/electronic_applications/docs/CTCAE_v5_Quick_Reference_8.5x11.pdf.
Niro G A, Smedile A. Current concept in the pathophysiology of hepatitis delta infection. Curr Infect Dis Rep. 2012 February; 14(1):9-14. doi: 10.1007/s11908-011-0233-5. PMID: 22161240.
Niro G A, Smedile A, Ippolito A M, Ciancio A, Fontana R, Olivero A, Valvano M R, Abate M L, Gioffreda D, Caviglia G P, Rizzetto M, Andriulli A. Outcome of chronic delta hepatitis in Italy: a long-term cohort study. J Hepatol. 2010 November; 53(5):834-40. doi: 10.1016/j.jhep.2010.06.008. Epub 2010 Jul. 29. PMID: 20800919.
Perrillo R, Buti M, Durand F, Charlton M, Gadano A, Cantisani G, et al. Entecavir and hepatitis B immune globulin in patients undergoing liver transplantation for chronic hepatitis B. Liver Transpl. 2013 August; 19(8):887-95.
Polaris Observatory Collaborators. Global prevalence, treatment, and prevention of hepatitis B virus infection in 2016: a modelling study. Lancet Gastroenterol Hepatol. 2018 June; 3(6):383-403. doi: 10.1016/S2468-1253(18)30056-6. Epub 2018 Mar. 27. PMID: 29599078.
Raimondo G, Smedile A, Gallo L, Balbo A, Ponzetto A, Rizzetto M. Multicentre study of prevalence of HBV-associated delta infection and liver disease in drug-addicts. Lancet. 1982 Jan. 30; 1(8266):249-51. doi: 10.1016/s0140-6736(82)90976-x. PMID: 6120275.
Reed W D, Eddleston A L, Cullens H, Williams R, Zuckerman A J, Peters D K, Williams D G, Maycock W A. Infusion of hepatitis-B antibody in antigen-positive active chronic hepatitis. Lancet. 1973 Dec. 15; 2(7842):1347-51. doi: 10.1016/s0140-6736(73)93321-7. PMID: 4128052.
Reilly M C, Zbrozek A S, Dukes E M. The validity and reproducibility of a work productivity and activity impairment instrument. Pharmacoeconomics. 1993 November; 4(5):353-65. doi: 10.2165/00019053-199304050-00006. PMID: 10146874.
Rizzetto M. Hepatitis D Virus: Introduction and Epidemiology. Cold Spring Harb Perspect Med. 2015 Jul. 1; 5(7):a021576. doi: 10.1101/cshperspect.a021576. PMID: 26134842; PMCID: PMC4484953.
Rizzetto M, Hamid S, Negro F. The changing context of hepatitis D. J Hepatol. 2021 May; 74(5):1200-1211. doi: 10.1016/j.jhep.2021.01.014. Epub 2021 Jan. 20. PMID: 33484770.
Romeo R, Del Ninno E, Rumi M, Russo A, Sangiovanni A, de Franchis R, Ronchi G, Colombo M. A 28-year study of the course of hepatitis Delta infection: a risk factor for cirrhosis and hepatocellular carcinoma. Gastroenterology. 2009 May; 136(5):1629-38. doi: 10.1053/j.gastro.2009.01.052. Epub 2009 Jan. 29. PMID: 19208358.
Sagnelli E, Felaco F M, Filippini P, Pasquale G, Peinetti P, Buonagurio E, Aprea L, Pulella C, Piccinino F, Giusti G. Influence of HDV infection on clinical, biochemical and histological presentation of HBsAg positive chronic hepatitis. Liver. 1989 August; 9(4):229-34. doi:10.1111/j.1600-0676.1989.tb00404.x. PMID: 2770435.
Sarin S K, Kumar M, Lau G K, Abbas Z, Chan H L, Chen C J, Chen D S, Chen H L, Chen P J, Chien R N, Dokmeci A K, Gane E, Hou J L, Jafri W, Jia J, Kim J H, Lai C L, Lee H C, Lim S G, Liu C J, Locarnini S, Al Mahtab M, Mohamed R, Omata M, Park J, Piratvisuth T, Sharma B C, Sollano J, Wang F S, Wei L, Yuen M F, Zheng S S, Kao J H. Asian-Pacific clinical practice guidelines on the management of hepatitis B: a 2015 update. Hepatol Int. 2016 January; 10(1):1-98. doi: 10.1007/s12072-015-9675-4. Epub 2015 Nov. 13. PMID: 26563120; PMCID: PMC4722087.
Shirvani-Dastgerdi E, Tacke F. Molecular interactions between hepatitis B virus and delta virus. World J Virol. 2015 May 12; 4(2):36-41. doi: 10.5501/wjv.v4.i2.36. PMID: 25964870; PMCID: PMC4419120.
Sleijfer S, Bannink M, Van Gool A R, Kruit W H, Stoter G. Side effects of interferon-alpha therapy. Pharm World Sci. 2005 December; 27(6):423-31. doi: 10.1007/s11096-005-1319-7. PMID: 16341948.
Stockdale A J, Kreuels B, Henrion M Y R, Giorgi E, Kyomuhangi I, de Martel C, Hutin Y, Geretti A M. The global prevalence of hepatitis D virus infection: Systematic review and meta-analysis. J Hepatol. 2020 September; 73(3):523-532. doi: 10.1016/j.jhep.2020.04.008. Epub 2020 Apr. 23. PMID: 32335166; PMCID: PMC7438974.
Sureau C, Negro F. The hepatitis delta virus: Replication and pathogenesis. J Hepatol. 2016 April; 64(1 Suppl):S102-S116. doi: 10.1016/j.jhep.2016.02.013. PMID: 27084031.
Terrault N A, Lok A S F, McMahon B J, Chang K M, Hwang J P, Jonas M M, Brown R S Jr, Bzowej N H, Wong J B. Update on prevention, diagnosis, and treatment of chronic hepatitis B: AASLD 2018 hepatitis B guidance. Hepatology. 2018 April; 67(4):1560-1599. doi: 10.1002/hep.29800. PMID: 29405329; PMCID: PMC5975958.
Tsuge M, Hiraga N, Uchida T, Kan H, Miyaki E, Masaki K, Ono A, Nakahara T, Abe-Chayama H, Zhang Y, Naswa M G, Kawaoka T, Miki D, Imamura M, Kawakami Y, Aikata H, Ochi H, Hayes C N, Chayama K. Antiviral effects of anti-HBs immunoglobulin and vaccine on HBs antigen seroclearance for chronic hepatitis B infection. J Gastroenterol. 2016 November; 51(11):1073-1080. doi: 10.1007/s00535-016-1189-x. Epub 2016 Mar. 4. PMID: 26943168.
Urban S, Neumann-Haefelin C, Lampertico P. Hepatitis D virus in 2021: virology, immunology and new treatment approaches for a difficult-to-treat disease. Gut. 2021 September; 70(9):1782-1794. doi: 10.1136/gutjnl-2020-323888. Epub 2021 Jun. 8. PMID: 34103404; PMCID: PMC8355886.
Vlachogiannakos J, Papatheodoridis G V. New epidemiology of hepatitis delta. Liver Int. 2020 February; 40 Suppl 1:48-53. doi: 10.1111/liv.14357. PMID: 32077599.
Wedemeyer H, et al. Treatment with bulevirtide improves patient reported outcomes in patients with chronic hepatitis delta (CHD): an interim exploratory analysis at week 24. The Liver Meeting, abstract 680, 2021.
Wedemeyer H, Yurdaydin C, Dalekos G N, Erhardt A, Qakaloglu Y, Degertekin H, Gurel S, Zeuzem S, Zachou K, Bozkaya H, Koch A, Bock T, Dienes H P, Manns M P; HIDIT Study Group. Peginterferon plus adefovir versus either drug alone for hepatitis delta. N Engl J Med. 2011 Jan. 27; 364(4):322-31. doi: 10.1056/NEJMoa0912696. PMID: 21268724.
WHO (World Health Organization). Hepatitis D. https://www.who.int/news-room/factsheets/detail/hepatitis-d. Published July 2021. Accessed Apr. 10, 2022.
WHO (World Health Organization). Hepatitis B. https://www.who.int/news-room/factsheets/detail/hepatitis-b. Published July 2021. Accessed Apr. 10, 2022.
Ye X, Tateno C, Thi E P, Kakuni M, Snead N M, Ishida Y, Barnard T R, Sofia M J, Shimada T, Lee A C H. Hepatitis B Virus Therapeutic Agent ARB-1740 Has Inhibitory Effect on Hepatitis Delta Virus in a New Dually-Infected Humanized Mouse Model. ACS Infect Dis. 2019 May 10; 5(5):738-749. doi: 10.1021/acsinfecdis.8b00192. Epub 2018 Nov. 20. PMID: 30408957.
Younossi Z M, Guyatt G, Kiwi M, Boparai N, King D. Development of a disease specific questionnaire to measure health related quality of life in patients with chronic liver disease. Gut. 1999 August; 45(2):295-300. doi: 10.1136/gut.45.2.295. PMID: 10403745; PMCID: PMC1727607.
Younossi Z M, Stepanova M, Younossi I, Racila A. Development and validation of a hepatitis B specific health-related quality-of-life instrument: CLDQ-HBV. J Viral Hepat. 2021 March; 28(3):484-492. doi: 10.1111/jvh.13451. Epub 2020 Dec. 20. PMID: 33306234.
Yuen M F, Lim Y S, Cloutier D, Thanawala V, et al. Preliminary Results From a Phase 2 Study Evaluating VIR-2218 Alone and in Combination With Pegylated Interferon Alfa-2a in Participants With Chronic Hepatitis B Infection. Oral Presentation. Presented at AASLD: The Liver Meeting (Virtual), Nov. 12-15, 2021.
Yurdaydin C, Abbas Z, Buti M, Cornberg M, Esteban R, Etzion O, Gane E J, Gish R G, Glenn J S, Hamid S, Heller T, Koh C, Lampertico P, Lurie Y, Manns M, Parana R, Rizzetto M, Urban S, Wedemeyer H; Hepatitis Delta International Network (HDIN). Treating chronic hepatitis delta: The need for surrogate markers of treatment efficacy. J Hepatol. 2019 May; 70(5):1008-1015. doi: 10.1016/j.jhep.2018.12.022. Epub 2018 Dec. 27. PMID: 30982526.
Zachou K, Yurdaydin C, Drebber U, Dalekos G N, Erhardt A, Cakaloglu Y, Degertekin H, Gurel S, Zeuzem S, Bozkaya H, Schlaphoff V, Dienes H P, Bock T C, Manns M P, Wedemeyer H; HIDT-1 Study Group. Quantitative HBsAg and HDV-RNA levels in chronic delta hepatitis. Liver Int. 2010 March; 30(3):430-7. doi: 10.1111/j.1478-3231.2009.02140.x. Epub 2009 Oct. 16. PMID: 19840253.

### Example 2

### Neutralization of HDV by Antibody Targeting HBsAG

### Background

Chronic hepatitis B virus (HBV) infection is a major global public health burden affecting approximately 296 million people worldwide, resulting in an estimated 820,000 deaths annually (Polaris Observatory Collaborators, 2018; WHO, Hepatitis B, 2021).

SIRNA01 is an investigational siRNA therapeutic that targets the HBx region of the HBV genome and demonstrates potent in vitro and in vivo antiviral activity. The sense strand of SIRNA01 is conjugated to an N-acetyl galactosamine (GalNAc) ligand to enable targeted delivery to the liver. AB01 is an investigational neutralizing monoclonal antibody targeting the antigenic loop of HBsAg with pan-genotypic neutralizing activity in vitro. Treatment with murinized AB01 inhibits viral spread and leads to elimination of HBsAg in vivo. The mAb carries an engineered Fc that extends serum half-life (LS mutation) and increases binding to activating FcgRs (FcgRIIa and IIIa) but decreases binding to inhibitory FcgRIIb (XX2/GAALIE mutation).

### Methods

Entry inhibition of HBV upon AB01 treatment was confirmed using primary human hepatocytes (PHH) infected with HBV (genotype D). HBeAg in the cell culture supernatant was quantified by chemiluminescence immunoassay (CLIA) as marker for infection.

To assess the in vitro activity of AB01 against eight different HBV genotypes, Hepatitis D virus (HDV), a satellite virus of HBV found in 5% of patients with chronic HBV (WHO, Hepatitis D, 2021), was used. As HDV requires the HBV envelope protein for entry into and secretion from hepatocytes, it can be used as a tool to study HBsAg from different HBV genotypes (Wang et al., 2021). Here, neutralization by AB01 was assessed in Huh7-NTCP cells that were infected with HDV pseudotyped with HBsAg from eight different HBV genotypes (A-H).

To evaluate the in vitro antiviral activity of SIRNA01 against representative HBV genotypes A through D, the HBV1.3-overlength genome system in which all viral RNAs are transcribed under the regulation of authentic HBV promoters was used. Huh7 cells were transfected with plasmids containing the HBV1.3 genome sequences from 13 isolates of HBV representing genotypes A through D, followed by transfection of SIRNA01 or control siRNA. HBsAg was used as readout.

To evaluate the combination activity of SIRNA01 and AB01 versus monotherapy, two in vivo studies were conducted using well-established mouse models of HBV infection: C57BL/6 mice transduced with AAV8-HBV (genotype D) or human liver-chimeric PXB-mice infected with HBV (genotype C). The mice were treated with the SIRNA01, HBC34-mu (a murinized version of AB01), entecavir (ETV, AAV8-HBV study only), or a combination of agents at different concentrations. Antiviral activity was determined by evaluation of viral serum/plasma markers including HBV DNA, HBsAg, and HBeAg.

### Results

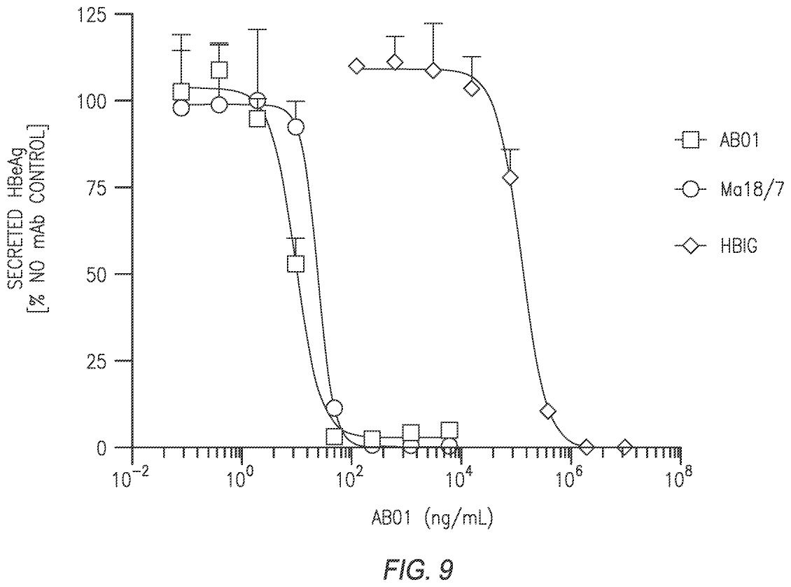

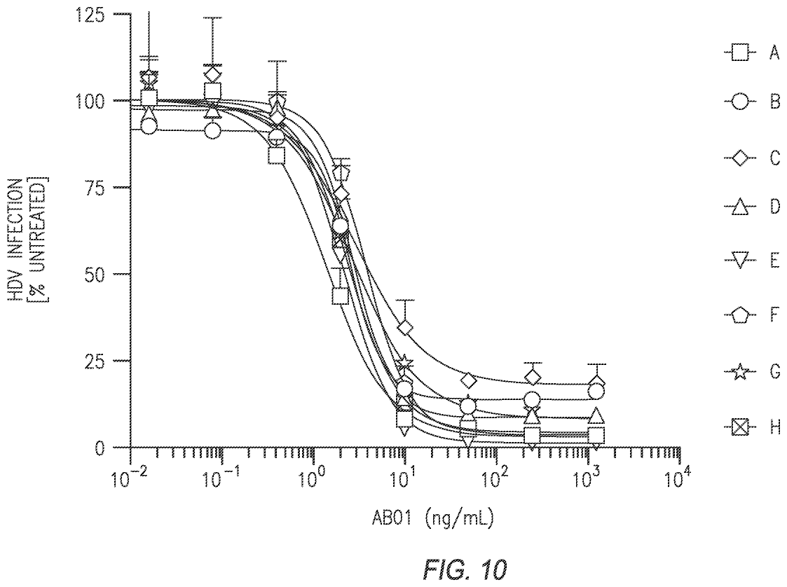

AB01 and SIRNA01 show potent activity against virus harboring all tested HBsAg genotypes. Primary human hepatocytes (PHH) were infected with HBV (genotype D) in the presence of AB01, preS1-targeting Ma18/7 mAb or polyclonal Hepatitis B Immune Globulin (HBIG). HBV neutralization activity was assessed by quantifying secreted HBeAg as a marker for infection 7 days post infection (FIG. 9). To evaluate the neutralization capacity of AB01 against different HBV genotypes in the context of infection, HDV enveloped with HBsAg from different HBV genotypes was utilized. Neutralization by AB01 was assessed in Huh7-NTCP cells that were infected with HDV pseudotyped with HBsAg from eight different HBV genotypes (A-H) (FIG. 10).

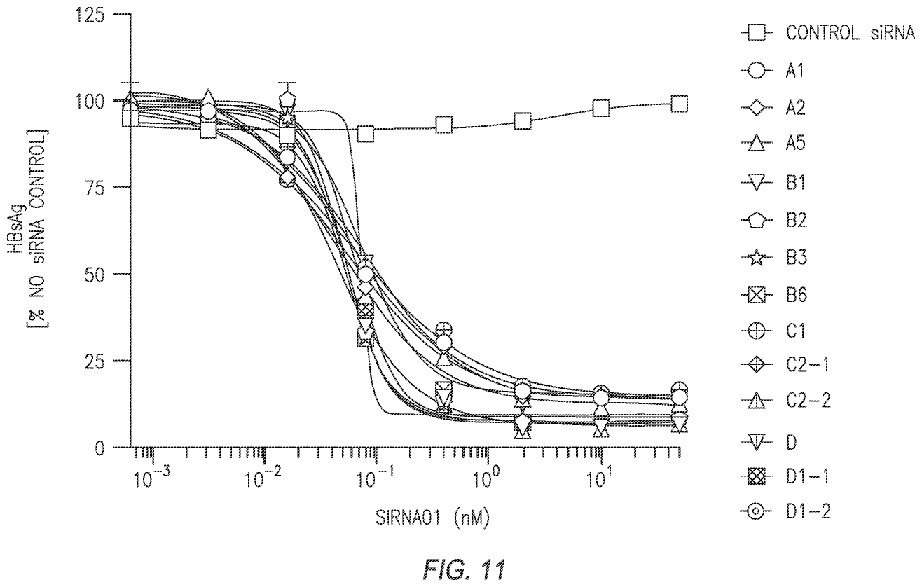

The activity of SIRNA01 against HBV genotypes A through D was assessed in HBV-genome transfected human hepatocarcinoma cells (Huh7) (FIG. 11).

### REFERENCES

Polaris Observatory Collaborators. Global prevalence, treatment, and prevention of hepatitis B virus infection in 2016: a modelling study. Lancet Gastroenterol Hepatol 2018; 3(6):383-403.
Hepatitis B. World Health Organization. Jul. 27, 2021. Accessed Apr. 14, 2022. https://www.who.int/news-room/fact-sheets/detail/hepatitis-b.
Hepatitis D. World Health Organization. Jul. 28, 2021. Accessed Apr. 29, 2022. https://www.who.int/news-room/fact-sheets/detail/hepatitis-d
Wang W. et. al.: Assembly and infection efficacy of hepatitis B virus surface protein exchanges in 8 hepatitis D virus genotype isolates; J Hepatol. 2021 August; 75(2):311-323. doi: 10.1016/j.jhep.2021.03.025

### Example 3

### Clinical Evaluation of Monotherapy or Combination Therapy to Treat Chronic HDV Infection

The safety, efficacy, and tolerability of an anti-HBV antibody in combination with an anti-HBV siRNA is evaluated in a Phase 2, multi-center, open-label study clinical study in noncirrhotic and compensated cirrhotic (CPT-A) human patients with chronic HBV/HDV coinfection on nucleoside reverse transcriptase inhibitors (NRTI) therapy.

The objectives of this study are to evaluate the safety of SIRNA01 and AB01 in participants with chronic HBV/HDV coinfection and evaluate whether monotherapy or combination therapy with the investigation agents can durably suppress HDV replication and normalize ALT when given on a biweekly, monthly, or bimonthly schedule to participants with varying degrees of liver fibrosis and compensated cirrhosis.

### Study Design

Table 6 shows the treatment groups for the study. The study will consist of cohorts receiving either SIRNA01 or AB01 monotherapy or combination therapy. For Cohorts 1a and 1b, the intervention period with SIRNA01 and AB01 monotherapy is composed of 2 periods: Induction (12 weeks) and Maintenance (up to 84 weeks). For Cohorts 2a, 2b1, 2b2, 2c and 3, the intervention period with SIRNA01 and/or AB01 consists of the Treatment Period only (up to 96 weeks). Cohort 4 participants will delay treatment for 12 weeks prior to starting combination therapy with SIRNA01 and AB01 for up to 96 weeks. Cohorts 2 through 4 will open following review of 12-week safety and efficacy data from Cohorts 1a and 1b. The study also includes 3 optional sub-studies collecting (1) liver tissue, (2) fine needle aspirate of the liver, and (3) blood samples for pharmacokinetic (PK) studies.

TABLE 6
Treatment groups for clinical study.

| | | | | Induction Period | | Maintenance Period | |
|---|---|---|---|---|---|---|---|
| | | | | Number | Frequency | Number | Frequency |
| Cohort | Intervention | Dose | Route | of Doses | of Dosing | of Doses | of Dosing |
| 1a ^{a, b} | SIRNA01 | 200 mg | SC | 3 | Every 4 | 10 | Every 8 |
| | | | | | weeks | | weeks |
| 1b ^{a, b} | AB01 | 300 mg | SC | 3 | Every 4 | 10 | Every 8 |
| | | | | | weeks | | weeks |

| | | | | Treatment Period | |
|---|---|---|---|---|---|
| Cohort | Intervention | Dose | Route | Number of Doses | Frequency of Dosing |
| 2a* ^{c, d} | SIRNA01 | 200 mg | SC | 13 or 24 | Every 4 weeks |
| 2b1* ^{c, d, e} | AB01 | 300 mg | SC | 13 or 24 | Every 4 weeks |
| 2b2* ^{c, d} | AB01 | 300 mg | SC | 13 or 24 | Every 4 weeks |
| 2c* ^{f, g} | SIRNA01 | 200 mg | SC | 13 or 24 | Every 4 weeks |
| | AB01 | 300 mg | SC | 13 or 24 | Every 4 weeks |
| 3* ^{c, d, e} | AB01 | 300 mg | SC | 25 or 48 | Every 2 weeks |
| 4 ^{h} | SIRNA01 | 200 mg | SC | 13 or 24 | Every 4 weeks |
| | AB01 | 300 mg | SC | 13 or 24 | Every 4 weeks |

SC = subcutaneous
*Combined endpoint defined as undetectable HDV RNA (<LOD) or ≥2 log₁₀ decrease in HDV RNA from baseline and ALT < ULN.
^{a} Participants achieving the combined endpoint at Week 12 will receive 10 additional doses every 8 weeks.
^{b} Participants NOT achieving the combined endpoint at Week 12 can enter Cohort 2c Day 1 or the Follow-up Period.
^{c} Participants achieving the combined endpoint at Week 48 will continue in monotherapy through Week 96.
^{d} Participants NOT achieving the combined endpoint at Week 48 can either enter the Follow-up Period or begin combination therapy with SIRNA01 + AB01 and follow investigational product (IP) administration in the Cohort 2c Schedule of Activities (SoA) from Week 52 to Week 96.
^{e} Participants meeting Virologic non-response criteria at Week 24 can begin combination therapy with SIRNA01 and follow IP administration in the Cohort 2c SoA from Week 28 to Week 96.
^{f} De novo participants, including participants entering from Cohort 4, achieving the combined endpoint at Week 48 will continue in combination therapy through Week 96.
^{g} De novo participants, including participants entering from Cohort 4, NOT achieving the combined endpoint at Week 48 will enter the Follow-up Period.
^{h} Participants will delay treatment for 12 weeks before transitioning to Cohort 2c Day 1.

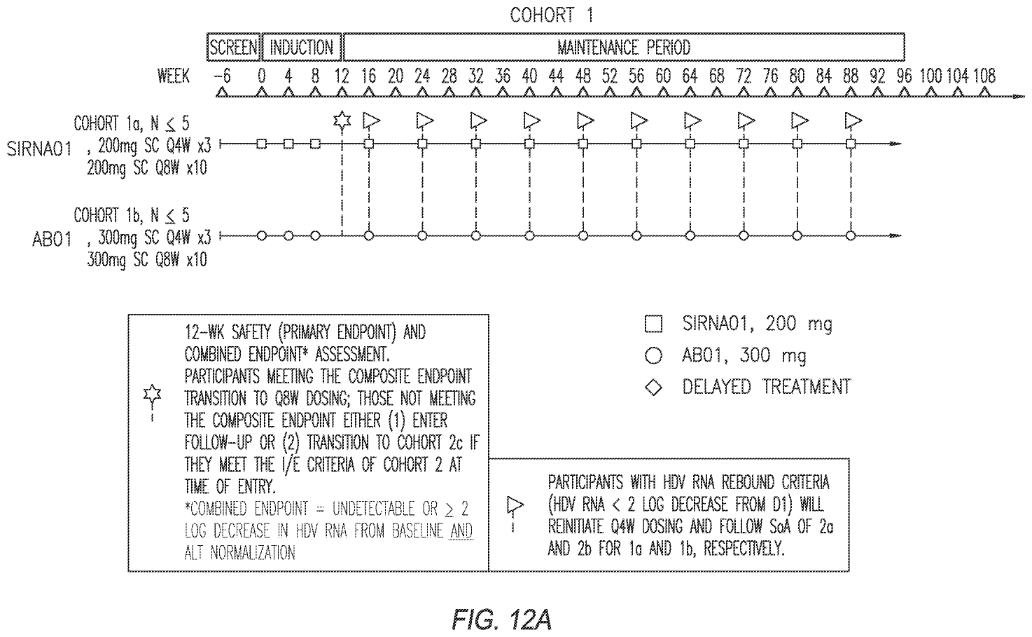

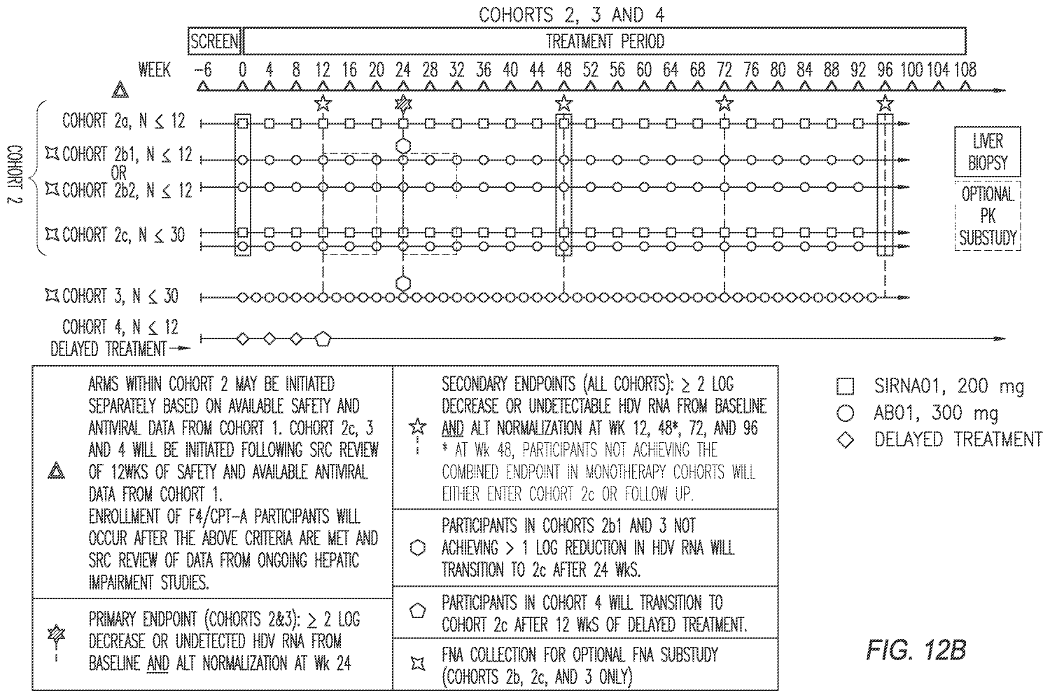

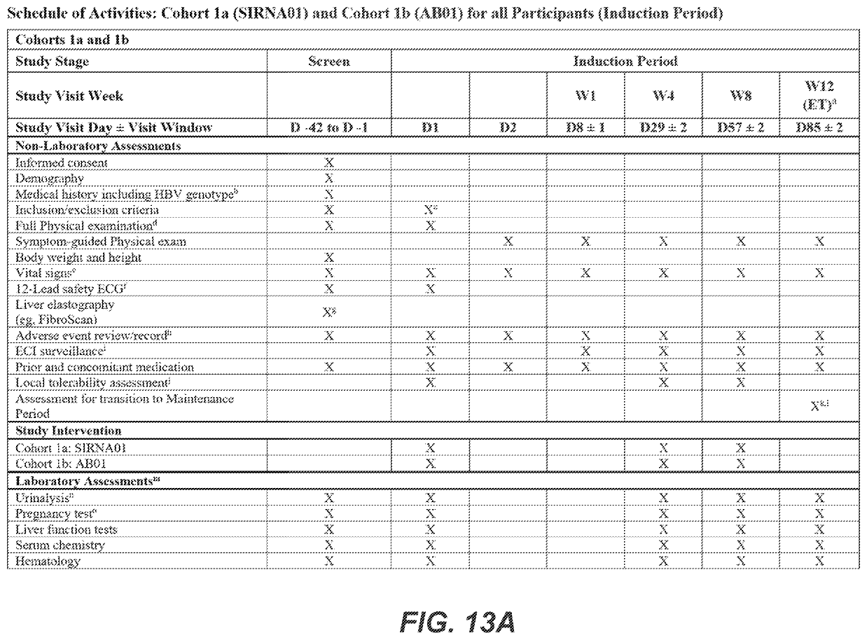

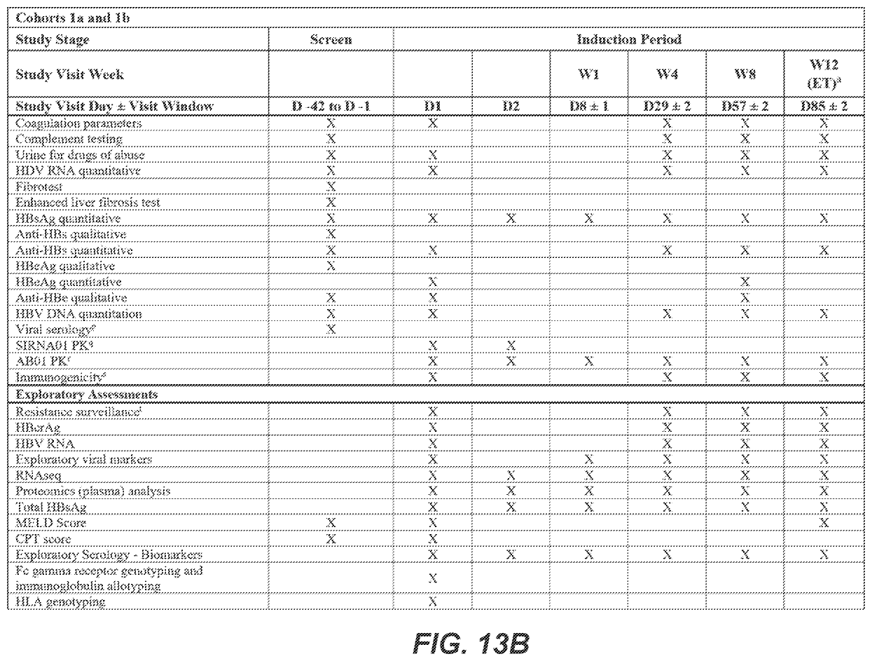

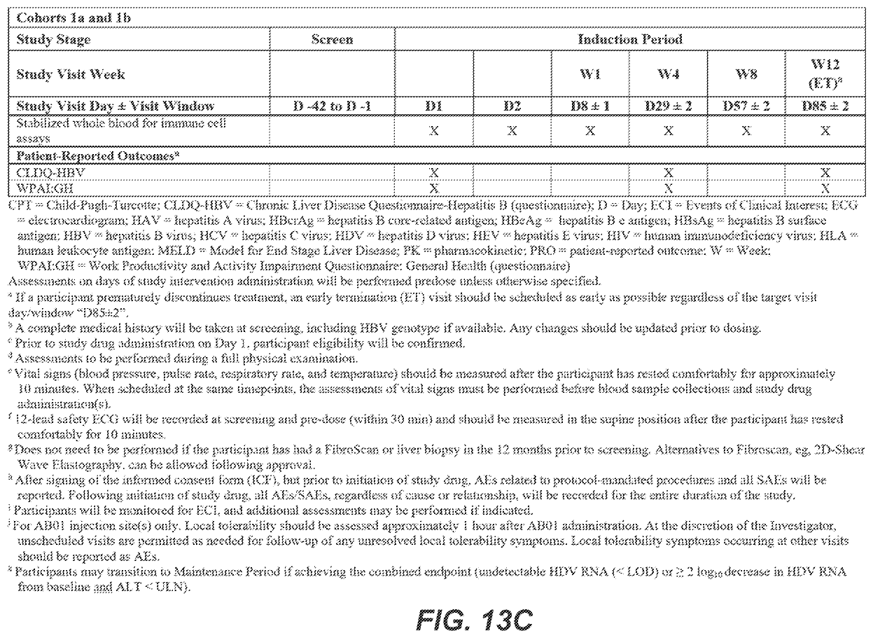

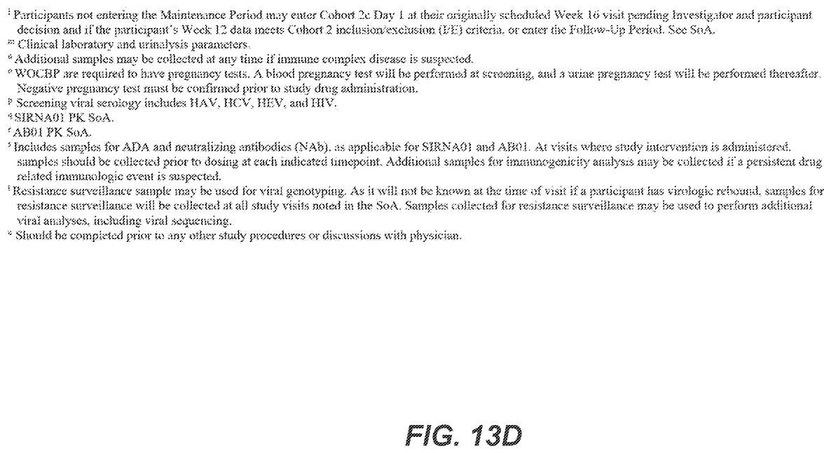

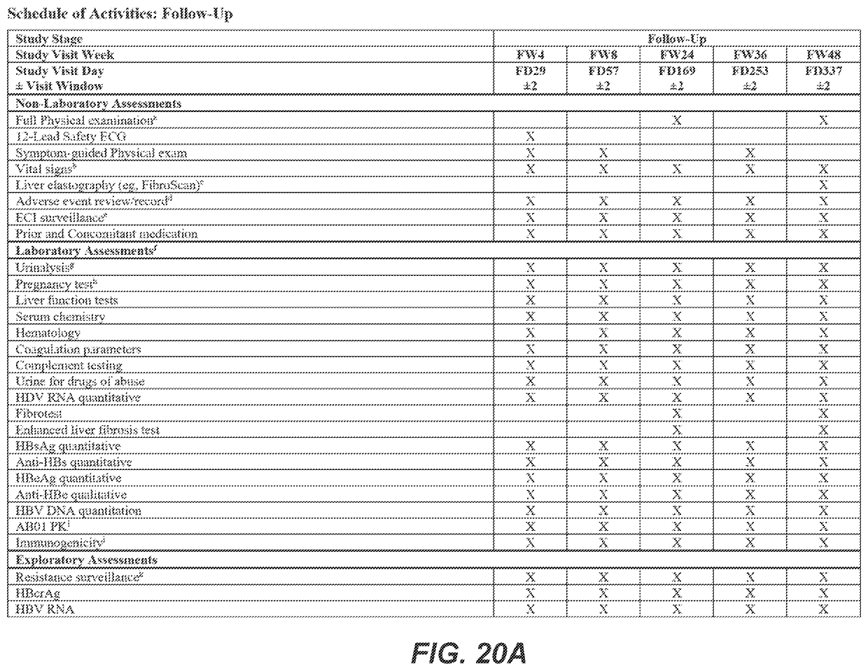

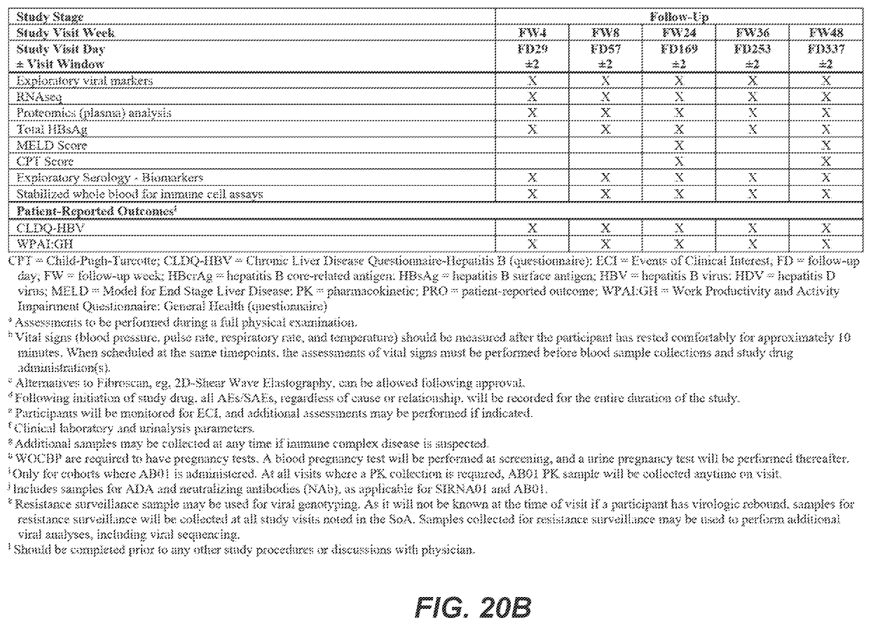

The study scheme for Cohort 1 is shown in FIG. 12A, and the study scheme for Cohorts 2, 3, and 4 is shown in FIG. 12B. The schedules of activities (“SoA”) for the cohorts and Follow-Up Period are provided in FIGS. 13A-20B.

Approximately up to 118 participants will be enrolled. This includes a pool of up to 24 floater participants total who may be added to any arm(s). Participants are planned to be enrolled in sites worldwide. The participants in the cohorts are as follows;
Cohorts 1a and 1b: Approximately 10 participants with HBV/HDV coinfection with liver fibrosis staging of Meta-Analysis of Histological Data in Viral Hepatitis (METAVIR) F0-F3: Approximately 5 participants per cohort.
Cohort 2 is comprised of 4 arms enrolling approximately up to 78 participants with either METAVIR F0-F3 or METAVIR F4, CPT-A liver disease. Up to approximately 50% of the participants in each cohort will have METAVIR F4, CPT-A liver disease. Each cohort will enroll approximately 50% participants with a baseline HBsAg<5000 IU/mL.
   Cohort 2a: planned enrollment of approximately 12 participants and a maximum, including floaters, of 36.
   EITHER Cohort 2b1: planned enrollment of approximately 12 participants and a maximum, including floaters, of 36.
   OR Cohort 2b2: planned enrollment of approximately 12 participants and a maximum, including floaters, of 36.
   Cohort 2c: planned enrollment up to approximately 30 de novo participants, inclusive of participants entering from Cohort 4 (if applicable). Participants entering from Cohort 1a or Cohort 1b will not contribute to the planned enrollment. Total cohort size, including floaters, of maximum 54.
Cohort 3: planned enrollment up to approximately 30 participants for a maximum, including floaters, of 54. Up to approximately 50% of the participants in this cohort will have METAVIR F4, CPT-A liver disease.
Cohort 4: planned enrollment up to approximately 12 participants. Up to approximately 50% of the participants in this cohort will have METAVIR F4, CPT-A liver disease.

The study will begin with Cohort 1. Cohort 1 participants who do not meet the combined endpoint at Week 12, may enter Cohort 2c Day 1 at their originally scheduled Week 16 visit pending Investigator and participant decision and if the participant's Week 12 data meets Cohort 2 inclusion/exclusion (I/E) criteria. Arms within Cohort 2 may be initiated separately based on available safety and antiviral data from Cohort 1. Cohort 3 will be initiated following review of 12-week safety and available antiviral data from Cohort 1b. Enrollment of de novo noncirrhotic participants into Cohorts 2c and 4 will begin only after review of a minimum of 12-week safety and antiviral data from Cohorts 1a (approx. n=5) and 1b (approx. n=5). Enrollment of CPT-A cirrhotic participants into Cohorts 2a, 2b1 or 2b2, and 3 will begin only after review of a minimum of 12-week safety and antiviral data from approximately 10 participants receiving SIRNA01 monotherapy (approx. n=5) or AB01 monotherapy (approx. n=5) and other available safety data. Enrollment of CPT-A cirrhotic participants into Cohorts 2c and 4 will begin only after review of a minimum of 12-week safety and antiviral data from approximately 5 noncirrhotic participants in Cohort 2c and other available safety data. Cohorts may be paused, closed or discontinued.

The maximum total study duration is planned to be up to 118 weeks. For Cohort 1, this includes a Screening Period (up to 6 weeks), Induction Period (12 weeks), and Maintenance Period (84 weeks). At the end of the Induction Period, participants may (1) transition to the Maintenance Period, (2) enter Cohort 2c, or (3) enter the Follow-Up Period at the next visit (Week 16). Participants who prematurely discontinue study treatments during the Induction Period will have an early termination (ET) visit then enter the Follow-Up Period 4 weeks later. Participants who prematurely discontinue study treatment during the Maintenance Period will have an ET visit then enter the Follow-Up Period for 48 weeks after the last dose of the study intervention or until Week 96 whichever is earlier. For Cohorts 2a, 2b1, 2b2, 2c, and 3, this includes a Screening Period (up to 6 weeks) and Treatment Period (48 to 96 weeks). Participants who prematurely discontinue study treatments will have an ET visit then enter the Follow-Up Period for 48 weeks after the last dose of the study intervention or until Week 96 whichever is earlier. For Cohort 4, this includes a Screening Period (up to 6 weeks) and 12 weeks of delayed treatment (while continuing to receive NRTI).

The Screening Period for all participants will be up to 42 days.

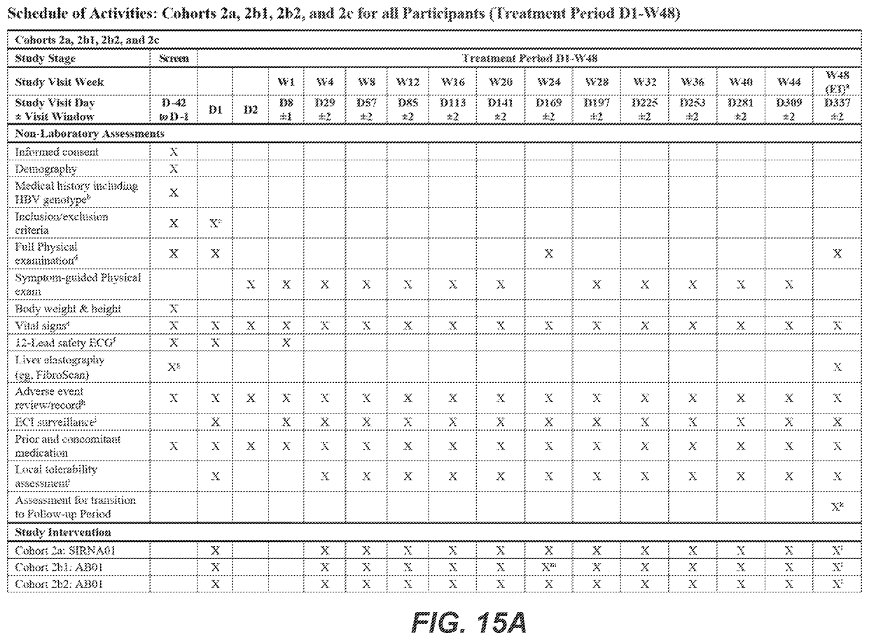

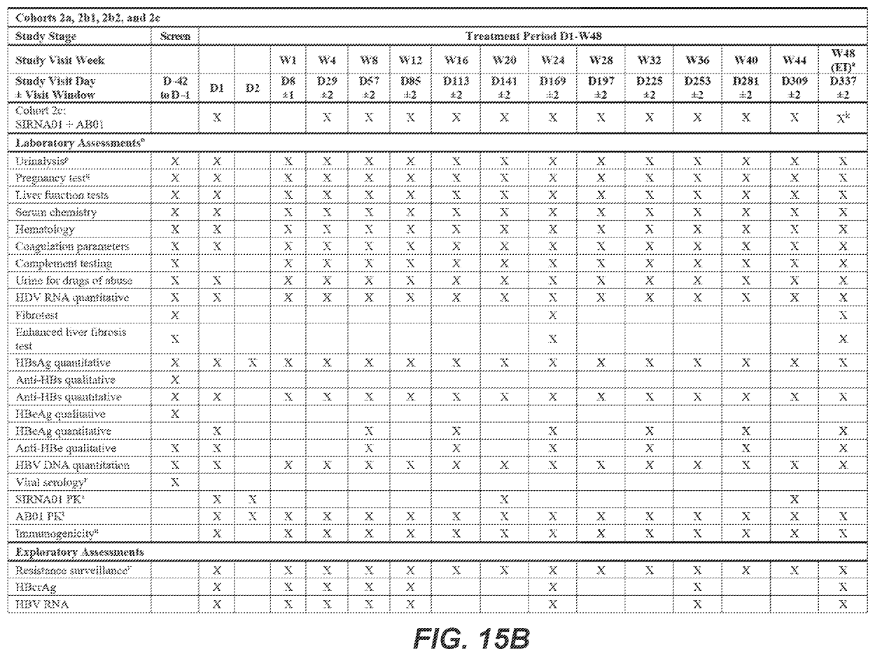

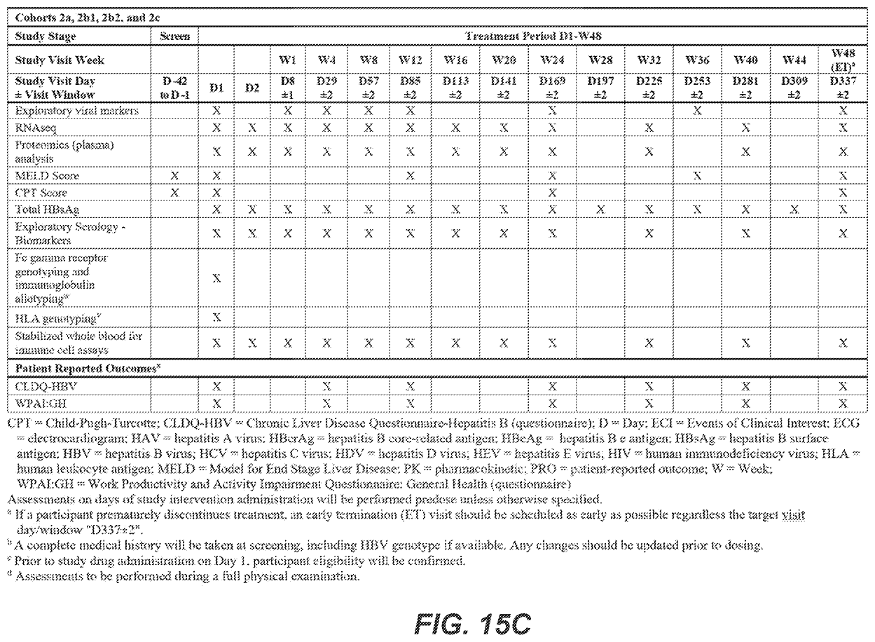

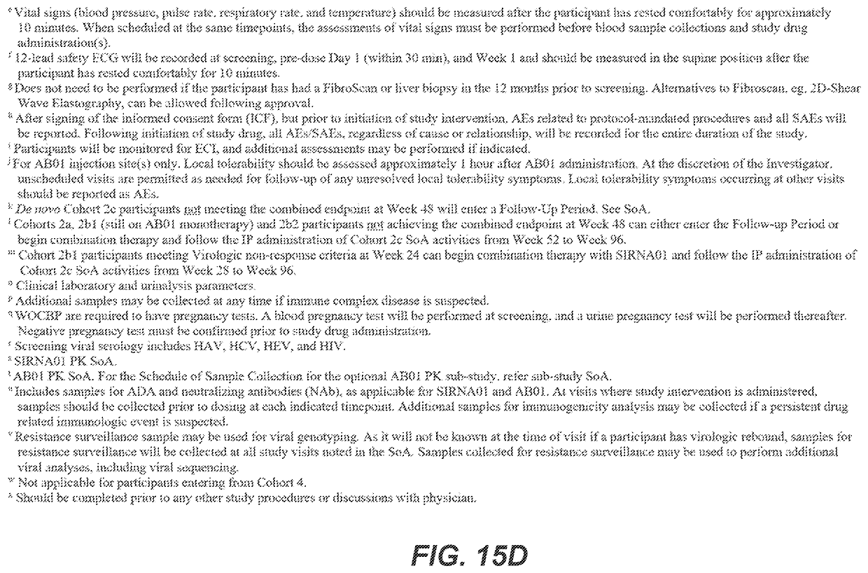

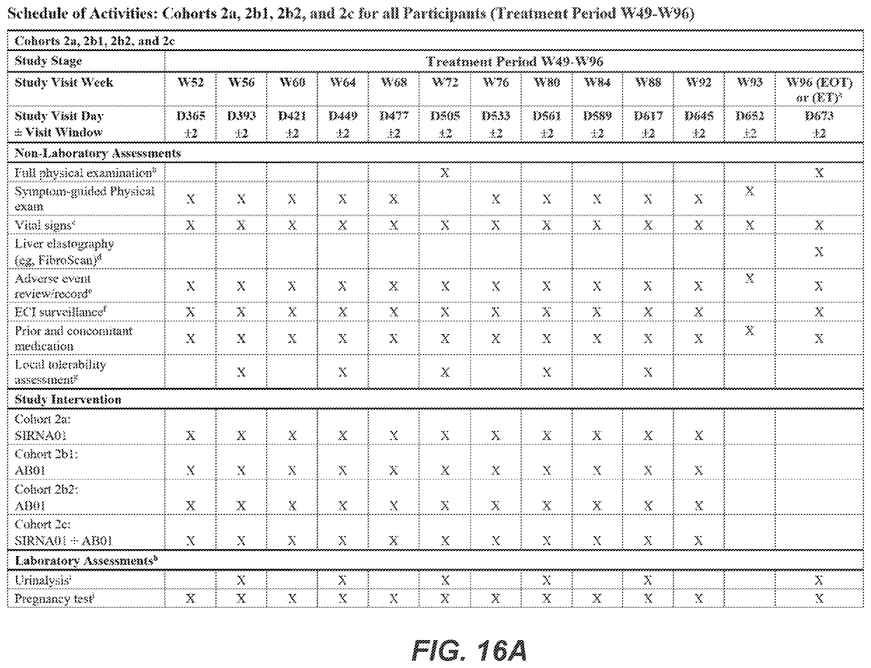

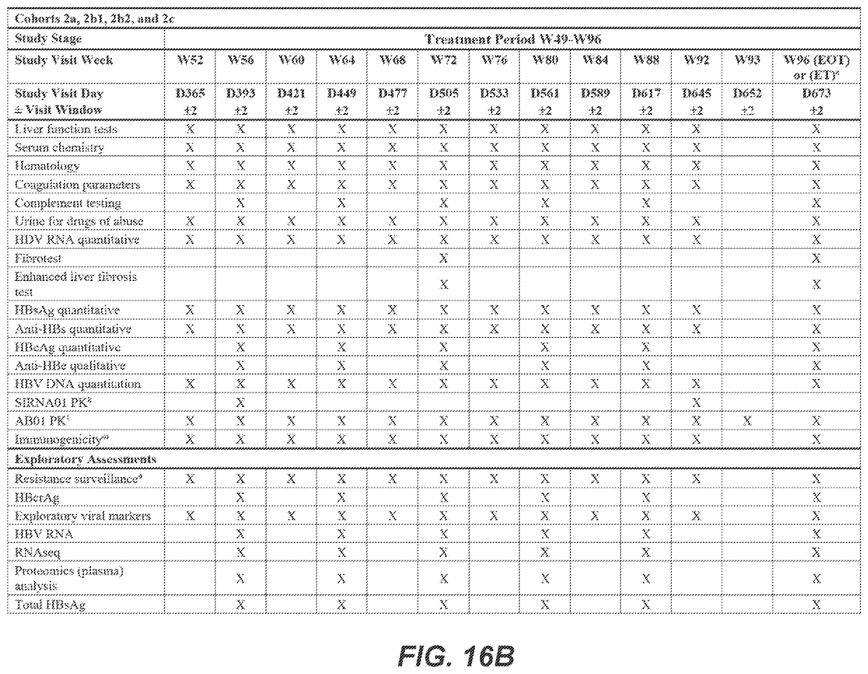

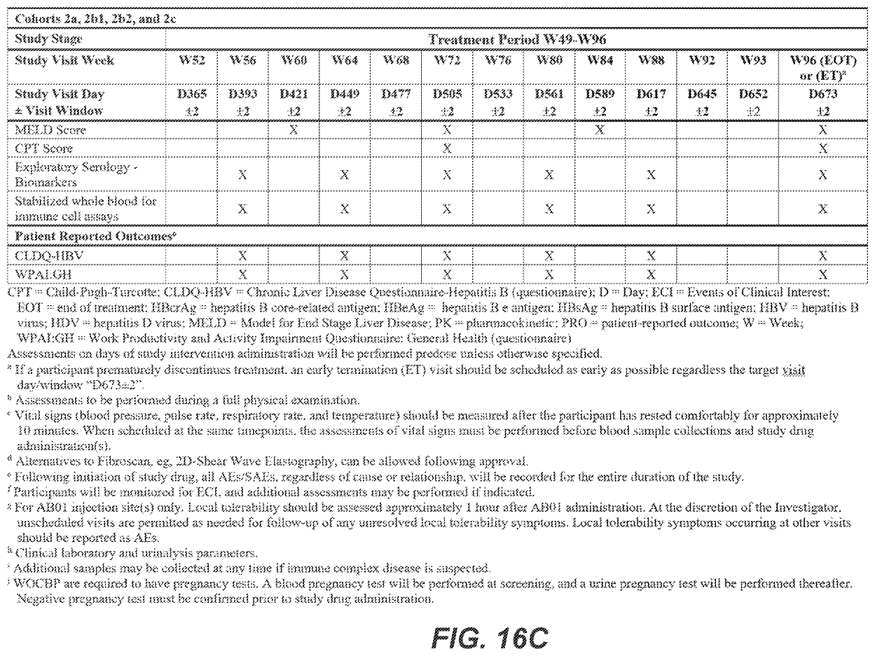

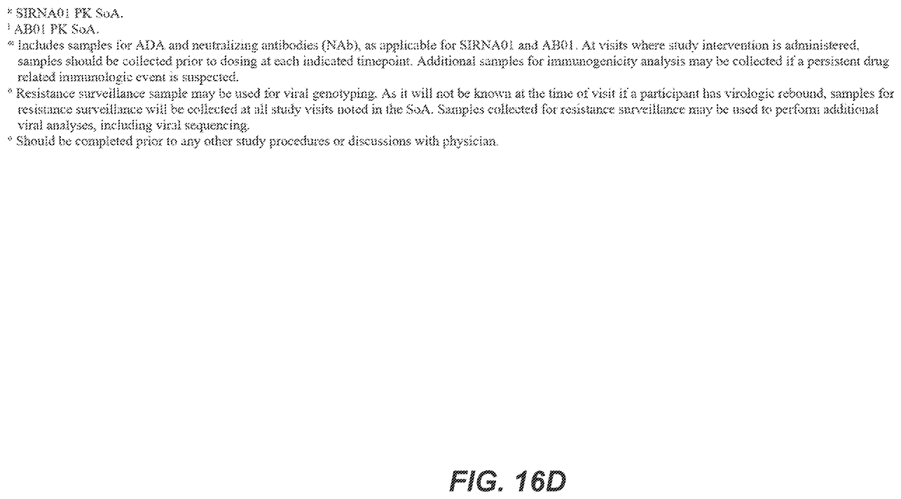

The Intervention period will consist of 2 Periods: Induction and Maintenance. Participants in Cohorts 1a and 1b will receive 3 doses of study treatment in the Induction Period (on Day 1, Week 4, and Week 8) and can continue into the Maintenance Period for an additional 84 weeks if achieving the combined endpoint (undetectable HDV RNA [<LOD] or ≥2 log₁₀ decrease in HDV RNA from baseline and ALT<ULN) at the Week 12 visit. Participants not meeting the combined endpoint at Week 12 can either enter the Follow-up Period or Cohort 2c, Day 1 (must meet the I/E criteria of Cohort 2 at Week 12) at Week 16. Cohorts 1a and 1b participants in the Maintenance Period with HDV RNA rebounding to within 2 log₁₀ IU/mL of their baseline will return to every 4-week dosing of SIRNA01 or AB01 through Week 92. In these cases, participants should follow the activities in the Cohort 2a or 2b SoA (see FIGS. 15A-D and 16A-D) with the next dose to be given within 4 weeks. Participants in Cohort 2a (SIRNA01 monotherapy), Cohorts 2b1 or 2b2 (AB01 monotherapy), and Cohort 2c (SIRNA01+AB01 combination therapy) will receive study treatment monthly for up to 96 weeks in the Treatment Period. Cohort 2a participants not achieving the combined endpoint at Week 48 can either enter the Follow-up Period or begin combination therapy at the Week 52 visit with AB01 added to SIRNA01 and follow investigational product (IP) administration in the Cohort 2c SoA (FIGS. 16A-16D) from Week 52 to Week 96. Cohort 2b1 or 2b2 participants not achieving the combined endpoint at Week 48 can either enter the Follow-up Period or begin combination therapy at the Week 52 visit with SIRNA01 added to AB01 and follow IP administration in the Cohort 2c SoA (FIGS. 16A-16D) from Week 52 to Week 96. Cohort 2b1 participants not achieving at least 1 log₁₀ IU/mL HDV RNA reduction at Week 24 can begin combination therapy and follow IP administration in the Cohort 2c SoA (FIGS. 15A-D and 16A-D) from Week 28 to Week 96. Participants enrolled de novo in Cohort 2c not meeting the combined endpoint at Week 48 will discontinue study treatments and enter the Follow-Up Period. Participants in Cohort 3 will receive AB01 monotherapy biweekly up to 96 weeks in the Treatment Period. Participants not achieving the combined endpoint at Week 48 can either enter the Follow-up Period or begin combination therapy with SIRNA01 added to AB01 and follow IP administration in the Cohort 2c SoA (FIGS. 16A-16D) from Week 52 to Week 96. Cohort 3 participants not achieving at least 1 log₁₀ IU/mL HDV RNA reduction at Week 24 can begin combination therapy and follow IP administration in the Cohort 2c SoA (FIGS. 15A-D and 16A-D) from Week 28 to Week 96. Participants in Cohort 4 will delay treatment for 12 weeks and continue NRTI standard of care after which they will be reassigned to Cohort 2c.

The maximum duration of the Follow-Up Period is 48 weeks after the last dose of the study intervention or Week 96 whichever is earlier. Participants will enter the Follow-Up Period if: Enrolled in Cohort 1 and not transitioning to the Maintenance Period after meeting the combined endpoint at Week 12; Enrolled in Cohort 1 and not entering Cohort 2c after failing to meet the combined endpoint at Week 12; Enrolled in Cohorts 2a, 2b1 or 2b2, and 3 and not entering combination therapy after failing to achieve the combined endpoint at Week 48; Enrolled in Cohorts 2b1 or 3 and not entering combination therapy after meeting virologic non response criteria at Week 24; Enrolled in Cohort 2c and failing to achieve the combined endpoint at Week 48; Failing to achieve the combined endpoint at Week 48 after transitioning from Cohort 4 to Cohort 2c; or Enrolled in any cohort and prematurely discontinuing study drugs. Those who continue to Week 96 will not have additional follow-up visits.

An optional liver biopsy sub-study will be conducted at selected countries and sites where and when available. All participants enrolled de novo to Cohort 2 at selected sites will be eligible to participate in the sub-study. The target enrollment is approximately 18 participants across all Cohort 2 arms. A pretreatment liver tissue sample will be collected during the screening window or up to Week 2 of the study. If the participant has a liver biopsy in the prior 12 months and the tissue block is available for research use and deemed usable, this sample can be used as the pretreatment sample. Follow-up liver tissue samples will be collected on treatment at approximately Week 48±2 weeks and/or Week 96±2 weeks. The tissue sample will be used to directly assess changes in liver fibrosis during treatment with the study drug. The sample will also be used for assays to assess HBV and HDV replication in hepatocytes as well as for exploratory studies.

Participants with baseline HBsAg>3000 IU/mL in Cohorts 2b1 or 2b2, and 2c may participate in the optional AB01 PK sub-study. This sub-study will have up to 2 additional study visits to collect AB01 PK samples. The first visit will occur 5 to 7 days following the third, fourth, or fifth dose of AB01, and the second optional visit will occur 5 to 7 days following the seventh, eighth, or ninth dose of AB01. Participants entering Cohort 2c from Cohorts 1a or 1b will be excluded from this sub-study. In addition to PK, HBsAg, HDV RNA, HBV DNA, and liver function tests will also be collected at the same visits. This optional PK sub-study will be closed after 30 participants enroll into the sub study.

An optional liver fine needle aspiration sub-study will be conducted at selected UK sites. All participants enrolled de novo to Cohorts 2b1/2b2, 2c, and 3 at selected sites, will be eligible to participate in the sub-study. The target enrollment is approximately up to 10 participants total. Liver FNA and peripheral blood mononuclear cells (PBMC) samples will be collected pretreatment, Week 24±2 weeks, and optionally at any visit between Weeks 48 to 96. This FNA sub-study will provide information on changes in the intrahepatic environment as well as detailed immunologic and virologic data in participants on treatment with a combination of siRNA and mAb targeting HDV replication.

AB01 is provided as a reconstituted lyophilized powder administered subcutaneously (SC) at 300 mg every 4 weeks or 8 weeks (see Table 6). AB01 (HBC34v35-MLNS-GAALIE) comprises a light chain amino acid sequence of SEQ ID NO:59, and a heavy chain amino acid sequence of SEQ ID NO:57. SIRNA01 is provided as a liquid administered SC at 200 mg every 4 weeks or 8 weeks (see Table 6). SIRNA01 has a sense strand comprising the nucleotide sequence of SEQ ID NO:5 and an antisense strand comprising the nucleotide sequence of SEQ ID NO:6.

Study objectives and associated endopoints are shown in Table 7.

TABLE 7
Study objectives and associated endpoints.

| | |
|---|---|
| Objectives | Endpoints |

Primary

| | |
|---|---|
| To evaluate the efficacy of SIRNA01 and | Proportion of participants with |
| AB01 in participants with chronic | undetectable HDV RNA |
| HBV/HDV coinfection | (<LOD) or ≥2 log₁₀ decrease in HDV |
| | RNA from baseline and alanine |
| | aminotransferase (ALT) |
| | normalization (ALT < upper limit of |
| | normal [ULN]) at Week 24 (Cohorts |
| | 2 and 3 only) |
| To evaluate the safety of SIRNA01 and | Treatment-emergent adverse events |
| AB01 in participants with chronic | (TEAEs) and serious adverse events |
| HBV/HDV coinfection | (SAEs) |

Secondary

| | |
|---|---|
| To evaluate the efficacy of SIRNA01 and | Proportion of participants with |
| AB01 in participants with chronic | undetectable HDV RNA (<LOD) |
| HBV/HDV coinfection on HDV RNA | or ≥2 log₁₀ decrease in HDV RNA from |
| and ALT normalization | baseline and ALT normalization at |
| | Week 12, Week 48, Week 72, and |
| | Week 96 |
| | Proportion of participants with |
| | undetectable HDV RNA (<LOD) |
| | or ≥2 log₁₀ decrease in HDV RNA from |
| | baseline at Week 12, Week 24, |
| | Week 48, Week 72, and Week 96 |
| | Proportion of participants with |
| | undetectable HDV RNA (<LOD) at |
| | Week 12, Week 24, Week 48, |
| | Week 72, and Week 96 |
| | Proportion of participants with HDV |
| | RNA < lower limit of quantitation |
| | (LLOQ) at Week 12, Week 24, |
| | Week 48, Week 72, and Week 96 |
| | Change from baseline in HDV RNA |
| | at Week 12, Week 24, Week 48, |
| | Week 72, and Week 96 |
| | Proportion of participants with ALT |
| | normalization at Week 12, Week 24, |
| | Week 48, Week 72, and Week 96 |
| To assess the immunogenicity of AB01 | Incidence of ADA and titers of ADA |
| (for cohorts with AB01) | to AB01 at each study visit up to |
| | Week 96 (for cohorts with AB01) |
| To assess the effects of SIRNA01 and | Change from baseline in liver fibrosis |
| AB01 on liver fibrosis and hepatic | at Week 48 and Week 96 |
| function | Change from baseline in Model for |
| | End Stage Liver Disease (MELD) |
| | score at Week 12, Week 24, |
| | Week 36, Week 48, Week 60, |
| | Week 72, Week 84, and Week 96 |
| | Change from baseline in CPT score at |
| | Week 24, Week 48, Week 72, and |
| | Week 96 |

Exploratory

| | |
|---|---|
| To evaluate the host immune response | Host immune responses and |
| and exploratory biomarkers related to | exploratory biomarkers related to |
| infection and/or investigational | HBV and/or investigational |
| therapy(ies) | therapy(ies), including but not limited |
| | to genetic, metabolic, cellular, and |
| | proteomic parameters |
| To assess the effects of SIRNA01 and | Change from baseline in serum |
| AB01 on serum HBsAg | HBsAg at Week 12, Week 24, |
| | Week 48, and Week 96 |
| | Number of participants with HBsAg |
| | loss at Week 12, Week 24, Week 48, |
| | and Week 96 |
| To evaluate the emergence of viral | HBV or HDV viral resistance to |
| resistance to investigational therapy(ies) | investigational therapy(ies) during |
| | treatment period |
| To evaluate additional viral parameters | Additional viral parameters |
| associated with HBV and/or | associated with HBV, HDV, and/or |
| investigational therapy(ies) | investigational therapy(ies) |
| To characterize the pharmacokinetics | AB01 PK (for cohorts with AB01) |
| (PK) of AB01 (for cohorts with AB01) | |
| To characterize the PK of SIRNA01 (for | SIRNA01 PK (for cohorts with |
| cohorts with SIRNA01) | SIRNA01) |
| To assess the immunogenicity of | Incidence of ADA and titers of ADA |
| SIRNA01 (for cohorts with SIRNA01) | to SIRNA01 at each study visit up to |
| | Week 96 (for cohorts with SIRNA01) |
| To assess the effects of SIRNA01 and | Incidence of HCC and progression to |
| AB01 on end stage liver disease | liver failure requiring transplantation |
| outcomes | or resulting in death during treatment |
| | period |
| To assess health-related quality of life | Change from baseline in health- |
| measurements | related quality of life as measured by |
| | the Chronic Liver Disease |
| | Questionnaire-Hepatitis B |
| | (CLDQ-HBV) questionnaire at each |
| | study visit up to Week 96 |
| To assess the change from baseline in | Change from baseline in work |
| work productivity and daily activities | productivity and daily activities as |
| measurements | measured by the Work Productivity |
| | and Activity Impairment (WPAI) |
| | questionnaire at each study visit up to |
| | Week 96 |

The Child-Pugh Turcotte (CPT) Score Assessment of Liver Disease is shown in Table 8.

TABLE 8
Child-Pugh Turcotte (CPT) Score Assessment of Liver Disease.

| | Points Scored for Observed Findings | | |
|---|---|---|---|
| | 1 | 2 | 3 |
| Encephalopathy grade* | none | 1 or 2 | 3 or 4 |
| Ascites | absent | slight | moderate |
| | | (diuretic | (diuretic |
| | | responsive) | unresponsive) |
| Serum bilirubin, mg/dL | <2 | 2 to 3 | >3 |
| Serum albumin, g/dL | >3.5 | 2.8 to 3.5 | <2.8 |
| Prothrombin time, sec | <4 | 4 to 6 | >6 |
| prolonged | | | |

*Grade 0: normal consciousness, personality, neurological examination electroencephalogram
Grade 1: restless, sleep disturbed, irritable/agitated, tremor, impaired handwriting, 5 cycle per second waves
Grade 2: lethargic, time-disoriented, inappropriate, asterixis, ataxia, slow triphasic waves
Grade 3: somnolent, stuporous, place-disoriented, hyperactive reflexes, rigidity, slower waves
Grade 4: unrousable coma, no personality/behavior, decerebrate, slow 2-3 cycle per second delta activity
Source: FDA 2003
Assessment: Class A (mild hepatic impairment) if 5 or 6 points; B (moderate hepatic impairment) if 7 to 9 points; and C (severe hepatic impairment) if 10 to 15 points.

ULN values for ALT may be, for example, 34 IU/mL for females and 43 IU/mL for males.

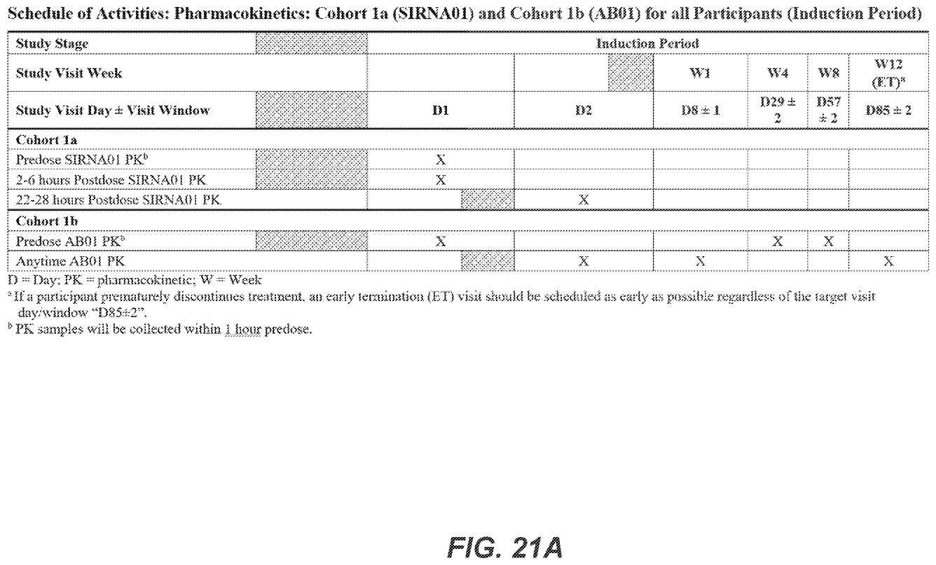

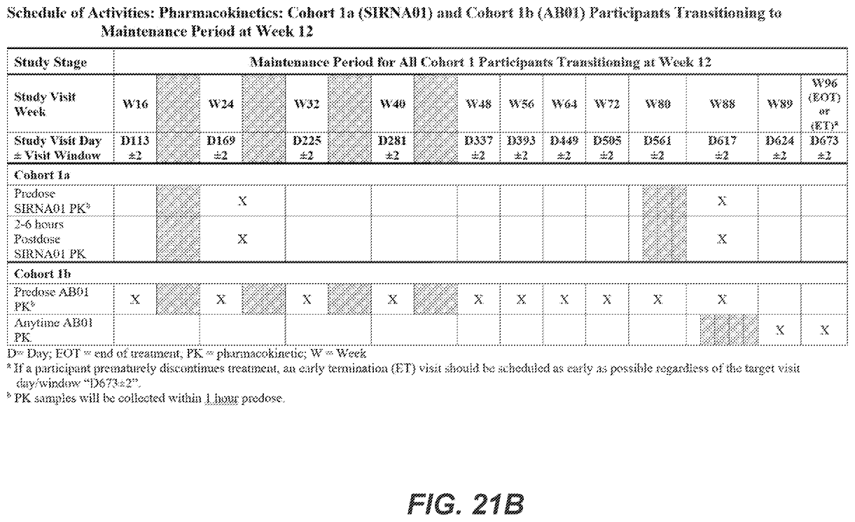

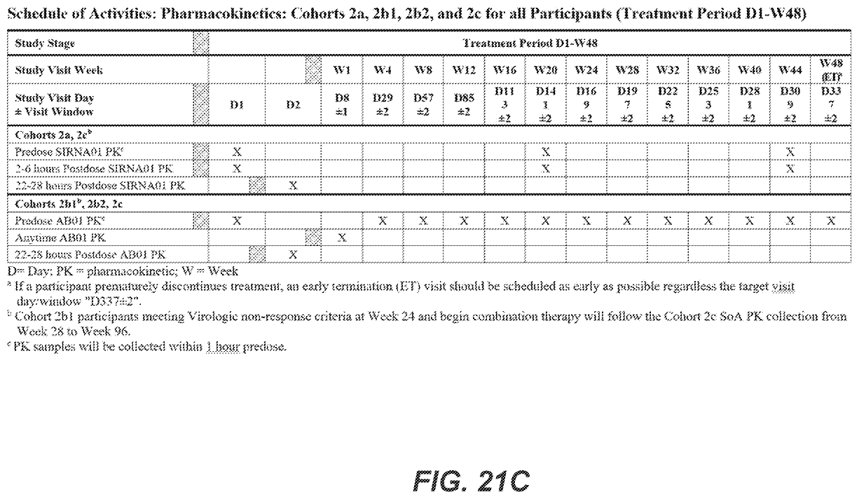

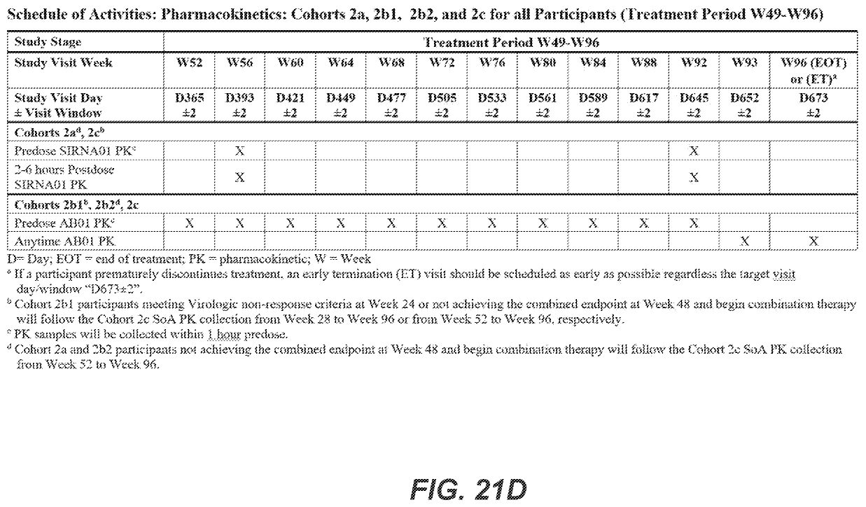

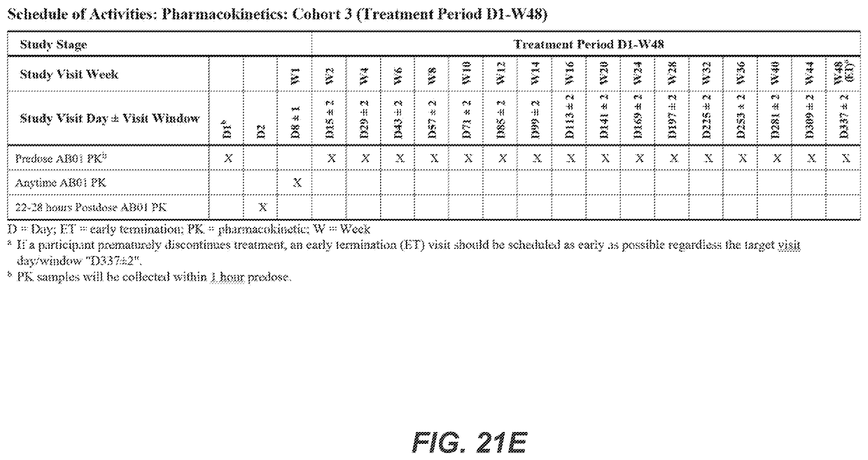

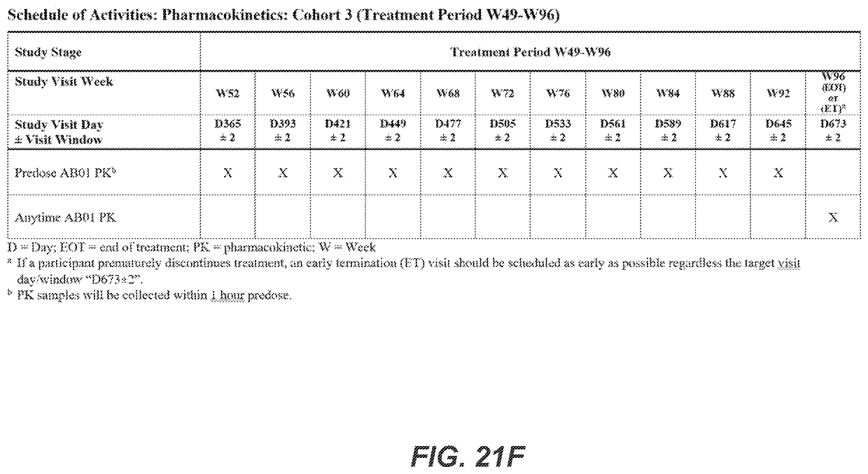

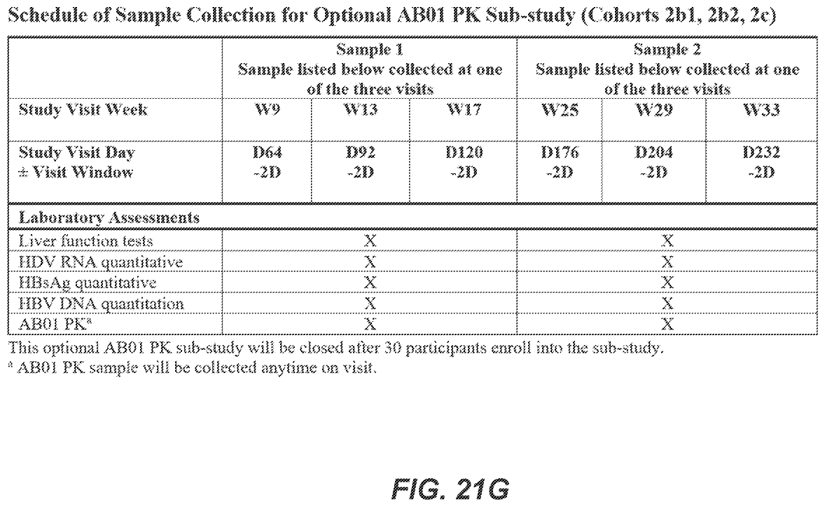

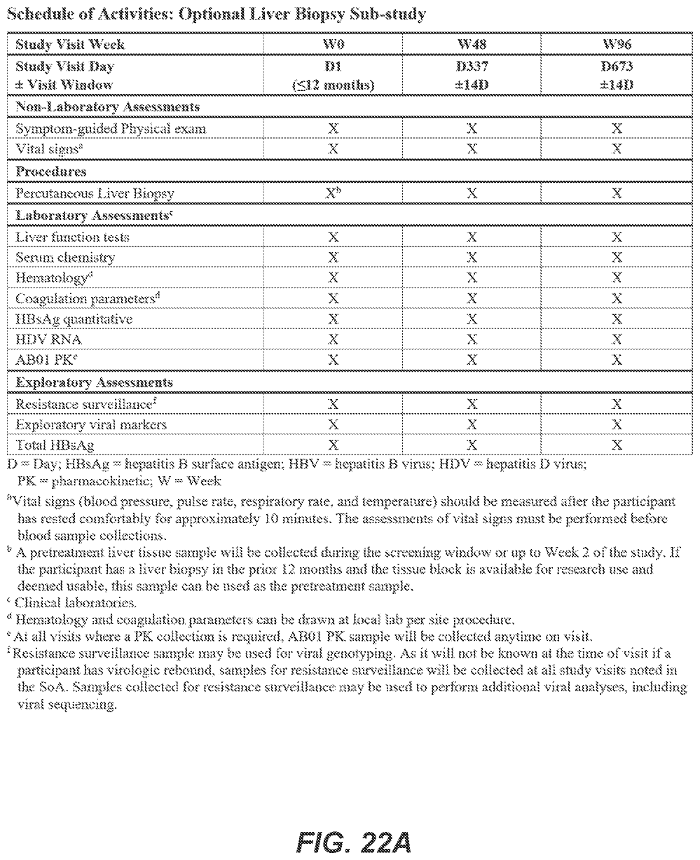

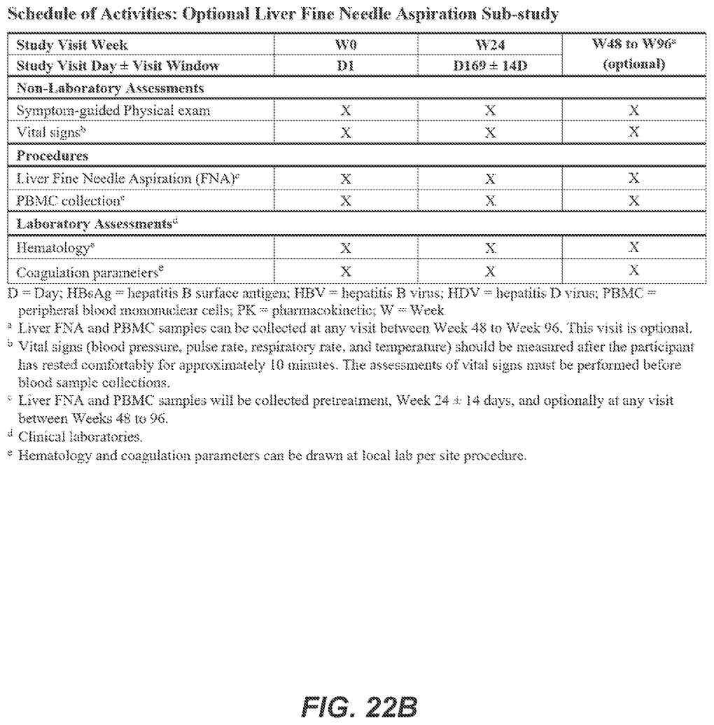

PK samples will be taken as in FIGS. 21A-21G, and PK parameters (free and total PK, as applicable) of SIRNA01 and AB01 will be computed. Parameters may include, but not be limited to, Cₘₐₓ, Cₗₐₛₜ, Tₘₐₓ, Tₗₐₛₜ, AUC_{inf}, AUCₗₐₛₜ, % AUCₑₓₚ, t_{1/2}, λ_{z}, V_{z}/F, and CL/F.

Immunogenicity data may include, but not be limited to, presence/absence and titers of anti-drug antibodies (ADA) and neutralization data.

### Participant Population

The study will enroll male and female participants 18 to 70 years of age with chronic HBV/HDV coinfection, both noncirrhotic and cirrhotic up to METAVIR-F4/CPT-A, currently on NRTI therapy. “Chronic HBV infection” for purposes of the study is defined as a positive serum HBsAg, HBV DNA, or HBeAg on 2 occasions at least 6 months apart based on previous (within the past 12 months) or current laboratory documentation (any combination of these tests performed 6 months apart is acceptable). Participants will be on locally approved NRTI therapy for at least 12 weeks prior to Day 1. Participants will also have HBsAg>0.05 IU/mL at screening; positive HDV antibody for at least 6 months prior to screening and HDV RNA≥500 IU/mL at screening; and serum alanine aminotransferase (ALT)>ULN and <5×ULN.

Participants are age≥18 (or age of legal consent, whichever is older) to <70 years at screening. Participants also have a Body Mass Index (BMI)≥18 kg/m² to ≤40 kg/m².

Additional inclusion criteria include the following:
Female participants must have a negative pregnancy test or confirmation of postmenopausal status. Postmenopausal status is defined as 12 months with no menses without an alternative medical cause. Women of childbearing potential (WOCBP) must have a negative blood pregnancy test at screening and a negative urine pregnancy test on Day 1, cannot be breast feeding, and must be willing to use highly effective methods of contraception 14 days before study drug administration through 48 weeks after the last dose of SIRNA01 or AB01. Female participants must also agree to refrain from egg donation and in vitro fertilization from the time of study drug administration through 48 weeks after the last dose of SIRNA01 or AB01.
Male participants with female partners of childbearing potential must agree to meet 1 of the following contraception requirements from the time of study drug administration through 48 weeks after the last dose of SIRNA01 or AB01: documentation of vasectomy or azoospermia, or male condom use plus partner use of 1 of the contraceptive options listed for contraception for WOCBP. Male participants must also agree to not donate sperm from the time of first study drug administration through 48 weeks after the last dose of SIRNA01 or AB01.
Capable of giving signed informed consent
12-lead electrocardiogram (ECG) within normal limits; or, with no clinically significant abnormalities at screening, as determined by the clinical investigator.
Agrees not to donate blood during the duration of the study and for an additional 3 months after the last dose study drug.

Cohort 1 specific inclusion criteria
Noncirrhotic
   Liver biopsy with METAVIR F0-F3 or Liver elastography (e.g., Fibroscan®)<12 kilopascal (kPa) within the 12 months prior to screening
   Creatinine clearance (CLcr)≥30 mL/min as calculated by the Cockcroft-Gault formula at screening
   Platelet count>150,000 cells/mm³ (/μL)

Cohorts 2a, 2b1, 2b2, 2c, 3, and 4 specific inclusion criteria
Noncirrhotic
   Liver biopsy with METAVIR F0-F3 or Liver elastography (e.g., Fibroscan®)<12 kPa within the 12 months prior to screening
   CLcr≥30 mL/min as calculated by the Cockcroft-Gault formula at screening
   Platelet count>150,000 cells/mm³ (@L)
CPT-A Cirrhotic
   Liver biopsy with METAVIR F4 or Liver elastography (e.g., Fibroscan®)≥12 kPa within the 12 months prior to screening
   CLcr≥60 mL/min as calculated by the Cockcroft-Gault formula at screening
   Platelet count>90,000 cells/mm³ (/μL)
   CPT score of 5 or 6, inclusive at screening and at start of study
Alternatives to Fibroscan®, e.g., 2D-Shear Wave Elastography, may be allowed.

Exclusion criteria include the following:
History of clinically significant liver disease from non-HBV and non-HDV etiology
History of clinically significant immune complex disease
History of clinically significant autoimmune disorder
History of HBV-related extrahepatic disease, including but not limited to HBV-related rash, arthritis, or glomerulonephritis
History of allergic reactions, hypersensitivity, or intolerance to study drug, its metabolites or excipients
Anti-HBs>10 mIU/mL at screening
Corrected QT interval (QTc)>450 milliseconds
ALT or AST≥5×ULN
Total bilirubin>2.0 mg/dL
Serum albumin<30 g/L
Absolute neutrophil count<1,000/mm³ (/L)
International normalized ratio (INR)>1.5
Hemoglobin<8 g/dL
History of anaphylaxis
History of malignancy diagnosed or treated within 5 years (localized treatment of squamous or noninvasive basal cell skin cancers is permitted; cervical carcinoma in situ is allowed if appropriately treated prior to screening); participants under evaluation for malignancy are not eligible
History of or listed for bone marrow or solid organ transplant
Known active infection other than chronic HBV and HDV infection or any clinically significant acute condition such as fever (>38° C.) or acute respiratory illness within 7 days prior to Day 1
Coinfection with human immunodeficiency virus (HIV), hepatitis A virus (HAV), hepatitis C virus (HCV), or hepatitis E virus (HEV); participants who are HCV antibody positive and HCV RNA negative are eligible; participants who are HAV or HEV immunoglobulin M antibody (IgM) positive are not eligible. Participants who are asymptomatic and HAV or HEV immunoglobulin G antibody (IgG) positive are eligible
Any clinically significant medical or psychiatric condition that may interfere with study intervention, assessment, or compliance with the protocol or otherwise makes the participant unsuitable for participation in the study; participants with controlled diabetes mellitus are eligible
Acute or worsening chronic hepatitis, fluctuating or rapidly deteriorating hepatic function, or use of any therapy known to exacerbate hepatic dysfunction
History or clinical evidence of alcohol or drug abuse within the 12 months before screening or a positive drug screen at screening unless it can be explained by a prescribed medication; cannabis use is permitted
Participants requiring paracentesis>1 time per month
Participants with refractory encephalopathy or significant Central Nervous System disease
History of gastric or esophageal variceal bleeding within the past 6 months
Participants with Transjugular Intrahepatic Portosystemic Shunt (TIPS) placement
Presence of hepatopulmonary or hepatorenal syndrome
Presence of primary cholestatic liver diseases
Inability or unwillingness to comply with dietary recommendations for liver cirrhosis and hepatic impairment

### Concomitant Therapy

The following concomitant therapies are not permitted during the study:
Therapy with an immunomodulatory agent, IFN-α (e.g., IFN-alfa-2a or IFN-alfa-2b, or pegylated IFN-alfa-2a or alfa 2b), immunosuppressants (e.g., disease-modifying antirheumatic drugs), cytotoxic or chemotherapeutic agent, or chronic systemic corticosteroids within 6 months of screening
Received an HDV active agent (including lonafarnib and bulevirtide) within 90 days or 5 half-lives (if known), whichever is longer, before study drug administration or are active in the Follow-Up Period of another clinical study involving interventional treatment; participants must also agree not to take part in any other interventional study at any time during their participation in this study, inclusive of the Follow-Up Period
Receipt of an oligonucleotide (e.g., siRNA, antisense oligonucleotide) with activity against HBV or HDV within 48 weeks before first study drug administration
Receipt of AB01 or any antibody targeting HBV or HDV within 24 weeks of first study drug administration
Participants must abstain from taking prescription drugs outside the care of a prescribing physician and nonprescription drugs (including vitamins, recreational drugs, and dietary or herbal supplements) within 7 days (or 14 days if the drug is a potential enzyme inducer) or 5 half-lives (whichever is longer) before the start of study intervention until completion of the follow-up visit, unless the medication will not interfere with the study
Participants with hepatic impairment with co-morbid diseases requiring medication(s) must be taking the medication(s) without a change in dose for >3 months prior to screening
Use of any of the following systemic medications is prohibited within 14 days before study drug administration and throughout the study: chronic systemic steroids (prednisone equivalent of >10 mg/day) or other immunosuppressive agents (Note: corticosteroid administration for the treatment of immune-mediated AEs and short courses of corticosteroids for chronic obstructive pulmonary disease or asthma exacerbations is allowed); paracetamol (acetaminophen)≥3 g/day; isoniazid; additionally, the administration of any potentially hepatotoxic medications during the study should be considered only if no therapeutic alternative can be identified and after a careful consideration of the potential risks and benefits for the participant (medications that are potentially hepatotoxic or associated with drug-induced liver injury include, but are not limited to, the following (Bjornsson 2016): aspirin>3 g/day or ibuprofen≥1.2 g/day, tricyclic antidepressants, valproate, phenytoin, amiodarone, anabolic steroids, allopurinol, amoxicillin-clavulanate, minocycline, nitrofurantoin, sulfamethoxazole/trimethoprim, erythromycin, rifampin, azole antifungals, and herbal or natural remedies)

### Preliminary Results

In Cohort 1b, non-cirrhotic participants with chronic HDV infection received AB01 300 mg subcutaneously every 4 weeks for a minimum of three doses. Safety, tolerability, and antiviral activity were assessed. Virologic response was defined as a ≥2 log₁₀ decrease or less than the lower limit of detection ([LOD], HDV RNA<14 IU/mL) in HDV RNA. ALT normalization was defined as less than the upper limit of normal (33 U/L for females; 40 U/L for males).

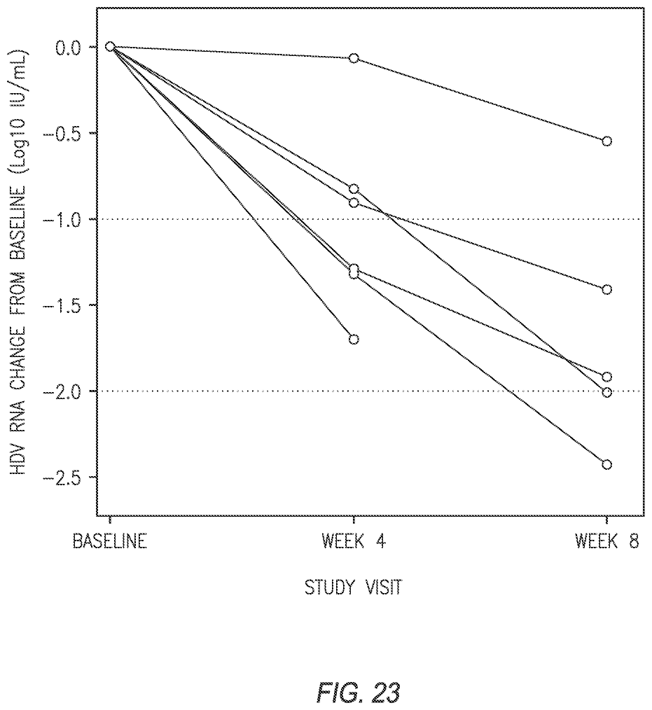

Six participants were enrolled in Cohort 1b and completed Study Week 8. All treatment emergent adverse events (TEAEs) were grade 1 or 2, and included myalgia, headache, and chills. No serious TEAEs were reported and no participants discontinued from the study. The mean reduction in HDV RNA at Week 8 was 1.7 log₁₀. Virologic response was observed in 3 of 6 participants and the remaining three participants achieved an at least 0.5 log₁₀ decrease in HDV RNA by Week 8, with one participant achieving 1.9 (FIG. 23). ALT normalization was observed in 3 of 6 by Week 8. Of these three participants, two achieved the combined endpoint of virologic response and ALT normalization by Week 8.

These preliminary data show that AB01 was generally well tolerated in non-cirrhotic participants with chronic HDV. The antiviral activity of AB01 was demonstrated by the rapid reductions in HDV RNA and was accompanied by normalization of ALT, reflecting improvement in liver inflammation. These data support the continued development of AB01 for the treatment of chronic HDV.

While specific embodiments have been illustrated and described, it will be readily appreciated that the various embodiments described above can be combined to provide further embodiments, and that various changes can be made therein without departing from the spirit and scope of the invention.

All of the U.S. patents, U.S. patent application publications, U.S. patent applications, foreign patents, foreign patent applications, and non-patent publications referred to in this specification, or listed in the Application Data Sheet, including U.S. Provisional Patent Application No. 63/343,908 filed May 19, 2022, are incorporated herein by reference, in their entirety, unless otherwise stated. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

SEQUENCES
(Hepatitis B Virus genome - NCBI Reference Sequence NC_003977.2
(GenBank Accession No. GI: 21326584))
SEQ ID NO: 1
AATTCCACAACCTTCCACCAAACTCTGCAAGATCCCAGAGTGAGAGGCCTG
TATTTCCCTGCTGGTGGCTCCAGTTCAGGAACAGTAAACCCTGTTCTGACTA
CTGCCTCTCCCTTATCGTCAATCTTCTCGAGGATTGGGGACCCTGCGCTGAA
CATGGAGAACATCACATCAGGATTCCTAGGACCCCTTCTCGTGTTACAGGC
GGGGTTTTTCTTGTTGACAAGAATCCTCACAATACCGCAGAGTCTAGACTCG
TGGTGGACTTCTCTCAATTTTCTAGGGGGAACTACCGTGTGTCTTGGCCAAA
ATTCGCAGTCCCCAACCTCCAATCACTCACCAACCTCTTGTCCTCCAACTTG
TCCTGGTTATCGCTGGATGTGTCTGCGGCGTTTTATCATCTTCCTCTTCATCC
TGCTGCTATGCCTCATCTTCTTGTTGGTTCTTCTGGACTATCAAGGTATGTTG
CCCGTTTGTCCTCTAATTCCAGGATCCTCAACAACCAGCACGGGACCATGCC
GGACCTGCATGACTACTGCTCAAGGAACCTCTATGTATCCCTCCTGTTGCTG
TACCAAACCTTCGGACGGAAATTGCACCTGTATTCCCATCCCATCATCCTGG
GCTTTCGGAAAATTCCTATGGGAGTGGGCCTCAGCCCGTTTCTCCTGGCTCA
GTTTACTAGTGCCATTTGTTCAGTGGTTCGTAGGGCTTTCCCCCACTGTTTGG
CTTTCAGTTATATGGATGATGTGGTATTGGGGGCCAAGTCTGTACAGCATCT
TGAGTCCCTTTTTACCGCTGTTACCAATTTTCTTTTGTCTTTGGGTATACATT
TAAACCCTAACAAAACAAAGAGATGGGGTTACTCTCTAAATTTTATGGGTT
ATGTCATTGGATGTTATGGGTCCTTGCCACAAGAACACATCATACAAAAAA
TCAAAGAATGTTTTAGAAAACTTCCTATTAACAGGCCTATTGATTGGAAAGT
ATGTCAACGAATTGTGGGTCTTTTGGGTTTTGCTGCCCCTTTTACACAATGT
GGTTATCCTGCGTTGATGCCTTTGTATGCATGTATTCAATCTAAGCAGGCTT
TCACTTTCTCGCCAACTTACAAGGCCTTTCTGTGTAAACAATACCTGAACCT
TTACCCCGTTGCCCGGCAACGGCCAGGTCTGTGCCAAGTGTTTGCTGACGCA
ACCCCCACTGGCTGGGGCTTGGTCATGGGCCATCAGCGCATGCGTGGAACC
TTTTCGGCTCCTCTGCCGATCCATACTGCGGAACTCCTAGCCGCTTGTTTTGC
TCGCAGCAGGTCTGGAGCAAACATTATCGGGACTGATAACTCTGTTGTCCTA
TCCCGCAAATATACATCGTTTCCATGGCTGCTAGGCTGTGCTGCCAACTGGA
TCCTGCGCGGGACGTCCTTTGTTTACGTCCCGTCGGCGCTGAATCCTGCGGA
CGACCCTTCTCGGGGTCGCTTGGGACTCTCTCGTCCCCTTCTCCGTCTGCCGT
TCCGACCGACCACGGGGCGCACCTCTCTTTACGCGGACTCCCCGTCTGTGCC
TTCTCATCTGCCGGACCGTGTGCACTTCGCTTCACCTCTGCACGTCGCATGG
AGACCACCGTGAACGCCCACCAAATATTGCCCAAGGTCTTACATAAGAGGA
CTCTTGGACTCTCAGCAATGTCAACGACCGACCTTGAGGCATACTTCAAAG
ACTGTTTGTTTAAAGACTGGGAGGAGTTGGGGGAGGAGATTAGGTTAAAGG
TCTTTGTACTAGGAGGCTGTAGGCATAAATTGGTCTGCGCACCAGCACCATG
CAACTTTTTCACCTCTGCCTAATCATCTCTTGTTCATGTCCTACTGTTCAAGC
CTCCAAGCTGTGCCTTGGGTGGCTTTGGGGCATGGACATCGACCCTTATAAA
GAATTTGGAGCTACTGTGGAGTTACTCTCGTTTTTGCCTTCTGACTTCTTTCC
TTCAGTACGAGATCTTCTAGATACCGCCTCAGCTCTGTATCGGGAAGCCTTA
GAGTCTCCTGAGCATTGTTCACCTCACCATACTGCACTCAGGCAAGCAATTC
TTTGCTGGGGGGAACTAATGACTCTAGCTACCTGGGTGGGTGTTAATTTGGA
AGATCCAGCGTCTAGAGACCTAGTAGTCAGTTATGTCAACACTAATATGGG
CCTAAAGTTCAGGCAACTCTTGTGGTTTCACATTTCTTGTCTCACTTTTGGAA
GAGAAACAGTTATAGAGTATTTGGTGTCTTTCGGAGTGTGGATTCGCACTCC
TCCAGCTTATAGACCACCAAATGCCCCTATCCTATCAACACTTCCGGAGACT
ACTGTTGTTAGACGACGAGGCAGGTCCCCTAGAAGAAGAACTCCCTCGCCT
CGCAGACGAAGGTCTCAATCGCCGCGTCGCAGAAGATCTCAATCTCGGGAA
TCTCAATGTTAGTATTCCTTGGACTCATAAGGTGGGGAACTTTACTGGGCTT
TATTCTTCTACTGTACCTGTCTTTAATCCTCATTGGAAAACACCATCTTTTCC
TAATATACATTTACACCAAGACATTATCAAAAAATGTGAACAGTTTGTAGG
CCCACTCACAGTTAATGAGAAAAGAAGATTGCAATTGATTATGCCTGCCAG
GTTTTATCCAAAGGTTACCAAATATTTACCATTGGATAAGGGTATTAAACCT
TATTATCCAGAACATCTAGTTAATCATTACTTCCAAACTAGACACTATTTAC
ACACTCTATGGAAGGCGGGTATATTATATAAGAGAGAAACAACACATAGCG
CCTCATTTTGTGGGTCACCATATTCTTGGGAACAAGATCTACAGCATGGGGC
AGAATCTTTCCACCAGCAATCCTCTGGGATTCTTTCCCGACCACCAGTTGGA
TCCAGCCTTCAGAGCAAACACCGCAAATCCAGATTGGGACTTCAATCCCAA
CAAGGACACCTGGCCAGACGCCAACAAGGTAGGAGCTGGAGCATTCGGGC
TGGGTTTCACCCCACCGCACGGAGGCCTTTTGGGGTGGAGCCCTCAGGCTC
AGGGCATACTACAAACTTTGCCAGCAAATCCGCCTCCTGCCTCCACCAATCG
CCAGTCAGGAAGGCAGCCTACCCCGCTGTCTCCACCTTTGAGAAACACTCA
TCCTCAGGCCATGCAGTGG
(Target sequence, nucleotides 1579-1597 of NC_003977.2
(GenBank Accession No. GI: 21326584))
SEQ ID NO: 2
GTGTGCACTTCGCTTCAC
(SIRNA01, sense strand, unmodified)
SEQ ID NO: 3
GUGUGCACUUCGCUUCACA
(SIRNA01, antisense strand, unmodified)
SEQ ID NO: 4
UGUGAAGCGAAGUGCACACUU
(SIRNA01, sense strand, modified)
SEQ ID NO: 5
gsusguGfcAfCfUfucgcuucacaL96
(SIRNA01, antisense strand, modified)
SEQ ID NO: 6
usGfsuga(Agn)gCfGfaaguGfcAfcacsusu
(SIRNA02, sense strand, modified)
SEQ ID NO: 7
gsusguGfcAfCfUfucgcuucacaL96
(SIRNA02, antisense strand, modified)
SEQ ID NO: 8
usGfsugaAfgCfGfaaguGfcAfcacsusu
(Membrane translocation sequence-containing peptide RFGF)
SEQ ID NO: 9
AAVALLPAVLLALLAP
(Membrane translocation sequence-containing peptide RFGF analogue)
SEQ ID NO: 10
AALLPVLLAAP
(HIV Tat protein)
SEQ ID NO: 11
GRKKRRQRRRPPQ
(Drosophila Antennapedia protein)
SEQ ID NO: 12
RQIKIWFQNRRMKWK
(HBsAg S domain)
SEQ ID NO: 13
MENITSGFLGPLLVLQAGFFLLTRILTIPQSLDSWWTSLNFLGGTTVCLGQNSQS
PTSNHSPTSCPPTCPGYRWMCLRRFIIFLFILLLCLIFLLVLLDYQGMLPVCPLIPG
SSTTSTGPCRTCMTTAQGTSMYPSCCCTKPSDGNCTCIPIPSSWAFGKFLWEWA
SARFSWLSLLVPFVQWFVGLSPTVWLSVIWMMWYWGPSLYSILSPFLPLLPIFF
CLWVYI
(J02203 (D, ayw3) HBsAg Antigenic Loop Sequence)
SEQ ID NO: 14
QGMLPVCPLIPGSSTTSTGPCRTCMTTAQGTSMYPSCCCTKPSDGNCTCIPIPSS
WAFGKFLWEWASARFSW
(FJ899792 (D, adw2) HBsAg Antigenic Loop Sequence)
SEQ ID NO: 15
QGMLPVCPLIPGSSTTGTGPCRTCTTPAQGTSMYPSCCCTKPSDGNCTCIPIPSS
WAFGKFLWEWASARFSW
(AM282986 (A) HBsAg Antigenic Loop Sequence)
SEQ ID NO: 16
QGMLPVCPLIPGTTTTSTGPCKTCTTPAQGNSMFPSCCCTKPSDGNCTCIPIPSS
WAFAKYLWEWASVRFSW
(D23678 (B1) HBsAg Antigenic Loop Sequence)
SEQ ID NO: 17
QGMLPVCPLIPGSSTTSTGPCKTCTTPAQGTSMFPSCCCTKPTDGNCTCIPIPSSW
AFAKYLWEWASVRFSW
(AB117758 (C1) HBsAg Antigenic Loop Sequence)
SEQ ID NO: 18
QGMLPVCPLLPGTSTTSTGPCKTCTIPAQGTSMFPSCCCTKPSDGNCTCIPIPSSW
AFARFLWEWASVRFSW
(AB205192(E) HBsAg Antigenic Loop Sequence)
SEQ ID NO: 19
QGMLPVCPLIPGSSTTSTGPCRTCTTLAQGTSMFPSCCCSKPSDGNCTCIPIPSSW
AFGKFLWEWASARFSWLS
(X69798 (F4) HBsAg Antigenic Loop Sequence)
SEQ ID NO: 20
QGMLPVCPLLPGSTTTSTGPCTCTTLAQGTSMFPSCCCSKPSDGNCTCIPIPSSW
ALGKYLWEWASARFSW
(AF1 60501 (G) HBsAg Antigenic Loop Sequence)
SEQ ID NO: 21
QGMLPVCPLIPGSSTTSTGPCTCTTPAQGNSMYPSCCCTPSDGNCTCIPIPSSWAF
AKYLWEWASVRFSW
(AY090454 (H) HBsAg Antigenic Loop Sequence)
SEQ ID NO: 22
QGMLPVCPLLPGSTTTSTGPCKTCTTLAQGTSMFPSCCCTKPSDGNCTCIPIPSS
WAFGKYLWEWASARFSW
(AF241409 (I) HBsAg Antigenic Loop Sequence)
SEQ ID NO: 23
QGMLPVCPLIPGSSTTSTGPCKTCTTPAQGNSMYPSCCCTKPSDGNCTCIPIPSS
WAFAKYLWEWASARFSW
(AB486012 (J) HBsAg Antigenic Loop Sequence)
SEQ ID NO: 24
QGMLPVCPLLPGSTTTSTGPCRTCTITAQGTSMFPSCCCTKPSDGNCTCIPIPSSW
AFAKFLWEWASVRFSW
(HBsAg Y100C/P120T HBsAg Antigenic Loop Sequence)
SEQ ID NO: 25
CQGMLPVCPLIPGSSTTGTGTCRTCTTPAQGTSMYPSCCCTKPSDGNCTCIPIPSS
WAFGFLWEWASARFSW
(HBsAg P120T HBsAg Antigenic Loop Sequence)
SEQ ID NO: 26
QGMLPVCPLIPGSSTTGTGTCRTCTTPAQGTSMYPSCCCTKPSDGNCTCIPIPSS
WAFGKFLWEWASARFSW
(HBsAg P120T/S143L HBsAg Antigenic Loop Sequence)
SEQ ID NO: 27
QGMLPVCPLIPGSSTTGTGTCRTCTTPAQGTSMYPSCCCTKPLDGNCTCIPIPSS
WAFGKFLWEWASARFSW
(HBsAg C121 S HBsAg Antigenic Loop Sequence)
SEQ ID NO: 28
QGMLPVCPLIPGSSTTGTGPSRTCTTPAQGTSMYPSCCCTKPSDGNCTCIPIPSSW
AFGKFLWEWASARFSW
(HBsAg R122D HBsAg Antigenic Loop Sequence)
SEQ ID NO: 29
QGMLPVCPLIPGSSTTGTGPCDTCTTPAQGTSMYPSCCCTKPSDGNCTCIPI
PSSWAFGKFLWEWASARFSW
(HBsAg R122I HBsAg Antigenic Loop Sequence)
SEQ ID NO: 30
QGMLPVCPLIPGSSTTGTGPCITCTTPAQGTSMYPSCCCTPSDGNCTCIPIPSSWA
FGKFLWEWASARFSW
(HBsAg T123N HBsAg Antigenic Loop Sequence)
SEQ ID NO: 31
QGMLPVCPLIPGSSTTGTGPCRNCTTPAQGTSMYPSCCCTKPSDGNCTCIPIPSS
WAFGKFLWEWASARFSW
(HBsAg Q129H HBsAg Antigenic Loop Sequence)
SEQ ID NO: 32
QGMLPVCPLIPGSSTTGTGPCRTCTTPAHGTSMYPSCCCTKPSDG NCTCI PI
PSSWAFGKFLWEWASARFSW
(HBsAg Q129L HBsAg Antigenic Loop Sequence)
SEQ ID NO: 33
QGMLPVCPLIPGSSTTGTGPCRTCTTPALGTSMYPSCCCTKPSDGNCTCIPIPSSW
AFGKFLWEWASARFSW
(HBsAg M1 33H HBsAg Antigenic Loop Sequence)
SEQ ID NO: 34
QGMLPVCPLIPGSSTTGTGPCRTCTTPAQGTSHYPSCCCTKPSDGNCTCIPIPSSW
AFGKFLWEWASARFSW
(HBsAg M1 33L HBsAg Antigenic Loop Sequence)
SEQ ID NO: 35
QGMLPVCPLIPGSSTTGTGPCRTCTTPAQGTSLYPSCCCTKPSDGNCTCIPIPSSW
AFGKFLWEWASARFSW
(HBsAg M133T HBsAg Antigenic Loop Sequence)
SEQ ID NO: 36
QGMLPVCPLIPGSSTTGTGPCRTCTTPAQGTSTYPSCCCTKPSDGNCTCIPIPSSW
AFGKFLWEWASARFSW
(HBsAg K141 E HBsAg Antigenic Loop Sequence)
SEQ ID NO: 37
QGMLPVCPLIPGSSTTGTGPCRTCTTPAQGTSMYPSCCCTEPSDGNCTCIPIPSSW
AFGKFLWEWASARFSW
(HBsAg P142S HBsAg Antigenic Loop Sequence)
SEQ ID NO: 38
QGMLPVCPLIPGSSTTGTGPCRTCTTPAQGTSMYPSCCCTKSSDGNCTCIPIPSS
WAFGKFLWEWASARFSW
(HBsAg S143K HBsAg Antigenic Loop Sequence)
SEQ ID NO: 39
QGMLPVCPLIPGSSTTGTGPCRTCTTPAQGTSMYPSCCCTKPKDGNCTCIPIPSS
WAFGKFLWEWASARFSW
(HBsAg D144A HBsAg Antigenic Loop Sequence)
SEQ ID NO: 40
QGMLPVCPLIPGSSTTGTGPCRTCTTPAQGTSMYPSCCCTPSAGNCTCIPIPSSW
AFGKFLWEWASARFSW
(HBsAg G145R HBsAg Antigenic Loop Sequence)
SEQ ID NO: 41
QGMLPVCPLIPGSSTTGTGPCRTCTTPAQGTSMYPSCCCTKPSDRNCTCIPIPSS
WAFGKFLWEWASARFSW
(HBsAg N146A HBsAg Antigenic Loop Sequence)
SEQ ID NO: 42
QGMLPVCPLIPGSSTTGTGPCRTCTTPAQGTSMYPSCCCTKPSDGACTCIPIPSS
WAFGKFLWEWASARFSW
(HBsAg Genotype D S domain (Genbank accession no. FJ899792))
SEQ ID NO: 43
MENVTSGFLGPLLVLQAGFFLLTRILTIPQSLDSWWTSLNFLGGTTVCLGQNSQ
SPTSNHSPTSCPPTCPGYRWMCLRRFIIFLFILLLCLIFLLVLLDYQGMLPVCPLIP
GSSTTGTGPCRTCTTPAQGTSMYPSCCCTKPSDGNCTCIPIPSSWAFGKFLWEW
ASARFSWLSLLVPFVQWFVGLSPTVWLSVIWMMWYWGPSLYSTLSPFLPLLPIF
FCLWVYI
(HBC34 CDRH1)
SEQ ID NO: 44
GRIFRSFY
(HBC34 short CDRH2)
SEQ ID NO: 45
NQDGSEK
(HBC34 long CDRH2)
SEQ ID NO: 46
INQDGSEK
(HBC34 CDRH3)
SEQ ID NO: 47
AAWSGNSGGMDV
(HBC34 CDRL1)
SEQ ID NO: 48
KLGNKN
(HBC34 short CDRL2)
SEQ ID NO: 49
EVK
(HBC34 long CDRL2)
SEQ ID NO: 50
VIYEVKYRP
(HBC34 CDRL3)
SEQ ID NO: 51
QTWDSTTVV
(HBC34v35 CDRL3)
SEQ ID NO: 52
QTFDSTTVV
(HBC34 VH)
SEQ ID NO: 53
ELQLVESGGGWVQPGGSQRLSCAASGRIFRSFYMSWVRQAPGKGLEWVATIN
QDGSEKLYVDSVKGRFTISRDNAKNSLFLQMNNLRVEDTAVYYCAAWSGNSG
GMDVWGQGTTVSVSS
(HBC34 VL)
SEQ ID NO: 54
SYELTQPPSVSVSPGQTVSIPCSGDKLGNKNVCWFQHKPGQSPVLVIYEVKYRP
SGIPERFSGSNSGNTATLTISGTQAMDEAAYFCQTWDSTTVVFGGGTRLTVL
(HBC34v35 VL)
SEQ ID NO: 55
SYELTQPPSVSVSPGQTVSIPCSGDKLGNKNVAWFQHKPGQSPVLVIYEVKYRP
SGIPERFSGSNSGNTATLTISGTQAMDEAAYFCQTFDSTTVVFGGGTRLTVL
(HBC34v35 HC)
SEQ ID NO: 56
ELQLVESGGGWVQPGGSQRLSCAASGRIFRSFYMSWVRQAPGKGLEWVATIN
QDGSEKLYVDSVKGRFTISRDNAKNSLFLQMNNLRVEDTAVYYCAAWSGNSG
GMDVWGQGTTVSVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGN
VFSCSVMHEALHNHYTQKSLSLSPGK
(HBC34v35-MLNS-GAALIE (g1M17, 1) HC)
SEQ ID NO: 57
ELQLVESGGGWVQPGGSQRLSCAASGRIFRSFYMSWVRQAPGKGLEWVATIN
QDGSEKLYVDSVKGRFTISRDNAKNSLFLQMNNLRVEDTAVYYCAAWSGNSG
GMDVWGQGTTVSVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNT
KVDKKVEPKSCDKTHTCPPCPAPELLAGPSVFLFPPKPKDTLMISRTPEVTCVV
VDVSHEDPEVKFNWYVDGVEVHNAKTKPREEQYNSTYRVVSVLTVLHQDWL
NGKEYKCKVSNKALPLPEEKTISKAKGQPREPQVYTLPPSRDELTKNQVSLTCL
VKGFYPSDIAVEWESNGQPENNYKTTPPVLDSDGSFFLYSKLTVDKSRWQQGN
VFSCSVLHEALHSHYTQKSLSLSPGK
(HBC34 LC)
SEQ ID NO: 58
SYELTQPPSVSVSPGQTVSIPCSGDKLGNKNVCWFQHKPGQSPVLVIYEVKYRP
SGIPERFSGSNSGNTATLTISGTQAMDEAAYFCQTWDSTTVVFGGGTRLTVLGQ
PKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECS
(HBC34v35 LC)
SEQ ID NO: 59
SYELTQPPSVSVSPGQTVSIPCSGDKLGNKNVAWFQHKPGQSPVLVIYEVKYRP
SGIPERFSGSNSGNTATLTISGTQAMDEAAYFCQTFDSTTVVFGGGTRLTVLGQ
PKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECS
(WT hIgG1 Fc)
SEQ ID NO: 60
APELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSL
SPGK
(Synthetic sequence chimeric hinge)
SEQ ID NO: 61
ESKYGPPCPPCPAPPVAGP

That which is claimed is:

1. A method of treating hepatitis D virus (HDV) infection or an HDV-associated disease in a subject in need thereof, comprising administering to the subject:
(a) an anti-HBV antibody; and
(b) an siRNA that targets an HBV mRNA;
wherein the subject has a Child-Pugh-Turcotte (CPT) score of 5 or higher prior to treatment.

2. The method according to claim 1, wherein the HDV-associated disease is chronic hepatitis; acute hepatitis D; acute fulminant hepatitis D; chronic hepatitis D; liver fibrosis; end-stage liver disease; or hepatocellular carcinoma.

3. The method according to claim 1, wherein the subject has chronic hepatitis B virus (HBV)/HDV coinfection.

4. The method according to claim 1, wherein the subject has a positive serum HBsAg, HBV DNA, or HBeAg on 2 occasions at least 6 months apart prior to treatment.

5. The method according to claim 1, wherein the subject has tested positive for HDV antibody or HDV RNA prior to treatment.

6. The method according to claim 1, wherein the subject has tested positive for HDV antibody and HDV RNA prior to treatment.

7. The method according to claim 1, wherein the subject has tested positive for HDV antibody for at least 6 months prior to treatment.

8. The method according to claim 1, wherein the subject has a HDV RNA≥500 IU/mL prior to treatment.

9. The method according to claim 1, wherein the subject has a HBsAg level>0.05 IU/mL prior to treatment.

10. The method according to claim 1, wherein the subject has a HBsAg level>10,000 IU/mL prior to treatment.

11. The method according to claim 1, wherein the subject has chronic hepatitis.

12. The method according to claim 1, wherein the subject has an alanine aminotransferase (ALT) level>the upper limit of normal (ULN) and <5 times ULN prior to treatment.

13. The method according to claim 1, wherein the subject has an (ALT) level>the upper limit of normal (ULN) and an aspartate aminotransferase (AST) level>ULN prior to treatment.

14. The method according to claim 1, wherein the subject has an ALT level<5 times ULN and an AST level<5 times ULN prior to treatment.

15. The method according to claim 1, wherein the subject has a liver biopsy with METAVIR F4 or Liver elastography (Fibroscan®)≥12 kilopascal (kPa) within the 12 months prior to treatment.

16. The method according to claim 1, wherein the subject has a creatine clearance (CLcr)≥60 mL/min as calculated by the Cockcroft-Gault formula prior to treatment.

17. The method according to claim 1, wherein the subject has a Child-Pugh-Turcotte (CPT) score of 5 or 6 prior to treatment.

18. The method according to claim 1, wherein the subject has a Child-Pugh-Turcotte (CPT) score of 7 or higher prior to treatment.

19. The method according to claim 1, wherein the subject has a Child-Pugh-Turcotte (CPT) score of 7 to 9 prior to treatment.

20. The method according to claim 1, wherein the subject has a Child-Pugh-Turcotte (CPT) score of ≥10 prior to treatment.

21. The method according to claim 1, wherein the subject has not previously been administered an anti-HIV antibody or an siRNA that targets an HBV mRNA.

22. The method according to claim 1, wherein the anti-HBV antibody is a human antibody.

23. The method according to claim 1, wherein the antibody is HBC34 or a non-natural variant of HBC34.

24. The method according to claim 1, wherein the anti-HBV antibody comprises:
(i) CDRH1, CDRH2, and CDRH3 amino acid sequences according to SEQ ID NOs:44, 45 or 46, and 47, respectively; and
(ii) CDRL1, CDRL2, and CDRL3 amino acid sequences according to SEQ ID NOs:48, 49 or 50, and 52, respectively.

25. The method according to claim 24, wherein the anti-HBV antibody comprises:
(i) CDRH1, CDRH2, and CDRH3 amino acid sequences according to SEQ ID NOs:44, 45, and 47, respectively; and
(ii) CDRL1, CDRL2, and CDRL3 amino acid sequences according to SEQ ID NOs:48, 49, and 52, respectively.

26. The method according to claim 24, wherein the anti-HBV antibody comprises:
(i) CDRH1, CDRH2, and CDRH3 amino acid sequences according to SEQ ID NOs:44, 46, and 47, respectively; and
(ii) CDRL1, CDRL2, and CDRL3 amino acid sequences according to SEQ ID NOs:48, 50, and 52, respectively.

27. The method according to claim 1, wherein the anti-HBV antibody comprises:
(a) a light chain variable domain (V_{L}) that is at least 90%, at least 95%, or 100% identical to the amino acid sequence according to SEQ ID NO:55; and (b) a heavy chain variable domain (V_{H}) that is at least 90%, at least 95%, or 100% identical to the amino acid sequence according to SEQ ID NO:53.

28. The method according to claim 1, wherein the anti-HBV antibody comprises:
(a) a light chain that is at least 90%, at least 95%, or 100% identical to the amino acid sequence set forth in SEQ ID NO:59, and (b) a heavy chain that is at least 90%, at least 95%, or 100% identical to the amino acid sequence according to SEQ ID NO:57.

29. The method according to claim 1, wherein the siRNA comprises a sense strand and an antisense strand forming a double-stranded region, wherein the sense strand comprises at least 15 contiguous nucleotides differing by no more than 3 nucleotides from nucleotides 1579-1597 of SEQ ID NO:1 wherein T is replaced with U.

30. The method according to claim 1, wherein the antisense strand of the siRNA comprises or consists of the nucleotide sequence of 5′-UGUGAAGCGAAGUGCACACUU-3′ (SEQ ID NO:4).

31. The method according to claim 30, wherein the sense strand of the siRNA comprises or consists of the nucleotide sequence of 5′-GUGUGCACUUCGCUUCACA-3′ (SEQ ID NO:3).

32. The method according to claim 31, wherein at least one strand of the siRNA comprises a 3′ overhang of at least 1 nucleotide.

33. The method according to claim 31, wherein the double-stranded region of the siRNA is 15-30 nucleotide pairs in length.

34. The method according to claim 31, wherein each strand of the RNAi agent has 15-30 nucleotides.

35. The method according to claim 31, wherein substantially all of the nucleotides of the sense strand of the siRNA and substantially all of the nucleotides of the antisense strand of the siRNA are modified nucleotides, and
wherein the sense strand is conjugated to a ligand attached at the 3′-terminus.

36. The method according to claim 35, wherein the ligand is one or more GalNAc derivatives attached through a monovalent linker, bivalent branched linker, or trivalent branched linker.

37. The method according to claim 36, wherein the ligand is

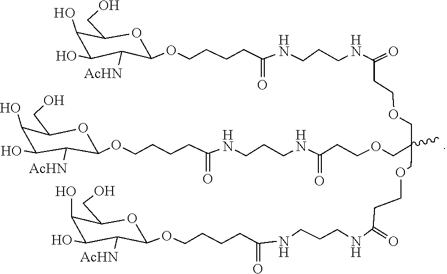 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

38. The method according to claim 37, wherein the siRNA is conjugated to the ligand as shown in the following structure: 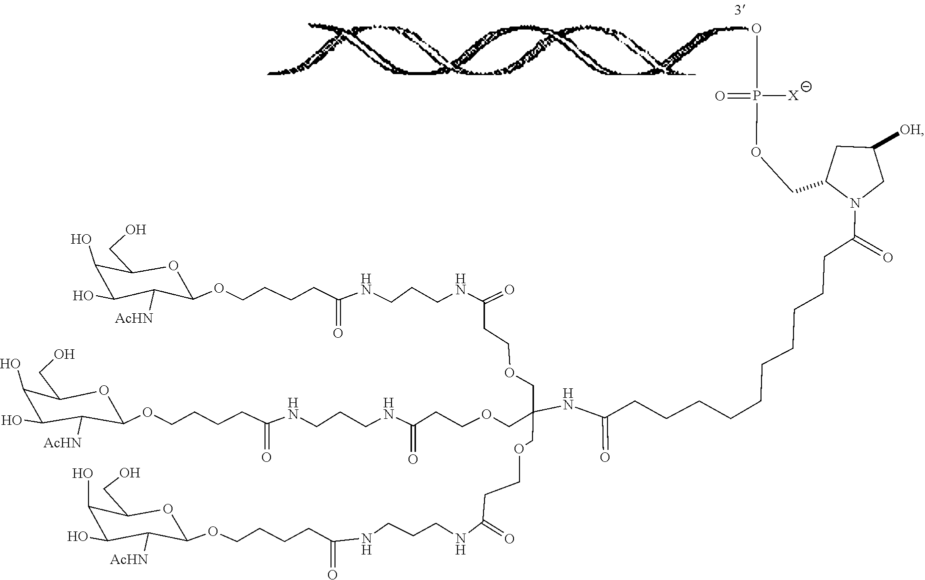
wherein X is O or S.

39. The method according to claim 38, wherein X is O.

40. The method according to claim 31, wherein at least one nucleotide of the siRNA is a modified nucleotide comprising a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, an adenosine-glycol nucleic acid, or a nucleotide comprising a 5′-phosphate mimic.

41. The method according to claim 31, wherein the siRNA comprises a phosphate backbone modification, a 2′ ribose modification, 5′ triphosphate modification, or a GalNAc conjugation modification.

42. The method according to claim 31, wherein all of the nucleotides of the sense strand of the siRNA and all of the nucleotides of the antisense strand are modified nucleotides.

43. The method according to claim 1, wherein the siRNA comprises a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6)
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
(Agn) is adenosine-glycol nucleic acid (GNA);
s is a phosphorothioate linkage; and
L96 is N-[tris(GalNAc-alkyl)-amidodecanoyl)]-4-hydroxyprolinol.

44. The method according to claim 43, wherein the L96 is conjugated to the sense strand as shown in the following structure: 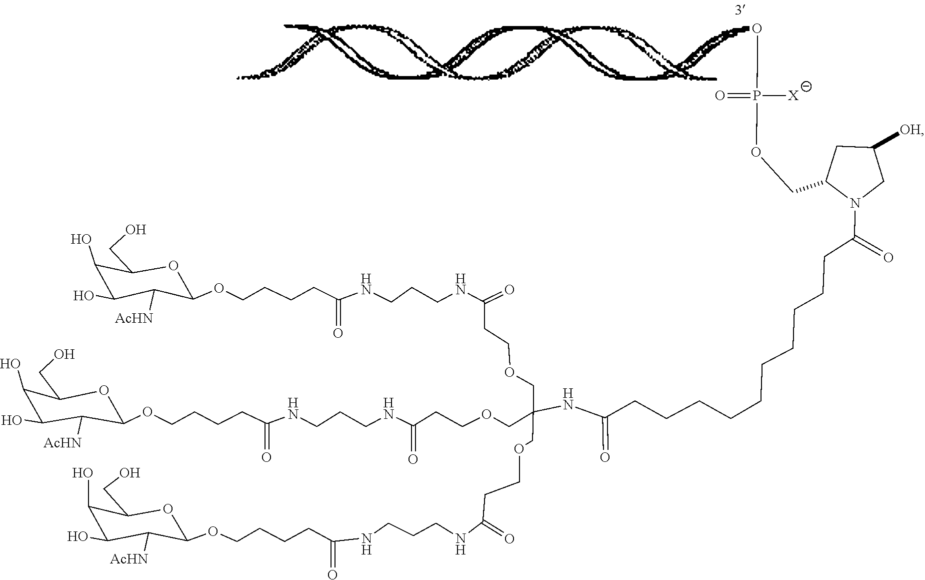
wherein X is O.

45. The method according to claim 1, further comprising administering to the subject a nucleos(t)ide reverse transcriptase inhibitor (NRTI).

46. The method according to claim 1, wherein the subject has previously been administered an NRTI.

47. The method according to claim 45, wherein the NRTI is tenofovir, tenofovir disoproxil fumarate (TDF), tenofovir disoproxil (TD), tenofovir alafenamide (TAF), lamivudine, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, and ganciclovir, besifovir (ANA-380/LB-80380), or tenofvir-exaliades (TLX/CMX157).

48. The method according to claim 45, wherein the NRTI is tenofovir, tenofovir disoproxil fumarate (TDF), or tenofovir disoproxil (TD).

49. The method according to claim 1, wherein:
(a) the anti-HBV antibody comprises or consists of: a light chain amino acid sequence according to SEQ ID NO: 59, and a heavy chain amino acid sequence according to SEQ ID NO:57; and
(b) the siRNA comprises or consists of: a sense strand comprising or consisting of 5′-gsusguGfcAfCfUfucgcuucacaL96-3′ (SEQ ID NO:5) and an antisense strand comprising or consisting of 5′-usGfsuga(Agn)gCfGfaaguGfcAfcacsusu-3′ (SEQ ID NO:6),
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
(Agn) is adenosine-glycol nucleic acid (GNA);
s is a phosphorothioate linkage; and
L96 is N-[tris(GalNAc-alkyl)-amidodecanoyl)]-4-hydroxyprolinol.

50. The method according to claim 45, wherein:
(a) the anti-HBV antibody comprises or consists of: a light chain amino acid sequence according to SEQ ID NO:59, and a heavy chain amino acid sequence according to SEQ ID NO:57;
(b) the siRNA comprises or consists of: a sense strand comprising or consisting of 5′-gsusguGfcAfCfUfucgcuucacaL96-3′ (SEQ ID NO:5) and an antisense strand comprising or consisting of 5′-usGfsuga(Agn)gCfGfaaguGfcAfcacsusu-3′ (SEQ ID NO:6),
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
(Agn) is adenosine-glycol nucleic acid (GNA);
s is a phosphorothioate linkage; and
L96 is N-[tris(GalNAc-alkyl)-amidodecanoyl)]-4-hydroxyprolinol; and
(c) the NRTI is tenofovir disoproxil fumarate (TDF) or entecavir.

51. The method according to claim 1, wherein the anti-HBV antibody is administered every 2 weeks, every 4 weeks, or every 8 weeks.

52. The method according to claim 1, wherein the siRNA is administered every 4 weeks or every 8 weeks.

53. The method according to claim 45, wherein the NRTI is administered daily.

54. The method according to claim 1, wherein the anti-HBV antibody is administered at a dose of 300 mg.

55. The method according to claim 1, wherein the siRNA is administered at a dose of 200 mg.

56. The method according to claim 45, wherein the NRTI is administered at a dose of 300 mg.

57. The method according to claim 45, wherein the NRTI is administered at a dose of 245 mg.

58. The method according to claim 1, wherein the subject is administered the siRNA and the anti-HBV antibody beginning on the same day.

59. The method according to claim 1, wherein the subject is administered the siRNA and the anti-HBV antibody for up to 96 weeks, 96 weeks, at least 96 weeks, or 96 weeks or longer.

60. The method according to claim 1, wherein the subject is a human.

61. An anti-HBV antibody; and an siRNA that targets an HBV mRNA; for use in the method according to any one of claims 1-60.

62. An anti-HBV antibody for use in the method according to any one of claims 1-60; wherein the subject is also administered an siRNA that targets an HBV mRNA.

63. Use of an anti-HBV antibody; and an siRNA that targets an HBV mRNA; in the manufacture of a medicament for use in the method according to any one of claims 1-60.

64. Use of an anti-HBV antibody in the manufacture of a first medicament; and use of an siRNA that targets an HBV mRNA in the manufacture of a second medicament; wherein the first and second medicaments are to be used in a combination therapy according to the method of any one of claims 1-60.

65. An anti-HBV antibody; an siRNA that targets an HBV mRNA; and a NRTI; for use in the method according to any one of claims 45-60.

66. Use of an anti-HBV antibody; an siRNA that targets an HBV mRNA; and a NRTI; in the manufacture of a medicament for use in the method according to any one of claims 45-60.

67. Use of an anti-HBV antibody in the manufacture of a first medicament; use of an siRNA that targets an HBV mRNA in the manufacture of a second medicament; and use of a NRTI in the manufacture of third medicament; wherein the first, second, and third medicaments are to be used in a combination therapy according to the method of any one of claims 45-60.

68. A kit comprising:
a pharmaceutical composition comprising an anti-HBV antibody, and a pharmaceutically acceptable excipient; and
a pharmaceutical composition comprising an siRNA that targets an HBV mRNA, and a pharmaceutically acceptable excipient.

69. A kit comprising:
a pharmaceutical composition comprising an anti-HBV antibody, and a pharmaceutically acceptable excipient;
a pharmaceutical composition comprising an siRNA that targets an HBV mRNA, and a pharmaceutically acceptable excipient; and
a pharmaceutical composition comprising a NRTI, and a pharmaceutically acceptable excipient.
